(19) United States
(12) Patent Application Publication
(10) Pub. No.: US 2010/0028913 A1
(43) Pub. Date: Feb. 4, 2010

(54) NOVEL CARBOHYDRATE FROM HUMAN CELLS AND METHODS FOR ANALYSIS AND MODIFICATION THEREOF

(75) Inventors: Jarmo Laine, Helsinki (FI); Tero Satomaa, Helsinki (FI); Jari Natunen, Vantaa (FI); Annamari Heiskanen, Helsinki (FI); Maria Blomqvist, Itasalmi (FI); Anne Olonen, Lahti (FI); Juhani Saarinen, Helsinki (FI); Sari Titinen, Vantaa (FI); Ulla Impola, Helsinki (FI); Olli Aitio, Helsinki (FI); Leena Valmu, Helsinki (FI); Heidi Anderson, Helsinki (FI); Virve Pitkänen, Helsinki (FI); Jukka Partanen, Helsinki (FI); Taina Jaatinen, Helsinki (FI)

Correspondence Address: BIRCH STEWART KOLASCH & BIRCH, PO BOX 747, FALLS CHURCH, VA, 22040-0747, US

(73) Assignees: SUOMEN PUNAINEN RISTI, VERIPALVELU, Helsink (FI); Glykos Finland Ltd., Helsinki (FI)

(21) Appl. No.: 12/523,586

(22) PCT Filed: Jan. 18, 2008
(86) PCT No.: PCT/FI2008/050017

§ 371 Date: Sep. 22, 2009

(30) Foreign Application Priority Data
Jan. 18, 2007 (FI) 20075033
Mar. 13, 2007 (FI) 20070205

(51) Int. Cl. G01N 33/569; G01N 33/53; G01N 33/577

(52) U.S. Cl. 435/7.21

(57) ABSTRACT

The invention describes reagents and methods for specific binders to glycan structures of stem cells. Furthermore the invention is directed to screening of additional binding reagents against specific glycan epitopes on the surfaces of the stem cells. The preferred binders of the glycans structures includes proteins such as enzymes, lectins and antibodies.

The invention revealed novel characteristic glycans useful for analysis of various human cell populations. The invention is directed to various methods for analysis of the cells based on the presence of the characteristic glycans.

### FIELD OF THE INVENTION

The invention describes reagents and methods for specific binders to glycan structures of stem cells. Furthermore the invention is directed to screening of additional binding reagents against specific glycan epitopes on the surfaces of the stem cells. The preferred binders of the glycans structures includes proteins such as enzymes, lectins and antibodies.

The invention describes novel compositions of glycans, glycomes, from stem cells in blood, especially cord blood (CB) derived stem cells, (most preferably CD133+ cells) and especially novel subcompositions of the glycomes with specific monosaccharide compositions and glycan structures. The invention is further directed to methods for modifying the glycomes and analysis of the glycomes and the modified glycomes. Furthermore, the invention is directed to stem cells carrying the modified glycomes on their surfaces. The glycomes are preferably analysed by profiling methods able to detect reproducibly and quantitatively numerous individual glycan structures at the same time. The most preferred type of the profile is a mass spectrometric profile. The invention specifically revealed novel target structures and is especially directed to the development of reagents recognizing the structures.

### BACKGROUND OF THE INVENTION

### Stem Cells

Stem cells are undifferentiated cells which can give rise to a succession of mature functional cells. For example, a hematopoietic stem cell may give rise to any of the different types of terminally differentiated blood cells. Embryonic stem (ES) cells are derived from the embryo and are pluripotent, thus possessing the capability of developing into any organ or tissue type or, at least potentially, into a complete embryo.

The first evidence for the existence of stem cells came from studies of embryonic carcinoma (EC) cells, the undifferentiated stem cells of teratocarcinomas, which are tumors derived from germ cells. These cells were found to be pluripotent and immortal, but possess limited developmental potential and abnormal karyotypes (Rossant and Papaioannou, Cell Differ 15,155-161, 1984). ES cells, on the other hand, are thought to retain greater developmental potential because they are derived from normal embryonic cells, without the selective pressures of the teratocarcinoma environment.

Pluripotent embryonic stem cells have traditionally been derived principally from two embryonic sources. One type can be isolated in culture from cells of the inner cell mass of a pre-implantation embryo and are termed embryonic stem (ES) cells (Evans and Kaufman, Nature 292,154-156, 1981; U.S. Pat. No. 6,200,806). A second type of pluripotent stem cell can be isolated from primordial germ cells (PGCS) in the mesenteric or genital ridges of embryos and has been termed embryonic germ cell (EG) (U.S. Pat. No. 5,453,357, U.S. Pat. No. 6,245,566). Both human ES and EG cells are pluripotent. This has been shown by differentiating cells in vitro and by injecting human cells into immunocompromised (SCUM) mice and analyzing resulting teratomas (U.S. Pat. No. 6,200,806). The term “stem cell” as used herein means stem cells including embryonic stem cells or embryonic type stem cells and stem cells differentiated thereof to more tissue specific stem cells, adults stem cells including mesenchymal stem cells and blood stem cells such as stem cells obtained from bone marrow or cord blood.

The present invention provides novel markers and target structures and binders to these for especially embryonic and adult stem cells, when these cells are not hematopoietic stem cells. From hematopoietic CD34+ cells certain terminal structures such as terminal sialylated type two N-acetyllactosamines such as NeuNAcα3Galβ4GlcNAc (Magnani J. U.S. Pat. No. 6,362,010) has been suggested and there is indications for low expression of Slex type structures NeuNAcα3Galβ4(Fucα3)GlcNAc (Xia L et al Blood (2004) 104 (10) 3091-6). The invention is also directed to the NeuNAcα3Galβ4GlcNAc non-polylactosamine variants separately from specific characteristic O-glycans and N-glycans. The invention further provides novel markers for CD133+ cells and novel hematopoietic stem cell markers according to the invention, especially when the structures does not include NeuNAcα3Galβ4(Fucα3)₀₋₁GlcNAc. Preferably the hematopoietic stem cell structures are non-sialylated, fucosylated structures Galβ1-3-structures according to the invention and even more preferably type 1 N-acetyllactosamine structures Galβ3GlcNAc or separately preferred Galβ3GalNAc based structures.

Human ES, EG and EC cells, as well as primate ES cells, express alkaline phosphatase, the stage-specific embryonic antigens SSEA-3 and SSEA-4, and surface proteoglycans that are recognized by the TRA-1-60; and TRA-1-81 antibodies. All these markers typically stain these cells, but are not entirely specific to stem cells, and thus cannot be used to isolate stem cells from organs or peripheral blood.

The SSEA-3 and SSEA-4 structures are known as galactosylgloboside and sialylgalactosylgloboside, which are among the few suggested structures on embryonal stem cells, though the nature of the structures in not ambiguous. An antibody called K21 has been suggested to bind a sulfated polysaccharide on embryonal carcinoma cells (Badcock G et al Cancer Res (1999) 4715-19. Due to cell type, species, tissue and other specificity aspects of glycosylation (Furukawa, K., and Kobata, A. (1992) Curr. Opin. Struct. Biol. 3, 554-559, Gagneux, and Varki, A. (1999) Glycobiology 9, 747-755; Gawlitzek, M. et al. (1995), J. Biotechnol. 42, 117-131; Goelz, S., Kumar, R., Potvin, B., Sundaram, S., Brickelmaier, M., and Stanley, P. (1994) J. Biol. Chem. 269, 1033-1040; Kobata, A (1992) Eur. J. Biochem. 209 (2) 483-501.) This result does not indicate the presence of the structure on native embryonal stem cells. The present invention is directed to human stem cells.

It appears that skilled artisan would consider the results of Venable et al such convenient colocalization of SSEA-4 and the lectin binding by binding of the lectins to the anti-SSEA-4 antibody. It appears that the more rare binding would reflect lower proportion of the terminal epitope per antibody molecule leading to lower density of the labellable antibodies. It is also realized that the non-controlled cell culture process with animal derived material would lead to contamination of the cells by N-glycolyl-neuraminic acid, which may be recognized by anti-mouse antibodies used as secondary antibody (not defined what kind of anti-mouse) used in purification and analysis of purity, which could lead to conveniently high cell purity.

The work is directed only to the “pluripotent” embryonal stem cells associated with SSEA-4 labelling and not to differentiated variants thereof as the present invention. The results indicated possible binding (likely on the antibodies) to certain potential monosaccharide epitopes (6^{th} page, Table 10, and column 2) such Gal and Galactosamine for RCA (ricin, inhabitable by Gal or lactose), GlcNAc for TL (tomato lectin), Man or Glc for ConA, Sialic acid/Sialic acid α6GalNAc for SNA, Manα for HHL; lectins with partial binding not correlating with SSEA-4: GalNAc/GalNAcβ4Gal (in text) WFA, Gal for PNA, and Sialic acid/Sialic acid α6GalNAc for SNA; and lectins associated by part of SSEA-4 cells were indicated to bind Gal by PHA-L and PHA-E, GalNAc by VVA and Fuc by UEA, and Gal by MAA (inhibited by lactose). UEA binding was discussed with reference as endothelial marker and O-linked fucose which is directly bound to Ser (Thr) on protein. The background has indicated a H type 2 specificity for the endothelial UEA receptor. The specifities of the lectins are somewhat unusual, but the product codes or isolectin numbers/names of the lectins were not indicated (except for PHA-E and PHA-L) and it is known that plants contain numerous isolectins with varying specificities.

The present invention revealed specific structures by mass spectrometric profiling, NMR spectrometry and binding reagents including glycan modifying enzymes. The lectins are in general low specificity molecules. The present invention revealed binding epitopes larger than the previously described monosaccharide epitopes. The larger epitopes allowed us to design more specific binding substances with typical binding specificities of at least disaccharides. The invention also revealed lectin reagents with specified with useful specificities for analysis of native embryonal stem cells without selection against an uncontrolled marker and/or coating with an antibody or two from different species. Clearly the binding to native embryonal stem cells is different as the binding with MAA was clear to most of cells, there was differences between cell line so that RCA, LTA and UEA was clearly binding a HESC cell line but not another.

Methods for separation and use of stem cells are known in the art.

Characterizations and isolation of hematopoietic stem cells are reported in U.S. Pat. No. 5,061,620. The hematopoietic CD34 marker is the most common marker known to identify specifically blood stem cells, and CD34 antibodies are used to isolate stem cells from blood for transplantation purposes. U.S. Pat. No. 5,677,136 discloses a method for obtaining human hematopoietic stem cells by enrichment for stem cells using an antibody which is specific for the CD59 stem cell marker. The CD59 epitope is highly accessible on stem cells and less accessible or absent on mature cells. U.S. Pat. No. 6,127,135 provides an antibody specific for a unique cell marker (EM10) that is expressed on stem cells, and methods of determining hematopoietic stem cell content in a sample of hematopoietic cells

There have been great efforts toward isolating pluripotent or multipotent stem cells, in earlier differentiation stages than hematopoietic stem cells, in substantially pure or pure form for diagnosis, replacement treatment and gene therapy purposes. Stem cells are important targets for gene therapy, where the inserted genes are intended to promote the health of the individual into whom the stem cells are transplanted. In addition, the ability to isolate stem cells may serve in the treatment of lymphomas and leukemias, as well as other neoplastic conditions where the stem cells are purified from tumor cells in the bone marrow or peripheral blood, and reinfused into a patient after myelosuppressive or myeloablative chemotherapy.

The possibility of recovering fetal cells from the maternal circulation has generated interest as a possible means, non-invasive to the fetus, of diagnosing fetal anomalies (Simpson and Elias, J. Am. Med. Assoc. 270, 2357-2361, 1993). Prenatal diagnosis is carried out widely in hospitals throughout the world. Existing procedures such as fetal, hepatic or chorionic biopsy for diagnosis of chromosomal disorders including Down's syndrome, as well as single gene defects including cystic fibrosis are very invasive and carry a considerable risk to the fetus. Amniocentesis, for example, involves a needle being inserted into the womb to collect cells from the embryonic tissue or amniotic fluid. The test, which can detect Down's syndrome and other chromosomal abnormalities, carries a miscarriage risk estimated at 1%. Fetal therapy is in its very early stages and the possibility of early tests for a wide range of disorders would undoubtedly greatly increase the pace of research in this area. Thus, relatively non-invasive methods of prenatal diagnosis are an attractive alternative to the very invasive existing procedures. A method based on maternal blood should make earlier and easier diagnosis more widely available in the first trimester, increasing options to parents and obstetricians and allowing for the eventual development of specific fetal therapy.

The present invention provides methods of identifying, characterizing and separating stem cells having characteristics of embryonic stem (ES) cells for diagnostic, therapy and tissue engineering. In particular, the present invention provides methods of identifying, selecting and separating embryonic stem cells or fetal cells from maternal blood and to reagents for use in prenatal diagnosis and tissue engineering methods. The present invention provides for the first time a specific marker/binder/binding agent that can be used for identification, separation and characterization of valuable stem cells from tissues and organs, overcoming the ethical and logistical difficulties in the currently available methods for obtaining embryonic stem cells.

The present invention overcomes the limitations of known binders/markers for identification and separation of embryonic or fetal stem cells by disclosing a very specific type of marker/binder, which does not react with differentiated somatic maternal cell types. In other aspect of the invention, a specific binder/marker/binding agent is provided which does not react, i.e. is not expressed on feeder cells, thus enabling positive selection of feeder cells and negative selection of stem cells.

By way of exemplification, the binder to Formula (I) are now disclosed as useful for identifying, selecting and isolating pluripotent or multipotent hematopoietic stem cells including blood derived stem cells, which have the capability of differentiating into varied cell lineages.

According to one aspect of the present invention a novel method for identifying pluripotent or multipotent hematopoietic stem cells in peripheral blood and other organs is disclosed. According to this aspect a hematopoietic stem cell binder/marker is selected based on its selective expression in stem cells and its absence in differentiated somatic cells and/or feeder/associated cells. Thus, glycan structures expressed in stem cells are used according to the present invention as selective binders/markers for isolation of pluripotent or multipotent hematopoietic stem cells from blood, tissue and organs. Preferably the blood cells and tissue samples are of mammalian origin, more preferably human origin.

According to a specific embodiment the present invention provides a method for identifying a selective hematopoietic stem cell binder/marker comprising the steps of:

A method for identifying a selective stem cell binder to a glycan structure of Formula (I) which comprises:

i. selecting a glycan structure exhibiting specific expression in/on stem cells and absence of expression in/on feeder cells and/or differentiated somatic cells; ii. and confirming the binding of binder to the glycan structure in/on stem cells.

By way of a non-limiting example, adult, mesenchymal, embryonal type, or hematopoietic stem cells selected using the binder may be used in regenerating the hematopoietic or other tissue system of a host deficient in any class of stem cells. A host that is diseased can be treated by removal of bone marrow, isolation of stem cells and treatment with drugs or irradiation prior to re-engraftment of stem cells. The novel markers of the present invention may be used for identifying and isolating various stem cells; detecting and evaluating growth factors relevant to stem cell self-regeneration; the development of stem cell lineages; and assaying for factors associated with stem cell development.

### BRIEF DESCRIPTION OF THE DRAWINGS

FIG. 1. The major N-glycan structures in cord blood-derived leucocytes obtained by proton NMR spectroscopy. A.) High-mannose type N-glycans were the most abundant structures in the neutral N-glycan fraction. B.) Biantennary complex-type N-glycans are the most abundant structures of the sialylated N-glycans. Monosaccharide symbols: N-acetylhexosamines (N): ▪, N-acetyl-D-glucosamine, GlcNAc; Hexoses (H): ◯, D-mannose, Man; [symbol] , D-galactose, Gal; , D-glucose, Glc; And deoxyhexoses (F): Δ, L-fucose, Fuc. Sialic acids (S): ♦, N-acetylneuraminic acid, Neu5Ac; and sulphate or phosphate esters (P). Glycosidic linkages are indicated by lines connecting the monosaccharides.

FIG. 2. Mass spectrometric profiling analysis of neutral N-glycans. A.) Positive-ion MALDI-TOF mass spectrum of CD133+ neutral N-glycan fraction, wherein major glycan signals arise from [M+Na]⁺ sodium adduct ions. B.) Comparison of processed neutral N-glycan profiles of CD133+ and CD133− cells, wherein relative abundance of each glycan signal is expressed as % of total profile, allowing direct comparison between cell types. Known interfering signals, adduct ion signals, and effect of isotope pattern overlapping present in the original mass spectra have been removed (see Materials and methods). Each glycan signal has been assigned a proposed monosaccharide composition based on the m/z of the detected ion. C.) Rearrangement analysis of the profile data based on biosynthetic classification rules for the amounts of H and N residues in the proposed monosaccharide compositions, as indicated in the figure. Within each proposed biosynthetic class, glycan signals are arranged in the order of relative abundance in CD133+ cells. Relative abundances of the proposed glycan structure groups are indicated as % of total profile. Monosaccharide symbols as in FIG. 1. Abbreviations: F; fucose, H; Hexose and N; N-acetyllhexoamine.

FIG. 3. Mass spectrometric profiling analysis of sialylated N-glycans. A.) Negative-ion MALDI-TOF mass spectrum of CD133+ acidic N-glycan fraction, wherein major glycan signals arise from [M-H]⁻ deprotonated ions. Asterisks mark known contaminating polyhexose series that has been removed from B and C. B.) Comparison of sialylated N-glycan profiles of CD133+ and CD133− cells. C.) rearrangement analysis of the profile data, performed similarly as in FIG. 2. Further monosaccharide composition features associated with either CD133+ or CD133− cells (Hex5HexNAc3 and Hex6HexNAc3) are treated as additional glycan signal structural groups and their interpretation is indicated. Monosaccharide symbols as in FIG. 1. Abbreviations: F; fucose, H; Hexose, N; N-acetyllhexoamine and S; sialic acid.

FIG. 4. Exoglycosidase digestion with α2,3-sialidase in sialylated CD133+ and CD133− cell N-glycans. Sialylated N-glycan samples were treated α2,3-sialidase, and mass spectra were recorded before (dashed bars) and after the treatment (solid bars). The data was processed into normalized glycan profiles similarly as in FIGS. 2 and 3. For clarity, only the major sialylated N-glycan signals with H5N4 core composition are presented here. Change in the relative abundances of the glycans is indicated by arrows. The sum of monosialylated (S1) relative to the corresponding disialylated (S2) glycan species was increased in CD133+ cells, whereas in CD133− cells no similar profile change was observed. Abbreviations: F; fucose, H; Hexose, N; N-acetyllhexoamine and S; sialic acid.

FIG. 5. Schematic representation of N-linked glycan structures according to their biosynthetic entities. N-linked glycans consist of distinct regions of N-glycan core, backbone and terminal epitopes that are synthesized by different glycosyltransferase and glycosidase families. The gene families encoding these enzymes analyzed in the present study are given in brackets. Monosaccharide symbols and schematic N-glycan structures are as presented in the legend of FIG. 1.

FIG. 6. Schematic representation of favored N-glycan structures in CD133+ cells. Favored N-glycan structures in CD133+ cells are shown in dark background. Overexpressed and underexpressed genes are marked with black arrows upwards and downwards to show the difference in gene expression compared to CD133− cells. A. N-glycan core structures in CD133+ cells are polarized into both high-mannose type N-glycans and biantennary N-glycan structures, correlating with the differential expression of N-glycan processing enzymes. B. α2,3- and α2,6-sialyltransferases compete for the same N-glycan substrates. Overexpression of ST3GAL6 is accompanied with increased α2,3-sialylation in CD133+ cells. Monosaccharide symbols and schematic N-glycan structures are as presented in the legend of FIG. 1.

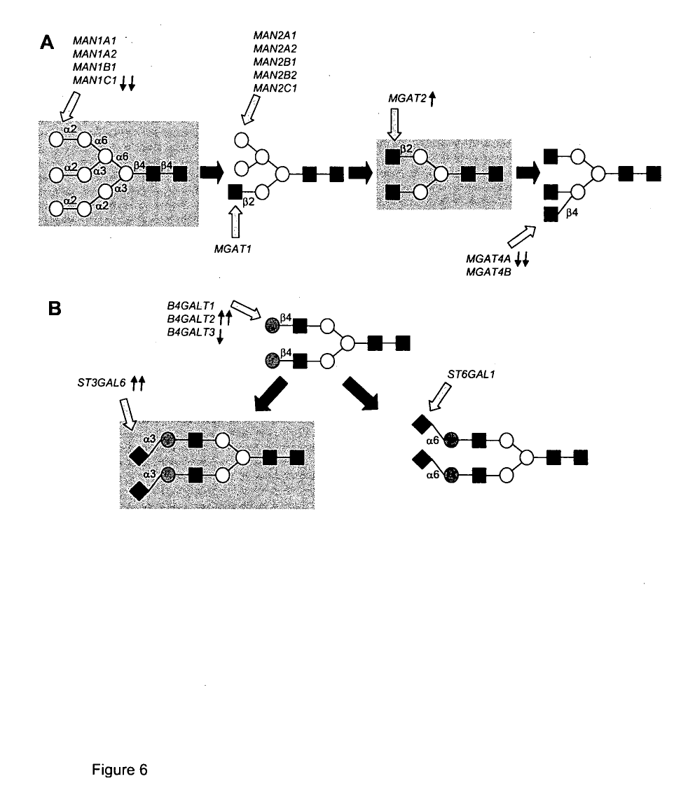

FIG. 7. Cord blood mononuclear cell sialylated N-glycan profiles before (light/blue columns) and after (dark/red columns) subsequent broad-range sialidase and α2,3-sialyltransferase reactions. The m/z values refer to Table 7.

FIG. 8. Cord blood mononuclear cell sialylated N-glycan profiles before (light/blue columns) and after (dark/red columns) subsequent α2,3-sialyltransferase and α1,3-fucosyltransferase reactions. The m/z values refer to Table 7.

FIG. 9. α2,3-sialidase analysis of sialylated N-glycans isolated from A. cord blood CD133⁺ cells and B. CD133⁻ cells. The columns represent the relative proportions of a monosialylated glycan signal at m/z 2076 (SA₁) and the corresponding disialylated glycan signal at m/z 2367 (SA₂), as described in the text. In cord blood CD133⁻ cells, the relative proportions of the SA₁ and SA₂ glycans do not change markedly upon α2,3-sialidase treatment (B), whereas in CD133+ cells the proportion of α2,3-sialidase resistant SA₂ glycans is significantly smaller than α2,3-sialidase resistant SA₁ glycans (A).

FIG. 10. Schematic view of preferred adult stem cells in bone marrow and blood, and cells which can be derived thereof, which are referred here also as blood derived stem cells.

FIG. 11. FACS analysis of seven cord blood mononuclear cell samples (parallel columns) by FITC-labelled lectins. The percentages refer to proportion of cells binding to lectin. For abbreviations of FITC-labelled lectins see text.

FIG. 12. MALDI-TOF mass spectrometric profile of isolated human stem cell neutral glycosphingolipid glycans. x-axis: approximate m/z values of [M+Na]⁺ ions as described in Table. y-axis: relative molar abundance of each glycan component in the profile. hESC, BMMSC, CB MSC, CB MNC: stem cell samples as described in the text.

FIG. 13. MALDI-TOF mass spectrometric profile of isolated human stem cell acidic glycosphingolipid glycans. x-axis: approximate m/z values of [M-H]⁻ ions as described in Table. y-axis: relative molar abundance of each glycan component in the profile. hESC, BMMSC, CB MSC, CB MNC: stem cell samples as described in the text.

FIG. 14. Lectin labeling of CB-MNC cells.

FIG. 15. FACS analysis of CB-MNC cells by specific binders.

FIG. 16. Cord blood mononuclear cells (CB MNC) selected and grown with beads coated by A) PNA lectin GF707 and B) LTA lectin GF 709.

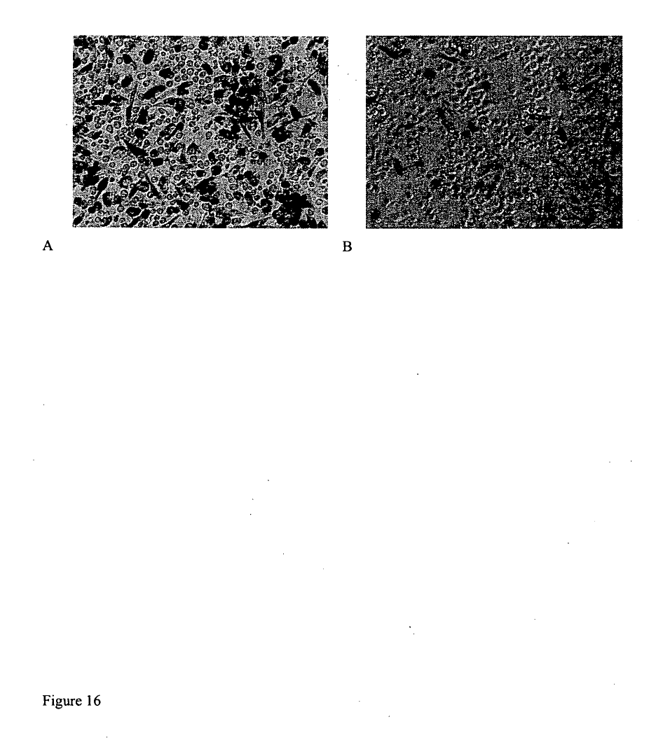

FIG. 17. A) Cord blood mononuclear cells and binder NPA GF711 on magnetic beads B) Selected lineage negative cells and magnetic beads coated with GF710.

### SUMMARY OF THE INVENTION

The present invention is directed to analysis of broad glycan mixtures from stem cell samples by specific binder (binding) molecules.

The present invention is specifically directed to glycomes of stem cells according to the invention comprising glycan material with monosaccharide composition for each of glycan mass components according to the Formula J:

R₁Hexβz{R₃})ₙ₁HexNAcXyR₂ (I),

wherein X is nothing or a glycosidically linked disaccharide epitope β4(Fucα6)ₙGN, wherein n is 0 or 1;

### Hex is Gal or Man or GlcA;

### HexNAc is GlcNAc or GalNAc;

y is anomeric linkage structure α and/or β or a linkage from a derivatized anomeric carbon,
z is linkage position 3 or 4, with the provision that when z is 4, then HexNAc is GlcNAc and Hex is Man or Hex is Gal or Hex is GlcA, and
when z is 3, then Hex is GlcA or Gal and HexNAc is GlcNAc or GalNAc;
R₁ indicates 1-4 natural type carbohydrate substituents linked to the core structures,
R₂ is reducing end hydroxyl, a chemical reducing end derivative or a natural asparagine linked N-glycoside derivative including asparagines, N-glycoside aminoacids and/or peptides derived from proteins, or a natural serine or threonine linked O-glycoside derivative including asparagines, N-glycoside aminoacids and/or peptides derived from proteins;
R3 is nothing or a branching structure representing GlcNAcβ6 or an oligosaccharide with GlcNAcβ6 at its reducing end linked to GalNAc, when HexNAc is GalNAc, or R3 is nothing or Fucα4, when Hex is Gal, HexNAc is GlcNAc, and z is 3, or R3 is nothing or Fucα3, when z is 4.

Typical glycomes comprise of subgroups of glycans, including N-glycans, O-glycans, glycolipid glycans, and neutral and acidic subglycomes.

The invention is directed to diagnosis of clinical state of stem cell samples, based on analysis of glycans present in the samples. The invention is especially directed to diagnosing cancer and the clinical state of cancer, preferentially to differentiation between stem cells and cancerous cells and detection of cancerous changes in stem cell lines and preparations.

The invention is further directed to structural analysis of glycan mixtures present in stem cell samples.

### DESCRIPTION OF THE INVENTION

Related data and specification was presented in PCT FJ 2006/050336

The present invention revealed novel stem cell specific glycans, with specific monosaccharide compositions and associated with differentiation status of stem cells and/or several types of stem cells and/or the differentiation levels of one stem cell type and/or lineage specific differences between stem cell lines.

N-Glycan Structures and Compositions Associated with Differentiation of Stem Cells

The invention revealed specific glycan monosaccharide compositions and corresponding structures, which associated with
i) Blood derived stem cells especially cord blood derived stem cells
ii) Differentiated mononuclear blood cells

The preferred blood stem cells are hematopoietic stem cells more preferably CD133 or CD34 positive stem cells, most preferably cord blood derived CD133 or CD34 positive stem cells.

Differentiated mononuclear blood cells are preferably CD133 or CD34 negative stem cells, most preferably cord blood derived CD133 or CD34 negative stem cells.

It is realized that the CD34+ cells resemble CD133+ cells, the invention also revealed that transferase expression of CD34+ cells was similar to the transferase expression of CD133+ cells.

The invention is in a preferred embodiment directed to the use of the preferred mRNA markers according to the invention for the analysis of CD34+ cells.

It is realized that the structures revealed are useful for the characterization of the cells at different stages of development. The invention is directed to the use of the structures as markers for differentiation of blood derived stem cells.

The invention is further directed to the use of the specific glycans as markers enriched or increased at specific level of differentiation for the analysis of the cells at specific differentiation level.

N-Glycan Structures and Compositions are Associated with Individual Specific Differences between Stem Cell Lines or Batches

The invention further revealed that specific glycan types are presented in the blood derived stem cell preparations on a specific differentiation stage in varying manner. It is realized that such individually varying glycans are useful for characterization of individual stem cell lines/preparations and batches. The specific structures of a individual cell preparation are useful for comparison and standardization of stem cell lines and cells prepared thereof.

The specific structures of a individual cell preparation are used for characterization of usefulness of specific stem cell line or batch or preparation for stem cell therapy in a patient, who may have antibodies or cell mediated immune defense recognizing the individually varying glycans.

The invention is especially directed to analysis of glycans with large and moderate variations as described in example 3. The invention is especially directed to the analysis of individual specific differences, when there is a difference in the level of fucosylation and/or sialylation or in the level of mannosylation.

### Analysis Methods by Mass Spectrometry or Specific Binding Reagents

The invention is specifically directed to the recognition of the terminal structures by either specific binder reagents and/or by mass spectrometric profiling of the glycan structures.

In a preferred embodiment the invention is directed to the recognition of the structures and/or compositions based on mass spectrometric signals corresponding to the structures.

The preferred binder reagents are directed to characteristic epitopes of the structures such as terminal epitopes and/or characteristic branching epitopes, such as monoantennary structures comprising a Manα-branch or not comprising a Manα-branch.

The preferred binder is an antibody, more preferably a monoclonal antibody.

In a preferred embodiment the invention is directed to a monoclonal antibody specifically recognizing at least one of the terminal epitope structures according to the invention.

Analysis of Glycosylation by mRNA Expression Related to N-Glycan Expression

The invention revealed that expression of certain glycosyltransferase mRNAs is related to or correlates with the expressed glycan structures. The invention is directed to the use of the expression mRNAs as shown in the Example 1, for the analysis of the glycosylation status hematopoietic stem cells on mRNA level.

The Preferred Glycosyltransferases for mRNA Analysis

The preferred enzymes for mRNA analysis includes groups of sialyltransferases, fucosyltransferases, galactosyltransferases, N-acetylglycosaminytransferases, and mannosidases involved in the synthesis of the preferred complex type N-glycans according to the invention.

### N-Acetylglycosaminytransferases

The preferred N-acetylglucosaminyltransferases to be analyzed in context of analysis of mRNA-level glycosylation analysis are shown in Table 1. Preferred N-acetylglucosaminyltransferases for mRNA analysis include MGAT2 and MGAT4. The biantennary type structures were increased on the CD133+ cells as shown in Example 1 and mRNA expression of the enzymes such as MGAT2 and MGAT4 was related to this.

### Mannosidases

The preferred mannosidases to be analyzed in context of analysis of mRNA-level glycosylation analysis are shown in Table 1. The most preferred altering mannosidase is Man1C1 for the characterization of the human blood derived stem cells, especially the cord blood cells. The mRNA of the α2-mannosidase (type I mannosidase) was absent in CD133+ cells, while present in the differentiated cells. The mannosidase expression reflects to the expression of large high-mannose N-glycans in the blood stem cells and lower size glycans in differentiated cells.

### Galactosyltransferases

The preferred galactosyltransferases, especially β4-galactosyltransferases β4GALT2 and β4GALT3, to be analyzed in context of analysis of mRNA-level glycosylation analysis are shown in Table 1. Terminal Galβ4GlcNAc structures were prominent on the CD133+ cells as shown in Example 1 and mRNA expression of the enzymes was related to this.

### Sialyltransferases

The preferred sialyltransferases, especially α3- and α6-sialyltransferases ST3GAL5 and ST6GAL1, to be analyzed in context of analysis of mRNA-level glycosylation analysis are shown in Table 1. The invention is further especially directed to the analysis of increased expression of ST3GAL6, which was observed to be associated with the blood stem cells.

### Fucosyltransferases

The preferred fucosyltransferases, especially α8-fucosyltransferase FUT8, to be analyzed in context of analysis of mRNA-level glycosylation analysis are shown in Table 1. The presence of FUT8 was especially characteristic for the blood derived stem cells. The presence of FUT4 and absence (low expression) of FUT7 were considered as characteristic features for both CD133+ and CD133− cells.

The invention is directed to the method of analyzing differentiation associated glycan expression according to the invention in blood stem cells, wherein mRNA expression or glycosylation enzymes being glycosyltransferases or glycosidases indicated to be related to the biosynthesis of the glycans is measured, optionally the analysis is performed together with analysis of the glycan structures.

The invention is directed to the method of analyzing mRNA, wherein the expression of glycosylation enzymes synthesizing the N-glycan core is measured, preferably mannosidases and/or N-actylglucosaminyltransferases of MGAT-family. Preferably the expression of at least one enzyme selected from the group MGAT2, MGAT4 and MAN1C1 is measured.

The invention is further directed to the method of analyzing mRNA, wherein the expression of enzymes synthesizing modification of N-glycans is used and the enzymes are selected from the group sialyltransferases, preferably α3- and/or α6-sialyltransferases; fucosyltransferases, preferably α3/4- and/or α8-fucosyltransferases; and galactosyltransferases, preferably β4-galactosyltransferases. Preferably the method is directed to the expression of at least one enzyme gene selected from the group FUT8, FUT4 or FUT7; or ST6GAL1, ST3GAL6, or ST3GAL5; or B4GALT1, B4GALT2 or B4GALT3, more preferably B4GALT2 or B4GALT3.

More preferably at least two enzymes of transferring different monosaccharide residues are measured most preferably at least two enzymes types from groups of sialyltransferases, fucosyltransferases and galactosyltransferases are measured, most preferably at least one enzyme from all of these groups, even more preferably two enzymes from each group is analyzed.

### Modulation of Glycosylation of Stem Cells

The invention further revealed that it is possible to modulate the differentiation status or process of stem cells by altering the glycosylation, which is altered when comparing stem cells and differentiated cells.

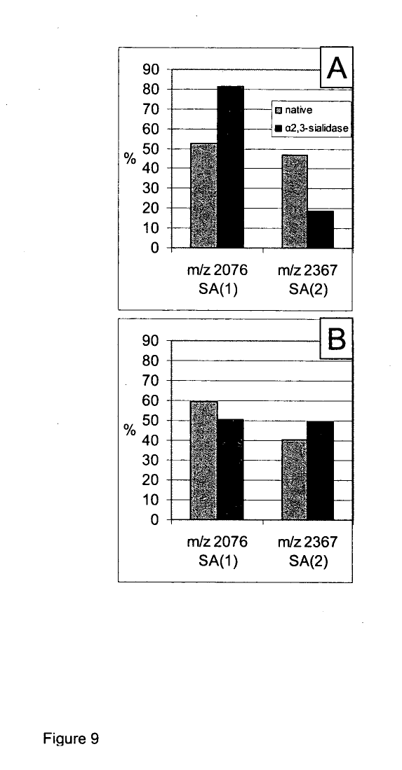

The invention is especially directed to the alteration of α3- and or α6-sialylation of the cells, which was shown to have major effects on the stem cells. The invention further revealed that the there is differentiation associated changes in α3- and α6-sialylation levels as shown in FIG. 9 and mRNA expression of the corresponding sialyltransferases.

### Altering the Glycosylation Enzymatically

The inventors revealed that it is possible to affect to the differentiation of stem cells by enzymatically altering the glycosylation on cell surface. In a preferred embodiment the invention is directed to the alteration of sialylation level of blood stem cells preferably by sialidase or sialyltransferase treatment, more preferably by sialidase, and thus modulating the cells. The invention revealed major effect of alteration of sialylation to the differentiation of blood stem cells as described in Example 4 and 5. The invention is directed to the alteration of the sialylation by α3-specific sialidases and/or by α6-specific sialidases.

### Other Methods for Altering the Glycosylation

Modulation of Stem Cell by Altering Glycosylation on mRNA Level

The invention is further directed to the modulation of stem cells by altering glycosylation on mRNA level, preferably by RNAi method. The methods for modification of mRNA expression are well-known in the art as described in Zheng G D et al (Stem Cells (2005) 23 (8) 1028-34) in context of stem cells and e.g. in Bjorklund M et al (Nature (2006) 439 (7079) 1009-13). RNAi reagents for the human transferases and mannosidases are available e.g. from iGene service of Invitrogen (www.igene.invitrogen.com/igene) or from Origene (shRNA,www.origene.com) by routine nucleotide synthesis services.

The invention is further directed to other methods for altering the glycosylation such as affecting the biosynthesis of glycans on other levels.

The invention is directed to a method affecting the differentiation status of stem cells, preferably blood stem cells by changing or modulating the differentiation associated glycan expression as described in the invention in blood stem cells.

The invention is especially directed to the method, wherein the amount of a differentiation associated glycan structure is either decreased or increased. In a preferred method, the amount of the glycan is changed by a glycosyltransferase or glycosidase capable of altering the glycosylation. In a preferred embodiment the amount of the glycan is changed in vitro by a glycosyltransferase or glycosidase capable of altering the glycosylation. More preferably the amount of sialylated glycans is changed, preferably the amount of α3- and or α6-sialylated glycans is changed in comparison to terminal Galβ-epitopes on cell surface, more preferably in comparison to Galβ4GlcNAc on cell surface. Even more preferably in vitro by sialyltransferases or sialidase capable of altering the sialylation on cell surfaces.

The invention is further directed to an in vivo method, wherein the amount of the glycan is changed altering the in vivo activity of a glycosylation enzyme being glycosyltransferase or glycosidase capable of altering the glycosylation. Preferably the glycosylation enzyme corresponds to N-acetylglucosaminyltransferase, mannosidase, galactosyltransferase, fucosyltransferase or sialyltransferase gene, preferably FUT8, FUT4 or FUT7; or ST6GAL1, ST3GAL6, or ST3GAL5; or B4GALT1, B4GALT2 or B4GALT3, more preferably B4GALT2 or B4GALT3 or MGAT2, MGAT4 and MAN1C1. In a preferred embodiment the amount of the glycan is changed altering the in vivo activity of sialyltransferases or sialidase capable of altering the sialylation. Preferably the alteration is performed by RNAi-methods, by transfection of enzyme to the cells and/or metabolic inhibition by inhibitors of the enzymes.

The invention is especially directed to affecting the differentiation of blood stem cells by sialyltransferases or sialidases as shown in examples 4 and 5.

### Preferred N-Glycan Structure Types

The invention revealed N-glycans with common core structure of N-glycans, which change according to differentiation and/or individual specific differences.

The N-glycans of stem cells comprise core structure comprising

Manβ4GlcNAc structure in the core structure of N-linked glycan according to the Formula CGN:

[Manα3]ₙ₁(Manα6)ₙ₂Manβ4GlcNAcβ4(Fucα6)ₙ₃GlcNAcxR,
wherein n1, n2 and n3 are integers 0 or 1, independently indicating the presence or absence of the residues, and
wherein the non-reducing end terminal Manα3/Manα6-residues can be elongated to the complex type, especially biantennary structures or to mannose type (high-Man and/or low Man) or to hybrid type structures (for the analysis of the status of stem cells and/or manipulation of the stem cells), wherein xR indicates reducing end structure of N-glycan linked to protein or peptide such as PAsn or PAsn-peptide or PAsn-protein, or free reducing end of N-glycan or chemical derivative of the reducing end produced for analysis.
   Mannose type Glycans

The preferred Mannose type glycans are according to the formula: Formula M2:

[Mα2]ₙ₁[Mα3]ₙ₂{[Mα2]ₙ₃[Mα6)]ₙ₄}[Mα6]ₙ₅{[Mα2]ₙ₆[Mα2]ₙ₇[Mα3]ₙ₈}Mβ4GNβ4[{Fucα6}]ₘGNyR₂

wherein n1, n2, n3, n4, n5, n6, n7, n8, and m are either independently 0 or 1; with the provision that when n2 is 0, also n1 is 0; when n4 is 0, also n3 is 0; when n5 is 0, also n1, n2, n3, and n4 are 0; when n7 is 0, also n6 is 0; when n8 is 0, also n6 and n7 are 0;
y is anomeric linkage structure α and/or β or linkage from derivatized anomeric carbon, and
R₂ is reducing end hydroxyl, chemical reducing end derivative or natural asparagine N-glycoside derivative such as asparagine N-glycosides including asparagines N-glycoside amino acid and/or peptides derived from protein;
[ ] indicates determinant either being present or absent depending on the value of n1, n2, n3, n4, n5, n6, n7, n8, and m; and
{ } indicates a branch in the structure;

### M is D-Man, GN is N-acetyl-D-glucosamine and Fuc is L-Fucose,

and the structure is optionally a high mannose structure, which is further substituted by glucose residue or residues linked to mannose residue indicated by n6.

### Low Man Glycans

Several preferred low mannose, low Man, glycans described above can be presented in a single Formula:

[Mα3]ₙ₂{[Mα6)]ₙ₄}[Mα6]ₙ₅{[Mα3]ₙ₈}Mβ4GNβ4[{Fucα6}]ₘGNyR₂

wherein n2, n4, n5, n8, and m are either independently 0 or 1; with the provision that when n5 is 0, also n2, and n4 are 0; the sum of n2, n4, n5, and n8 is less than or equal to (m+3); [ ] indicates determinant either being present or absent depending on the value of n2, n4, n5, n8, and m; and
{ } indicates a branch in the structure;
y and R2 are as indicated above.

Preferred non-fucosylated low-mannose glycans are according to the formula:

[Mα3]ₙ₂([Mα6)]ₙ₄)[Mα6]ₙ₅{[Mα3]ₙ₈}Mβ4GNβ4GNyR₂

wherein n2, n4, n5, n8, and m are either independently 0 or 1,
with the provision that when n5 is 0, also n2 and n4 are 0, and preferably either n2 or n4 is 0,
[ ] indicates determinant either being present or absent
depending on the value of, n2, n4, n5, n8,
{ } and ( ) indicates a branch in the structure,
y and R2 are as indicated above.

### Preferred Individual Structures of Non-Fucosylated Low-Mannose Glycans

### Special Small Structures

Small non-fucosylated low-mannose structures are especially unusual among known N-linked glycans and characteristic glycan group useful for separation of cells according to the present invention. These include:

Mβ4GNβ4GNyR₂, Mα6Mβ4GNβ4GNyR₂, Mα3 Mβ4GNβ4GNyR₂ and Mα6{Mα3}Mβ4GNβ4GNyR₂. Mβ4GNβ4GNyR₂ trisaccharide epitope is a preferred common structure alone and together with its mono-mannose derivatives Mα6Mβ4GNβ4GNyR₂ and/or Mα3Mβ4GNβ4GNyR₂, because these are characteristic structures commonly present in glycomes according to the invention. The invention is specifically directed to the glycomes comprising one or several of the small non-fucosylated low-mannose structures. The tetrasaccharides are in a specific embodiment preferred for specific recognition directed to α-linked, preferably α3/6-linked Mannoses as preferred terminal recognition element.

### Special Large Structures

The invention further revealed large non-fucosylated low-mannose structures that are unusual among known N-linked glycans and have special characteristic expression features among the preferred cells according to the invention. The preferred large structures include [Mα3]ₙ₂([Mα6]ₙ₄)Mα6{Mα3}Mβ4GNβ4GNyR₂ more preferably Mα6Mα6{Mα3}Mβ4GNβ4GNyR₂Mα3Mα6{Mα3}Mβ4GNβ4GNyR₂ and Mα3 (Mα6)Mα6{Mα3}Mβ4GNβ4GNyR₂.

The hexasaccharide epitopes are preferred in a specific embodiment as rare and characteristic structures in preferred cell types and as structures with preferred terminal epitopes. The heptasaccharide is also preferred as a structure comprising a preferred unusual terminal epitope Mα3(Mα6)Mα useful for analysis of cells according to the invention.

Preferred fucosylated low-mannose glycans are derived according to the formula:

[Mα3]ₙ₂{[Mα6]ₙ₄}[Mα6]ₙ₅{[Mα3]ₙ₈}Mβ4GNβ4(Fucα6)GNyR₂

wherein n2, n4, n5, n8, and m are either independently 0 or 1, with the provision that when n5 is 0, also n2 and n4 are 0, and preferably at least one of n2, n4 or n8 is 0, more preferably n2 or n4.
[ ] indicates determinant either being present or absent
depending on the value of n2, n4, n5, n8, and m;
{ } and ( ) indicate a branch in the structure.

### Preferred Individual Structures of Fucosylated Low-Mannose Glycans

Small fucosylated low-mannose structures are especially unusual among known N-linked glycans and form a characteristic glycan group useful for separation of cells according to the present invention. These include:

Mβ4GNβ4(Fucα6)GNyR₂, Mα6Mβ4GNβ4(Fucα6)GNyR₂, Mα3Mβ4GNβ4(Fucα6)GNyR₂ and Mα6{Mα3}Mβ4GNβ4(Fucα6)GNyR₂, and Mβ4GNβ4(Fucα6)GNyR₂ tetrasaccharide epitope is a preferred common structure alone and together with its monomannose derivatives Mα6Mβ4GNβ4(Fucα6)GNyR₂ and/or Mα3Mβ4GNβ4(Fucα6)GNyR₂, because these are commonly present characteristic structures in glycomes according to the invention. The invention is specifically directed to the glycomes comprising one or several of the small fucosylated low-mannose structures. The tetrasaccharides are in a specific embodiment preferred for specific recognition directed to α-linked, preferably α3/6-linked Mannoses as preferred terminal recognition element.

### Special Large Structures

The invention further revealed large fucosylated low-mannose structures that are unusual among known N-linked glycans and have special characteristic expression features among the preferred cells according to the invention. The preferred large structures include [Mα3]ₙ₂([Mα6]ₙ₄)Mα6{Mα3}M4GNβ4(Fucα6)GNyR₂, more specifically Mα6Mα6{Mα3}M4GNβ4(Fucα6)GNyR₂, Mα3 Mα6{Mα3}Mβ4GNβ4(Fucα6)GNyR₂ and Mα3(Mα6)Mα6{Mα3}Mβ4GNβ4(Fucα6)GNyR₂. The heptasaccharide epitopes are preferred in a specific embodiment as rare and characteristic structures in preferred cell types and as structures with preferred terminal epitopes. The octasaccharide is also preferred as structure comprising a preferred unusual terminal epitope Mα3(Mα6)Mα useful for analysis of cells according to the invention.

### Preferred Non-Reducing End Terminal Mannose-Epitopes

The inventors revealed that mannose-structures can be labeled and/or otherwise specifically recognized on cell surfaces or cell derived fractions/materials of specific cell types. The present invention is directed to the recognition of specific mannose epitopes on cell surfaces by reagents binding to specific mannose structures on cell surfaces.

The preferred reagents for recognition of any structures according to the invention include specific antibodies and other carbohydrate recognizing binding molecules. It is known that antibodies can be produced for the specific structures by various immunization and/or library technologies such as phage display methods representing variable domains of antibodies. Similarly with antibody library technologies, including aptamer technologies and including phage display for peptides, exist for synthesis of library molecules such as polyamide molecules including peptides, especially cyclic peptides, or nucleotide type molecules such as aptamer molecules.

The invention is specifically directed to specific recognition of high-mannose and low-mannose structures according to the invention. The invention is specifically directed to recognition of non-reducing end terminal Manα-epitopes, preferably at least disaccharide epitopes, according to the formula:

[Mα2]ₘ₁[Mαx]ₘ₂[Mα6]ₘ₃{{[Mα2]ₘ₉[Mα2]ₘ₈[Mα3]ₘ₇}ₘ₁₀(Mβ4[GN]ₘ₄)ₘ₅}ₘ₆yR₂

wherein m1, m2, m3, m4, m5, m6, m7, m8, m9 and m10 are independently either 0 or 1; with the provision that when m3 is 0, then m¹ is 0, and when m7 is 0 then either m1-5 are 0 and m8 and m9 are 1 forming a Mα2Mα2-disaccharide, or both m8 and m9 are 0;
y is anomeric linkage structure α and/or β or linkage from derivatized anomeric carbon, and
R₂ is reducing end hydroxyl or chemical reducing end derivative
and x is linkage position 3 or 6 or both 3 and 6 forming branched structure,
{ } indicates a branch in the structure.

The invention is further directed to terminal Mα2-containing glycans containing at least one Mα2-group and preferably Mα2-group on each branch so that m1 and at least one of m8 or m9 is 1. The invention is further directed to terminal Mα3 and/or Mα6-epitopes without terminal Mα2-groups, when all m1, m8 and m9 are 1.

The invention is further directed in a preferred embodiment to the terminal epitopes linked to a Mβ-residue and for application directed to larger epitopes. The invention is especially directed to Mβ4GN-comprising reducing end terminal epitopes.

The preferred terminal epitopes comprise typically 2-5 monosaccharide residues in a linear chain. According to the invention short epitopes comprising at least 2 monosaccharide residues can be recognized under suitable background conditions and the invention is specifically directed to epitopes comprising 2 to 4 monosaccharide units and more preferably 2-3 monosaccharide units, even more preferred epitopes include linear disaccharide units and/or branched trisaccharide non-reducing residue with natural anomeric linkage structures at reducing end. The shorter epitopes may be preferred for specific applications due to practical reasons including effective production of control molecules for potential binding reagents aimed for recognition of the structures.

The shorter epitopes such as Mα2M is often more abundant on target cell surface as it is present on multiple arms of several common structures according to the invention.

### Preferred Disaccharide Epitopes Include

Manα2Man, Manα3Man, Manα6Man, and more preferred anomeric forms Manα2Manα, Manα3Manβ, Manα6Manβ, Manα3Manα and Manα6Manα.

Preferred branched trisaccharides include Manα3(Manα6)Man, Manα3(Manα6)Manβ, and Manα3(Manα6)Manα.

The invention is specifically directed to the specific recognition of non-reducing terminal Manα2-structures especially in context of high-mannose structures.

The invention is specifically directed to following linear terminal mannose epitopes:

a) preferred terminal Manα2-epitopes including following oligosaccharide sequences: Manα2Man, Manα2Manα, Manα2Manα2Man, Manα2Manα3Man, Manα2Manα6Man, Manα2Manα2Manα, Manα2Manα3Manβ, Manα2Manα6Manα, Manα2Manα2Manα3Man, Manα2Manα3Manα6Man, Manα2Manα6Manα6Man Manα2Manα2Manα3Manβ, Manα2Manα3Manα6Manβ, Manα2Manα6Manα6Manβ;

The invention is further directed to recognition of and methods directed to non-reducing end terminal Manα3- and/or Manα6-comprising target structures, which are characteristic features of specifically important low-mannose glycans according to the invention. The preferred structural groups include linear epitopes according to b) and branched epitopes according to the c3) especially depending on the status of the target material.

b) preferred terminal Manα3- and/or Manα6-epitopes including following oligosaccharide sequences:
Manα3Man, Manα6Manβ, Manα3Manβ, Manα6Manβ, Manα3Manα, Manα6Manα, Manα3Manα6Man, Manα6Manα6Man, Manα3Manα6Manβ, Manα6Manα6Manβ and to following:
c) branched terminal mannose epitopes are preferred as characteristic structures of especially high-mannose structures (c1 and c2) and low-mannose structures (c3), the preferred branched epitopes including:
c1) branched terminal Manα2-epitopes
Manα2Manα3(Manα2Manα6)Man, Manα2Manα3 (Manα2Manα6)Manα, Manα2Manα3(Manα2Manα6)Manα6Man, Manα2Manα3 (Manα2Manα6)Manα6Manβ, Manα2Manα3(Manα2Manα6)Manα6(Manα2Manα3)Man, Manα2Manα3(Manα2Manα6)Manα6(Manα2Manα2Manα3)Man, Manα2Manα3(Manα2Manα6)Manα6(Manα2Manα3)Man, Manα2Manα3(Manα2Manα6)Manα6(Manα2Manα3)Man,
c2) branched terminal Manα2- and Manα3 or Manα6-epitopes
according to formula when m1 and/or m8 and/m9 is 1 and the molecule comprise at least one nonreducing end terminal Manα3 or Manα6-epitope
c3) branched terminal Manα3 or Manα6-epitopes

### Manα3 (Manα6)Man, Manα3 (Manα6)Manβ, Manα3(Manα6)Manα, Manα3 (Manα6)Manα6Man, Manα3 (Manα6)Manα6Manβ, Manα3 (Manα6)Manα6(Manα3)Man, Manα3(Manα6)Manα6(Manα3)Manβ

The present invention is further directed to increase the selectivity and sensitivity in recognition of target glycans by combining recognition methods for terminal Manα2 and Manα3 and/or Manα6-comprising structures. Such methods would be especially useful in context of cell material according to the invention comprising both high-mannose and low-mannose glycans.

### Complex Type N-Glycans

According to the present invention, complex-type structures are preferentially identified by mass spectrometry, preferentially based on characteristic monosaccharide compositions, wherein HexNAc≧4 and Hex≧3. In a more preferred embodiment of the present invention, 4≦HexNAc≦20 and 3≦Hex≦21, and in an even more preferred embodiment of the present invention, 4≦HexNAc≦10 and 3≦Hex≦11. The complex-type structures are further preferentially identified by sensitivity to endoglycosidase digestion, preferentially N-glycosidase F detachment from glycoproteins. The complex-type structures are further preferentially identified in NMR spectroscopy based on characteristic resonances of the Manα3(Manα6)Manβ4GlcNAcβ4GlcNAc N-glycan core structure and GlcNAc residues attached to the Manα3 and/or Manα6 residues.

Beside Mannose-type glycans the preferred N-linked glycomes include GlcNAcβ2-type glycans including Complex type glycans comprising only GlcNAcβ2-branches and Hybrid type glycan comprising both Mannose-type branch and GlcNAcβ2-branch.

### GlcNAcβ2-Type Glycans

The invention revealed GlcNAcβ2Man structures in the glycomes according to the invention. Preferably GlcNAcβ2Man-structures comprise one or several of GlcNAcβ2Manα-structures, more preferably GlcNAcβ2Manα3- or GlcNAcβ2Manα6-structure.

The Complex type glycans of the invention comprise preferably two GlcNAcβ2Manα structures, which are preferably GlcNAcβ2Manα3 and GlcNAcβ2Manα6. The Hybrid type glycans comprise preferably GlcNAcβ2Manα3-structure.

The invention revealed characteristic complex type glycan with common core structures referred in general formula for complex type glycan (CO1), this formula is also referred as GNβ2, because the presence of the epitope.

The present invention is directed to at least one of natural oligosaccharide sequence structures and structures truncated from the reducing end of the N-glycan according to the Formula CO1 (also referred as Formula GNβ2):

[R₁GNβ2]ₙ₁[Mα3]ₙ₂{[R₃]ₙ₃[GNβ2]ₙ₄Mα6}ₙ₅Mβ4GNXyR₂,

with optionally one or two or three additional branches according to formula [RₓGNβz]ₙₓ linked to Mα6-, Mα3-, or Mβ4, and Rₓ may be different in each branch
wherein n1, n2, n3, n4, n5 and nx, are either 0 or 1, independently,
with the provision that when n2 is 0 then n1 is 0 and when n3 is 1 and/or n4 is 1 then n5 is also 1, and at least one of n1, or n4, or nx, or n3 is 1, preferably at least one of n1, or n4, or nx, is 1 when n4 is 0 and n3 is 1 then R₃ is a mannose type substituent or nothing and
wherein X is a glycosidically linked disaccharide epitope β4(Fucα6)ₙGN, wherein n is 0 or 1, or X is nothing and
y is anomeric linkage structure α and/or β or linkage from derivatized anomeric carbon, and
R₁, Rₓ and R₃ indicate independently one, two or three natural substituents linked to the core structure,
R₂ is reducing end hydroxyl, chemical reducing end derivative or natural asparagine N-glycoside derivative such as asparagine N-glycosides including asparagines N-glycoside amino acids and/or peptides derived from protein; [ ] indicate groups either present or absent in a linear sequence, and { } indicates branching which may be also present or absent.

### Elongation of GlcNAcβ2-Type Structures Forming Complex/Hybrid Type Structures

The substituents R₁, Rₓ and R₃ may form elongated structures. In the elongated structures R₁, and Rₓ represent substituents of GlcNAc (GN) and R₃ is either substituent of GlcNAc or when n4 is 0 and n3 is 1 then R₃ is a mannose type substituent linked to Manα6-branch forming a Hybrid type structure. The substituents of GN are monosaccharide Gal, GalNAc, or Fuc and/or acidic residue such as sialic acid or sulfate or phosphate ester.

GlcNAc or GN may be elongated to N-acetyllactosaminyl also marked as GalβGN or di-N-acetyllactosdiaminyl GalNAcβGlcNAc, preferably GalNAcβ4GlcNAc. LNβ2M can be further elongated and/or branched with one or several other monosaccharide residues such as galactose, fucose, SA or LN-unit(s) which may be further substituted by SAα-structures, and/or Mα6 residue and/or Mα3 residue can be further substituted by one or two β6-, and/or β4-linked additional branches according to the formula;

and/or either of Mα6 residue or Mα3 residue may be absent;
and/or Mα6—residue can be additionally substituted by other Manα units to form a hybrid type structures;
and/or Manβ4 can be further substituted by GNβ4,
and/or SA may include natural substituents of sialic acid and/or it may be substituted by other SA-residues preferably by α8- or α9-linkages.

The SAα-groups are linked to either 3- or 6-position of neighboring Gal residue or on 6-position of GlcNAc, preferably 3- or 6-position of neighboring Gal residue. In separately preferred embodiments the invention is directed to structures comprising solely 3-linked SA or 6-linked SA, or mixtures thereof.

### Preferred Complex Type Structures

### Incomplete Monoantennary N-Glycans

The present invention revealed incomplete Complex monoantennary N-glycans, which are unusual and useful for characterization of glycomes according to the invention. The most of the incomplete monoantennary structures indicate potential degradation of biantennary N-glycan structures and are thus preferred as indicators of cellular status. The incomplete Complex type monoantennary glycans comprise only one GNβ2-structure.

The invention is specifically directed to structures according to the Formula CO1 or Formula GNb2 above when only n1 is 1 or n4 is 1 and mixtures of such structures.

The preferred mixtures comprise at least one monoantennary complex type glycans

A) with a single branch likely from a degradative biosynthetic process:

### R₁GNβ2Mα3βGNXyR₂

### R₃GNβ2Mα6Mβ4GNXyR₂ and

B) with two branches comprising mannose branches

B1) R₁GNβ2Mα3{Mα6}ₙ₅Mβ4GNXyR₂

B2) Mα3{R₃GNβ2Mα6}ₙ₅ Mβ4GNXyR₂

The structure B2 is preferred over A structures as product of degradative biosynthesis, it is especially preferred in context of lower degradation of Manα3-structures. The structure B1 is useful for indication of either degradative biosynthesis or delay of biosynthetic process.

### Biantennary and Multiantennary Structures

The inventors revealed a major group of biantennary and multiantennary N-glycans from cells according to the invention. The preferred biantennary and multiantennary structures comprise two GNβ2 structures. These are preferred as an additional characteristic group of glycomes according to the invention and are represented according to the Formula CO2:

R₁GNβ2Mα3{R₃GNβ2Mα6}Mβ4GNXyR₂

with optionally one or two or three additional branches according to formula [RₓGNβz]ₙₓ linked to Mα6-, Mα3-, or Mβ4 and Rₓ may be different in each branch
wherein nx is either 0 or 1,
and other variables are according to the Formula CO1.

### Preferred Biantennary Structure

A biantennary structure comprising two terminal GNβ-epitopes is preferred as a potential indicator of degradative biosynthesis and/or delay of biosynthetic process. The more preferred structures are according to the Formula CO2 when R₁ and R₃ are nothing.

### Elongated Structures

The invention revealed specific elongated complex type glycans comprising Gal and/or GalNAc-structures and elongated variants thereof. Such structures are especially preferred as informative structures because the terminal epitopes include multiple informative modifications of lactosamine type, which characterize cell types according to the invention.

The present invention is directed to at least one of natural oligosaccharide sequence structure or group of structures and corresponding structure(s) truncated from the reducing end of the N-glycan according to the Formula CO3:

[R₁Gal[NAc]ₒ₂βz2]ₒ₁ GNβ2Mα3{[R₁Gal[NAc]ₒ₄βz2]ₒ₃GNβ2Mα6}Mβ4GNXyR₂,

with optionally one or two or three additional branches according to formula [RₓGNβz1]ₙₓ linked to Mα6-, Mα3-, or Mβ4 and Rₓ may be different in each branch
wherein nx, o1, o2, o3, and o4 are either 0 or 1, independently,
with the provision that at least o1 or o3 is 1, in a preferred embodiment both are 1;
z2 is linkage position to GN being 3 or 4, in a preferred embodiment 4;
z1 is linkage position of the additional branches;
R₁, Rx and R₃ indicate one or two a N-acetyllactosamine type elongation groups or nothing,
{ } and ( ) indicates branching which may be also present or absent,
other variables are as described in Formula GNb2.

### Galactosylated Structures

The inventors characterized useful structures especially directed to digalactosylated structure GalβzGNβ2Mα3{GalβzGNβ2Mα6}Mβ4GNXyR₂, and monogalactosylated structures: GalβzGNβ2Mα3{GNβ2Mα6}Mβ4GNXyR₂, GNβ2Mα3{GalβzGNβ2Mα6}Mβ4GNXyR₂, and/or elongated variants thereof preferred for carrying additional characteristic terminal structures useful for characterization of glycan materials R₁GalβzGNβ2Mα3{R₃GalβzGNβ2Mα6}Mβ4GNXyR₂, R₁GalβzGNβ2Mα3{GNβ2Mα6}Mβ4GNX yR₂, and GNβ2Mα3 {R₃GalβzGNβ2Mα6}Mβ4GNXyR₂. Preferred elongated materials include structures wherein R₁ is a sialic acid, more preferably NeuNAc or NeuGc.

### LacdiNAc-Structure Comprising N-Glycans

The present invention revealed for the first time LacdiNAc, GalNAcβGlcNAc structures from the cell according to the invention. Preferred N-glycan lacdiNAc structures are included in structures according to the Formula CO1, when at least one the variable o2 and o4 is 1.

### The Major Acidic Glycan Types

The acidic glycomes mean glycomes comprising at least one acidic monosaccharide residue such as sialic acids (especially NeuNAc and NeuGc) forming sialylated glycome, HexA (especially GlcA, glucuronic acid) and/or acid modification groups such as phosphate and/or sulphate esters.

According to the present invention, presence of sulphate and/or phosphate ester (SP) groups in acidic glycan structures is preferentially indicated by characteristic monosaccharide compositions containing one or more SP groups. The preferred compositions containing SP groups include those formed by adding one or more SP groups into non-SP group containing glycan compositions, while the most preferential compositions containing SP groups according to the present invention are selected from the compositions described in the acidic N-glycan fraction glycan group Tables of the present invention. The presence of phosphate and/or sulphate ester groups in acidic glycan structures is preferentially further indicated by the characteristic fragments observed in fragmentation mass spectrometry corresponding to loss of one or more SP groups, the insensitivity of the glycans carrying SP groups to sialidase digestion. The presence of phosphate and/or sulphate ester groups in acidic glycan structures is preferentially also indicated in positive ion mode mass spectrometry by the tendency of such glycans to form salts such as sodium salts as described in the Examples of the present invention. Sulphate and phosphate ester groups are further preferentially identified based on their sensitivity to specific sulphatase and phosphatase enzyme treatments, respectively, and/or specific complexes they form with cationic probes in analytical techniques such as mass spectrometry.

### Sialylated Complex N-Glycan Glycomes

The present invention is directed to at least one of natural oligosaccharide sequence structures and structures truncated from the reducing end of the N-glycan according to the Formula

[{SAα3/6}ₛ₁LNβ2]ᵣ₁Mα3{({SAα3/6}ₛ₂LNβ2)ᵣ₂Mα6}ᵣ₈{M[β4GN[β4{Fucα6}ᵣ₃GN]ᵣ₄]ᵣ₅}ᵣ₆ (I)

with optionally one or two or three additional branches according to formula

{SAα3/6}ₛ₃LNβ, (IIb)

wherein r1, r2, r3, r4, r5, r6, r7 and r8 are either 0 or 1, independently,
wherein s1, s2 and s3 are either 0 or 1, independently,
with the provision that at least r1 is 1 or r2 is 1, and at least one of s1, s2 or s3 is 1.
LN is N-acetyllactosaminyl also marked as GalβGN or di-N-acetyllactosdiaminyl GalNAcβGlcNAc preferably GalNAcβ4GlcNAc, GN is GlcNAc, M is mannosyl-,
with the provision that LNβ2M or GNβ2M can be further elongated and/or branched with one or several other monosaccharide residues such as galactose, fucose, SA or LN-unit(s) which may be further substituted by SAα-structures,
and/or one LNβ can be truncated to GNP
and/or Mα6 residue and/or Mα3 residue can be further substituted by one or two β6-, and/or β4-linked additional branches according to the formula,
and/or either of Mα6 residue or Mα3 residue may be absent;
and/or Mα6-residue can be additionally substituted by other Manα units to form a hybrid type structures
and/or Manβ4 can be further substituted by GNβ4,
and/or SA may include natural substituents of sialic acid and/or it may be substituted by other SA-residues preferably by α8- or α9-linkages.
( ), { }, [ ] and [ ] indicate groups either present or absent in a linear sequence. { }indicates branching which may be also present or absent.

The SAα-groups are linked to either 3- or 6-position of neighboring Gal residue or on 6-position of GlcNAc, preferably 3- or 6-position of neighboring Gal residue. In separately preferred embodiments the invention is directed structures comprising solely 3-linked SA or 6-linked SA, or mixtures thereof. In a preferred embodiment the invention is directed to glycans wherein r6 is 1 and r5 is 0, corresponding to N-glycans lacking the reducing end GlcNAc structure.

The LN unit with its various substituents can be represented in a preferred general embodiment by the formula:

[Gal(NAc)ₙ₁α3]ₙ₂{Fucα2}ₙ₃Gal(NAc)ₙ₄β3/4{Fucα4/3}ₙ₅GlcNAcβ

wherein n1, n2, n3, n4, and n5 are independently either 1 or 0,
with the provision that the substituents defined by n2 and n3 are alternative to the presence of SA at the non-reducing end terminal structure;
the reducing end GlcNAc-unit can be further β3- and/or β6-linked to another similar LN-structure forming a poly-N-acetyllactosamine structure with the provision that for this LN-unit n2, n3 and n4 are 0,
the Gal(NAc)β and GlcNAcβ units can be ester linked a sulphate ester group;
( ) and [ ] indicate groups either present or absent in a linear sequence; { }indicates branching which may be also present or absent.

LN unit is preferably Galβ4GN and/or Galβ3GN. The inventors revealed that stem cells can express both types of N-acetyllactosamine, and therefore the invention is especially directed to mixtures of both structures, but type II was especially common in blood stem cells. Furthermore, the invention is directed to type 2 N-acetyllactosamines, Galβ4GlcNAc, novel characteristic markers of the blood stem cells.

### Hybrid Type Structures

According to the present invention, hybrid-type or monoantennary structures are preferentially identified by mass spectrometry, preferentially based on characteristic monosaccharide compositions, wherein HexNAc=3 and Hex≧2. In a more preferred embodiment of the present invention 2≦Hex≦11, and in an even more preferred embodiment of the present invention 2≦Hex≦9. The hybrid-type structures are further preferentially identified by sensitivity to exoglycosidase digestion, preferentially α-mannosidase digestion when the structures contain non-reducing terminal α-mannose residues and Hex≧3, or even more preferably when Hex≧4, and to endoglycosidase digestion, preferentially N-glycosidase F detachment from glycoproteins. The hybrid-type structures are further preferentially identified in NMR spectroscopy based on characteristic resonances of the Manα3(Manα6)Manβ4GlcNAcβ4GlcNAc N-glycan core structure, a GlcNAcβ residue attached to a Manα residue in the N-glycan core, and the presence of characteristic resonances of non-reducing terminal α-mannose residue or residues.

The monoantennary structures are further preferentially identified by insensitivity to α-mannosidase digestion and by sensitivity to endoglycosidase digestion, preferentially N-glycosidase F detachment from glycoproteins. The monoantennary structures are further preferentially identified in NMR spectroscopy based on characteristic resonances of the Manα3Manβ4GlcNAcβ4GlcNAc N-glycan core structure, a GlcNAcβ residue attached to a Manα residue in the N-glycan core, and the absence of characteristic resonances of further non-reducing terminal α-mannose residues apart from those arising from a terminal α-mannose residue present in a ManαManβ sequence of the N-glycan core.

The invention is further directed to the N-glycans when these comprise hybrid type structures according to the Formula HY1:

R₁GNβ2Mα3{[R₃]ₙ₃Mα6}Mβ4GNXyR₂,

wherein n3, is either 0 or 1, independently,
and wherein X is glycosidically linked disaccharide epitope β4(Fucα6)ₙGN, wherein n is 0 or 1, or
X is nothing and
y is anomeric linkage structure α and/or β or linkage from derivatized anomeric carbon, and
R₁ indicate nothing or substituent or substituents linked to GlcNAc,
R₃ indicates nothing or Mannose-substituent(s) linked to mannose residue, so that each of R₁, and R₃ may correspond to one, two or three, more preferably one or two, and most preferably at least one natural substituents linked to the core structure,
R₂ is reducing end hydroxyl, chemical reducing end derivative or natural asparagine N-glycoside derivative such as asparagine N-glycosides including asparagines N-glycoside amino acids and/or peptides derived from protein; [ ] indicate groups either present or absent in a linear sequence, and { } indicates branching which may be also present or absent.

### Preferred Hybrid Type Structures

The preferred hybrid type structures include one or two additional mannose residues on the preferred core structure.

R₁GNβ2Mα3{[Mα3]ₘ₁([Mα6])ₘ₂Mα6}Mβ4GNXyR₂, Formula HY2

wherein and m1 and m2 are either 0 or 1, independently,
{ } and ( ) indicates branching which may be also present or absent,
other variables are as described in Formula HY1.

Furthermore the invention is directed to structures comprising additional lactosamine type structures on GNβ2-branch. The preferred lactosamine type elongation structures includes N-acetyllactosamines and derivatives, galactose, GalNAc, GlcNAc, sialic acid and fucose.

Preferred structures according to the formula HY2 include:

Structures containing non-reducing end terminal GlcNAc as a specific preferred group of glycans GNβ2Mα3{Mα3Mα6}Mβ4GNXyR₂, GNβ2Mα3{Mα6Mα6}Mβ4GNXyR₂, GNβ2Mα3{Mα3(Mα6)Mα6}Mβ4GNXyR₂, and/or elongated variants thereof R₁GNβ2Mα3{Mα3Mα6}Mβ4GNXyR₂, R₁GNβ2Mα3{Mα6Mα6}Mβ4GNXyR₂, R₁GNβ2Mα3{Mα3(Mα6)Mα6}Mβ4GNXyR₂,

[R₁Gal[NAc]ₒ₂βz]ₒ₁GNβ2Mα3{[Mα3]ₘ₁[(Mα6)]ₘ₂Mα6}ₙ₅Mβ4GNXyR₂, Formula HY3

wherein n5, m1, m2, o1 and o2 are either 0 or 1, independently,
z is linkage position to GN being 3 or 4, in a preferred embodiment 4,
R₁ indicates one or two a N-acetyllactosamine type elongation groups or nothing,
{ } and ( ) indicates branching which may be also present or absent,
other variables are as described in Formula HY1.

Preferred structures according to the formula HY3 include especially structures containing non-reducing end terminal Galβ, preferably Galβ3/4 forming a terminal N-acetyllactosamine structure. These are preferred as a special group of Hybrid type structures, preferred as a group of specific value in characterization of balance of Complex N-glycan glycome and High mannose glycome:

GalβzGNβ2Mα3{Mα3Mα6}Mβ4GNXyR₂, GalβzGNβ2Mα3{Mα6Mα6}M4GNXyR₂, GalβzGNβ2Mα3{Mα3(Mα6)Mα6}Mβ4GNXyR₂,
and/or elongated variants thereof preferred for carrying additional characteristic terminal structures useful for characterization of glycan materials
R₁GalβzGNβ2Mα3{Mα3Mα6})Mβ4GNXyR₂, R₁GalβzGNβ2Mα3{Mα6Mα6}Mβ4GNXyR₂, R₁GalβzGNβ2Mα3{Mα3(Mα6)Mα6}Mβ4GNXyR₂. Preferred elongated materials include structures wherein R₁ is a sialic acid, more preferably NeuNAc or NeuGc.
Structures Associated with Blood Derived Stem Cells

The Tables 3 and 4 show specific structure groups with specific monosaccharide compositions associated with the differentiation status of human blood derived stem cells in comparison to the mononuclear cells from blood.

### The Structures Present and Enriched in Blood Stem Cell Cells

The invention revealed novel structures present in higher amounts in blood stem cell than in corresponding differentiated cells.

### Structures in Specific CD133 Selected Blood Stem Cell Populations

CD133 is a commonly used marker for hematopoietic and other stem cells. The invention revealed especially variation CD133+ cells in comparison to CD133− cells.

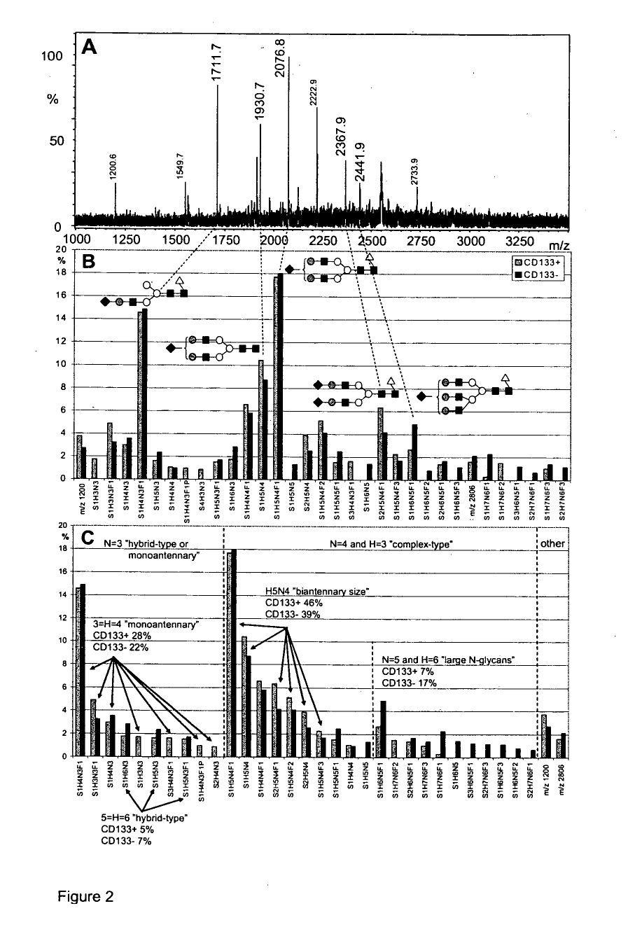

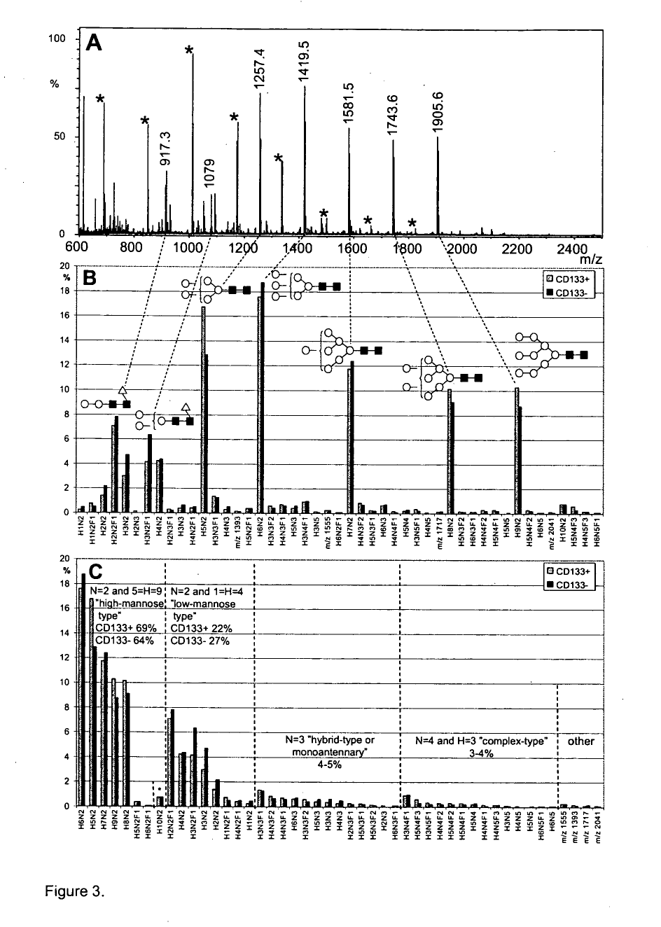

Major N-glycans in CD133+ and CD133− cells were high-mannose and biantennary complex-type structures. CD133+ and CD133− cells also had monoantennary, hybrid, low-mannose and large complex-type N-glycans (FIGS. 2 and 3), for details see example 1, showed polarization towards high-mannose type N-glycans (FIG. 2C), biantennary complex-type N-glycans with core composition 5-hexose 4-N-acetylhexosamine and sialylated monoantennary N-glycans (FIG. 3C). In contrast, CD133− cells had increased amounts of large complex-type N-glycans with core composition 6-hexose 5-N-acetylhexosamine or larger, sialylated hybrid-type N-glycans and low-mannose type N-glycans.

CD133+ Associated N-Glycan Groups CD133+i)-CD133+iii):

The invention revealed 3 groups of glycan compositions and glycan, named CD133+i)-CD133+iii, which are especially characteristic for the CD133 positive cells.

All the groups share common N-glycan core structure according to Formula CCN and the glycan groups are further divided to specific Complex type and Mannose type structures. The differences in the expression are shown in Tables 3 and 4.

Complex Type Glycans Compositions and Structures Associated with CD133+ Cells

### N-Glycan Group CD133+i),

Biantennary-Size Complex-Type Sialylated N-Glycans with Core H5N4

A preferred group of specific expression blood derived stem cells, especially CD133+ cells, was revealed to be a specific group of Biantennary-size complex-type sialylated N-glycans with composition feature H₅N₄, preferably including S1H5N4F1, S1H5N4, S2H5N4F1, S1H5N4F2, S2H5N4, and S1H5N4F3.

Preferred subgroups of sialylated structures include mono- and disialyl-structures with low fucosylation (none or one) S1H5N4F1, S1H5N4, S2H5N4F1, S2H5N4, and monosialylated structures with high fucosylation S1H5N4F2, and S1H5N4F3.

The preferred structures are according to the formula:

SₖH5N4F_{q}

wherein
k is an integer being 1 or 2, preferably 1 for high fucosylation group and
q is an integer being 0-3, preferably 0 or 1 for low fucosylation group, and 2 or 3 for high fucosylation group.
Preferred Biantennary Structures with Low Fucosylation

The preferred biantennary structures according to the invention include structures according to the Formula:

[NeuAcα]₀₋₁GalβGNβ2Manα3([NeuAcα]₀₋₁GalβGNβ2Manα6)Manβ4GNβ4(Fucα6)₀₋₁GN,

The GalβGlcNAc structures are preferably Galβ4GlcNAc-structures (type II N-acetyllactosamine antennae). The presence of type 2 structures was revealed by specific β4-linkage cleaving galactosidase (D. pneumoniae).

In a preferred embodiment the sialic acid is NeuAcα6- and the glycan comprises the NeuAc linked to Manα3-arm of the molecule. The assignment is based on the presence of α6-linked sialic acid revealed by specific sialidase digestion and the known branch specificity of the α6-sialyltransferase (ST6GalI). NeuAcα6GalβGNβ2Manα3([NeuAcα]₀₋₁GalβGNβ2Manα6)Manβ4GNβ4(Fucα6)₀₋₁GN, more preferably type II structures: NeuAcα6Galβ4GNβ2Manα3([NeuAcα]₀₋₁Galβ4GNβ2Manα6)Manβ4GNβ4(Fucα6)₀₋₁GN.

The invention thus revealed preferred terminal epitopes, NeuAcα6GalβGN, NeuAcα6GalβGNβ2Man, NeuAcα6GalβGNβ2Manα3, to be recognized by specific binder molecules. It is realized that higher specificity preferred for application in context of similar structures can be obtained by using binder recognizing longer epitopes and thus differentiating e.g. between N-glycans and other glycan types in context of the terminal epitopes.

Preferred Biantennary Structures with High Fucosylation

The invention is preferably directed to biantennary structures with high fucosylation, preferably with two (difucosylated) or three fucose (trifucosylated) structures.

### Preferred Difucosylated and Sialylated Structures

Preferred difucosylated sialylated structures include structures, wherein one fucose is in the core of the N-glycan and

a) one fucose on one arm of the molecule, and sialic acid is on the other arm (antenna of the molecule and the fucose is in Lewis x or H-structure:
Galβ4(Fucα3)GNβ2Manα3/6(NeuNAcαGalβGNβ2Manα6/3)Manβ4GNβ4(Fucα6)GN, and/or Fucα2GalβGNβ2Manα3/6(NeuNAcαGalβGNβ2Manα6/3)Manβ4GNβ4(Fucα6)GN, and when the sialic acid is α6-linked preferred antennary structures contain preferably the sialyl-lactosamine on α3-linked arm of the molecule according to formula:

Galβ4(Fucα3)GNβ2Manα6(NeuNAcα6Galβ4GNβ2Manα3)Manβ4GNβ4(Fucα6)GN,

and/or

Fucα2GalβGNβ2Manα6(NeuNAcα6Galβ4GNβ2Manα3)Manβ4GNβ4(Fucα6)GN.

It is realized that the structures, wherein the sialic acid and fucose are on different arms of the molecules can be recognized as characteristic specific epitopes.

b) Fucose and NeuAc are on the same arm in a structure:
NeuNAcα3 Galβ3/4(Fucα4/3)GNβ2Manα3/6(GalβGNβ2Manα6/3)Manβ4GNβ4(Fucα6)GN, and more preferably sialylated and fucosylated sialyl-Lewis x structures are preferred as a characteristic and bioactive structures:

### NeuNAcα3Galβ4(Fucα3)GNβ2Manα3/6(Galβ4GNβ2Manα6/3)Manβ4GNβ4(Fucα6)GN.

### Preferred Sialylated Trifucosylated Structures

Preferred sialylated trifucosylated structures include glycans comprising core fucose and the terminal sialyl-Lewis x or sialyl-Lewis a, preferably sialyl-Lewis x due to relatively large presence of type 2 lactosamines, or Lewis y on either arm of the biantennary N-glycan according to the formulae:

NeuNAcα3Galβ4(Fucα3)GNβ2Manα3/6([Fucα]GalβGNβ2Manα6/3)Manβ4GNβ4(Fucα6)GN,

and/or

Fucα2Galβ4(Fucα3)GNβ2Manα3/6(NeuNAcα3/6GalβGNβ2Manα6/3)Manβ4GNβ4(Fucα6)GN.

NeuNAc is preferably α-linked on the same arm as fucose due to known biosynthetic preference. When the structure comprises NeuNAcα6, this is preferably linked to form NeuNAcα6Galβ4GlcNAcβ2Manα3-arm of the molecule. Galβ groups are preferably type II N-acetyllactosamine structures Galβ4-groups for blood stem cells.

### N-Glycan Group CD133+ii)

### Monoantennary-Size Sialylated N-Glycans

The invention further revealed characteristic unusual glycans with monoantennary type glycan compositions.

This preferred group includes of CD133+ cell associated structures includes:

Monoantennary-size sialylated N-glycans with composition feature 3≦H≦4,
preferably including S1H3N3F1, S1H3N3, S3H4N₃F1, S1H4N3F1SP, S2H4N3, and optionally also S1H4N3F1 and/or S1H4N3.

Including linear monoantennary glycans S1H3N3F1, and S1H3N3 and branched monoantennary/hybrid type preferably with multiple charges S3H4N3F1, S1H4N3F1SP, S2H4N3,

and optionally also S1H4N3F1 and/or S1H4N3.

The preferred structures have monosacharide composition to the formula:

SₖHₘN₄F_{q}

wherein
k is an integer being 1, 2, or 3,
m is an integer being 3 or 4,
q is an integer being 0 or 1.

The preferred structures are according to the formula:

(NeuAc)ₙNeuAcα3/6GalβGlcNAcβ2Manα3(Manα6)₀₋₁Manβ4GlcNAcβ4(Fucα6)₀₋₁GlcNAc,

where in is 1 or 2, and the terminal sialic acids are preferably α8- or α9-linked, more preferably a8-linked more preferentially with type II N-acetyllactosamine antennae, wherein galactose residues are β1,4-linked (NeuAc)ₙNeuAcα3/6Galβ4GlcNAcβ2Manα3 (Manα6)₀₋₁Manβ4GlcNAcβ4(Fucα6)₀₋₁GlcNAc.

The preferred branched structures are according to the formula

(NeuAc)ₙNeuAcα3/6Galβ4GlcNAcβ2Manα3 (Manα6)Manβ4GlcNAcβ4(Fucα6)₀₋₁GlcNAc and

preferred linear structures are according to the formula

(SP)₀₋₁(NeuAc)ₙNeuAcα3/6Galβ4GlcNAcβ2Manα3 Manβ4GlcNAcβ4(Fucα6)₀₋₁GlcNAc,

optionally including in a specific embodiment a SP-structure (sulfate or phosphate structure).
Mannose Type Glycans Compositions and Structures Associated with CD133+ Cells
N-Glycan Group CD133+iii)

### High-Mannose Type Neutral N-Glycans

The preferred high-mannose type neutral N-glycans with composition feature N=2 and 5≦H≦9, preferably including H5N2, H9N2, and H8N2.

The preferred structures are according to the formula:

[Mα2]ₙ₁Mα3{[Mα2]ₙ₃Mα6}Mα6{[Mα2]ₙ₆[Mα2]ₙ₇Mα3}Mβ4GNβ4GNyR₂

wherein n1, n3, n6, and n7 are either independently 0 or 1;
y is anomeric linkage structure α and/or β or linkage from derivatized anomeric carbon, and
R₂ is reducing end hydroxyl, chemical reducing end derivative or natural asparagine N-glycoside derivative such as asparagine N-glycosides including aminoacid and/or peptides derived from protein;
[ ] indicates determinant either being present or absent depending on the value of n1, n3, n6, n7; and
{ } indicates a branch in the structure;
M is D-Man, GN is N-acetyl-D-glucosamine, y is anomeric structure or linkage type, preferably beta to Asn.
y is anomeric linkage structure α and/or β or linkage from derivatized anomeric carbon, and
R₂ is reducing end hydroxyl, chemical reducing end derivative or natural asparagine N-glycoside derivative such as asparagine N-glycosides including aminoacid and/or peptides derived from protein;

Preferably the invention is directed to the High mannose type neutral glycans according to the formula with the provision that

all n1, n3, n6, and n7 are 1 (composition is H9N2) or
all n1, n3, n6, and n7 are 0 (composition is H₅N₂) or
one of n1, n3, n6 is 0, and others are 1, and n7 is 1, more preferably n3 is 0 (composition is H8N5).

The preferred structures in this group include:

### Manα2Manα6(Manα2Manα3)Manα6(Manα2Manα2Manα3)Manβ4GlcNAcβ4GlcNAc, or Manα2Manα6(Manα3)Manα6(Manα2Manα2Manα3)Manβ4GlcNAcβ4GlcNAc, Manα6(Manα3)Manα6(Manα3)Manβ4GlcNAcβ4GlcNAc.

Structures and Compositions Associated with Differentiated Mononuclear Cells Cell Types from Blood

The invention revealed novel structures present in higher amount in differentiated mononuclear cells than in corresponding blood derived stem cells.

CD133-Associated N-Glycan Groups CD133-i)-CD133-iii):

The invention revealed 3 groups of glycan compositions and glycan, named CD133-i)-CD133-iii, which are especially characteristic for the CD133 negative cells.

All the groups share common N-glycan core structure according to Formula CCN and the glycan groups are further devided to specific Complex type and Mannose type structures. The differences in the expression are shown in Tables 3 and 4.

Complex Type Glycans Compositions and Structures Associated with CD133− cells N-Glycan Group CD133-i)

### Large Complex-Type Sialylated N-Glycans

The compositions indicate additional N-acetyllactosamine units in comparison to the biantennary N-glycans enriched in CD 133+ cells.

The invention is especially directed to large complex-type sialylated N-glycans with composition feature N≧5 and H≧6,

preferably including S1H6N5F1, S2H6N5F1, S1H7N6F3, S1H7N6F1, S1H6N5, S3H₆N₅F1, S2H7N6F3, S1H6N5F3, S2H6N5F2, and S2H7N6F1. The glycans are further divided to groups of tri-LacNAc-glycans, comprising triantennary glycans, with core composition H6N5 and larger tetra-LacNAc glycans optionally including tetra-antennary glycans with core composition H7N6.

Preferred monosaccharide compositions are

### the Formula

SₖHₙNₚF_{q}

wherein
k is integer from 1 to 3,
n is integer from 6 to 7,
p is integer from 5 to 6, and
q is integer being 0-3,
S is Neu5Ac, G is Neu5Gc, H is hexose selected from group D-Man or D-Gal, N is N-D-acetylhexosamine, preferably GlcNAc or GalNAc, more preferably GlcNAc, and F is L-fucose. The invention is directed compositions with n is 6 and p is 5 for tri LacNAc-structures, and with n is 7 and p is 6 for tetra-LacNAc-structures.

The preferred tri- or tetraantennary structures are according to the formula:

{SAα3/6}ₛ₁LNβ2Mα3{{SAα3/6}ₛ₂LNβ2Mα6}Mβ4GNβ4{Fucα6}GN (I)

with one or two additional branch according to formula

{SAα3/6}ₛ₃LNβ, (IIb)

wherein s1, s2 and s3 are either 0 or 1, independently,
with the provision at least one of s1, s2 or s3 is 1.
LN is N-acetyllactosaminyl also marked as GalβGN, GN is GlcNAc, M is mannosyl-, with the provision that LNβ2M can be further elongated and/or branched with one or several other monosaccharide residues such as galactose, fucose, SA or LN-unit(s) which may be further substituted by SAα-strutures,
is further substituted by one or two β6-, and/or β4-linked additional branches according to the formula IIb,
{ }, indicate groups present in a linear sequence, and { }indicates branching.

The SAα-groups are linked to either 3- or 6-position of neighboring Gal residue or on 6-position of GlcNAc, preferably 3- or 6-position of neighboring Gal residue.

### Preferred Tri-LacNAc and Triantennary Glycans

The invention is especially directed to tri-LacNAc, preferably triantennary N-glycans having compositions S1H6N5F1, S2H6N5F1, S1H6N5, S3H6N5F1, S1H6N5F3, and S2H6N5F2. Presence of triantennary structures was revealed by specific galactosidase digestions. A preferred type of triantennary N-glycans includes one synthesized by MGAT4. The triantennary N-glycan comprises in a preferred embodiment a core fucose residue. The preferred terminal epitopes include Lewis x, sialyl-Lewis x, H- and Lewis y antigens.

The preferred triantennary structures are according to the Formula Tri1

{SAα3/6}ₛ₁LNβ2Mα3{{SAα3/6}ₛ₂LNβ2({SAα3/6}ₛ₃LNβ4)Mα6}Mα4GNβ4{Fucα6})GN,

wherein ( ) indicates branch and other variables are as described above for Formula I.

The invention especially revealed triantennary structures, which are specific for CD133 negative cells.

### Preferred Tetra-LacNAc and Tetraantennary Glycans

The invention is especially directed to tri-LacNAc, preferably triantennary N-glycans having compositions S1H7N6F3, S1H7N6F1, S2H7N6F3, and S2H7N6F1.

Preferred Tetra-LacNAc Including Tetraantennary and/or Polylactosamine Structures

The invention is further directed to monosaccharide compositions and glycan corresponding to monosaccharide compositions S1H7N6F2, and S1H7N₆F3, which were assigned to correspond to tetra-antennary and/or poly-N-acetyllactosamine epitope comprising N-glycans such as ones with terminal GalβGlcNAcβ3GalβGlcNAcβ-, more preferably type 2 structures Galβ4GlcNAcβ3Galβ4GlcNAcβ-.

The preferred tetra-antennary structures are according to the Formula Tet1

{SAα3/6}ₛ₁LNβ2({SAα3/6}ₛ₄LNβ4/6)Mα3{{SAα3/6}ₛ₂LNβ2({SAα3/6}ₛ₃LNβ4)Mα6}Mβ4GNβ4{Fucα6}GN,

wherein ( ) indicates branch, s4 is 0 or 1 and other variables are as described above for Formula I.

### N-Glycan Group CD133-ii)

### Hybrid-Type Sialylated N-Glycans

The invention is especially directed to hybrid-type sialylated N-glycans with composition feature 5≦H≦6, preferably including S1H6N3, S1H5N3, and S1H6N3F1.

Preferred monosaccharide compositions are

### the Formula

S₁HₙN₃F_{q}

wherein
n is integer being 5 or 6, and
q is integer being 0 or 1.

The preferred structures are according to the formula:

NeuNAcα3/6Gaβ4GNβ2Mα3{[Mα3]ₘ₁[(Mα6)]ₘ₂Mα6}Mβ4GNXyR₂,

wherein m1, m2, are either 0 or 1, independently,
z is linkage position to GN being 3 or 4, in a preferred embodiment 4,
R₁ indicates one or two N-acetyllactosamine type elongation groups; NeuAcα3/6 or nothing,
{ } and ( ) indicates branching which may be also present or absent,
other variables are as described in Formula HY1.

More preferably the structures are

NeuNAcα3/6Gaβ4GNβ2Mα3{[Mα3]ₘ₁[(Mα6)ₘ₂Mα6}Mβ4GNXyR₂,

And hex5 structures

NeuNAcα3/6Gaβ4GNβ2Mα3{Mα3Mα6}Mβ4GNXyR₂, and

NeuNAcα3/6Gaβ4GNβ2Mα3{Mα6Mα6}Mβ4GNXyR₂.

### N-Glycan Group CD133-iv)

The Table 4 and FIG. 2 indicate that terminal HexNAc group structures with compositions SH5N5 and SH5N5F are especially specific for the differentiated blood cells, preferably CD133− cells. The invention is directed to the corresponding biantennary N-glycans with two lactosamines and terminal GlcNAc structures comprising GlcNAc substitutions such as bisecting GlcNAc in the N-glycan core Manβ4GlcNAc epitope.

Mannose Type Glycans Compositions and Structures Associated with CD133− cells
N-Glycan Group CD133-iii)

### Low-Mannose Type Neutral N-Glycans

The invention is especially directed to low-mannose type neutral N-glycans with composition feature N=2 and 1≦H≦4,

preferably including H3N2F1, H3N2, H2N2F1, H2N2, H1N2, and H4N2.

Preferred monosaccharide compositions are

### the Formula

HₙN₂F_{q}

wherein
n is integer from 1 to 3,
q is integer being 0 or 1.

The preferred structures are according to the Formula:

[Mα3]ₙ₂{[Mα6)]ₙ₄}[Mα6]ₙ₅{[Mα3]ₙ₈}Mβ4GNβ4[{Fucα6}]ₘGNyR₂

wherein n2, n4, n5, n8, and m are either independently 0 or 1; [ ] indicates determinant being either present or absent depending on the value of n2, n4, n5, n8 and m, { } indicates a branch in the structure;
y and R2 are as indicated for Formula M2.
and with the provision that at least one of n2, n4 and n8 is 0.

Preferred non-fucosylated Low mannose N-glycans are according to the Formula:

Mα6Mβ4GNβ4GNyR₂

Mα3Mβ4GNβ4GNyR₂ and

Mα6{Mβ3}Mβ4GNβ4GNyR₂.

Mα6Mα6{Mα3}Mβ4GNβ4GNyR₂

Mα3Mα6{Mα3}Mβ4GNβ4GNyR₂

### Preferred Individual Structures of Fucosylated Low-Mannose Glycans

Small fucosylated low-mannose structures are especially unusual among known N-linked glycans and form a characteristic glycan group useful for the methods according to the invention, especially analysis and/or separation of cells according to the present invention. These include:

Mβ4GNβ4(Fucα6)GNyR₂

Mα6Mβ4GNβ4(Fucα6)GNyR₂,

Mα3Mβ4GNβ4(Fucα6)GNyR₂,

Mα6Mα6{Mα3)}Mβ4GNβ4(Fucα6)GNyR₂, and

Mα3Mα6{Mα3)}Mβ4GNβ4(Fucα6)GNyR₂.

In a specific embodiment the low mannose glycans include rare structures based on unusual mannosidase degradation

Manα2Manα2Manα3Manβ4GNβ4(Fucα6)₀₋₁GN, and

Manα2Manα3Manβ4GNβ4(Fucα6)₀₋₁GN.

Novel Terminal HexNAc N-Glycan Compositions from Stem Cells

The inventors studied human stem cells. The data revealed a specific group of altering glycan structures referred as terminal HexNAc. The data reveals changes of preferred signals in context of differentiation. The terminal HexNAc structures were assigned to include terminal N-acetylglucosamine structures by cleavage with N-acetylglucosamidase enzymes.

Preferred N-Glycans According to Structural Subgroups with Terminal HexNac

The inventors found that there are differentiation stage specific differences with regard to terminal HexNAc containing N-glycans characterized by the formulae: n_{HexNAc}=n_{Hex}≧5 and n_{dHex}≧1 (group I), or: n_{HexNAc}=n_{Hex}≧5 and n_{dHex}=0 (group II). The present data demonstrated that these glycans were 1) detected in various N-glycan samples isolated from both stem cells, including, cord blood and bone marrow hematopoietic stem cells (CB and BM HSC), and CB HSC further including CD34+, CD133+, and lin− (lineage negative) cells, and cells directly or indirectly differentiated from these cell types; and 2) overexpressed in the analyzed differentiated cells when compared to the corresponding stem cells. There was independent expression between groups I and group II and therefore, the N-glycan structure group determined by the formula n_{HexNAc}=n_{Hex}≧5 is divided into two independently expressed subgroups I and II as described above.

The inventors also found differential expression of glycan signals corresponding to N-glycans Hex₃HexNAc₅ and Hex₃HexNAc₅dHex₁ that have the same compositional feature that the groups II and I above, respectively. Specifically, in analysis of HSC isolated from different sources it was found that Hex₃HexNAc₅dHex, was highly expressed in CD133+ and lin− cells, moderately expressed in all other CB MNC fractions including CD34+ and CD34− cells, and no expression was detected in CD34+ cells isolated from adult peripheral blood.

Based on the known specificities of the biosynthetic enzymes synthesizing N-glycan core α1,6-linked fucose and β1,4-linked bisecting GlcNAc, group II preferably corresponds to bisecting GlcNAc type N-glycans while group I preferentially corresponds to other terminal HexNAc containing N-glycans, preferentially with a branching HexNAc in the N-glycan core structure, more preferentially including structures with a branching GlcNAc in the N-glycan core structure. In a specific embodiment the glycan structures of this group includes core fucosylated bisecting GlcNAc comprising N-glycan, wherein the additional GlcNAc is GlcNAcβ4 linked to Manβ4GlcNAc epitope forming epitope structure GlcNAcβ4Manβ4GlcNAc preferably between the complex type N-glycan branches.

In a preferred embodiment of the present invention, such structures include GlcNAc linked to the 2-position of the β1,4-linked mannose. In a further preferred embodiment of the present invention, such structures include GlcNAc linked to the 2-position of the β1,4-linked mannose as described for LEC14 structure (Raju and Stanley J. Biol Chem (1996) 271, 7484-93), this is specifically preferred embodiment, supported by analysis of gene expression data and glycosyltransferase specificities. In a further preferred embodiment of the present invention, such structures include GlcNAc linked to the 6-position of the β1,4-linked GlcNAc of the N-glycan core as described for LEC14 structure (Raju, Ray and Stanley J. Biol Chem (1995) 270, 30294-302).

The invention is specifically directed to further analysis of the subtypes of the group I glycans comprising structures according to the group I. The invention is further directed to production of specific binding reagents against the N-glycan core marker structures and use of these for analysis of the preferred cancer marker structures. The invention is further directed to the analysis of LEC14 and/or 18 structures by negative recognition by lectins PSA (pisum sativum) or lntil (Lens culinaris) lectin or core Fuc specific monoclonal antibodies, which binding is prevented by the GlcNAcs.

Invention is specifically directed to N-glycan core marker structure, wherein the disaccharide epitope is Manβ4GlcNAc structure in the core structure of N-linked glycan according to the Formula CGN.

The invention is further directed to the N-glycan core marker structure and marker glycan compositions comprising structures of Formula CGN, wherein Manα3/Manα6-residues are elongated to the complex type, especially biantennary structures and n3 is 1 and wherein the Manβ4GlcNAc-epitope comprises the GlcNAc substitutions.

The invention is further directed to the N-glycan core marker structure and marker glycan compositions comprising structures of Formula CGN, wherein Manα3/Manα6-residues are elongated to the complex type, especially biantennary structures and n3 is 1 and wherein the Manβ4GlcNAc-epitope comprises between 1-8% of the GlcNAc substitutions.

The invention is further directed to the N-glycan core marker structure and marker glycan compositions comprising structures of Formula CGN, wherein the structure is selected from the group:

[GlcNAcβ2Manα3](GlcNAcβ2Manα6) Manβ4GlcNAcβ4(Fucα6)ₙ₃GlcNAcxR, [Galβ4GlcNAcβ2Manα3](Galβ4GlcNAcβ2Manα6)Manβ4GlcNAcβ4(Fucα6)ₙ₃GlcNAcxR, and sialylated variants thereof when SA is α3 and or α6-linked to one or two Gal residues and Manβ4 or GlcNAcβ4 is substituted by GlcNAc.

The invention is further directed to the N-glycan core marker structure and marker glycan compositions comprising of Formula CGN, wherein the Manβ4GlcNAc-epitope comprises and the GlcNAc residue is β2-linked to Manβ4 forming epitope GlcNAcβ2Manβ4.

The invention is further directed to the N-glycan core marker structure and marker glycan compositions comprising of Formula CGN, wherein the Manβ4GlcNAc-epitope comprises and the GlcNAc residue is 6-linked to GlcNAc of the epitope forming epitope Manβ4(GlcNAc6)GlcNAc.

The invention is further directed to the N-glycan core marker structure and marker glycan compositions comprising of Formula CGN, wherein the Manβ4GlcNAc-epitope comprises and the GlcNAc residue is 4-linked to GlcNAc of the epitope forming epitope GlcNAcβ4Manβ4GlcNAc.

Glycomes—Novel Glycan Mixtures from Stem Cells

The present invention revealed novel glycans of different sizes from stem cells. The stem cells contain glycans ranging from small oligosaccharides to large complex structures. The analysis reveals compositions with substantial amounts of numerous components and structural types. Previously the total glycomes from these rare materials has not been available and nature of the releasable glycan mixtures, the glycomes, of stem cells has been unknown.

The invention revealed that the glycan structures on cell surfaces vary between the various populations of the early human cells, the preferred target cell populations according to the invention. It was revealed that the cell populations contained specifically increased “reporter structures”.

The glycan structures on cell surfaces in general have been known to have numerous biological roles. Thus the knowledge about exact glycan mixtures from cell surfaces is important for knowledge about the status of cells. The invention revealed that multiple conditions affect the cells and cause changes in their glycomes. The present invention revealed novel glycome components and structures from human stem cells. The invention revealed especially specific terminal Glycan epitopes, which can be analyzed by specific binder molecules.

Recognition of Structures from Glycome Materials and on Cell Surfaces by Binding Methods

The present invention revealed that beside the physicochemical analysis by NMR and/or mass spectrometry several methods are useful for the analysis of the structures. The invention is especially directed to a method:
i) Recognition by molecules binding glycans referred as the binders
These molecules bind glycans and include property allowing observation of the binding such as a label linked to the binder. The preferred binders include
   a) Proteins such as antibodies, lectins and enzymes
   b) Peptides such as binding domains and sites of proteins, and synthetic library derived analogs such as phage display peptides
   c) Other polymers or organic scaffold molecules mimicking the peptide materials

The peptides and proteins are preferably recombinant proteins or corresponding carbohydrate recognition domains derived thereof, when the proteins are selected from the group of monoclonal antibody, glycosidase, glycosyl transferring enzyme, plant lectin, animal lectin or a peptide mimetic thereof, and wherein the binder may include a detectable label structure.

The genus of enzymes in carbohydrate recognition is continuous to the genus of lectins (carbohydrate binding proteins without enzymatic activity).

a) Native glycosyltransferases (Rauvala et al.(1983) PNAS (USA) 3991-3995) and glycosidases (Rauvala and Hakomori (1981) J. Cell Biol. 88, 149-159) have lectin activities.

b) The carbohydrate binding enzymes can be modified to lectins by mutating the catalytic amino acid residues (see WO9842864; Aalto J. et al. Glycoconjugate J. (2001, 18(10); 751-8; Mega and Hase (1994) BBA 1200 (3) 331-3).

c) Natural lectins, which are structurally homologous to glycosidases are also known indicating the continuity of the genus enzymes and lectins (Sun, Y-J. et al. J. Biol. Chem. (2001) 276 (20) 17507-14).

The genus of the antibodies as carbohydrate binding proteins without enzymatic activity is also very close to the concept of lectins, but antibodies are usually not classified as lectins.

Obviousness of the Peptide Concept and Continuity with the Carbohydrate Binding Protein Concept

It is further realized that proteins consist of peptide chains and thus the recognition of carbohydrates by peptides is obvious. E.g. it is known in the art that peptides derived from active sites of carbohydrate binding proteins can recognize carbohydrates (e.g. Geng J-G. et al (1992) J. Biol. Chem. 19846-53).

As described above antibody fragment are included in description and genetically engineed variants of the binding proteins. The obvious geneticall engineered variants would included truncated or fragment peptides of the enzymes, antibodies and lectins.

### Revealing Cell or Differentiation and Individual Specific Terminal Variants of Structures

The invention is directed use the glycomics profiling methods for the revealing structural features with on-off changes as markers of specific differentiation stage or quantitative difference based on quantitative comparison of glycomes. The individual specific variants are based on genetic variations of glycosyltransferases and/or other components of the glycosylation machinery preventing or causing synthesis of individual specific structure.

### Terminal Structural Epitopes

We have previously revealed glycome compositions of human glycomes, here we provide structural terminal epitopes useful for the characterization of stem cell glycomes, especially by specific binders.

The examples of characteristic altering terminal structures includes expression of competing terminal epitopes created as modification of key homologous core Galβ-epitopes, with either the same monosaccharides with difference in linkage position Galβ3GlcNAc, and analogue with either the same monosaccharides with difference in linkage position Galβ4GlcNAc; or the with the same linkage but 4-position epimeric backbone Galβ3GalNAc. These can be presented by specific core structures modifying the biological recognition and function of the structures. Another common feature is that the similar Galβ-structures are expressed both as protein linked (O- and N-glycan) and lipid linked (glycolipid structures). As an alternative for α2-fucosylation the terminal Gal may comprise NAc group on the same 2 position as the fucose. This leads to homologous epitopes GalNAcβ4GlcNAc and yet related GalNAcβ3Gal-structure on characteristic special glycolipid according to the invention.

The invention is directed to novel terminal disaccharide and derivative epitopes from human stem cells, preferably from human embryonal stem cells or adult stem cells, when these are not hematopoietic stem cells, which are preferably mesenchymal stem cells. It should realized that glycosylations are species, cell and tissue specific and results from cancer cells usually differ dramatically from normal cells, thus the vast and varying glycosylation data obtained from human embryonal carcinomas are not actually relevant or obvious to human embryonal stem cells (unless accidentally appeared similar). Additionally the exact differentiation level of teratocarcinomas cannot be known, so comparison of terminal epitope under specific modification machinery cannot be known. The terminal structures by specific binding molecules including glycosidases and antibodies and chemical analysis of the structures.

The present invention reveals group of terminal Gal(NAc)β1-3/4Hex(NAc) structures, which carry similar modifications by specific fucosylation/NAc-modification, and sialylation on corresponding positions of the terminal disaccharide epitopes. It is realized that the terminal structures are regulated by genetically controlled homologous family of fucosyltransferases and sialyltransferases. The regulation creates a characteristic structural patterns for communication between cells and recognition by other specific binder to be used for analysis of the cells. The key epitopes are presented in the TABLE 15. The data reveals characteristic patterns of the terminal epitopes for each types of cells, such as for example expression on hESC-cells generally much Fucα-structures such as Fucα2-structures on type 1 lactosamine (Galβ3GlcNAc), similarly β3-linked core I Galβ3GlcNAcα, and type 4 structure which is present on specific type of glycolipids and expression of α3-fucosylated structures, while α6-sialic on type II N-acetylalactosamine appear on N-glycans of embryoid bodies and st3 embryonal stem cells. E.g. terminal type lactosamine and poly-lactosamines differentiate mesenchymal stem cells from other types. The terminal Galb-information is preferably combined with information about

The invention is directed especially to high specificity binding molecules such as monoclonal antibodies for the recognition of the structures.

The structures can be presented by Formula T1. the formula describes first monosaccharide residue on left, which is a β-D-galactopyranosyl structure linked to either 3 or 4-position of the α- or β-D-(2-deoxy-2-acetamido)galactopyranosyl structure, when R₅ is OH, or β-D-(2-deoxy-2-acetamido)glucopyranosyl, when R₄ comprises O—. The unspecified stereochemistry of the reducing end in formulas T1 and T2 is indicated additionally (in claims) with curved line. The sialic acid residues can be linked to 3 or 6-position of Gal or 6-position of GlcNAc and fucose residues to position 2 of Gal or 3- or 4-position of GlcNAc or position 3 of Glc. The invention is directed to Galactosyl-globoside type structures comprising terminal Fucα2-revealed as novel terminal epitope Fucα2Galβ3GalNAc, or Galβ3GalNAcβGalα3-comprising isoglobotructures revealed from the embryonal type cells.

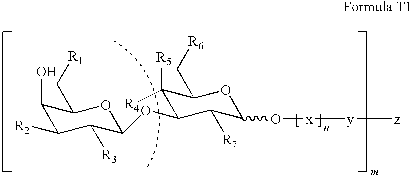

wherein
X is linkage position
R₁, R₂, and R₆ are OH or glycosidically linked monosaccharide residue Sialic acid, preferably Neu5Acα2 or Neu5Gc α2, most preferably Neu5Acα2 or
R₃, is OH or glycosidically linked monosaccharide residue Fucα1 (L-fucose) or N-acetyl (N-acetamido, NCOCH₃);
R₄, is H, OH or glycosidically linked monosaccharide residue Fucα1 (L-fucose),
R₅ is OH, when R₄ is H, and R₅ is H, when R₄ is not H;

### R7 is N-acetyl or OH

X is natural oligosaccharide backbone structure from the cells, preferably N-glycan, O-glycan or glycolipid structure; or X is nothing, when n is 0,
Y is linker group preferably oxygen for O-glycans and O-linked terminal oligosaccharides and glycolipids and N for N-glycans or nothing when n is 0;
Z is the carrier structure, preferably natural carrier produced by the cells, such as protein or lipid, which is preferably a ceramide or branched glycan core structure on the carrier or H;
The arch indicates that the linkage from the galactopyranosyl is either to position 3 or to position 4 of the residue on the left and that the R4 structure is in the other position 4 or 3;
n is an integer 0 or 1, and m is an integer from 1 to 1000, preferably 1 to 100, and most preferably 1 to 10 (the number of the glycans on the carrier),
With the provisions that one of R2 and R3 is OH or R3 is N-acetyl,
R6 is OH, when the first residue on left is linked to position 4 of the residue on right:
X is not Galα4Galβ4Glc, (the core structure of SSEA-3 or 4) or R3 is Fucosyl
R7 is preferably N-acetyl, when the first residue on left is linked to position 3 of the residue on right:
Preferred terminal β3-linked subgroup is represented
by Formula T2 indicating the situation, when the first residue on the left is linked to the 3 position with backbone structures Gal(NAc)β3 Gal/GlcNAc.

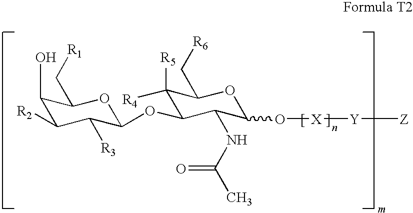

Wherein the variables including R₁ to R₇

are as described for T1

Preferred terminal β4-linked subgroup is represented by the Formula 3

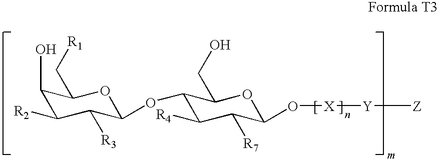

Wherein the variables including R1 to R4 and R7

are as described for T1 with the provision that
R₄, is OH or glycosidically linked monosaccharide residue Fucα1 (L-fucose),

Alternatively the epitope of the terminal structure can be represented by Formulas T4 and T5

Core Galβ-epitopes formula T4:

Galβ1-xHex(NAc)ₚ,

x is linkage position 3 or 4,

### and Hex is Gal or Glc

with provision
p is 0 or 1
when x is linkage position 3, p is 1 and HexNAc is GlcNAc or GalNAc,
and when x is linkage position 4, Hex is Glc.

The core Galβ1-3/4 epitope is optionally substituted to hydroxyl

by one or two structures SAα or Fucα, preferably selected from the group
Gal linked SAα3 or SAα6 or Fucα2, and
Glc linked Fucα3 or GlcNAc linked Fucα3/4.

[Mα]ₘGalβ1-x[Nα].Hex(NAc)ₚ, Formula T5

wherein m, n and p are integers 0, or 1, independently

### Hex is Gal or Glc,

X is linkage position
M and N are monosaccharide residues being
independently nothing (free hydroxyl groups at the positions) and/or
SA which is Sialic acid linked to 3-position of Gal or/and 6-position of HexNAc and/or
Fuc (L-fucose) residue linked to 2-position of Gal
and/or 3 or 4 position of HexNAc, when Gal is linked to the other position (4 or 3),
and HexNAc is GlcNAc, or 3-position of Glc when Gal is linked to the other position (3), with the provision that sum of m and n is 2
preferably m and n are 0 or 1, independently.

The exact structural details are essential for optimal recognition by specific binding molecules designed for the analysis and/or manipulation of the cells.

The terminal key Galβ-epitopes are modified by the same modification monosaccharides NeuX (X is 5 position modification Ac or Gc of sialic acid) or Fuc, with the same linkage type alfa (modifying the same hydroxyl-positions in both structures. NeuXα3, Fucα2 on the terminal Galβ of all the epitopes and

NeuXα6 modifying the terminal Galβ of Galβ4GlcNAc, or HexNAc, when linkage is 6 competing or Fucα modifying the free axial primary hydroxyl left in GlcNAc (there is no free axial hydroxyl in GalNAc-residue).

The preferred structures can be divided to preferred Galβ1-3 structures analogously to T2,

[Mα]ₘGalβ1-3[Nα]ₙHexNAc, Formula T6

Wherein the variables are as described for T5.

The preferred structures can be divided to preferred Galβ1-4 structures analogously to T4,

[Mα]mGalβ1-4[Nα]ₙGlc(NAc)ₚ, Formula T7

Wherein the variables are as described for T5.

These are preferred type II N-acetyllactosamine structures and related lactosylderivatives, in a preferred embodiment p is 1 and the structures includes only type 2 N-acetyllactosamines. The invention revealed that the these are very useful for recognition of specific subtypes of stem cells, preferably mesenchymal stem cells, or embryonal type stem cells or differentiated variants thereof (tissue type specifically differentiated mesenchymal stem cells or various stages of embryonal stem cells). It is notable that various fucosyl- and or sialic acid modification created characteristic pattern for the stem cell type.

### Preferred Type I and Type II N-Acetyllactosamine Structures

The preferred structures can be divided to preferred type one (I) and type two (II) N-acetyllactosamine structures comprising oligosaccharide core sequence Galβ1-3/4 GlcNAc structures analogously to T4,

[Mα]ₘGalβ1-3/4[Nα]ₙGlcNAc, Formula T8

Wherein the variables are as described for T5.

The preferred structures can be divided to preferred Galβ1-3 structures analogously to T8,

[Mα]ₘGalβ1-3[Nα]ₙGlcNAc Formula T9

Wherein the variables are as described for T5.

These are preferred type I N-acetyllactosamine structures. The invention revealed that the these are very useful for recognition of specific subtypes of stem cells, preferably mesenchymal stem cells, or embryonal type stem cells or differentiated variants thereof (tissue type specifically differentiated mesenchymal stem cells or various stages of embryonal stem cells). It is notable that various fucosyl- and or sialic acid modification created characteristic pattern for the stem cell type.

The preferred structures can be divided to preferred Galβ1-4GlcNAc core sequence comprising structures analogously to T8,

[Mα]mGalβ1-4[Nα]ₙGlcNAc Formula T10

Wherein the variables are as described for T5.

These are preferred type II N-acetyllactosamine structures. The invention revealed that the these are very useful for recognition of specific subtypes of stem cells, preferably mesenchymal stem cells, or embryonal type stem cells or differentiated variants thereof (tissue type specifically differentiated mesenchymal stem cells or various stages of embryonal stem cells).

It is notable that various fucosyl- and or sialic acid modificationally N-acetyllactosamine structures create especially characteristic pattern for the stem cell type. The invention is further directed to use of combinations binder reagents recognizing at least two different type I and type II acetyllactosamines including at least one fucosylated or sialylated variant and more preferably at least two fucosylated variants or two sialylated variants

Preferred structures comprising terminal Fucα2/3/4-structures

The invention is further directed to use of combinations binder reagents recognizing:
a) type I and type II acetyllactosamines and their fucosylated variants, and in a preferred embodiment
b) non-sialylated fucosylated and even more preferably
c) fucosylated type I and type II N-acetyllactosamine structures preferably comprising Fucα2-terminal and/or Fucα3/4-branch structure and even more preferably
d) fucosylated type I and type II N-acetyllactosamine structures preferably comprising Fucα2-terminal
for the methods according to the invention of various stem cells especially embryonal type and mesenchymal stem cells and differentiated variants thereof.

Preferred subgroups of Fucα2-structures includes monofucosylated H type and H type II structures, and difucosylated Lewis b and Lewis y structures.

Preferred subgroups of Fucα3/4-structures includes monofucosylated Lewis a and Lewis x structures, sialylated sialyl-Lewis a and sialyl-Lewis x-structures and difucosylated Lewis b and Lewis y structures.

Preferred type II N-acetyllactosamine subgroups of Fucα3-structures includes monofucosylated Lewis x structures, and sialyl-Lewis x-structures and Lewis y structures.

Preferred type I N-acetyllactosamine subgroups of Fucα4-structures includes monofucosylated Lewis a sialyl-Lewis a and difucosylated Lewis b structures.

The invention is further directed to use of at least two differently fucosylated type one and or and two N-acetyllactosamine structures preferably selected from the group monofucosylated or at least two difucosylated, or at least one monofucosylated and one difucosylated structures.

The invention is further directed to use of combinations binder reagents recognizing fucosylated type I and type II N-acetyllactosamine structures together with binders recognizing other terminal structures comprising Fucα2/3/4-comprising structures, preferably Fucα2-terminal structures, preferably comprising Fucα2Galβ3GalNAc-terminal, more preferably Fucα2Galβ3GalNAcα/β and in especially preferred embodiment antibodies recognizing Fucα2Galβ3GalNAcβ-preferably in terminal structure of Globo- or isoglobotype structures.

### Preferred Globo- and Ganglio Core Type-Structures

The invention is further directed to general formula comprising globo and gangliotype Glycan core structures according to formula

[M]ₘGalβ1-x[Nα]ₙHex(NAc)ₚ, Formula T1

wherein m, n and p are integers 0, or 1, independently Hex is Gal or Glc, X is linkage position;
M and N are monosaccharide residues being
independently nothing (free hydroxyl groups at the positions) and/or
SAα which is Sialic acid linked to 3-position of Gal or/and 6-position of HexNAc
Galα linked to 3 or 4-position of Gal, or
GalNAcβ linked to 4-position of Gal and/or
Fuc (L-fucose) residue linked to 2-position of Gal
and/or 3 or 4 position of HexNAc, when Gal is linked to the other position (4 or 3),
and HexNAc is GlcNAc, or 3-position of Glc when Gal is linked to the other position (3),
with the provision that sum of m and n is 2
preferably m and n are 0 or 1, independently, and
with the provision that when M is Galα then there is no sialic acid linked to Galβ1, and
n is 0 and preferably x is 4.
with the provision that when M is GalNAcβ, then there is no sialic acid α6-linked to Galβ1, and n is 0 and x is 4.

The invention is further directed to general formula comprising globo and gangliotype Glycan core structures according to formula

[M][SAα3]ₙGalβ1-4Glc(NAc)ₚ Formula T12

wherein n and p are integers 0, or 1, independently
M is Galα linked to 3 or 4-position of Gal, or GalNAcβ linked to 4-position of Gal
and/or SAα is Sialic acid branch linked to 3-position of Gal
with the provision that when M is Galα then there is no sialic acid linked to Galβ1 (n is 0).

The invention is further directed to general formula comprising globo and gangliotype Glycan core structures according to formula

[M][SAα]ₙGalβ1-4Glc, Formula T13

wherein n and p are integer 0, or 1, independently
M is Galα linked to 3 or 4-position of Gal, or
GalNAcβ linked to 4-position of Gal and/or
SAα which is Sialic acid linked to 3-position of Gal
with the provision that when M is Galα then there is no sialic acid linked to Galβ1 (n is 0).

The invention is further directed to general formula comprising globo type Glycan core structures according to formula

Galα3/4Galβ1-4Glc. Formula T14

The preferred Globo-type structures includes Galα3/4Galβ1-4Glc, GalNAcβ3Galα3/4Galβ4Glc, Galα4Galβ4Glc (globotriose, Gb3), Galα3Galβ4Glc (isoglobotriose), GalNAcβ3Galα4Galβ4Glc (globotetraose, Gb4 (or G14)), and Fucα2Galβ3GalNAcβ3Galα3/4Galβ4Glc. or

when the binder is not used in context of non-differentiated embryonal or mesenchymal stem cells or the binder is used together with another preferred binder according to the invention, preferably an other globo-type binder the preferred binder targets further includes Galβ3GalNAcβ3Galα4Galβ4Glc (SSEA-3 antigen) and/or NeuAcα3Galβ3GalNAcβ3Galα4Galβ4Glc (SSEA-4 antigen) or terminal non-reducing end di or trisaccharide epitopes thereof.

The preferred globotetraosylceramide antibodies does not recognize non-reducing end elongated variants of GalNAcβ3Galα4Galβ4Glc. The antibody in the examples has such specificity as

The invention is further directed to binders for specific epitopes of the longer oligosaccharide sequences including preferably NeuAcα3Galβ3GalNAc, NeuAcα3Galβ3GalNAcβ, NeuAcα3Galβ3GalNAcβ3Galα4Gal when these are not linked to glycolipids and novel fucosylated target structures:

### Fucα2Galβ3GalNAcβ3 Galα3/4GalβFucα2Galβ3 GalNAcβ3Galα, Fucα2Galβ3 GalNAcβ3Gal, Fucα2Galβ3 GalNAcβ3, and Fucα2Galβ3GalNAc.

The invention is further directed to general formula comprising globo and gangliotype Glycan core structures according to formula

[GalNAcβ4][SAα]ₙGalβ1-4Glc, Formula T15

wherein n and p are integer 0, or 1, independently GalNAcβ linked to 4-position of Gal and/or SAα which is Sialic acid branch linked to 3-position of Gal.

The preferred Ganglio-type structures includes GalNAcβ4Galβ1-4Glc, GalNAcβ4[SAα3]Galβ1-4Glc, and Galβ3GalNAcβ4[SAα3]Galβ1-4Glc.

The preferred binder target structures further include glycolipid and possible glycoprotein conjugates of the preferred oligosaccharide sequences. The preferred binders preferably specifically recognizes at least di- or trisaccharide epitope

### GalNAcα-Structures

The invention is further directed to recognition of peptide/protein linked GalNAcα-structures according to the Formula T16:[SAα6]mGalNAcα[Ser/Thr]ₙ-[Peptide]ₚ, wherein m, n and p are integers 0 or 1, independently,

wherein SA is sialic acid preferably NeuAc₁Ser/Thr indicates linking serine or threonine residues, Peptide indicates part of peptide sequence close to linking residue,

with the provisio that either m or n is 1.

Ser/Thr and/or Peptide are optionally at least partiallt necessary for recognition for the binding by the binder. It is realized that when Peptide is included in the specificity, the antibody have high specificity involving part of a protein structure. The preferred antigen sequences of sialyl-Tn: SAα6GalNAcα, SAα6GalNAcαSer/Thr, and SAα6GalNAcαSer/Thr-Peptide and Tn-antigen: GalNAcαSer/Thr, and GalNAcαSer/Thr-Peptide. The invention is further directed to the use of combinations of the GalNAcα-structures and combination of at least one GalNAcα-structure with other preferred structures.

### Combinations of Preferred Binder Groups

The present invention is especially directed to combined use of at least

a) fucosylated, preferably α2/3/4-fucosylated structures and/or b) globo-type structures and/or c) GalNAcα-type structures. It is realized that using a combination of binders recognizing structures involving different biosynthesis and thus having characteristic binding profile with a stem cell population. More preferably at least one binder for a fucosylated structure and globostructures, or fucosylated structure and GalNAcα-type structure is used, most preferably fucosylated structure and globostructure are used.

### Fucosylated and Non-Modified Structures

The invention is further directed to the core disaccharide epitope structures when the structures are not modified by sialic acid (none of the R-groups according to the Formulas T1-T3 or M or N in formulas T4-T7 is not sialic acid.

The invention is in a preferred embodiment directed to structures, which comprise at least one fucose residue according to the invention. These structures are novel specific fucosylated terminal epitopes, useful for the analysis of stem cells according to the invention. Preferably native stem cells are analyzed.

The preferred fucosylated structures include novel α3/4fucosylated markers of human stem cells such as (SAα3)₀ₒᵣ₁Galβ3/4(Fucα4/3)GlcNAc including Lewis x and sialylated variants thereof.

Among the structures comprising terminal Fucα1-2 the invention revealed especially useful novel marker structures comprising Fucα2Galβ3GalNAcα/β and Fucα2Galβ3(Fucα4)₀ₒᵣ₁GlcNAcβ, these were found useful studying embryonal stem cells. A especially preferred antibody/binder group among this group is antibodies specific for Fucα2Galβ3GlcNAcβ, preferred for high stem cell specificity. Another preferred structural group includes Fucα2Gal comprising glycolipids revealed to form specific structural group, especially interesting structure is globo-H-type structure and glycolipids with terminal Fucα2Galβ3GalNAcβ, preferred with interesting biosynthetic context to earlier speculated stem cell markers.

Among the antibodies recognizing Fucα2Galβ4GlcNAc, substantial variation in binding was revealed likely based on the carrier structures, the invention is especially directed to antibodies recognizing this type of structures, when the specificity of the antibody is similar to the ones binding to the embryonal stem cells as shown in Example 13 with fucose recognizing antibodies.

The invention is preferably directed to antibodies recognizing Fucα2Galβ4GlcNAcβ on N-glycans, revealed as common structural type in terminal epitope Table 15. In a separate embodiment the antibody of the non-binding clone is directed to the recognition of the feeder cells.

The preferred non-modified structures includes Galβ4Glc, Galβ3GlcNAc, Galβ3GalNAc, Galβ4GlcNAc, Galβ3GlcNAcβ, Galβ3GalNAcβ/α, and Galβ4GlcNAcβ. These are preferred novel core markers characteristics for the various stem cells. The structure Galβ3GlcNAc is especially preferred as novel marker observable in hESC cells. Preferably the structure is carried by a glycolipid core structure according to the invention or it is present on an O-glycan. The non-modified markers are preferred for the use in combination with at least one fucosylated or/and sialylated structure for analysis of cell status.

Additional preferred non-modified structures includes GalNAcβ-structures includes terminal LacdiNAc, GalNAcβ4GlcNAc, preferred on N-glycans and GalNAcβ3Gal GalNAcβ3Gal present in globoseries glycolipids as terminal of globotetraose structures.

Among these characteristic subgroup of Gal(NAc)β3-comprising Galβ3GlcNAc, Galβ3GalNAc, Galβ3GlcNAcβ, Galβ3GalNAcβ/α, and GalNAcβ3Gal GalNAcβ3Gal and

the characteristic subgroup of Gal(NAc)β4-comprising Galβ4Glc, Galβ4GlcNAc, and Galβ4GlcNAc are separately preferred.

### Preferred Sialylated Structures

The preferred sialylated structures includes characteristic SAα3Galβ-structures SAα3Galβ4Glc, SAα3Galβ3GlcNAc, SAα3Galβ3 GalNAc, SAα3Galβ4GlcNAc, SAα3Galβ3GlcNAcβ, SAα3Galβ3GalNAcβ/α, and SAα3Galβ4GlcNAcβ; and biosynthetically partially competing SAα6Galβ-structures SAα6Galβ4Glc, SAα6Galβ4Glcβ; SAα6Galβ4GlcNAc and SAα6Galβ4GlcNAc,; and disialo structures SAα3Galβ3 (SAα6)GalNAcβ/α,

The invention is preferably directed to specific subgroup of Gal(NAc)β3-comprising SAα3Galβ3GlcNAc, SAα3Galβ3 GalNAc, SAα3Galβ4GlcNAc, SAα3Galβ3GlcNAcβ, SAα3Galβ3GalNAcβ/α and SAα3Galβ3 (SAα6)GalNAcβ/α, and Gal(NAc)β4-comprising sialylated structures. SAα3Galβ4Glc, and SAα3Galβ4GlcNAcβ; and SAα6Galβ4Glc, SAα6Galβ4Glcβ; SAα6Galβ4GlcNAc and SAα6Galβ4GlcNAcβ

These are preferred novel regulated markers characteristics for the various stem cells.

Use Together with a Terminal ManαMan-Structure

The terminal non-modified or modified epitopes are in preferred embodiment used together with at least one ManoxMan-structure. This is preferred because the structure is in different N-glycan or glycan subgroup than the other epitopes.

### Preferred Structural Groups for Hematopoietic Stem Cells.

The present invention provides novel markers and target structures and binders to these for especially embryonic and adult stem cells, when these cells are not heamtopoietic stem cells. From hematopoietic CD34+ cells certain terminal structures such as terminal sialylated type two N-acetyllactosamines such as NeuNAcα3Galβ4GlcNAc (Magnani J. U.S. Pat. No. 6,362,010) has been suggested and there is indications for low expression of Slex type structures NeuNAcα3Galβ4(Fucα3)GlcNAc (Xia L et al Blood (2004) 104 (10) 3091-6). The invention is also directed to the NeuNAcα3Galβ4GlcNAc non-polylactosamine variants separately from specific characteristic O-glycans and N-glycans. The invention further provides novel markers for CD133+ cells and novel hematopoietic stem cell markers according to the invention, especially when the structures does not include NeuNAcα3Galβ4(Fucα3)₀₋₁GlcNAc. Preferably the hematopoietic stem cell structures are non-sialylated, fucosylated structures Galβ1-3-structures according to the invention and even more preferably type 1 N-acetyllactosamine structures Galβ3GlcNAc or separately preferred Galβ3GalNAc based structures.

### Core Structures of the Terminal Epitopes

It is realized that the target epitope structures are most effectively recognized on specific N-glycans, O-glycan, or on glycolipid core structures.

### Elongated Epitopes—Next Monosaccharide/Structure on the Reducing End of the Epitope

The invention is especially directed to optimized binders and production thereof, when the binding epitope of the binder includes the next linkage structure and even more preferably at least part of the next structure (monosaccharide or aminoacid for O-glycans or ceramide for glycaolipid) on the reducing side of the target epitope. The invention has revealed the core structures for the terminal epitopes as shown in the Examples and ones summarized in Table 15.

It is realized that antibodies with longer binding epitopes have higher specificity and thus will recognize that desired cells or cell derived components more effectively. In a preferred embodiment the antibodies for elongated epitopes are selected for effective analysis of embryonal type stem cells.

The invention is especially directed to the methods of antibody selection and optionally further purification of novel antibodies or other binders using the elongated epitopes according to the invention. The preferred selection is performed by contacting the glycan structure (synthetic or isolated natural glycan with the specific sequence) with a serum or an antibody or an antibody library, such as a phage display library. Data about these methods are well known in the art and available from internet for example by searching pubmed-medical literature database (www.ncbi.nlm.nih.gov/entrez) or patents e.g. in espacenet (fi.espacenet.com).

The specific antibodies are especially preferred for the use of the optimized recognition of the glycan type specific terminal structures as shown in the Examples and ones summarized in the Table 15.

It is further realized that part of the antibodies according to the invention and shown in the examples have specificity for the elongated epitopes. The inventors found out that for example Lewis x epiotpe can be recognized on N-glycan by certain terminal Lewis x specific antibodies, but not so effectively or at all by antibodies recognizing Lewis xβ1-3Gal present on poly-N-acetyllactosamines or neolactoseries glycolipids.

### N-Glycans

The invention is especially directed to recognition of terminal N-glycan epitopes on biantennary N-glycans. The preferred non-reducing end monosaccharide epitope for N-glycans comprise β2Man and its reducing end further elongated variants

### β2Man, β2Manα, β2Manα3, and β2Manα6

The invention is especially directed to recognition of lewis x on N-glycan by N-glycan Lewis x specific antibody described by Ajit Varki and colleagues Glycobiology (2006) Abstracts of Glycobiology society meeting 2006 Los Angeles, with possible implication for neuronal cells, which are not directed (but disclaimed) with this type of antibody by the present invention. Invention is further directed to antibodies with specificity of type 2 N-acetyllactosamine β2Man recognizing biantennary N-glycan directed antibody as described in Ozawa H et al (1997) Arch Biochem Biophys 342, 48-57.

### O-Glycans, Reducing End Elongated Epitopes

The invention is especially directed to recognition of terminal O-glycan epitopes as terminal core I epitopes and as elongated variants of core I and core II O-glycans.

The preferred non-reducing end monosaccharide epitope for O-glycans comprise:

a) Core I epitopes linked to αSer/Thr-[Peptide]₀₋₁,
wherein Peptide indicates peptide which is either present or absent. The invention is preferably
b) Preferred core II-type epitopes
R1β6[R2β3Galβ3]ₙGalNAcαSer/Thr, wherein n is =or 1 indicating possible branch in the structure and R1 and R2 are preferred positions of the terminal epitopes, R1 is more preferred
c) Elongated Core I epitope
β3Gal and its reducing end further elongated variants β3Galβ3GalNAcα, β3Galβ3GalNAcαSer/Thr

O-glycan core I specific and ganglio/globotype core reducing end epitopes have been described in (Saito S et al. J Biol Chem (1994) 269, 5644-52), the invention is preferably directed to similar specific recognition of the epitopes according to the invention.

O-glycan core II sialyl-Lewis x specific antibody has nbeen described in Walcheck B et al. Blood (2002) 99, 4063-69.

Peptide specificity including antibodies for recognition of O-glycans includes mucin specific antibodies further recognizing GalNAcalfa (Tn) or Galb3GalNAcalfa (T/TF) structures (Hanisch F-G et al (1995) cancer Res. 55, 4036-40; Karsten U et al. Glycobiology (2004) 14, 681-92;

### Glycolipid Core Structures

The invention is furthermore directed to the recognition of the structures on lipid structures. The preferred lipid corestructures include:
a) βCer (ceramide) for Galβ4Glc and its fucosyl or sialyl derivatives
b) β3/6Gal for type I and type II N-acetyllactosamines on lactosyl Cer-glycolipids, preferred elongated variants includes β3/6[Rβ6/3]ₙGalβ, β3/6[RP6/3]ₙGalβ4 and β3/6[Rβ6/3]ₙGalβ4Glc, which may be further banched by another lactosamine residue which may be partially recognized as larger epitope and n is 0 or 1 indicating the branch, and R1 and R2 are preferred positions of the terminal epitopes. Preferred linear (non-branched) common structures include β3Gal, β3Galβ, β3Galβ4 and β3Galβ4Glc
c) α3/4Gal, for globoseries epitopes, and elongated variants α3/4Galβ, α3/4Galβ4Glc preferred globoepitopes have elongated epitopes α4Gal, α4Galβ, α4Galβ4Glc, and preferred isogloboepitopes have elongated epitopes α3Gal, α3Galβ, α3Galβ4Glc
d) β4Gal for ganglio-series epitopes comprising, and preferred elongated variants include β4Galβ, and β4Galβ4Glc

O-glycan core specific and ganglio/globotype core reducing end epitopes have been described in (Saito S et al. J Biol Chem (1994) 269, 5644-52), the invention is preferably directed to similar specific recognition of the epitopes according to the invention.

### Poly-N-Acetyllactosamines

Poly-N-acetyllactosamine backbone structures on O-glycans, N-glycans, or glycolipids comprise characteristic structures similar to lactosyl(cer) core structures on type I (lactoseries) and type II (neolacto) glycolipids, but terminal epitopes are linked to another type I or type II N-acetyllactosamine, which may from a branched structure. Preferred elongated epitopes include: β3/6Gal for type I and type II N-acetyllactosamines epitope, preferred elongated variants includes R1β3/6[R2β6/3]ₙGalβ, R1β3/6[R2β6/3]ₙGalβ3/4 and R1β3/6[R2β6/3],Galβ3/4GlcNAc, which may be further banched by another lactosamine residue which may be partially recognized as larger epitope and n is 0 or 1 indicating the branch, and R1 and R2 are preferred positions of the terminal epitopes. Preferred linear (non-branched) common structures include β3Gal, β3Galβ, β3Galβ4 and β3GalβB4GlcNAc.

Numerous antibodies are known for linear (i-antigen) and branched poly-N-acetyllactosamines (1-antigen), the invention is further directed to the use of the lectin PWA for recognition of 1-antigens. The inventors revealed that poly-N-acetyllactosamines are characteristic structures for specific types of human stem cells. Another preferred binding regent, enzyme endo-beta-galactosidase was used for characterization poly-N-acetyllactosamines on glycolipids and on glycoprotein of the stem cells. The enzyme revealed characteristic expression of both linear and branched poly-N-acetyllactosamine, which further comprised specific terminal modifications such as fucosylation and/or sialylation according to the invention on specific types of stem cells.

### Combinations of Elongated Core Epitopes

It is realized that stronger labeling may be obtained if the same terminal epitope is recognized by antibody binding to target structure present on two or three of the major carrier types O-glycans, N-glycans and glycolipids. It is further realized that in context of such use the terminal epitope must be specific enough in comparison to the epitopes present on possible contaminating cells or cell materials. It is further realized that there is highly terminally specific antibodies, which allow binding to on several elongation structures.

The invention revealed each elongated binder type useful in context of stem cells. Thus the invention is directed to the binders recognizing the terminal structure on one or several of the elongating structures according to the invention

### Preferred Group of Monosaccharide Elongation Structures

The invention is directed to use of binders with elongated specificity, when the binders recognize or is able to bind at least one reducing end elongation monosaccharide epitope according to the formula E1

AxHex(NAc)ₙ, wherein A is anomeric structure alfa or beta, X is linkage position 2, 3, 4, or 6 And Hex is hexopyranosyl residue Gal, or Man, and n is integer being 0 or 1, with the provisions that when n is 1 then AxHexNAc is β4GalNAc or β6GalNAc, when Hex is Man, then AxHex is β2Man, and when Hex is Gal, then AxHex is β3Gal or β6Gal.

Beside the monosaccharide elongation structures αSer/Thr are preferred reducing end elongation structures for reducing end GalNAc-comprising O-glycans and βCer is preferred for lactosyl comprising glycolipid epitopes. Elongated terminal epitopes of formulas are obtained by adding E1 to the reducing end of a Formula T1-end of formulas as shown below.

The preferred subgroups of the elongation structures includes i) similar structural epitopes present on O-glycans, polylactosamine and glycolipid cores: β3/6Gal or β6GalNAc; with preferred further subgroups ia) β6GalNAc/β6Gal and ib) β3Gal; ii) N-glycan type epitope β2Man; and iii) globoseries epitopes αc3Gal or α4Gal. The groups are preferred for structural similarity on possible cross reactivity within the groups, which can be used for increasing labeling intensity when background materials are controlled to be devoid of the elongated structure types.

The invention is directed to method of evaluating the status of a human blood related, preferably hematopietic, stem cell preparation comprising the step of detecting the presence of an elongated glycan structure or a group, at least two, of glycan structures in said preparation, wherein said glycan structure or a group of glycan structures is according to Formula T1

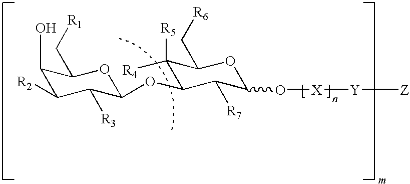

wherein X is linkage position
R₁, R₂, and R₆ are OH or glycosidically linked monosaccharide residue Sialic acid, preferably Neu5Acα2 or Neu5Gcα2, most preferably Neu5Acα2 or
R₃, is OH or glycosidically linked monosaccharide residue Fucα1 (L-fucose) or N-acetyl (N-acetamido, NCOCH₃);
R₄, is H, OH or glycosidically linked monosaccharide residue Fucα1 (L-fucose),
R₅ is OH, when R₄ is H₁ and R₅ is H, when R₄ is not H;

### R7 is N-acetyl or OH

X is natural oligosaccharide backbone structure from the cells, preferably N-glycan, O-glycan or glycolipid structure; or X is nothing, when n is 0,
Y is linker group preferably oxygen for O-glycans and O-linked terminal oligosaccharides and glycolipids and N for N-glycans or nothing when n is 0;
Z is the carrier structure, preferably natural carrier produced by the cells, such as protein or lipid, which is preferably a ceramide or branched glycan core structure on the carrier or H;
The arch indicates that the linkage from the galactopyranosyl is either to position 3 or to position 4 of the residue on the left and that the R4 structure is in the other position 4 or 3;
n is an integer 0 or 1, and m is an integer from 1 to 1000, preferably 1 to 100, and most preferably 1 to 10 (the number of the glycans on the carrier),
With the provisions that one of R2 and R3 is OH or R3 is N-acetyl,
R6 is OH, when the first residue on left is linked to position 4 of the residue on right:
X is not Galα4Galβ4Glc, (the core structure of SSEA-3 or 4) or R3 is Fucosyl, for the analysis of the status of stem cells and/or manipulation of the stem cells, and wherein said cell preparation is embryonic type stem cell preparation.
and when the glycan structure is an elongated structure, wherein the binder binds to the structure and additionally to at least one reducing end elongation epitope, preferably monosaccharide epitope, (replacing X and/or Y) according to the Formula E1:
AxHex(NAc)ₙ, wherein A is anomeric structure alfa or beta, X is linkage position 2, 3, or 6; and
Hex is hexopyranosyl residue Gal, or Man, and n is integer being 0 or 1,
with the provisions that
when n is 1 then AxHexNAc is β4GalNAc or β6GalNAc,
when Hex is Man, then AxHex is O₂Man, and
when Hex is Gal, then AxHex is β3Gal or β6Gal or α3Gal or α4Gal; or
the binder epitope binds additionally to reducing end elongation epitope
Ser/Thr linked to reducing end GalNAcox-comprising structures or
βCer linked to Galβ4Glc comprising structures, and the glycan structure is the stem cell population determined from associated or contaminating cell population.

The invention is directed to method for the analysis of the status of the stem cells and/or for
manipulation of stem cells comprising a step of detecting an elongated glycan structure or at least two glycan structures from a sample of stem cells, wherein said glycan structure is selected from the group consisting of: a terminal lactosamine structure
   (R1)ₙ₁Gal(NAc)ₙ₃β3/4(Fucα4/3)ₙ₂GlcNAcβR wherein R1 is Fucα2, or SAα3, or SAα6 linked to Galβ4GlcNAc, and
   R is the reducing end core structure of N-glycan, O-glycan and/or glycolipid; a, or structure
(SAα3)ₙ₁Galβ3(SAα6)ₙ₂GalNAc; wherein
   n1, n2 and n3 are 0 or 1 indicating presence or absence of a structure wherein SA is a sialic acid; or branched epitope
Galβ3(GlcNAcβ6)GalNAc or
R₁Galβ4(R₃)GlcNAcβ6(R₂Galβ3)GalNAc,
   wherein R₁ and R₂ are independently either nothing or SAα3; and R₃ is independently either nothing or Fucα3; or
Manβ4GlcNAc structure in the core structure of N-linked glycan; or epitope Galβ4Glc,
or terminal mannose
or terminal SAα3/6Gal, wherein SA is a sialic acid, with the provisions that
   i) the stem cells are not cells of a cancer cell line and
   ii) cells are not hematopoietic CD34+ cells and when the structure is comprises N-acetyllactosamine it is specific elongated structure being fucosylated or not SAα3Galβ4GlcNAcβ3 Gal structure.

The invention is directed to methods and binding agents recognizing type II Lactosmine based structures according to the structure according to the Formula T8Ebeta

[Mα]ₘGalβ1-3/4[Nα]ₙGlcNAcβxHex(NAc)ₚ

wherein
wherein x is linkage position 2, 3, or 6
wherein m, n and p are integers 0, or 1, independently
M and N are monosaccharide residues being
i) independently nothing (free hydroxyl groups at the positions) and/or
ii) SA which is Sialic acid linked to 3-position of Gal or/and 6-position of GlcNAc and/or
iii) Fuc (L-fucose) residue linked to 2-position of Gal and/or 3 or 4 position of GlcNAc,
when Gal is linked to the other position (4 or 3) of GlcNAc,
with the provision that m, n and p are 0 or 1, independently.
Hex is hexopyranosyl residue Gal, or Man,
with the provisions that when p is 1 then βxHexNAc is β6GalNAc,
when p is 0
then Hex is Man and βxHex is β2Man, or Hex is Gal and βxHex is β3Gal or β6Gal.

The invention is directed to methods and binding agents recognizing type II Lactosmine based structures according to the

[Mαx]ₘGalβ1-4[Nα]ₙGlcNAcβHex(NAc)ₚ Formula T10E

with the provisions that when p is 1 then βxHexNAc is β6GalNAc,
when p is 0, then Hex is Man and βxHex is β2Man, or Hex is Gal and βxHex is β6Gal.

The invention is directed to methods and binding agents recognizing type II Lactosmine based structures according to the

[Mα]ₘGalβ1-4[Nα]ₙGlcNAcβ2Man, Formula T10EMan:

wherein the variables are as described for Formula T8Ebeta in claim 2.

An embodiment of the invention is directed to a method of evaluating the status of a human blood related, preferably hematopietic, stem cell preparation and/or contaminating cell population comprising the step of detecting the presence of an elongated glycan structure or a group, at least two, of glycan structures in said preparation, wherein said glycan structure or a group of glycan Tn and sialyl-Tn structures is according to Formula MUC

(R)ₙGalNAcα(Ser/Thr)ₘ

wherein n and m are 0 or 1, independently and R is SAα6 or Galβ3, SAis sialic acid preferably Neu5Ac, and when R is Galβ3 n is 1, preferably Tn antiges:

(SAα6)ₙGalNAcα(Ser/Thr)ₘ,

wherein n and m are 0 or 1, independently and SA is sialic acid preferably Neu5Ac, or TF antigen

### Galβ3GalNAcα(Ser/Thr)ₘ.

Useful binder specifities including lectin and elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and L is, Halina) Kluwer Academic publishers Dordrecht, The Neatherlands and internet databases such as pubmed/espacenet or antibody databases such as www.glyco.is.ritsumei.ac.ip/epitope/, which list monoclonal antibody glycan specificities).

### Preferred Binder Molecules

The present invention revealed various types of binder molecules useful for characterization of cells according to the invention and more specifically the preferred cell groups and cell types according to the invention. The preferred binder molecules are classified based on the binding specificity with regard to specific structures or structural features on carbohydrates of cell surface. The preferred binders recognize specifically more than single monosaccharide residue.

It is realized that most of the current binder molecules such as all or most of the plant lectins are not optimal in their specificity and usually recognize roughly one or several monosaccharides with various linkages. Furthermore the specificities of the lectins are usually not well characterized with several glycans of human types.

The preferred high specificity binders recognize
A) at least one monosaccharide residue and a specific bond structure between those to another monosaccharides next monosaccharide residue referred as MS1B1-binder,
B) more preferably recognizing at least part of the second monosaccharide residue referred as MS2B1-binder,
C) even more preferably recognizing second bond structure and or at least part of third mono saccharide residue, referred as MS3B2-binder, preferably the MS3B2 recognizes a specific complete trisaccharide structure.
D) most preferably the binding structure recognizes at least partially a tetrasaccharide with three bond structures, referred as MS4B3-binder, preferably the binder recognizes complete tetrasaccharide sequences.

The preferred binders includes natural human and or animal, or other proteins developed for specific recognition of glycans. The preferred high specificity binder proteins are specific antibodies preferably monoclonal antibodies; lectins, preferably mammalian or animal lectins; or specific glycosyltransferring enzymes more preferably glycosidase type enzymes, glycosyltransferases or transglycosylating enzymes.

### Modulation of Cells by the Binders

The invention revealed that the specific binders directed to a cell type can be used to modulate cells.

In a preferred embodiment the (stem) cells are modulated with regard to carbohydrate mediated interactions. The invention revealed specific binders, which change the glycan structures and thus the receptor structure and function for the glycan, these are especially glycosidases and glycosyltransferring enzymes such as glycosyltransferases and/or transglycosylating enzymes. It is further realized that the binding of a non-enzymatic binder as such select and/or manipulate the cells. The manipulation typically depend on clustering of glycan reseptors or affect of the interactions of the glycan receptors with counter receptors such as lectins present in a biological system or model in context of the cells. The invention further reveled that the modulation by the binder in context of cell culture has effect about the growth velocity of the cells.

### Preferred Combinations of the Binders

The invention revealed useful combination of specific terminal structures for the analysis of status of a cells. In a preferred embodiment the invention is directed to measuring the level of two different terminal structures according to the invention, preferably by specific binding molecules, preferably at least by two different binders. In a preferred embodiment the binder molecules are directed to structures indicating modification of a terminal receptor glycan structures, preferably the structures represent sequential (substrate structure and modification thereof, such as terminal Gal-structure and corresponding sialylated structure) or competing biosynthetic steps (such as fucosylation and sialylation of terminal Galp or terminal Galβ3GlcNAc and Galβ4GlcNAc). In another embodiment the binders are directed to three different structures representing sequential and competing steps such as such as terminal Gal-structure and corresponding sialylated structure and corresponding sialylated structure.

The invention is further directed to recognition of at least two different structures according to the invention selected from the groups of non-modified (non-sialylated or non-fucosylated) Gal(NAc)β3/4-core structures according to the invention, preferred fucosylated structures and preferred sialylated structures according to the invention. It is realized that it is useful to recognize even 3, and more preferably 4 and even more preferably five different structures, preferably within a preferred structure group.

### Target Structures for Specific Binders and Examples of the Binding Molecules

Combination of Terminal Structures with Specific Glycan Core Structures

It is realized that part of the structural elements are specifically associated with specific glycan core structure. The recognition of terminal structures linked to specific core structures are especially preferred, such high specificity reagents have capacity of recognition almost complete individual glycans to the level of physicochemical characterization according to the invention. For example many specific mannose structures according to the invention are in general quite characteristic for N-glycan glycomes according to the invention. The present invention is especially directed to recognition terminal epitopes.

### Common Terminal Structures on Several Glycan Core Structures

The present invention revealed that there are certain common structural features on several glycan types and that it is possible to recognize certain common epitopes on different glycan structures by specific reagents when specificity of the reagent is limited to the terminal without specificity for the core structure. The invention especially revealed characteristic terminal features for specific cell types according to the invention. The invention realized that the common epitopes increase the effect of the recognition. The common terminal structures are especially useful for recognition in the context with possible other cell types or material, which do not contain the common terminal structure in substantial amount.

The invention revealed the presence of the terminal structures on specific core structures such as N-glycan, O-glycan and/or glycolipids. The invention is preferably directed to the selection of specific binders for the structures including recognition of specific glycan core types.

The invention is further directed to glycome compositions of protein linked glycomes such as N-glycans and O-glycans and glycolipids each composition comprising specific amounts of glycan subgroups. The invention is further directed to the compositions when these comprise specific amount of Defined terminal structures.

### Specific Preferred Structural Groups

The present invention is directed to recognition of oligosaccharide sequences comprising specific terminal monosaccharide types, optionally further including a specific core structure. The preferred oligosaccharide sequences are in a preferred embodiment classified based on the terminal monosaccharide structures.

The invention further revealed a family of terminal (non-reducing end terminal) disaccharide epitopes based on β-linked galactopyranosylstructures, which may be further modified by fucose and/or sialic acid residues or by N-acetylgroup, changing the terminal Gal residue to GalNAc. Such structures are present in N-glycan, O-glycan and glycolipid subglycomes. Furthermore the invention is directed to terminal disaccharide epitopes of N-glycans comprising terminal ManαMan.

The structures were derived by mass spectrometric and optionally NMR analysis and by high specificity binders according to the invention, for the analysis of glycolipid structures permethylation and fragmentation mass spectrometry was used. Biosynthetic analysis including known biosynthetic routes to N-glycans, O-glycans and glycolipids was additionally used for the analysis of the glycan compositions and additional support, though not direct evidence due to various regulation levels after mRNA, for it was obtained from gene expression profiling data of Skottman, H. et al. (2005) Stem cells and similar data obtained from the mRNA profiling for cord blood cells and used to support the biosynthetic analysis using the data of Jaatinen T et al. Stem Cells (2006) 24 (3) 631-41.

Structures with Terminal Mannose Monosaccharide

Preferred mannose-type target structures have been specifically classified by the invention. These include various types of high and low-mannose structures and hybrid type structures according to the invention.

### The Preferred Terminal Manα-Target Structure Epitopes

The invention revealed the presence of Manα on low mannose N-glycans and high mannose N-glycans. Based on the biosynthetic knowledge and supporting this view by analysis of mRNAs of biosynthetic enzymes and by NMR-analysis the structures and terminal epitopes could be revealed: Manα2Man, Manα3Man, Manα6Man and Manα3(Manα6)Man, wherein the reducing end Man is preferably either α- or β-linked glycoside and α-linked glycoside in case of Manα2Man:

The general structure of terminal Manα-structures is

Manαx(Manαy)_{z}Manα/β

Wherein x is linkage position 2, 3 or 6, and y is linkage position 3 or 6,
z is integer 0 or 1, indicating the presence or the absence of the branch,
with the provision that x and y are not the same position and
when x is 2, the z is 0 and reducing end Man is preferably α-linked;

The low-mannose structures includes preferably non-reducing end terminal epitopes with structures with α3- and/or α6-mannose linked to another mannose residue

Manαx(Manαy)_{z}Manα/β

wherein x and y are linkage positions being either 3 or 6,
z is integer 0 or 1, indicating the presence or the absence of the branch,

The high mannose structure includes terminal α2-linked Mannose:

Manα2Man(α) and optionally on or several of the terminal α3- and/or α6-mannose-structures as above.

The presence of terminal Manα-structures is regulated in stem cells and the proportion of the high-Man-structures with terminal Manα2-structures in relation to the low Man structures with Manα3/6- and/or to complex type N-glycans with Gal-backbone epitopes varies cell type specifically.

The data indicated that binder revealing specific terminal Manα2Man and/or Manα3/6Man is very useful in characterization of stem cells. The prior science has not characterized the epitopes as specific signals of cell types or status.

The invention is especially directed to the measuring the levels of both low-Man and high-Man structures, preferably by quantifying two structure type the Manα2Man-structures and the Manα3/6Man-structures from the same sample.

The invention is especially directed to high specificity binders such as enzymes or monoclonal antibodies for the recognition of the terminal Manox-structures from the preferred stem cells according to the invention, more preferably from differentiated embryonal type cells, more preferably differentiated beyond embryoid bodies such as stage 3 differentiatated cells, most preferably the structures are recognized from stage 3 differentiated cells. The invention is especially preferably directed to detection of the structures from adult stem cells more preferably mesenchymal stem cells, especially from the surface of mesenchymal stem cells and in separate embodiment from blood derived stem cells, with separately preferred groups of cord blood and bone marrow stem cells. In a preferred embodiment the cord blood and/or peripheral blood stem cell is not hematopoietic stem cell.

### Low or Uncharacterised Specificity Binders

preferred for recognition of terminal mannose structures includes mannose-monosaccharide binding plant lectins. The invention is in preferred embodiment directed to the recognition of stem cells such as embryonal type stem cells by a Manox-recognizing lectin such as lectin PSA. In a preferred embodiment the recognition is directed to the intracellular glycans in permeabilized cells. In another embodiment the Manα-binding lectin is used for intact non-permeabilized cells to recognize terminal Manα-from contaminating cell population such as fibroblast type cells or feeder cells as shown in corresponding Examples.

### Preferred High Specific High Specificity Binders

include

i) Specific mannose residue releasing enzymes such as linkage specific mannosidases, more preferably an α-mannosidase or β-mannosidase.

Preferred α-mannosidases includes linkage specific α-mannosidases such as (x-Mannosidases cleaving preferably non-reducing end terminal, an example of preferred mannosidases is jack bean α-mannosidase (Canavalia ensiformis; Sigma, USA) and homologous α-mannosidases α2-linked mannose residues specifically or more effectively than other linkages, more preferably cleaving specifically Manα2-structures; or

α3-linked mannose residues specifically or more effectively than other linkages, more preferably cleaving specifically Manα3-structures; or
α6-linked mannose residues specifically or more effectively than other linkages, more preferably cleaving specifically Manα6-structures;
Preferred β-mannosidases includes β-mannosidases capable of cleaving β4-linked mannose from non-reducing end terminal of N-glycan core Manβ4GlcNAc-structure without cleaving other β-linked monosaccharides in the glycomes.

ii) Specific binding proteins recognizing preferred mannose structures according to the invention. The preferred reagents include antibodies and binding domains of antibodies (Fab-fragments and like), and other engineered carbohydrate binding proteins. The invention is directed to antibodies recognizing MS2B1 and more preferably MS3B2-structures.

Mannosidase analyses of neutral N-glycans Examples of detection of mannosylated by α-mannosidase binder and mass spectrometric profiling of the glycans cord blood and peripheral blood mesenchymal cells in Examples; for cord blood cells in example 14, indicates presence of all types of Manβ4, Manα3/6 terminal structures of Man₁₋₄GlcNAcβ4(Fucα6)₀₋₁GlcNAc-comprising low Mannose glycans as described by the invention.

### Lectin Binding

α-linked mannose was demonstrated in Examples for human mesenchymal cell by lectins Hippeastrum hybrid (HHA) and Pisum sativum (PSA) lectins suggests that they express mannose, more specifically α-linked mannose residues on their surface glycoconjugates such as N-glycans. Possible α-mannose linkages include α1→2, α1→3, and α1→6. The lower binding of Galanthus nivalis (GNA) lectin suggests that some α-mannose linkages on the cell surface are more prevalent than others. The combination of the terminal Manα-recognizing low affinity reagents appears to be useful and correspond to results obtained by mannosidase screening; NMR and mass spectrometric results. Lectin binding of cord blood cells is in example 8. PSA has specificity for complex type N-glycans with core Fucα6-epitopes.

Mannose-binding lectin labelling. Labelling of the mesenchymal cells in Examples was also detected with human serum mannose-binding lectin (MBL) coupled to fluorescein label. This indicate that ligands for this innate immunity system component may be expressed on in vitro cultured BM MSC cell surface.

The present invention is especially directed to analysis of terminal Manox-on cell surfaces as the structure is ligand for MBL and other lectins of innate immunity. It is further realized that terminal Manα-structures would direct cells in blood circulation to mannose receptor comprising tissues such as Kupfer cells of liver. The invention is especially directed to control of the amount of the structure by binding with a binder recognizing terminal Manα-structure.

In a preferred embodiment the present invention is directed to the testing of presence of ligands of lectins present in human, such as lectins of innate immunity and/or lectins of tissues or leukocytes, on stem cells by testing of the binding of the lectin (purified or preferably a recombinant form of the lectin, preferably in lableed form) to the stem cells. It is realized that such lectins includes especially lectins binding Manα and Galβ/GalNAcβ-structures (terminal non-reducing end or even α6-sialylated forms according to the invention.

### Mannose Binding Antibodies

A high-mannose binding antibody has been described for example in Wang L X et al (2004) 11 (1) 127-34. Specific antibodies for short mannosylated structures such as the trimannosyl core structure have been also published.

Structures with Terminal Gal-Monosaccharide

Preferred galactose-type target structures have been specifically classified by the invention. These include various types of N-acetyllactosamine structures according to the invention.

### Low or Uncharacterised Specificity Binders for Terminal Gal

Prereferred for recognition of terminal galactose structures includes plant lectins such as ricin lectin (ricinus communis agglutinin RCA), and peanut lectin (/agglutinin PNA). The low resolution binders have different and broad specificities.

### Preferred High Specific High Specificity Binders Include

i) Specific galactose residue releasing enzymes such as linkage specific galactosidases, more preferably α-galactosidase or β-galactosidase.

Preferred α-galactosidases include linkage galactosidases capable of cleaving Galα3Gal-structures revealed from specific cell preparations

Preferred β-galactosidases includes β-galactosidases capable of cleaving β4-linked galactose from non-reducing end terminal Galβ4GlcNAc-structure without cleaving other β-linked monosaccharides in the glycomes and

β3-linked galactose from non-reducing end terminal Galβ3GlcNAc-structure without cleaving other β-linked monosaccharides in the glycomes

ii) Specific binding proteins recognizing preferred galactose structures according to the invention. The preferred reagents include antibodies and binding domains of antibodies (Fab-fragments and like), and other engineered carbohydrate binding proteins and animal lectins such as galectins.

### Specific Binder Experiments and Examples for Galβ-Structures

Specific exoglycosidase and glycosyltransferase analysis for the structures are included in Examples for embryonal stem cells and differentiated cells; for cord blood cells in example 14 and in example 4 on cell surface and including glycosyltransferases, and for glycolipids in Example 10. Sialylation level analysis related to terminal Galβ and Sialic acid expression is in Example 9.

Preferred enzyme binders for the binding of the Galβ-epitopes according to the invention includes β1,4-galactosidase e.g. from S. pneumoniae (rec. in E. coli, Calbiochem, USA), β1,3-galactosidase (e.g. rec. in E. coli, Calbiochem); glycosyltransferases: α-2,3-(N)-sialyltransferase (rat, recombinant in S. frugiperda, Calbiochem), α-1,3-fucosyltransferase VI (human, recombinant in S. frugiperda, Calbiochem), which are known to recognize specific N-acetyllactosamine epitopes, Fuc-TVI especially Galβ4GlcNAc.

Plant low specificity lectin, such as RCA, PNA, ECA, STA, and

PWA, data is in Examples for hESC, Examples for MSCs, Example 8 for cord blood, effects of the lectin binders for the cell proliferation is in Examples, cord blood cell selection is in Example 11. Human lectin analysis by various galectin expression is Example 12 from cord blood and embryonal cells. In example 13 there is antibody labeling of especially fucosylated and galactosylated structures.

Poly-N-acetyllactosamine sequences. Labelling of the cells by pokeweed (PWA) and less intense labelling by Solanum tuberosum (STA) lectins suggests that the cells express poly-N-acetyllactosamine sequences on their surface glycoconjugates such as N- and/or O-glycans and/or glycolipids. The results further suggest that cell surface poly-N-acetyllactosamine chains contain both linear and branched sequences.

Structures with Terminal GalNAc-Monosaccharide

Preferred GalNAc-type target structures have been specifically revealed by the invention. These include especially LacdiNAc, GalNAcβGlcNAc-type structures according to the invention.

### Low or Uncharacterised Specificity Binders for Terminal GalNac

Several plant lectins has been reported for recognition of terminal GalNAc. It is realized that some GalNAc-recognizing lectins may be selected for low specificity reconition of the preferred LacdiNAc-structures.

β-linked N-acetylgalactosamine. Abundant labelling of hESC by Wisteria floribunda lectin (WFA) suggests that hESC express β-linked non-reducing terminal N-acetylgalactosamine residues on their surface glycoconjugates such as N- and/or O-glycans. The absence of specific binding of WFA to mEF suggests that the lectin ligand epitopes are less abundant in mEF.

The low specificity binder plant lectins such as Wisteria floribunda agglutinin and Lotus tetragonolobus agglutinin bind to oligosaccharide sequences Srivatsan J. et al. Glycobiology (1992) 2 (5) 445-52: Do, K Y et al. Glycobiology (1997) 7 (2) 183-94; Yan, L., et al (1997) Glycoconjugate J. 14 (1) 45-55. The article also shows that the lectins are useful for recognition of the structures, when the cells are verified not to contain other structures recognized by the lectins.

In a preferred embodiment a low specificity leactin reagent is used in combination with another reagent verifying the binding.

### Preferred High Specific High Specificity Binders Include

i) The invention revealed that β-linked GalNAc can be recognized by specific β-N-acetylhexosaminidase enzyme in combination with β-N-acetylhexosaminidase enzyme. This combination indicates the terminal monosaccharide and at least part of the linkage structure.

Preferred β-N-acetylehexosaminidase, includes enzyme capable of cleaving β-linked GalNAc from non-reducing end terminal GalNAcβ4/3-structures without cleaving α-linked HexNAc in the glycomes; preferred N-acetylglucosaminidases include enzyme capable of cleaving β-linked GlcNAc but not GalNAc.

Specific binding proteins recognizing preferred GalNAcβ4, more preferably GalNAcβ4GlcNAc, structures according to the invention. The preferred reagents include antibodies and binding domains of antibodies (Fab-fragments and like), and other engineered carbohydrate binding proteins.

Examples antibodies recognizing LacdiNAc-structures includes publications of Nyame A. K. et al. (1999) Glycobiology 9 (10) 1029-35; van Remoortere A. et al (2000) Glycobiology 10 (6) 601-609; and van Remoortere A. et al (2001) Infect. Immun. 69 (4) 2396-2401. The antibodies were characterized in context of parasite (Schistosoma) infection of mice and humans, but according to the present invention these antibodies can also be used in screening stem cells. The present invention is especially directed to selection of specific clones of LacdiNac recognizing antibodies specific for the subglycomes and glycan structures present in N-glycomes of the invention.

The articles disclose antibody binding specificities similar to the invention and methods for producing such antibodies, therefore the antibody binders are obvious for person skilled in the art. The immunogenicity of certain LacdiNAc-structures are demonstrated in human and mice.

The use of glycosidase in recognition of the structures in known in the prior art similarity as in the present invention for example in Srivatsan J. et al. (1992) 2 (5) 445-52.

Structures with Terminal GlcNAc-Monosaccharide

Preferred GlcNAc-type target structures have been specifically revealed by the invention. These include especially GlcNAcβ-type structures according to the invention.

### Low or Uncharacterised Specificity Binders for Terminal GlcNAc

Several plant lectins has been reported for recognition of terminal GlcNAc. It is realized that some GlcNAc-recognizing lectins may be selected for low specificity recognition of the preferred GlcNAc-structures.

### Preferred High Specific High Specificity Binders Include

i) The invention revealed that β-linked GlcNAc can be recognized by specific β-N-acetylglucosaminidase enzyme.

Preferred β-N-acetylglucosaminidase includes enzyme capable of cleaving β-linked GlcNAc from non-reducing end terminal GlcNAcβ2/3/6-structures without cleaving β-linked GalNAc or α-linked HexNAc in the glycomes;

ii) Specific binding proteins recognizing preferred GlcNAcβ2/3/6, more preferably GlcNAcβ2Manox, structures according to the invention. The preferred reagents include antibodies and binding domains of antibodies (Fab-fragments and like), and other engineered carbohydrate binding proteins.

### Specific Binder Experiments and Examples for Terminal HexNAc(GalNAc/GlcNAc and GlcNAc Structures

Specific exoglycosidase analysis for the structures are included for cord blood cells in example 14 and for glycolipids in Example 10.

Plant low specificity lectin, such as WFA and GNAII, and data is in Examples for hESC, Examples for MSCs, Example 8 for cord blood, effects of the lectin binders for the cell proliferation is in Examples, cord blood cell selection is in Example 11.

Preferred enzymes for the recognition of the structures includes general hexosaminidase β-hexosaminidase from Jack beans (C. ensiformis, Sigma, USA) and specific N-acetylglucosaminidases or N-acetylgalactosaminidases such as β-glucosaminidase from S. pneumoniae (rec. in E. coli, Calbiochem, USA). Combination of these allows determination of LacdiNAc.

The invention is further directed to analysis of the structures by specific monoclonal antibodies recognizing terminal GlcNAcβ-structures such as described in Holmes and Greene (1991) 288 (1) 87-96, with specificity for several terminal GlcNAc structures.

The invention is specifically directed to the use of the terminal structures according to the invention for selection and production of antibodies for the structures.

Verification of the target structures includes mass spectrometry and permethylation/fragmentation analysis for glycolipid structures

Structures with Terminal Fucose-Monosaccharide

Preferred fucose-type target structures have been specifically classified by the invention. These include various types of N-acetyllactosamine structures according to the invention. The invention is further more directed to recognition and other methods according to the invention for lactosamine similar α6-fucosylated epitope of N-glycan core, GlcNAcβ4(Fucα6)GlcNAc. The invention revealed such structures recognizable by the lectin PSA (Kornfeld (1981) J Biol Chem 256, 6633-6640; Cummings and Kornfeld (1982) J Biol Chem 257, 11235-40) are present e.g. in embryonal stem cells and mesenchymal stem cells.

### Low or Uncharacterised Specificity Binders for Terminal Fuc

Preferred for recognition of terminal fucose structures includes fucose monosaccharide binding plant lectins. Lectins of Ulex europeaus and Lotus tetragonolobus has been reported to recognize for example terminal Fucoses with some specificity binding for α2-linked structures, and branching α3-fucose, respectively. Data is in Example 8 for cord blood, effects of the lectin binders for the cell proliferation is for cord blood cell selection is in Example 11.

### Preferred High Specific High Specificity Binders Include

i) Specific fucose residue releasing enzymes such as linkage fucosidases, more preferably α-fucosidase.

Preferred α-fucosidases include linkage fucosidases capable of cleaving Fucα2Gal-, and Galβ4/3(Fucα3/4)GlcNAc-structures revealed from specific cell preparations.

Specific exoglycosidase and for the structures are included for cord blood cells in example 14 and in example 4 on cell surface for glycolipids in Example 10. Preferred fucosidases includes α1,3/4-fucosidase e.g. α1,3/4-fucosidase from Xanthomonas sp. (Calbiochem, USA), and α1,2-fucosidase e.g. α1,2-fucosidase from X. manihotis (Glyko),

ii) Specific binding proteins recognizing preferred fucose structures according to the invention. The preferred reagents include antibodies and binding domains of antibodies (Fab-fragments and like), and other engineered carbohydrate binding proteins and animal lectins such as selectins recognizing especially Lewis type structures such as Lewis x, Galβ4(Fucα3)GlcNAc, and sialyl-Lewis x, SAα3Galβ4(Fucα3)GlcNAc.

The preferred antibodies includes antibodies recognizing specifically Lewis type structures such as Lewis x, and sialyl-Lewis x. More preferably the Lewis x-antibody is not classic SSEA-1 antibody, but the antibody recognizes specific protein linked Lewis x structures such as Galβ4(Fucα3)GlcNAcβ2Manα-linked to N-glycan core.

iii) the invention is further directed to recognition of α6-fucosylated epitope of N-glycan core, GlcNAcβ4(Fucα6)GlcNAc. The invention directed to recognition of such structures by structures by the lectin PSA or lentil lectin (Komfeld (1981) J Biol Chem 256, 6633-6640) or by specific monoclonal antibodies (e.g. Srikrishna G. et al (1997) J Biol Chem 272, 25743-52). The invention is further directed to methods of isolation of cellular glycan components comprising the glycan epitope and isolation stem cell N-glycans, which are not bound to the lectin as control fraction for further characterization.

Structures with Terminal Sialic Acid-Monosaccharide

Preferred sialic acid-type target structures have been specifically classified by the invention.

### Low or Uncharacterised Specificity Binders for Terminal Sialic Acid

Preferred for recognition of terminal sialic acid structures includes sialic acid monosaccharide binding plant lectins.

### Preferred High Specific High Specificity Binders Include

i) Specific sialic acid residue releasing enzymes such as linkage sialidases, more preferably α-sialidases.

Preferred α-sialidases include linkage sialidases capable of cleaving SAα3Gal- and SAα6Gal-structures revealed from specific cell preparations by the invention.

Preferred low specificity lectins, with linkage specificity include the lectins, that are specific for SAα3Gal-structures, preferably being Maackia amurensis lectin and/or lectins specific for SAα6Gal-structures, preferably being Sambucus nigra agglutinin.

ii) Specific binding proteins recognizing preferred sialic acid oligosaccharide sequence structures according to the invention. The preferred reagents include antibodies and binding domains of antibodies (Fab-fragments and like), and other engineered carbohydrate binding proteins and animal lectins such as selectins recognizing especially Lewis type structures such as sialyl-Lewis x, SAα3Galβ4(Fucα3)GlcNAc or sialic acid recognizing Siglec-proteins.

The preferred antibodies includes antibodies recognizing specifically sialyl-N-acetyllactosamines, and sialyl-Lewis x.

Preferred antibodies for NeuGc-structures includes antibodies recognizes a structure NeuGcα3Galβ4Glc(NAc)_{0 or 1} and/or GalNAcβ4[NeuGcα3]Galβ4Glc(NAc)o or 1, wherein [ ] indicates branch in the structure and ( )_{0 or 1} a structure being either present or absent. In a preferred embodiment the invention is directed recognition of the N-glycolyl-Neuraminic acid structures by antibody, preferably by a monoclonal antibody or human/humanized monoclonal antibody. A preferred antibody contains the variable domains of P3-antibody.

### Specific Binder Experiments and Examples for α3/6 Sialylated Structures

Specific exoglycosidase analysis for the structures are included for cord blood cells in example 14 and in example 4 on cell surface and including glycosyltransferases, for glycolipids in Example 10. Sialylation level analysis related to terminal Galβ and Sialic acid expression is in Example 9.

Preferred enzyme binders for the binding of the Sialic acid epitopes according to the invention includes: sialidases such as general sialidase α2,3/6/8/9-sialidase from A. ureafaciens (Glyko), and α2,3-Sialidases such as: α2,3-sialidase from S. pneumoniae (Calbiochem, USA). Other useful sialidases are known from E. coli, and Vibrio cholerae.

α1,3-fucosyltransferase VI (human, recombinant in S. frugiperda, Calbiochem), which are known to recognize specific N-acetyllactosamine epitopes, Fuc-TVI especially including SAα3Galβ4GlcNAc.

Plant low specificity lectin, such as MAA and SNA, and data is in Examples for hESC, Examples for MSCs, Example 8 for cord blood, effects of the lectin binders for the cell proliferation is in Examples, cord blood cell selection is in Example 11. In example 13 there is antibody labeling of sialylstructures.

Preferred Uses for Stem Cell Type Specific Galectins and/or Galectin Ligands

As described in the Examples, the inventors also found that different stem cells have distinct galectin expression profiles and also distinct galectin (glycan) ligand expression profiles. The present invention is further directed to using galactose-binding reagents, preferentially galactose-binding lectins, more preferentially specific galectins; in a stem cell type specific fashion to modulate or bind to certain stem cells as described in the present invention to the uses described. In a further preferred embodiment, the present invention is directed to using galectin ligand structures, derivatives thereof, or ligand-mimicking reagents to uses described in the present invention in stem cell type specific fashion. The preferred galectins are listed in Example 12.

The invention is in a preferred embodiment directed to the recognition of terminal N-acetyllactosamines from cells by galectins as described above for recognition of Galβ4GlcNAc and Galβ3GlcNAc structures: The results indicate that both CB CD34+/CD133+ stem cell populations and hESC have an interesting and distinct galectin expression profiles, leading to different galectin ligand affinity profiles (Hirabayashi et al., 2002). The results further correlate with the glycan analysis results showing abundant galectin ligand expression in these stem cells, especially non-reducing terminal β-Gal and type II LacNAc, poly-LacNAc, β1,6-branched poly-LacNAc, and complex-type N-glycan expression.

### Specific Technical Aspects of Stem Cell Glycome Analysis

### Isolation of Glycans and Glycan Fractions

Glycans of the present invention can be isolated by the methods known in the art. A preferred glycan preparation process consists of the following steps:

1° isolating a glycan-containing fraction from the sample,
2° . . . . Optionally purification the fraction to useful purity for glycome analysis

The preferred isolation method is chosen according to the desired glycan fraction to be analyzed. The isolation method may be either one or a combination of the following methods, or other fractionation methods that yield fractions of the original sample:

1° extraction with water or other hydrophilic solvent, yielding water-soluble glycans or glycoconjugates such as free oligosaccharides or glycopeptides,
2° extraction with hydrophobic solvent, yielding hydrophilic glycoconjugates such as glycolipids,
3° N-glycosidase treatment, especially Flavobacterium meningosepticum N-glycosidase F treatment, yielding N-glycans,
4° alkaline treatment, such as mild (e.g. 0.1 M) sodium hydroxide or concentrated ammonia treatment, either with or without a reductive agent such as borohydride, in the former case in the presence of a protecting agent such as carbonate, yielding β-elimination products such as O-glycans and/or other elimination products such as N-glycans,
5° endoglycosidase treatment, such as endo-β-galactosidase treatment, especially Escherichia freundii endo-β-galactosidase treatment, yielding fragments from poly-N-acetyllactosamine glycan chains, or similar products according to the enzyme specificity, and/or
6° protease treatment, such as broad-range or specific protease treatment, especially trypsin treatment, yielding proteolytic fragments such as glycopeptides.

The released glycans are optionally divided into sialylated and non-sialylated subfractions and analyzed separately. According to the present invention, this is preferred for improved detection of neutral glycan components, especially when they are rare in the sample to be analyzed, and/or the amount or quality of the sample is low. Preferably, this glycan fractionation is accomplished by graphite chromatography.

According to the present invention, sialylated glycans are optionally modified in such manner that they are isolated together with the non-sialylated glycan fraction in the non-sialylated glycan specific isolation procedure described above, resulting in improved detection simultaneously to both non-sialylated and sialylated glycan components. Preferably, the modification is done before the non-sialylated glycan specific isolation procedure. Preferred modification processes include neuraminidase treatment and derivatization of the sialic acid carboxyl group, while preferred derivatization processes include amidation and esterification of the carboxyl group.

### Glycan Release Methods

The preferred glycan release methods include, but are not limited to, the following methods:

Free glycans—extraction of free glycans with for example water or suitable water-solvent mixtures.

Protein-linked glycans including O- and N-linked glycans—alkaline elimination of protein-linked glycans, optionally with subsequent reduction of the liberated glycans.

Mucin-type and other Ser/Thr O-linked glycans—alkaline β-elimination of glycans, optionally with subsequent reduction of the liberated glycans.

N-glycans—enzymatic liberation, optionally with N-glycosidase enzymes including for example N-glycosidase F from C. meningosepticum, Endoglycosidase H from Streptomyces, or N-glycosidase A from almonds.

Lipid-linked glycans including glycosphingolipids—enzymatic liberation with endoglycoceramidase enzyme; chemical liberation; ozonolytic liberation.

Glycosaminoglycans—treatment with endo-glycosidase cleaving glycosaminoglycans such as chondroinases, chondroitin lyases, hyalurondases, heparanases, heparatinases, or keratanases/endo-beta-galactosidases; or use of O-glycan release methods for O-glycosidic Glycosaminoglycans; or N-glycan release methods for N-glycosidic glycosaminoglycans or use of enzymes cleaving specific glycosaminoglycan core structures; or specific chemical nitrous acid cleavage methods especially for amine/N-sulphate comprising glycosaminoglycans

Glycan fragments—specific exo- or endoglycosidase enzymes including for example keratanase, endo-β-galactosidase, hyaluronidase, sialidase, or other exo- and endoglycosidase enzyme; chemical cleavage methods; physical methods

### Preferred Target Cell Populations and Types for Analysis According to the Invention

### Early Human Cell Populations

### Human Stem Cells and Multipotent Cells

Under broadest embodiment the present invention is directed to all types of human stem cells, meaning fresh and cultured human stem cells. The stem cells according to the invention do not include traditional cancer cell lines, which may differentiate to resemble natural cells, but represent non-natural development, which is typically due to chromosomal alteration or viral transfection. Stem cells include all types of non-malignant multipotent cells capable of differentiating to other cell types. The stem cells have special capacity stay as stem cells after cell division, the self-reneval capacity.

Under the broadest embodiment for the human stem cells, the present invention describes novel special glycan profiles and novel analytics, reagents and other methods directed to the glycan profiles. The invention shows special differences in cell populations with regard to the novel glycan profiles of human stem cells.

The present invention is further directed to the novel structures and related inventions with regard to the preferred cell populations according to the invention. The present invention is further directed to specific glycan structures, especially terminal epitopes, with regard to specific preferred cell population for which the structures are new.

### Preferred Types of Early Human Cells

The invention is directed to specific types of early human cells based on the tissue origin of the cells and/or their differentiation status.

The present invention is specifically directed to early human cell populations meaning multipotent cells and cell populations derived thereof based on origins of the cells including the age of donor individual and tissue type from which the cells are derived, including preferred cord blood as well as bone marrow from older individuals or adults.

Preferred differentiation status based classification includes preferably “solid tissue progenitor” cells, more preferably “mesenchymal-stem cells”, or cells differentiating to solid tissues or capable of differentiating to cells of either ectodermal, mesodermal, or endodermal, more preferentially to mesenchymal stem cells.

The invention is further directed to classification of the early human cells based on the status with regard to cell culture and to two major types of cell material. The present invention is preferably directed to two major cell material types of early human cells including fresh, frozen and cultured cells.

### Cord Blood Cells, Embryonal-Type Cells and Bone Marrow Cells

The present invention is specifically directed to early human cell populations meaning multipotent cells and cell populations derived thereof based on the origin of the cells including the age of donor individual and tissue type from which the cells are derived.
a) from early age-cells such 1) as neonatal human, directed preferably to cord blood and related material, and 2) embryonal cell-type material
b) from stem and progenitor cells from older individuals (non-neonatal, preferably adult), preferably derived from human “blood related tissues” comprising, preferably bone marrow cells.

### Cells Differentiating to Solid Tissues, Preferably to Mesenchymal Stem Cells

The invention is specifically under a preferred embodiment directed to cells, which are capable of differentiating to non-hematopoietic tissues, referred as “solid tissue progenitors”, meaning to cells differentiating to cells other than blood cells. More preferably the cell population produced for differentiation to solid tissue are “mesenchymal-type cells”, which are multipotent cells capable of effectively differentiating to cells of mesodermal origin, more preferably mesenchymal stem cells. Most of the prior art is directed to hematopoietic cells with characteristics quite different from the mesenchymal-type cells and mesenchymal stem cells according to the invention.

Preferred solid tissue progenitors according to the invention includes selected multipotent cell populations of cord blood, mesenchymal stem cells cultured from cord blood, mesenchymal stem cells cultured/obtained from bone marrow and embryonal-type cells. In a more specific embodiment the preferred solid tissue progenitor cells are mesenchymal stem cells, more preferably “blood related mesenchymal cells”, even more preferably mesenchymal stem cells derived from bone marrow or cord blood.

Under a specific embodiment CD34+ cells as a more hematopoietic stem cell type of cord blood or CD34+ cells in general are excluded from the solid tissue progenitor cells.

### Early Blood Cell Populations and Corresponding Mesenchymal Stem Cells

### Cord Blood

The early blood cell populations include blood cell materials enriched with multipotent cells. The preferred early blood cell populations include peripheral blood cells enriched with regard to multipotent cells, bone marrow blood cells, and cord blood cells. In a preferred embodiment the present invention is directed to mesenchymal stem cells derived from early blood or early blood derived cell populations, preferably to the analysis of the cell populations.

### Bone Marrow

Another separately preferred group of early blood cells is bone marrow blood cells. These cell do also comprise multipotent cells. In a preferred embodiment the present invention is directed to directed to mesenchymal stem cells derived from bone marrow cell populations, preferably to the analysis of the cell populations.

### Preferred Subpopulations of Early Human Blood Cells

The present invention is specifically directed to subpopulations of early human cells. In a preferred embodiment the subpopulations are produced by selection by an antibody and in another embodiment by cell culture favouring a specific cell type. In a preferred embodiment the cells are produced by an antibody selection method preferably from early blood cells. Preferably the early human blood cells are cord blood cells.

The CD34 positive cell population is relatively large and heterogenous. It is not optimal for several applications aiming to produce specific cell products. The present invention is preferably directed to specifically selected non-CD34 populations meaning cells not selected for binding to the CD34− marker, called homogenous cell populations. The homogenous cell populations may be of smaller size mononuclear cell populations for example with size corresponding to CD133+ cell populations and being smaller than specifically selected CD34+ cell populations. It is further realized that preferred homogenous subpopulations of early human cells may be larger than CD34+ cell populations.

The homogenous cell population may a subpopulation of CD34+ cell population, in preferred embodiment it is specifically a CD133+ cell population or CD133− type cell population. The “CD133-type cell populations” according to the invention are similar to the CD133+ cell populations, but preferably selected with regard to another marker than CD133. The marker is preferably a CD133-coexpressed marker. In a preferred embodiment the invention is directed to CD133+ cell population or CD133+ subpopulation as CD133-type cell populations. It is realized that the preferred homogeneous cell populations further includes other cell populations than which can be defined as special CD133-type cells.

Preferably the homogenous cell populations are selected by binding a specific binder to a cell surface marker of the cell population. In a preferred embodiment the homogenous cells are selected by a cell surface marker having lower correlation with CD34-marker and higher correlation with CD133 on cell surfaces. Preferred cell surface markers include α3-sialylated structures according to the present invention enriched in CD133-type cells. Pure, preferably complete, CD133+ cell population are preferred for the analysis according to the present invention.

The present invention is directed to essential mRNA-expression markers, which would allow analysis or recognition of the cell populations from pure cord blood derived material. The present invention is specifically directed to markers specifically expressed on early human cord blood cells.

The present invention is in a preferred embodiment directed to native cells, meaning non-genetically modified cells. Genetic modifications are known to alter cells and background from modified cells. The present invention further directed in a preferred embodiment to fresh non-cultivated cells.

The invention is directed to use of the markers for analysis of cells of special differentiation capacity, the cells being preferably human blood cells or more preferably human cord blood cells.

Preferred Purity of Reproducibly Highly Purified Mononuclear Complete Cell Populations from Human Cord Blood

The present invention is specifically directed to production of purified cell populations from human cord blood. As described above, production of highly purified complete cell preparations from human cord blood has been a problem in the field. In the broadest embodiment the invention is directed to biological equivalents of human cord blood according to the invention, when these would comprise similar markers and which would yield similar cell populations when separated similarly as the CD133+ cell population and equivalents according to the invention or when cells equivalent to the cord blood is contained in a sample further comprising other cell types. It is realized that characteristics similar to the cord blood can be at least partially present before the birth of a human. The inventors found out that it is possible to produce highly purified cell populations from early human cells with purity useful for exact analysis of sialylated glycans and related markers.

### Preferred Bone Marrow Cells

The present invention is directed to multipotent cell populations or early human blood cells from human bone marrow. Most preferred are bone marrow derived mesenchymal stem cells. In a preferred embodiment the invention is directed to mesenchymal stem cells differentiating to cells of structural support function such as bone and/or cartilage.

A variety of factors previously mentioned influence ability of stem cells to survive, replicate, and differentiate. For example, in terms of nutrients the amino acid taurine under certain conditions preferentially inhibits murine bone marrow cells from forming osteoclasts (Koide, et al., 1999, Arch Oral Biol 44:711-719), the amino acid L-arginine stimulates erythrocyte differentiation and proliferation of erythroid progenitors (Shima, et al., 2006, Blood 107:1352-1356), extracellular ATP acting through P2Y receptors mediates a wide variety of changes to both hematopoietic and non-hematopoietic stem cells (Lee, et al., 2003, Genes Dev 17:1592-1604), arginine-glycine-aspartic acid attached to porous polymer scaffolds increase differentiation and survival of osteoblast progenitors (Hu, et al., 2003, J Biomed Mater Res A 64:583-590), each of which is incorporated by reference herein in its entirety. Accordingly, one skilled in the art would know to use various types of nutrients for inducing differentiation, or maintaining viability, of certain types of stem cells and/or progeny thereof.

### Embryonal-Type Cell Populations

The present invention is specifically directed to methods directed to embryonal-type cell populations, preferably when the use does not involve commercial or industrial use of human embryos nor involve destruction of human embryos. The invention is under a specific embodiment directed to use of embryonal cells and embryo derived materials such as embryonal stem cells, whenever or wherever it is legally acceptable. It is realized that the legislation varies between countries and regions.

The present invention is further directed to use of embryonal-related, discarded or spontaneously damaged material, which would not be viable as human embryo and cannot be considered as a human embryo. In yet another embodiment the present invention is directed to use of accidentally damaged embryonal material, which would not be viable as human embryo and cannot be considered as human embryo.

It is further realized that early human blood derived from human cord or placenta after birth and removal of the cord during normal delivery process is ethically uncontroversial discarded material, forming no part of human being.

The invention is further directed to cell materials equivalent to the cell materials according to the invention. It is further realized that functionally and even biologically similar cells may be obtained by artificial methods including cloning technologies.

### Mesenchymal Multipotent Cells

The present invention is further directed to mesenchymal stem cells or multipotent cells as preferred cell population according to the invention. The preferred mesencymal stem cells include cells derived from early human cells, preferably human cord blood or from human bone marrow. In a preferred embodiment the invention is directed to mesenchymal stem cells differentiating to cells of structural support function such as bone and/or cartilage, or to cells forming soft tissues such as adipose tissue.

### Control of Cell Status and Potential Contaminations by Glycosylation Analysis

### Control of Cell Status

### Control of Raw Material Cell Population

The present invention is directed to control of glycosylation of cell populations to be used in therapy.

The present invention is specifically directed to control of glycosylation of cell materials, preferably when
1) there is difference between the origin of the cell material and the potential recipient of transplanted material. In a preferred embodiment there are potential inter-individual specific differences between the donor of cell material and the recipient of the cell material. In a preferred embodiment the invention is directed to animal or human, more preferably human specific, individual person specific glycosylation differences. The individual specific differences are preferably present in mononuclear cell populations of early human cells, early human blood cells and embryonal type cells. The invention is preferably not directed to observation of known individual specific differences such as blood group antigens changes on erythrocytes.
2) There is possibility in variation due to disease specific variation in the materials. The present invention is specifically directed to search of glycosylation differences in the early cell populations according to the present invention associated with infectious disease, inflammatory disease, or malignant disease. Part of the inventors have analysed numerous cancers and tumors and observed similar types glycosylations as certain glycosylation types in the early cells.
3) There is for a possibility of specific inter-individual biological differences in the animals, preferably humans, from which the cell are derived for example in relation to species, strain, population, isolated population, or race specific differences in the cell materials.
4) When it has been established that a certain cell population can be used for a cell therapy application, glycan analysis can be used to control that the cell population has the same characteristics as a cell population known to be useful in a clinical setting.

### Time Dependent Changes During Cultivation of Cells

Furthermore during long term cultivation of cells spontaneous mutations may be caused in cultivated cell materials. It is noted that mutations in cultivated cell lines often cause harmful defects on glycosylation level.

It is further noticed that cultivation of cells may cause changes in glycosylation. It is realized that minor changes in any parameter of cell cultivation including quality and concentrations of various biological, organic and inorganic molecules, any physical condition such as temperature, cell density, or level of mixing may cause difference in cell materials and glycosylation. The present invention is directed to monitoring glycosylation changes according to the present invention in order to observe change of cell status caused by any cell culture parameter affecting the cells.

The present invention is in a preferred embodiment directed to analysis of glycosylation changes when the density of cells is altered. The inventors noticed that this has a major impact of the glycosylation during cell culture.

It is further realized that if there is limitations in genetic or differentiation stability of cells, these would increase probability for changes in glycan structures. Cell populations in early stage of differentiation have potential to produce different cell populations. The present inventors were able to discover glycosylation changes in early human cell populations.

### Differentiation of Cell Lines

The present invention is specifically directed to observe glycosylation changes according to the present invention when differentiation of a cell line is observed. In a preferred embodiment the invention is directed to methods for observation of differentiation from early human cell or another preferred cell type according to the present invention to mesodermal types of stem cell

In case there is heterogeneity in cell material this may cause observable changes or harmful effects in glycosylation.

Furthermore, the changes in carbohydrate structures, even non-harmful or functionally unknown, can be used to obtain information about the exact genetic status of the cells.

The present invention is specifically directed to the analysis of changes of glycosylation, preferably changes in glycan profiles, individual glycan signals, and/or relative abundancies of individual glycans or glycan groups according to the present invention in order to observe changes of cell status during cell cultivation.

### Analysis of Supporting/Feeder Cell Lines

The present invention is specifically directed to observe glycosylation differences according to the present invention, on supporting/feeder cells used in cultivation of stem cells and early human cells or other preferred cell type. It is known in the art that some cells have superior activities to act as a support/feeder cells than other cells. In a preferred embodiment the invention is directed to methods for observation of differences on glycosylation on these supporting/feeder cells. This information can be used in design of novel reagents to support the growth of the stem cells and early human cells or other preferred cell type.

### Contaminations or Alterations in Cells Due to Process Conditions

### Conditions and Reagents Inducing Harmful Glycosylation or Harmful Glycosylation Related Effects to Cells During Cell Handling

The inventors further revealed conditions and reagents inducing harmful glycans to be expressed by cells with same associated problems as the contaminating glycans. The inventors found out that several reagents used in a regular cell purification processes caused changes in early human cell materials.

It is realized, that the materials during cell handling may affect the glycosylation of cell materials. This may be based on the adhesion, adsorption, or metabolic accumulation of the structure in cells under processing.

In a preferred embodiment the cell handling reagents are tested with regard to the presence glycan component being antigenic or harmful structure such as cell surface NeuGc, Neu-O-Ac or mannose structure. The testing is especially preferred for human early cell populations and preferred subpopulations thereof.

The inventors note effects of various effector molecules in cell culture on the glycans expressed by the cells if absortion or metabolic transfer of the carbohydrate structures have not been performed. The effectors typically mediate a signal to cell for example through binding a cell surface receptor.

The effector molecules include various cytokines, growth factors, and their signalling molecules and co-receptors. The effector molecules may be also carbohydrates or carbohydrate binding proteins such as lectins.

Controlled Cell Isolation/Purification and Culture Conditions to Avoid Contaminations with Harmful Glycans or Other Alteration in Glycome Level

### Stress Caused by Cell Handling

It is realized that cell handling including isolation/purification, and handling in context of cell storage and cell culture processes are not natural conditions for cells and cause physical and chemical stress for cells. The present invention allows control of potential changes caused by the stress. The control may be combined by regular methods may be combined with regular checking of cell viability or the intactness of cell structures by other means.

Examples of Physical and/or Chemical Stress in Cell Handling Step

Washing and centrifuging cells cause physical stress which may break or harm cell membrane structures. Cell purifications and separations or analysis under non-physiological flow conditions also expose cells to certain non-physiological stress. Cell storage processes and cell preservation and handling at lower temperatures affects the membrane structure. All handling steps involving change of composition of media or other solution, especially washing solutions around the cells affect the cells for example by altered water and salt balance or by altering concentrations of other molecules effecting biochemical and physiological control of cells.

### Observation and Control of Glycome Changes by Stress in Cell Handling Processes

The inventors revealed that the method according to the invention is useful for observing changes in cell membranes which usually effectively alter at least part of the glycome observed according to the invention. It is realized that this related to exact organization and intact structures cell membranes and specific glycan structures being part of the organization.

The present invention is specifically directed to observation of total glycome and/or cell surface glycomes, these methods are further aimed for the use in the analysis of intactness of cells especially in context of stressful condition for the cells, especially when the cells are exposed to physical and/or chemical stress. It is realized that each new cell handling step and/or new condition for a cell handling step is useful to be controlled by the methods according to the invention. It is further realized that the analysis of glycome is useful for search of most effectively altering glycan structures for analysis by other methods such as binding by specific carbohydrate binding agents including especially carbohydrate binding proteins (lectins, antibodies, enzymes and engineered proteins with carbohydrate binding activity).

Controlled Cell Preparation (Isolation or Purification) with Regard to Reagents

The inventors analysed process steps of common cell preparation methods. Multiple sources of potential contamination by animal materials were discovered.

The present invention is specifically directed to carbohydrate analysis methods to control of cell preparation processes. The present invention is specifically directed to the process of controlling the potential contaminations with animal type glycans, preferably N-glycolylneuraminic acid at various steps of the process.

The invention is further directed to specific glycan controlled reagents to be used in cell isolation

The glycan-controlled reagents may be controlled on three levels:
1. Reagents controlled not to contain observable levels of harmful glycan structure, preferably N-glycolylneuraminic acid or structures related to it
2. Reagents controlled not to contain observable levels of glycan structures similar to the ones in the cell preparation
3. Reagent controlled not to contain observable levels of any glycan structures.

The control levels 2 and 3 are useful especially when cell status is controlled by glycan analysis and/or profiling methods. In case reagents in cell preparation would contain the indicated glycan structures this would make the control more difficult or prevent it. It is further noticed that glycan structures may represent biological activity modifying the cell status.

### Cell Preparation Methods Including Glycan-Controlled Reagents

The present invention is further directed to specific cell purification methods including glycan-controlled reagents.

### Preferred Controlled Cell Purification Process

When the binders are used for cell purification or other process after which cells are used in method where the glycans of the binder may have biological effect the binders are preferably glycan controlled or glycan neutralized proteins.

The present invention is especially directed to controlled production of human early cells containing one or several following steps. It was realized that on each step using regular reagents in following process there is risk of contamination by extragenous glycan material. The process is directed to the use of controlled reagents and materials according to the invention in the steps of the process. Preferred purification of cells includes at least one of the steps including the use of controlled reagent, more preferably at least two steps are included, more preferably at least 3 steps and most preferably at least steps 1, 2, 3, 4, and 6.
1. Washing cell material with controlled reagent.
2. When antibody based process is used cell material is in a preferred embodiment blocked with controlled Fc-receptor blocking reagent. It is further realized that part of glycosylation may be needed in a antibody preparation, in a preferred embodiment a terminally depleted glycan is used.
3. Contacting cells with immobilized cell binder material including controlled blocking material and controlled cell binder material. In a more preferred the cell binder material comprises magnetic beads and controlled gelatin material according the invention. In a preferred embodiment the cell binder material is controlled, preferably a cell binder antibody material is controlled. Otherwise the cell binder antibodies may contain even N-glycolylneuraminic acid, especially when the antibody is produced by a cell line producing N-glycolylneuraminic acid and contaminate the product.
4. Washing immobilized cells with controlled protein preparation or non-protein preparation. In a preferred process magnetic beads are washed with controlled protein preparation, more preferably with controlled albumin preparation.
5. Optional release of cells from immobilization.
6. Washing purified cells with controlled protein preparation or non-protein preparation.

In a preferred embodiment the preferred process is a method using immunomagnetic beads for purification of early human cells, preferably purification of cord blood cells.

The present invention is further directed to cell purification kit, preferably an immunomagnetic cell purification kit comprising at least one controlled reagent, more preferably at least two controlled reagents, even more preferably three controlled reagents, even preferably four reagents and most preferably the preferred controlled reagents are selected from the group: albumin, gelatin, antibody for cell purification and Fc-receptor blocking reagent, which may be an antibody.

Contaminations with Harmful Glycans Such as Antigenic Animal Type Glycans

Several glycans structures contaminating cell products may weaken the biological activity of the product.

The harmful glycans can affect the viability during handling of cells, or viability and/or desired bioactivity and/or safety in therapeutic use of cells.

The harmful glycan structures may reduce the in vitro or in vivo viability of the cells by causing or increasing binding of destructive lectins or antibodies to the cells. Such protein material may be included e.g. in protein preparations used in cell handling materials. Carbohydrate targeting lectins are also present on human tissues and cells, especially in blood and endothelial surfaces. Carbohydrate binding antibodies in human blood can activate complement and cause other immune responses in vivo. Furthermore immune defense lectins in blood or leukocytes may direct immune defense against unusual glycan structures.

Additionally harmful glycans may cause harmful aggregation of cells in vivo or in vitro. The glycans may cause unwanted changes in developmental status of cells by aggregation and/or changes in cell surface lectin mediated biological regulation.

Additional problems include allergenic nature of harmful glycans and misdirected targeting of cells by endothelial/cellular carbohydrate receptors in vivo.

### Common Structural Features of all Glycomes and Preferred Common Subfeatures

The present invention reveals useful glycan markers for stem cells and combinations thereof and glycome compositions comprising specific amounts of key glycan structures. The invention is furthermore directed to specific terminal and core structures and to the combinations thereof.

The preferred glycome glycan structure(s) and/or glycomes from cells according to the invention comprise structure(s) according to the formula C0:

R₁Hexβz{R₃)}ₙ₁Hex(NAc)ₙ₂XyR₂,

Wherein X is glycosidically linked disaccharide epitope β4(Fucα6)ₙGN, wherein n is 0 or 1, or X is nothing and

### Hex is Gal or Man or GlcA,

### HexNAc is GlcNAc or GalNAc,

y is anomeric linkage structure α and/or β or linkage from derivatized anomeric carbon,
z is linkage position 3 or 4, with the provision that when z is 4 then HexNAc is GlcNAc and then Hex is Man or Hex is Gal or Hex is GlcA, and
when z is 3 then Hex is GlcA or Gal and HexNAc is GlcNAc or GalNAc;
n1 is 0 or 1 indicating presence or absence of R3;
n2 is 0 or 1, indicating the presence or absence of NAc, with the proviso that n2 can be 0 only when Hexβz is Galβ4, and n2 is preferably 0, n2 structures are preferably derived from glycolipids;
R₁ indicates 1-4, preferably 1-3, natural type carbohydrate substituents linked to the core structures or nothing;
R₂ is reducing end hydroxyl, chemical reducing end derivative or natural asparagine N-glycoside derivative such as asparagine N-glycosides including asparagine N-glycoside aminoacids and/or peptides derived from protein, or natural serine or threonine linked O-glycoside derivative such as serine or threonine linked O-glycosides including asparagine N-glycoside aminoacids and/or peptides derived from protein, or when n2 is 1 R2 is nothing or a ceramide structure or a derivative of a ceramide structure, such as lysolipid and amide derivatives thereof,
R3 is nothing or a branching structure representing a GlcNAcβ6 or an oligosaccharide with GlcNAcβ6 at its reducing end linked to GalNAc (when HexNAc is GalNAc); or when Hex is Gal and HexNAc is GlcNAc, and when z is 3 then R3 is Fucα4 or nothing, and when z is 4 R3 is Fucα3 or nothing.

The preferred disaccharide epitopes in the glycan structures and glycomes according to the invention include structures Galβ4GlcNAc, Manβ4GlcNAc, GlcAβ4GlcNAc, Galβ3GlcNAc, Galβ3GalNAc, GlcAβ3GlcNAc, GlcAβ3GalNAc, and Galβ4Glc, which may be further derivatized from reducing end carbon atom and non-reducing monosaccharide residues and is in a separate embodiment branched from the reducing end residue. Preferred branched epitopes include Galβ4(Fucα3)GlcNAc, Galβ3(Fucα4)GlcNAc, and Galβ3(GlcNAcβ6)GalNAc, which may be further derivatized from reducing end carbon atom and non-reducing monosaccharide residues.

### Preferred Epitopes for Methods According to the Invention

### N-acetyllactosamine Galβ3/4GlcNAc Terminal Epitopes

The two N-acetyllactosamine epitopes Galβ4GlcNAc and/or Galβ3GlcNAc represent preferred terminal epitopes present on stem cells or backbone structures of the preferred terminal epitopes for example further comprising sialic acid or fucose derivatisations according to the invention. In a preferred embodiment the invention is directed to fucosylated and/or non-substituted glycan non-reducing end forms of the terminal epitopes, more preferably to fucosylated and non-substituted forms. The invention is especially directed to non-reducing end terminal (non-substituted) natural Galβ4GlcNAc and/or Galβ3GlcNAc-structures from human stem cell glycomes. The invention is in a specific embodiment directed to non-reducing end terminal fucosylated natural Galβ4GlcNAc and/or Galβ3GlcNAc-structures from human stem cell glycomes.

### Preferred Fucosylated N-Acetyllactosamines

The preferred fucosylated epitopes are according to the Formula TF:

(Fucα2)ₙ₁Galβ3/4(Fucα4/3)ₙ₂GlcNAcβ-R

### Wherein

n1 is 0 or 1 indicating presence or absence of Fucα2;
n2 is 0 or 1, indicating the presence or absence of Fucα4/3 (branch), and
R is the reducing end core structure of N-glycan, O-glycan and/or glycolipid.

The preferred structures thus include type 1 lactosamines (Galβ3GlcNAc based): Galβ3(Fucα4)GlcNAc (Lewis a), Fucα2Galβ3GlcNAc H-type 1, structure and, Fucα2Galβ3(Fucα4)GlcNAc (Lewis b) and

type 2 lactosamines (Galβ4GlcNAc based): Galβ4(Fucα3)GlcNAc (Lewis x), Fucα2Galβ4GlcNAc H-type 2, structure and, Fucα2Galβ4(Fucα3)GlcNAc (Lewis y).

The type 2 lactosamines (fucosylated and/or terminal non-substituted) form an especially preferred group in context of adult stem cells and differentiated cells derived directly from these. Type 1 lactosamines (Galβ3GlcNAc-structures) are especially preferred in context of embryonal-type stem cells.

### Lactosamines Galβ3/4GlcNAc and Glycolipid Structures Comprising Lactose Structures (Galβ4Glc)

The lactosamines form a preferred structure group with lactose-based glycolipids. The structures share similar features as products of β3/4Gal-transferases. The β3/4 galactose based structures were observed to produce characteristic features of protein linked and glycolipid glycomes.

The invention revealed that furthermore Galβ3/4GlcNAc-structures are a key feature of differentiation related structures on glycolipids of various stem cell types. Such glycolipids comprise two preferred structural epitopes according to the invention. The most preferred glycolipid types include thus lactosylceramide based glycosphingolipids and especially lacto-(Galβ3GlcNAc), such as lactotetraosylceramide Galβ3GlcNAcβ3Galβ4GlcβCer, preferred structures further including its non-reducing terminal structures selected from the group: Galβ3(Fucα4)GlcNAc (Lewis a), Fucα2Galβ3GlcNAc (H-type 1), structure and, Fucα2Galβ3(Fucα4)GlcNAc (Lewis b) or sialylated structure SAα3Galβ3GlcNAc or SAα3Galβ3(Fucα4)GlcNAc, wherein SA is a sialic acid, preferably Neu5Ac preferably replacing Galβ3GlcNAc of lactotetraosylceramide and its fucosylated and/or elongated variants such as preferably according to the Formula:

(Sacα3)ₙ₅(Fucα2)ₙ₁Galβ3(Fucα4)ₙ₃GlcNAcβ3[Galβ3/4(Fucα4/3)ₙ₂GlcNAcβ3]ₙ₄Galβ4GlcβCer

wherein
n1 is 0 or 1, indicating presence or absence of Fucα2;
n2 is 0 or 1, indicating the presence or absence of Fucα4/3 (branch),
n3 is 0 or 1, indicating the presence or absence of Fucα4 (branch)
n4 is 0 or 1, indicating the presence or absence of (fucosylated) N-acetyllactosamine elongation;
n5 is 0 or 1, indicating the presence or absence of Sacα3 elongation;
Sac is terminal structure, preferably sialic acid, with α3-linkage, with the proviso that when Sac is present, n5 is 1, then n1 is 0 and
neolacto (Galβ4GlcNAc)-comprising glycolipids such as
   neolactotetraosylceramide Galβ4GlcNAcβ3Galβ4GlcβCer, preferred structures further including its non-reducing terminal Galβ4(Fucα3)GlcNAc (Lewis x), Fucα2Galβ4GlcNAc H-type 2, structure and, Fucα2Galβ4(Fucα3)GlcNAc (Lewis y) and
   its fucosylated and/or elongated variants such as preferably

(Sacα3/6)ₙ₅(Fucα2)ₙ₁Galβ4(Fucα3)ₘ₃GlcNAcβ3[Galβ4(Fucα3)ₙ₂GlcNAcβ3]ₙ₄Galβ4GlcβCer

n1 is 0 or 1 indicating presence or absence of Fucα2;
n2 is 0 or 1, indicating the presence or absence of Fucα3 (branch),
n3 is 0 or 1, indicating the presence or absence of Fucα3 (branch)
n4 is 0 or 1, indicating the presence or absence of (fucosylated) N-acetyllactosamine elongation,
n5 is 0 or 1, indicating the presence or absence of Sacα3/6 elongation;
Sac is terminal structure, preferably sialic acid (SA) with α3-linkage, or sialic acid with α6-linkage, with the proviso that when Sac is present, n5 is 1, then n1 is 0, and when sialic acid is bound by α6-linkage preferably also n3 is 0.

### Preferred Stem Cell Glycosphingolipid Glycan Profiles, Compositions, and Marker Structures

The inventors were able to describe stem cell glycolipid glycomes by mass spectrometric profiling of liberated free glycans, revealing about 80 glycan signals from different stem cell types. The proposed monosaccharide compositions of the neutral glycans were composed of 2-7 Hex, 0-5 HexNAc, and 0-4 dHex. The proposed monosaccharide compositions of the acidic glycan signals were composed of 0-2 NeuAc, 2-9 Hex, 0-6 HexNAc, 0-3 dHex, and/or 0-1 sulphate or phosphate esters. The present invention is especially directed to analysis and targeting of such stem cell glycan profiles and/or structures for the uses described in the present invention with respect to stem cells.

The present invention is further specifically directed to glycosphingolipid glycan signals specific tostem cell types as described in the Examples. In a preferred embodiment, glycan signals typical to hESC, preferentially including 876 and 892 are used in their analysis, more preferentially FucHexHexNAcLac, wherein α1,2-Fuc is preferential to α1,3/4-Fuc, and Hex₂HexNAc₁Lac, and more preferentially to Galβ3[Hex₁HexNAc₁]Lac. In another preferred embodiment, glycan signals typical to MSC, especially CB MSC, preferentially including 1460 and 1298, as well as large neutral glycolipids, especially Hex₂₋₃HexNAc₃Lac, more preferentially poly-N-acetyllactosamine chains, even more preferentially β1,6-branched, and preferentially terminated with type II LacNAc epitopes as described above, are used in context of MSC according to the uses described in the present invention.

Terminal glycan epitopes that were demonstrated in the present experiments in stem cell glycosphingolipid glycans are useful in recognizing stem cells or specifically binding to the stem cells via glycans, and other uses according to the present invention, including terminal epitopes: Gal, Galβ4Glc (Lac), Galβ4GlcNAc (LacNAc type 2), Galβ3, Non-reducing terminal HexNAc, Fuc, α1,2-Fuc, α1,3-Fuc, Fucα2Gal, Fucα2Galβ4GlcNAc (H type 2), Fucα2Galβ4Glc (2′-fucosyllactose), Fucα3GlcNAc, Galβ4(Fucα3)GlcNAc (Lex), Fucα3Glc, Galβ4(Fucα3)Glc (3-fucosyllactose), Neu5Ac, Neu5Acα2,3, and Neu5Acα2,6. The present invention is further directed to the total terminal epitope profiles within the total stem cell glycosphingolipid glycomes and/or glycomes.

The inventors were further able to characterize in hESC the corresponding glycan signals to SSEA-3 and SSEA-4 developmental related antigens, as well as their molar proportions within the stem cell glycome. The invention is further directed to quantitative analysis of such stem cell epitopes within the total glycomes or subglycomes, which is useful as a more efficient alternative with respect to antibodies that recognize only surface antigens. In a further embodiment, the present invention is directed to finding and characterizing the expression of cryptic developmental and/or stem cell antigens within the total glycome profiles by studying total glycan profiles, as demonstrated in the Examples for α1,2-fucosylated antigen expression in hESC in contrast to SSEA-1 expression in mouse ES cells.

The present invention revealed characteristic variations (increased or decreased expression in comparison to similar control cell or a contamination cell or like) of both structure types in various cell materials according to the invention. The structures were revealed with characteristic and varying expression in three different glycome types: N-glycans, O-glycans, and glycolipids. The invention revealed that the glycan structures are a characteristic feature of stem cells and are useful for various analysis methods according to the invention. Amounts of these and relative amounts of the epitopes and/or derivatives varies between cell lines or between cells exposed to different conditions during growing, storage, or induction with effector molecules such as cytokines and/or hormones.

### Preferred Epitopes and Antibody Binders Especially for Analysis of Embryonal Stem Cells

The antibody labelling experiment Tables with embryonal stem cells revealed specific of type 1 N-acetyllactosamine antigen recognizing antibodies recognizing non-modified disaccharide Galβ3GlcNAc (Le c, Lewis c), and fucosylated derivatives H type and Lewis b. The antibodies were effective in recognizing hESC cell populations in comparison to mouse feeder cells mEF used for cultivation of the stem cells.

Specific different H type 2 recognizing antibodies were revealed to recognize different subpopulations of embryonal stem cells and thus usefulness for defining subpopulations of the cells.

The invention further revealed a specific Lewis x and sialyl-Lewis x structures on the embryonal stem cells.

Other preferred binders and/or antibodies comprise of binders which bind to the same epitope than GF 287 (H type 1). In a preferred embodiment, an antibody binds to Fucα2Galβ3GlcNAc epitope. A more preferred antibody comprises of the antibody of clone 17-206 (ab3355) by Abcam. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonic stem cells from a mixture of cells comprising feeder and stem cells.

Other preferred binders and/or antibodies comprise of binders which bind to the same epitope than GF 279 (Lewis c, Galβ3GlcNAc). In a preferred embodiment, an antibody binds to Galβ3GlcNAc epitope in glycoconjugates, more preferably in glycoproteins and glycolipids such as lactotetraosylceramide. A more preferred antibody comprises of the antibody of clone K21 (ab3352) by Abcam. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonic stem cells from a mixture of cells comprising feeder and stem cells.

Other preferred binders and/or antibodies comprise of binders which bind to the same epitope than GF 288 (Globo H). In a preferred embodiment, an antibody binds to Fucα2Galβ3GalNAcβ epitope, more preferably Fucα2Galβ3GalNAcβ3GalαLacCer epitope. A more preferred antibody comprises of the antibody of clone A69-A/E8 (MAB-S206) by Glycotope. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonic stem cells from a mixture of cells comprising feeder and stem cells.

Other preferred binders and/or antibodies comprise of binders which bind to the same epitope than GF 284 (H type 2). In a preferred embodiment, an antibody binds to Fucα2Galβ4GlcNAc epitope. A more preferred antibody comprises of the antibody of clone B393 (DM3015) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonic stem cells from a mixture of cells comprising feeder and stem cells.

Other preferred binders and/or antibodies comprise of binders which bind to the same epitope than GF 283 (Lewis b). In a preferred embodiment, an antibody binds to Fucα2Galβ3(Fucα4)GlcNAc epitope. A more preferred antibody comprises of the antibody of clone 2-25LE (DM3122) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonic stem cells from a mixture of cells comprising feeder and stem cells.

Other preferred binders and/or antibodies comprise of binders which bind to the same epitope than GF 286 (H type 2). In a preferred embodiment, an antibody binds to Fucα2Galβ4GlcNAc epitope. A more preferred antibody comprises of the antibody of clone B393 (BM258P) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonic stem cells from a mixture of cells comprising feeder and stem cells.

Other preferred binders and/or antibodies comprise of binders which bind to the same epitope than GF 290 (H type 2). In a preferred embodiment, an antibody binds to Fucα2Galβ4GlcNAc epitope. A more preferred antibody comprises of the antibody of clone A51-B/A6 (MAB-S204) by Glycotope. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonic stem cells from a mixture of cells comprising feeder and stem cells.

Other binders binding to feeder cells, preferably mouse feeder cells, comprise of binders which bind to the same epitope than GF 285 (H type 2). In a preferred embodiment, an antibody binds to Fucα2Galβ4GlcNAc, Fucα2Galβ3(Fucα4)GlcNAc, Fucα2Galβ4(Fucα3)GlcNAc epitope. A more preferred antibody comprises of the antibody of clone B389 (DM3014) by Acris. This epitope is suitable and can be used to detect, isolate and evaluate of feeder cells, preferably mouse feeder cells in culture with human embryonic stem cells. The detection can be performed in vitro, for FACS purposes and/or for cell lineage specific purposes. This antibody can be used to positively isolate and/or separate and/or enrich feeder cells (negatively select stem cells), preferably mouse embryonic feeder cells from a mixture of cells comprising feeder and stem cells.

Other binders binding to stem cells, preferably human stem cells, comprise of binders which bind to the same epitope than GF 289 (Lewis y). In a preferred embodiment, an antibody binds to Fucα2Galβ4(Fucα3)GlcNAc epitope. A more preferred antibody comprises of the antibody of clone A70-C/C8 (MAB-S201) by Glycotope. This epitope is suitable and can be used to detect, isolate and evaluate of stem cells, preferably human stem cells in culture with feeder cells. The detection can be performed in vitro, for FACS purposes and/or for cell lineage specific purposes. This antibody can be used to positively isolate and/or separate and/or enrich stem cells (negatively select feeder cells), preferably human stem cells from a mixture of cells comprising feeder and stem cells.

The staining intensity and cell number of stained stem cells, i.e. glycan structures of the present invention on stem cells indicates suitability and usefulness of the binder for isolation and differentiation marker. For example, low relative number of a glycan structure expressing cells may indicate lineage specificity and usefulness for selection of a subset and when selected/isolated from the colonies and cultured. Low number of expression is less than 5%, less than 10%, less than 15%, less than 20%, less than 30% or less than 40%. Further, low number of expression is contemplated when the expression levels are between 1-10%, 10%-20%, 15-25%, 20-40%, 25-35% or 35-50%. Typically, FACS analysis can be performed to enrich, isolate and/or select subsets of cells expressing a glycan structure(s).

High number of glycan expressing cells may indicate usefulness in pluripotency/multipotency marker and that the binder is useful in identifying, characterizing, selecting or isolating pluripotent or multipotent stem cells in a population of mammalian cells. High number of expression is more than 50%, more preferably more than 60%, even more preferably more than 70%, and most preferably more than 80%, 90 or 95%. Further, high number of expression is contemplated when the expression levels are between 50-60, 55%-65%, 60-70%, 70-80, 80-90%, 90-100 or 95-100%. Typically, FACS analysis can be performed to enrich, isolate and/or select subsets of cells expressing a glycan structure(s).

The epitopes recognized by the binders GF 279, GF 287, and GF 289 and the binders are particularly useful in characterizing pluripotency and multipotency of stem cells in a culture. The epitopes recognized by the binders GF 283, GF 284, GF 286, GF 288, and GF 290 and the binders are particularly useful for selecting or isolating subsets of stem cells. These subset or subpopulations can be further propagated and studied in vitro for their potency to differentiate and for differentiated cells or cell committed to a certain differentiation path.

The percentage as used herein means ratio of how many cells express a glycan structure to all the cells subjected to an analysis or an experiment. For example, 20% stem cells expressing a glycan structure in a stem cell colony means that a binder, e.g. an antibody staining can be observed in about 20% of cells when assessed visually.

In colonies a glycan structure bearing cells can be distributed in a particular regions or they can be scattered in small patch like colonies. Patch like observed stem cells are useful for cell lineage specific studies, isolation and separation. Patch like characteristics were observed with GF 283, GF 284, GF 286, GF 288, and GF 290.

For positive selection of feeder cells, preferably mouse feeder cells, most preferably embryonic fibroblasts, GF 285 is useful. This antibody has lower specificity and may have binding to e.g. Lewis y, which has been observed also in mEF cells. It stains almost all feeder cells whereas very little if at all staining is found in stem cells. The antibody was however under optimized condition revealed to bind to thin surface of embryonal bodies, this was in complementary to Lewis y antibody to the core of embryoid body. For all percentages of expression, see Tables.

### Mesenchymal Stem Cells and Differentiated Tissue Type Stem Cells Derived Thereof.

Antibodies useful for evaluation of differentiation status of mesenchymal stem cells

Example 13 shows labelling of mesenchymal stem cells and differentiated mesenchymal stem cells.

Invention revealed that structures recognized by antibody GF303, preferably Fucα2Galβ3GlcNAc, and GF276 appear during the differentiation of mesenchymal stem cells to osteogenically differentiated stem cells. It was further revealed, that the GalNAcα-group structures GF278, corresponding to Tn-antigen, and GF277, sialyl-Tn increase simultaneously.

The invention is further directed to the preferred uses according to the invention for binders to several target structures, which are characteristic to both mesenchymal stem cells (especially bone marrow derived) and the osteogenically differentiated mesenchymal stem cells. The preferred target structures include one GalNAcα-group structure recognizable by the antibody GF275, the antigen of the antibody is preferably sialylated O-glycan glycopeptide epitope as known for the antibody. The epitopes expressed in both mesenchymal and the osteonically differentiated stem cells further includes two characteristic globo-type antigen structures: the antigen of GF298, which binding correspond to globotriose(Gb3)-type antigens, and the antigen of GF297, which correspond to globotetraose(Gb4) type antigens. The invention has further revealed that terminal type two lactosamine epitopes are especially expressed in both types of mesenchymal stem cells and this was exemplified by staining both cell by antibody recognizing H type II antigen in Example 13.

The invention is further directed to the preferred uses according to the invention for binders to several target structures which are substantially reduced or practically diminished/reduced to non-observable level when mesenchymal stem cells (especially bone marrow derived) differentiates to more differentiated, preferably osteogenically differentiated mesenchymal stem cells. These target structures include two globoseries structures, which are preferably Galactosyl-globoside type structure, recognized as antigen SSEA-3, and sialyl-galactosylgloboside type structure, recognized as antigen SSEA-4. The preferred reducing target structures further include two type two N-acetyllactosamine target structures Lewis x and sialyl-Lewis x. Globoside-type glycosphingolipid structures were detected by the inventors in MSC in minor but significant amounts compared to hESC in direct structural analysis, more specifically glycan signals corresponding to SSEA-3 and SSEA-4 glycan antigen monosaccharide compositions. These antigens were also detected by monoclonal antibodies in MSC. The present invention is therefore specifically directed to these globoside structures in context of MSC and cells derived from them in uses described in the invention.

In a preferred embodiment of the present invention, the antibodies or binders which bind to the same epitope than GF275, GF277, GF278, GF297, GF298, GF302, GF305, GF307, GF353, or GF354 are useful to detect/recognize, preferably bone marrow derived, mesenchymal stem cells (corresponding epitopes recognized by the antibodies are listed in Example 13). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow from mixture of cells comprising other, bone marrow derived, cells.

Other binders binding to stem cells, preferably human stem cells, comprise of binders which bind to the same epitope than GF275 (sialylated carbohydrate epitope of the MUC-1 glycoprotein). A more preferred antibody comprises of the antibody of clone BM3359 by Acris. This epitope is suitable and can be used to detect, isolate and evaluate of (mesenchymal) stem cells, preferably bone marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow, or differentiated in osteogenic direction from mixture of cells comprising other, bone marrow derived, cells.

Other binders binding to stem cells, preferably human stem cells, comprise of binders which bind to the same epitope than GF305 (Lewis x). A more preferred antibody comprises of the antibody of clone CBL144 by Chemicon. This epitope is suitable and can be used to detect, isolate and evaluate of (mesenchymal) stem cells, preferably bone marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow from mixture of cells.

Other binders binding to stem cells, preferably human stem cells, comprise of binders which bind to the same epitope than GF307 (sialyl lewis x). A more preferred antibody comprises of the antibody of clone MAB2096 by Chemicon. This epitope is suitable and can be used to detect, isolate and evaluate of (mesenchymal) stem cells, preferably bone marrow derived, in culture or in vivo. The detection can be performed in vitro, for FACS purposes and/or for cell lineage specific purposes. The antibodies or binders can be used to positively isolate and/or separate and/or enrich stem cells, preferably mesenchymal and/or derived from bone marrow from mixture of cells.

In a preferred embodiment, the antibodies or binders which bind to the same epitope than GF305, GF307, GF353 or GF354 are useful for positive selection and/or enrichment of mesenchymal stem cells (corresponding epitopes recognized by the antibodies are listed in Example 13).

In another preferred embodiment of the present invention, antibodies or binders which bind to the same epitope than GF275, GF276, GF277, GF278, GF297, GF298, GF302, GF303, GF307 or GF353 are useful to detect/recognize differentiated, preferably bone marrow derived, mesenchymal stem cells and/or differentiated in osteogenic direction (corresponding epitopes recognized by the antibodies are listed in Example 13). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow from mixture of cells comprising other, bone marrow derived, cells.

Other binders binding to stem cells, preferably human stem cells, comprise of binders which bind to the same epitope than GF297 (globoside GL4). A more preferred antibody comprises of the antibody of clone ab23949 by Abcam. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF298 (human CD77; GB3). A more preferred antibody comprises of the antibody of clone SM1160 by Acris. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF302 (H type 2 blood antigen). In a preferred embodiment, an antibody binds to Fucα2Galβ4GlcNAc epitope. A more preferred antibody comprises of the antibody of clone DM3015 by Acris. This epitope is suitable and can be used to detect, isolate and evaluate of undifferentiated (mesenchymal) stem cells, preferably bone marrow derived, and differentiated ones, preferably for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

In a preferred embodiment of the present invention, antibodies or binders which bind to the same epitope than GF276, GF277, GF278, GF303, GF305, GF307, GF353, or GF354 are useful to detect/recognize, preferably bone marrow derived, mesenchymal stem cells and differentiated in osteogenic direction (corresponding epitopes recognized by the antibodies are listed in Example 13). These epitopes are suitable and can be used to detect, isolate and evaluate of (mesenchymal) stem cells, preferably bone marrow derived, in culture or in vivo. The detection can be performed in vitro, for FACS purposes and/or for cell lineage specific purposes. These antibodies can be used to positively isolate and/or separate and/or enrich stem cells, preferably mesenchymal and/or derived from bone marrow, or differentiated in osteogenic direction from mixture of cells comprising other, bone marrow derived, cells.

Further, the binders which bind to the same epitope than GF276 or GF303, or antibodies GF276 and/or GF303 are particularly useful to detect, isolate and evaluate of osteogenically differentiated stem cells, in culture or in vivo (corresponding epitopes recognized by the antibodies are listed in Example 13).

Other binders binding to stem cells, preferably human stem cells, comprise of binders which bind to the same epitope than GF276 (oncofetal antigen). A more preferred antibody comprises of the antibody of clone DM288 by Acris. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF277 (human sialosyl-Tn antigen; STn, sCD175). A more preferred antibody comprises of the antibody of clone DM3197 by Acris. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF278 (human sialosyl-Tn antigen; STn, sCD175 B1.1). A more preferred antibody comprises of the antibody of clone DM3218 by Acris. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Other binders binding to stem cells, preferably human stem cells, comprise of binders which bind to the same epitope than GF303 (blood group H1 antigen, BG4). In a preferred embodiment, an antibody binds to Fucα2Galβ3GlcNAc epitope. A more preferred antibody comprises of the antibody of clone ab3355 by Abcam. This epitope is suitable and can be used to detect, isolate and evaluate of differentiated (mesenchymal) stem cells, preferably bone marrow derived and for osteogenic direction, in culture or in vivo. The detection can be performed in vitro, for FACS purposes and/or for cell lineage specific purposes. The antibodies or binders can be used to positively isolate and/or separate and/or enrich cells, preferably mesenchymal stem cells in osteogenic direction from mixture of cells.

Further, the antibodies or binders are useful to isolate and enrich stem cells for osteogenic lineage. This can be performed with positive selection, for example, with antibodies GF276, GF277, GF278, and GF303 (corresponding epitopes recognized by the antibodies are listed in Example 13). For negative depletion, a preferred epitope is the same as recognized with the antibodies GF296, GF300, GF304, GF305, GF307, GF353, or GF354. For negative depletion, a preferred epitope is the same as recognized with the antibody GF354 (SSEA-4) or GF307 (Sialyl Lewis x).

### Comparison Between Different Stem Cell Types

The present data revealed that comparison of a group of type 1 and type two N-acetyllactosamines is useful method for characterization stem cells such as mesenchymal stem cells and embryonal stem cells and or separating the cells from contaminating cell populations such as fibroblasts like feeder cells. The non-differentiated mesenchymal cell were devoid of type I N-acetyllactosamine antigens revealed from the hESC cells, while both cell types and potential contaminating fibroblast have variable labelling with type II N-acetyllactosamine recognizing antibodies.

The term “mainly” indicates preferably at least 60%, more preferably at least 75% and most preferably at least 90%. In the context of stem cells, the term “mainly” indicates preferably at least 60%, more preferably at least 75% and most preferably at least 90% of cells expressing a glycan structure and useful for identifying, characterizing, selecting or isolating pluripotent or multipotent stem cells in a population of mammalian cells.

### Uses of the Binders for Isolation of Cellular Components and Mixtures Thereof.

The invention revealed novel binding reagents are in a preferred embodiment used for isolation of cellular components from stem cells comprising the novel target/marker structures. The isolated cellular are preferably free glycans or glycans conjugated to proteins or lipids or fragment thereof.

The invention is especially directed to isolation of the cellular components comprising the structures when the structures comprises one or several types glycan materials sele
a) Free glycans released from the stem cell materials and/or
b) Glycan conjugate material such as
   b1) glycoamino acid materials including
      b1a) glycoproteins
      b1b) glycopeptides including glyco-oligopeptides and glycopolypeptides and/or
   b2) lipid linked materials comprising the preferred carbohydrate structures revealed by the invention.

### General Method for Isolation Cellular Components Comprising the Target Structures

The isolation of cellular components according to the invention means production of a molecular fraction comprising increased (or enriched) amount of the glycans comprising the target structures according to the invention in method comprising the step of binding of the binder molecule according to the invention to the corresponding target structures, which are glycan structures bound by the specific binder.

The process of isolation the fraction involving the contacting the binder molecule according to the invention with the corresponding target structures derived from stem cells and isolating the enriched target structure composition.

The preferred method to isolate cellular component includes following steps

1) Providing a stem cell sample.

2) Contacting the binder molecule according to the invention with the corresponding target structures.

3) Isolating the complex of the binder and target structure at least from part of cellular materials.

It is realized that the components are in general enriched in specific fractions of cellular structures such as cellular membrane fractions including plasma membrane and organelle fractions and soluble glycan comprising fractions such as soluble protein, lipid or free glycans fractions. It is realized that the binder can be used to total cellular fractions.

In a preferred embodiment the target structures are enriched within a fraction of cellular proteins such as cell surface proteins releasable by protease or detergent soluble membrane proteins. The preferred target structure composition comprise glycoproteins or glycopeptides comprising glycan structure corresponding to the binder structure and peptide or protein epitopes specifically expressed in stem cells or in proportions characteristic to stem cells.

More preferably the invention is directed to purification of the target structure fraction in the isolation step. The purification is in a preferred mode of invention is at least partial purification. Preferably the target glycan containing material is purified at least two fold, preferably among the components of cell fraction wherein it is expressed. More preferred purification levels includes 5-fold and 10 fold purification, more preferably 100, and even more preferably 1000-fold purification. Preferably the purified fraction comprises at least 10% of the target glycan comprising molecules, even more preferably at least 30%, even more preferably at least 50%, even more preferably at least 70% pure and most preferably at least 90% pure. Preferably the % value is mole percent in comparison to other non-target glycan comprising glycaconjugate molecules, more preferably the material is essentially devoid of other major organic contaminating molecules.

### Preferred Purified Target Glycan Compositions and Target Glycan-Binder Complexes

The invention is also directed to isolated or purified target glycan-binder complexes and isolated target glycan molecule compositions, wherein the target glycans are enriched with a specific target structures according to the invention.

Preferably the purified target glycan-binder complex compositions comprises at least 10% of the target glycan comprising molecules in complex with binder, even more preferably at least 30%, even more preferably at least 50%, even more preferably at least 70% pure and most preferably at least 90% pure target glycan comprising molecules in complex with binder.

Preferably the purified target glycan composition comprises at least 10% of the target glycan comprising molecules, even more preferably at least 30%, even more preferably at least 50%, even more preferably at least 70% pure and most preferably at least 90% pure target glycan comprising molecules.

The invention is further directed to the enriched target glycan composition produced by the process of isolation the fraction involving the steps of the contacting the binder molecule according to the invention with the corresponding target structures derived from stem cell and isolating the enriched target structure.

### Binder Technology for Purification of Target Glycans

The methods for affinity purification of cellular glycoproteins, glycopeptides, free oligosaccharides and other glycan conjugates are well-known in the art. The preferred methods include solid phase involving binder technologies such as affinity chromatography, precipitation such as immunoprecipitation, binder-magnetic methods such as immunomagnetic bead methods. Affinity chromatographies has been described for purification of glycopeptides by using lectins (Wang Y et al (2006) Glycobiology 16 (6) 514-23) or by antibodies or purification of glycoproteins/peptides by using antibodies (e.g. Prat M et al cancer Res (1989) 49, 1415-21; Kim Y D et al et al Cancer Res (1989) 49, 2379) and/or lectins (e.g. Cumming and Kornfeld (1982) J Biol Chem 257, 11235-40; Yae E et al. (1991) 1078 (3) 369-76; Shibuya N et al (1988) 267 (2) 676-80; Gonchoroff D G et al. 1989, 35, 29-32; Hentges and Bause (1997) Biol Chem 378 (9) 1031-8). Specific methods have been developed for weakly binding antibodies even for recognition of free oligosaccharides as described e.g. in (Ohlson S et al. J Chromatogr A (1997) 758 (2) 199-208), Ohlson S et al. Anal Biochem (1988) 169 (1) 204-8). The methods may involve multiple steps by binders of different specificities as shown e.g. in (Cummings and Komfeld (1982) J Biol Chem 257, 11235-40). Antibody or protein (lectin) binder affinity chromatography for oligosaccharide mixtures has been also described e.g. in (Kitagawa H et al. (1991) J Biochem 110 (49 598-604; Kitagawa H et al. (1989) Biochemistry 28 (22) 8891-7; Dakour J et al Arch Biochem Biophys (1988) 264, 203-13) and for glycolipids e.g. in (Bouhours D et al (1990) Arch Biochem Biophys 282 (1) 141-6). Further information of glycan directed affinity chromatography and/or useful lectin and antibody specificities is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and L is, Halina) Kluwer Academic publishers Dordrecht, The Neatherlands).

The methods includes normal pressure or in HPLC chromatographies and may include additional steps using traditional chromatographic methods or other protein and peptide purification methods, a preferred additional isolation methods is gel filtration (size exclusion) chromatography for isolation of especially lower Mw glycans and conjugates, preferably glycopeptides.

It is further known that isolated proteins and peptides can be recognized by mass spectrometric methods e.g. (Wang Y et al (2006) Glycobiology 16 (6) 514-23). The invention is specifically directed to use of the binders according to the invention for purification of glycans and/or their conjugates and recognition of the isolated component by methods such as mass spectrometry, peptide sequencing, chemical analysis, array analysis or other methods known in the art.

### Revealing Presence Trypsin Sensitive Forms of Glycan Targets

The invention reveals in Examples that part of the target structures of present glycan binders, especially monoclonal antibodies are trypsin sensitive. The antigen structures are essentially not observed or these are observed in reduced amount in FACS analysis of cell surface antigens when cells are treated (released from cultivation) by trypsin but observable after Versene treatment (0.02% EDTA in PBS). This was observed for example for labelling of mesenchymal stem cells by the antibody GF354, which has been indicated to bind SSEA-4 antigen. This target antigen structure has been traditionally considered to be sialyl-galactosylgloboside glycolipid, but obviously the antibody recognizes only an epitope at the non-reducing end of glycan sequence. The present invention is now especially directed to methods of isolation and characterization of mesenchymal stem cell glycopeptide bound glycan structure(s), which can be bound and enriched by the SSEA-4 antibodies, and to characterization of corresponding glycopeptides and glycoproteins. The invention is further directed to analysis of trypsin insensitive glycan materials from stem cell especially mesenchymal stem cells and embryonal stem cells.

The invention revealed also that major part of the sialyl-mucin type target of ab GF 275 is trypsin sensitive and minor part is not trypsin sensitive. The invention is directed to isolation of both trypsin sensitive and trypsin insensitive glycan fractions, preferably glycoprotein(s) and glycopeptides, by methods according to the invention. The invention is further directed to isolation and characterization of protein degrading enzyme (protease) sensitive likely glycopeptides and glycoproteins bound by antibody GF 302, preferably when the materials are isolated from mesenchymal stem cells.

As used herein, “binder”, “binding agent” and “marker” are used interchangeably.

### Antibodies

Information about useful lectin and antibody specificities useful according to the invention and for reducing end elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and L is, Halina) Kluwer Academic publishers Dordrecht, The Netherlands and internet databases such as pubmed/espacenet or antibody databases such as www.glyco.is.ritsumei.ac.ip/epitope/, which list monoclonal antibody specificities).

Various procedures known in the art may be used for the production of polyclonal antibodies to peptide motifs and regions or fragments thereof. For the production of antibodies, any suitable host animal (including but not limited to rabbits, mice, rats, or hamsters) are immunized by injection with a peptide (immunogenic fragment). Various adjuvants may be used to increase the immunological response, depending on the host species, including but not limited to Freund's (complete and incomplete) adjuvant, mineral gels such as aluminum hydroxide, surface active substances such as lysolecithin, pluronic polyols, polyanions, oil emulsions, keyhole limpet hemocyanins, dinitrophenol, and potentially useful human adjuvants such as BCG {Bacille Calmette-Guerin) and Corynebacterium parvum.

A monoclonal antibody to a peptide motif(s) may be prepared by using any technique which provides for the production of antibody molecules by continuous cell lines in culture. These include but are not limited to the hybridoma technique originally described by Kδhler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 41, Sp210-Ag14, FO, NSO/U, MPC-11, MPC11-X45-GTG 1.7 and S194/5XX0 BuI; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al, Proc Natl Acad Sci 81: 6851-6855, 1984; Neuberger et al, Nature 312: 604-608, 1984; Takeda et al, Nature 314: 452-454; 1985). Alternatively, techniques described for the production of single-chain antibodies (U.S. Pat. No. 4,946,778) can be adapted to produce influenza-specific single chain antibodies.

Antibody fragments that contain the idiotype of the molecule may be generated by known techniques. For example, such fragments include, but are not limited to, the F(ab′)2 fragment which may be produced by pepsin digestion of the antibody molecule; the Fab′ fragments which may be generated by reducing the disulfide bridges of the F(ab′)2 fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgG1, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. {Nature 321: 522-525, 1986), Riechmann et al, {Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165, 1994. Further changes can then be introduced into the antibody framework to modulate affinity or immunogenicity.

Likewise, using techniques known in the art to isolate CDRs, compositions comprising CDRs are generated. Complementarity determining regions are characterized by six polypeptide loops, three loops for each of the heavy or light chain variable regions. The amino acid position in a CDR and framework region is set out by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from residues 49-59 and from residues 92-103 of the heavy and light chain variable regions (Janeway and Travers, Immunobiology, 2nd Edition, Garland Publishing, New York, 1996). The CDR regions in any given antibody may be found within several amino acids of these approximated residues set forth above. An immunoglobulin variable region also consists of “framework” regions surrounding the CDRs. The sequences of the framework regions of different light or heavy chains are highly conserved within a species, and are also conserved between human and murine sequences.

Compositions comprising one, two, and/or three CDRs of a heavy chain variable region or a light chain variable region of a monoclonal antibody are generated. Polypeptide compositions comprising one, two, three, four, five and/or six complementarity determining regions of a monoclonal antibody secreted by a hybridoma are also contemplated. Using the conserved framework sequences surrounding the CDRs, PCR primers complementary to these consensus sequences are generated to amplify a CDR sequence located between the primer regions. Techniques for cloning and expressing nucleotide and polypeptide sequences are well-established in the art [see e.g., Sambrook et al., Molecular Cloning: A Laboratory Manual, 2nd Edition, Cold Spring Harbor, N.Y. (1989)]. The amplified CDR sequences are ligated into an appropriate plasmid. The plasmid comprising one, two, three, four, five and/or six cloned CDRs optionally contains additional polypeptide encoding regions linked to the CDR.

Preferably, the antibody is any antibody specific for a glycan structure of Formula (I) or a fragment thereof. The antibody used in the present invention encompasses any antibody or fragment thereof, either native or recombinant, synthetic or naturally-derived, monoclonal or polyclonal which retains sufficient specificity to bind specifically to the glycan structure according to Formula (I) which is indicative of stem cells. As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′).sub.2 fragments, and Fv fragments.

The antibodies can be conjugated to other suitable molecules and compounds including, but not limited to, enzymes, magnetic beads, colloidal magnetic beads, haptens, fluorochromes, metal compounds, radioactive compounds, chromatography resins, solid supports or drugs. The enzymes that can be conjugated to the antibodies include, but are not limited to, alkaline phosphatase, peroxidase, urease and .beta.-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P. Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m, .sup. 125 I and amino acids comprising any radionuclides, including, but not limited to .sup.14 C, .sup.3H and .sup.35 S.

Antibodies to glycan structure(s) of Formula (I) may be obtained from any source. They may be commercially available. Effectively, any means which detects the presence of glycan structure(s) on the stem cells is with the scope of the present invention. An example of such an antibody is a H type 1 (clone 17-206; GF 287) antibody from Abcam.

### HSCs

The methods outlined herein are particularly useful for identifying HSCs or progeny thereof from a population of cells. However, additional markers may be used to further distinguish subpopulations within the general HSC, or stem cell, population.

The various sub-populations may be distinguished by levels of binders to glycan structures of Formula (I) on stem cells. This may manifest on the stem cell surface (or on feeder cell if feeder cell specific binder is used) which may be detected by the methods outlined herein. However, the present invention may be used to distinguish between various phenotypes of the stem cell or HSC population including, but not limited to, the CD34.sup.+, CD38.sup.−, CD90.sup.+(thy1) and Lin.sup.− cells. Preferably the cells identified are selected from the group including, but not limited to, CD34.sup.+, CD38.sup.−, CD90+ (thy 1), or Lin.sup.−.

The present invention thus encompasses methods of enriching a population for stem and/or HSCs or progeny thereof. The methods involve combining a mixture of HSCs or progeny thereof with an antibody or marker or binding protein/agent or binder that recognizes and binds to glycan structure according to Formula (I) on stem cell(s) under conditions which allow the antibody or marker or binder to bind to glycan structure according to Formula (I) on stem cell(s) and separating the cells recognized by the antibody or marker to obtain a population substantially enriched in stem cells or progeny thereof. The methods can be used as a diagnostic assay for the number of HSCs or progeny thereof in a sample. The cells and antibody or marker are combined under conditions sufficient to allow specific binding of the antibody or marker to glycan structure according to Formula (I) on stem cell(s) which are then quantitated. The HSCs or stem cells or progeny thereof can be isolated or further purified.

As discussed above the cell population may be obtained from any source of stem cells or HSCs or progeny thereof including those samples discussed above.

The detection for the presence of glycan structure(s) according to Formula (I) on stem cell(s) may be conducted in any way to identify glycan structure according to Formula (I) on stem cell(s). Preferably the detection is by use of a marker or binding protein for glycan structure according to Formula (I) on stem cell(s). The binder/marker for glycan structure according to Formula (I) on stem cell(s) may be any of the markers discussed above. However, antibodies or binding proteins to glycan structure according to Formula (I) on stem cell(s) are particularly useful as a marker for glycan structure according to Formula (I) on stem cell(s).

Various techniques can be employed to separate or enrich the cells by initially removing cells of dedicated lineage. Monoclonal antibodies, binding proteins and lectins are particularly useful for identifying cell lineages and/or stages of differentiation. The antibodies can be attached to a solid support to allow for crude separation. The separation techniques employed should maximize the retention of viability of the fraction to be collected. Various techniques of different efficacy can be employed to obtain “relatively crude” separations. The particular technique employed will depend upon efficiency of separation, associated cytotoxicity, ease and speed of performance, and necessity for sophisticated equipment and/or technical skill.

Procedures for separation or enrichment can include, but are not limited to, magnetic separation, using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique.

The use of separation or enrichment techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye, Hoescht 33342).

Techniques providing accurate separation include, but are not limited to, FACS, which can have varying degrees of sophistication, e.g., a plurality of color channels, low angle and obtuse light scattering detecting channels, impedance channels, etc. Any method which can isolate and distinguish these cells according to levels of expression of glycan structure according to Formula (I) on stem cell(s) may be used.

In a first separation, typically starting with about 1.times.10.sup. 10, preferably at about 5.times.10.sup.8-9 cells, antibodies or binding proteins or lectins to glycan structure according to Formula (I) on stem cell(s) can be labeled with at least one fluorochrome, while the antibodies or binding proteins for the various dedicated lineages, can be conjugated to at least one different fluorochrome. While each of the lineages can be separated in a separate step, desirably the lineages are separated at the same time as one is positively selecting for glycan structure according to Formula (I) on stem cell markers. The cells can be selected against dead cells, by employing dyes associated with dead cells (including but not limited to, propidium iodide (PI)).

To further enrich for any cell population, specific markers for those cell populations may be used. For instance, specific markers for specific cell lineages such as lymphoid, myeloid or erythroid lineages may be used to enrich for or against these cells. These markers may be used to enrich for HSCs or progeny thereof by removing or selecting out mesenchymal or keratinocyte stem cells.

The methods described above can include further enrichment steps for cells by positive selection for other stem cell specific markers. Suitable positive stem cell markers include, but are not limited to, SSEA-3, SSEA-4, Tra 1-60, CD34.sup.+, Thy-1.sup.+, and c-kit.sup.+. By appropriate selection with particular factors and the development of bioassays which allow for self-regeneration of HSCs or progeny thereof and screening of the HSCs or progeny thereof as to their markers, a composition enriched for viable HSCs or progeny thereof can be produced for a variety of purposes.

Once the stem cells or HSC or progeny thereof population is isolated, further isolation techniques may be employed to isolate sub-populations within the HSCs or progeny thereof. Specific markers including cell selection systems such as FACS for cell lineages may be used to identify and isolate the various cell lineages.

In yet another aspect of the present invention there is provided a method of measuring the content of stem cells or HSC or their progeny said method comprising

obtaining a cell population comprising stem cells or progeny thereof,
combining the cell population with a binding protein or binder for glycan structure according to Formula (I) on stem cell(s) thereof,
selecting for those cells which are identified by the binding protein for glycan structure according to Formula (I) on stem cell(s) thereof, and
quantifying the amount of selected cells relative to the quantity of cells in the cell population prior to selection with the binding protein.

### Binder-Label Conjugates

The present invention is specifically directed to the binding of the structures according to the present invention, when the binder is conjugated with “a label structure”. The label structure means a molecule observable in a assay such as for example a fluorescent molecule, a radioactive molecule, a detectable enzyme such as horse radish peroxidase or biotin/streptavidin/avidin. When the labelled binding molecule is contacted with the cells according to the invention, the cells can be monitored, observed and/or sorted based on the presence of the label on the cell surface. Monitoring and observation may occur by regular methods for observing labels such as fluorescence measuring devices, microscopes, scintillation counters and other devices for measuring radioactivity.

### Use of Binder and Labelled Binder-Conjugates for Cell Sorting

The invention is specifically directed to use of the binders and their labelled conjugates for sorting or selecting human stem cells from biological materials or samples including cell materials comprising other cell types. The preferred cell types includes cord blood, peripheral blood and embryonal stem cells and associated cells. The labels can be used for sorting cell types according to invention from other similar cells. In another embodiment the cells are sorted from different cell types such as blood cells or in context of cultured cells preferably feeder cells, for example in context of embryonal stem cells corresponding feeder cells such as human or mouse feeder cells. A preferred cell sorting method is FACS sorting. Another sorting methods utilized immobilized binder structures and removal of unbound cells for separation of bound and unbound cells.

### Use of Immobilized Binder Structures

In a preferred embodiment the binder structure is conjugated to a solid phase. The cells are contacted with the solid phase, and part of the material is bound to surface. This method may be used to separation of cells and analysis of cell surface structures, or study cell biological changes of cells due to immobilization. In the analytics involving method the cells are preferably tagged with or labelled with a reagent for the detection of the cells bound to the solid phase through a binder structure on the solid phase. The methods preferably further include one or more steps of washing to remove unbound cells.

Preferred solid phases include cell suitable plastic materials used in contacting cells such as cell cultivation bottles, petri dishes and microtiter wells; fermentor surface materials, etc.

### Specific Recognition Between Preferred Stem Cells and Contaminating Cells

The invention is further directed to methods of recognizing stem cells from differentiated cells such as feeder cells, preferably animal feeder cells and more preferably mouse feeder cells. It is further realized, that the present reagents can be used for purification of stem cells by any fractionation method using the specific binding reagents.

Preferred fractionation methods includes fluorescence activated cell sorting (FACS), affinity chromatography methods, and bead methods such as magnetic bead methods.

Preferred reagents for recognition between preferred cells, preferably embryonal type cells, and contaminating cells, such as feeder cells, most preferably mouse feeder cells, include reagents according to the Tables, more preferably proteins with similar specificity with lectins PSA, MAA, and PNA.

The invention is further directed to positive selection methods including specific binding to the stem cell population but not to contaminating cell population. The invention is further directed to negative selection methods including specific binding to the contaminating cell population but not to the stem cell population. In yet another embodiment of recognition of stem cells the stem cell population is recognized together with a homogenous cell population such as a feeder cell population, preferably when separation of other materials is needed. It is realized that a reagent for positive selection can be selected so that it binds stem cells as in the present invention and not to the contaminating cell population and a reagent for negative selection by selecting opposite specificity. In case of one population of cells according to the invention is to be selected from a novel cell population not studied in the present invention, the binding molecules according to the invention maybe used when verified to have suitable specificity with regard to the novel cell population (binding or not binding). The invention is specifically directed to analysis of such binding specificity for development of a new binding or selection method according to the invention.

### Manipulation of Cells by Binders

The invention is specifically directed to manipulation of cells by the specific binding proteins. It is realized that the glycans described have important roles in the interactions between cells and thus binders or binding molecules can be used for specific biological manipulation of cells. The manipulation may be performed by free or immobilized binders. In a preferred embodiment cells are used for manipulation of cell under cell culture conditions to affect the growth rate of the cells.

### Stem Cell Nomenclature

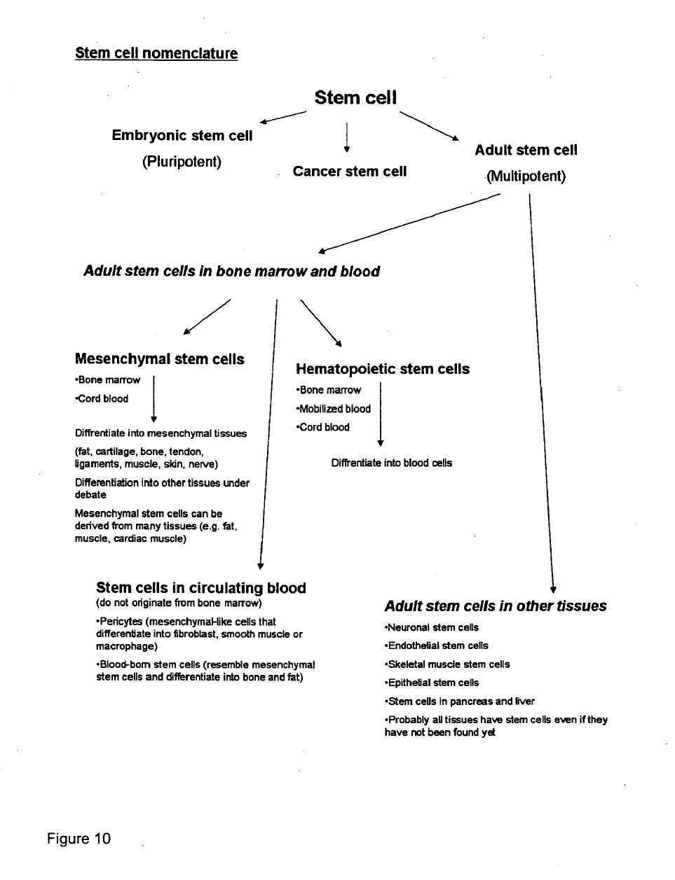

The present invention is directed to analysis of all stem cell types, preferably human stem cells. A general nomenclature of the stem cells is described in FIG. 10. The alternative nomenclature of the present invention describe early human cells which are in a preferred embodiment equivalent of adult stem cells (including cord blood type materials) as shown in FIG. 10. Adult stem cells in bone marrow and blood is equivalent for stem cells from “blood related tissues”.

### Lectins for Manipulation of Stem Cells, Especially Under Cell Culture Conditions

The present invention is especially directed to use of lectins as specific binding proteins for analysis of status of stem cells and/or for the manipulation of stems cells.

The invention is specifically directed to manipulation of stem cells under cell culture conditions growing the stem cells in presence of lectins. The manipulation is preferably performed by immobilized lectins on surface of cell culture vessels. The invention is especially directed to the manipulation of the growth rate of stem cells by growing the cells in the presence of lectins, as show in Tables.

The invention is in a preferred embodiment directed to manipulation of stem cells by specific lectins recognizing specific glycan marker structures according to invention from the cell surfaces. The invention is in a preferred embodiment directed to use of Gal recognizing lectins such as ECA-lectin or similar human lectins such as galectins for recognition of galectin ligand glycans identified from the cell surfaces. It was further realized that there is specific variations of galectin expression in genomic levels in stem cells, especially for galectins-1, -3, and -8. The present invention is especially directed to methods of testing of these lectins for manipulation of growth rates of embryonal type stem cells and for adult stem cells in bone marrow and blood and differentiating derivatives thereof.

### Sorting of Stem Cells by Specific Lectins

The invention revealed use of specific lectin types recognizing cell surface glycan epitopes according to the invention for sorting of stem cells, especially by FACS methods, most preferred cell types to be sorted includes adult stem cells in blood and bone marrow, especially cord blood cells. Preferred lectins for sorting of cord blood cells include GNA, STA, GS-II, PWA, HHA, PSA, RCA, and others as shown in Example 11. The relevance of the lectins for isolating specific stem cell populations was demonstrated by double labeling with known stem cells markers, as described in Example 11.

### Preferred Structures of O-Glycan Glycomes of Stem Cells

The present invention is especially directed to following O-glycan marker structures of stem cells: Core 1 type O-glycan structures following the marker composition NeuAc₂Hex₁HexNAc₁, preferably including structures SAα3Galβ3GalNAc and/or SAα3Galβ3(Saα6)GalNAc; and Core 2 type O-glycan structures following the marker composition NeuAc₀₋₂Hex₂HexNAc₂dHex₀₋₁, more preferentially further including the glycan series NeuAc₀₋₂Hex₂₊ₙHexNAc₂₊ₙdHex₀₋₁, wherein n is either 1, 2, or 3 and more preferentially n is 1 or 2, and even more preferentially n is 1;

more specifically preferably including R₁Galβ4(R₃)GlcNAcβ6(R₂Galβ3)GalNAc,
wherein R₁ and R₂ are independently either nothing or sialic acid residue, preferably α2,3-linked sialic acid residue, or an elongation with HexₙHexNAcₙ, wherein n is independently an integer at least 1, preferably between 1-3, most preferably between 1-2, and most preferably 1, and the elongation may terminate in sialic acid residue, preferably α2,3-linked sialic acid residue; and R₃ is independently either nothing or fucose residue, preferably α1,3-linked fucose residue.

It is realized that these structures correlate with expression of β6GlcNAc-transferases synthesizing core 2 structures.

### Preferred Branched N-Acetyllactosamine Type Glycosphingolipids

The invention further revealed branched, 1-type, poly-N-acetyllactosamines with two terminal Galβ4-residues from glycolipids of human stem cells. The structures correlate with expression of β6GlcNAc-transferases capable of branching poly-N-acetyllactosamines and further to binding of lectins specific for branched poly-N-acetylalctosamines. It was further noticed that PWA-lectin had an activity in manipulation of stem cells, especially the growth rate thereof.

### Preferred Qualitative and Quantitative Complete N-Glycomes of Stem Cells

### Preferred Binders for Stem Cell Sorting and Isolation

As described in the Examples, the inventors found that especially the mannose-specific and especially α1,3-linked mannose-binding lectin GNA was suitable for negative selection enrichment of CD34+stem cells from CB MNC. In addition, the poly-LacNAc specific lectin STA and the fucose-specific and especially α1,2-linked fucose-specific lectin UEA were suitable for positive selection enrichment of CD34+ stem cells from CB MNC.

The present invention is specifically directed to stem cell binding reagents, preferentially proteins, preferentially mannose-binding or α1,3-linked mannose-binding, poly-LacNAc binding, LacNAc-binding, and/or fucose- or preferentially α1,2-linked fucose-binding; in a preferred embodiment stem cell binding or nonbinding lectins, more preferentially GNA, STA, and/or UEA; and in a further preferred embodiment combinations thereof, to uses described in the present invention taking advantage of glycan-binding reagents that selectively either bind to or do not bind to stem cells.

Preferred Uses for Stem Cell Type Specific Galectins and/or Galectin Ligands

As described in the Examples, the inventors also found that different stem cells have distinct galectin expression profiles and also distinct galectin (glycan) ligand expression profiles. The present invention is further directed to using galactose-binding reagents, preferentially galactose-binding lectins, more preferentially specific galectins; in a stem cell type specific fashion to modulate or bind to certain stem cells as described in the present invention to the uses described. In a further preferred embodiment, the present invention is directed to using galectin ligand structures, derivatives thereof, or ligand-mimicking reagents to uses described in the present invention in stem cell type specific fashion.

Analysis and Utilization of Poly-N-Acetyllactosamine Sequences and Non-Reducing Terminal Epitopes Associated with Different Glycan Types

The present invention is directed to poly-N-acetyllactosamine sequences (poly-LacNAc) associated with cell types according to the present invention. The inventors found that different types of poly-LacNAc are characteristic to different cell types, as described in the Examples of the present invention. In particular, CB MNC are characterized by linear type 2 poly-LacNAc; MSC, especially CB MSC, are characterized by branched type 2 poly-LacNAc; and hESC are characterized by type 1 terminating poly-LacNAc. The present invention is especially directed to the analysis and utilization of these glycan characteristics according to the present invention. The present invention is further directed to the analysis and utilization of the specific cell-type associated glycan sequences revealed in the present Examples according to the present invention.

The present invention is directed to non-reducing terminal epitopes in different glycan classes including N- and O-glycans, glycosphingolipid glycans, and poly-LacNAc. The inventors found that especially the relative amounts of β1,4-linked Gal, β1,3-linked Gal, α1,2-linked Fuc, α1,3/4-linked Fuc, α-linked sialic acid, and α2,3-linked sialic acid are characteristically different between the studied cell types; and the invention is especially directed to the analysis and utilization of these glycan characteristics according to the present invention.

The present invention is further directed to analyzing fucosylation degree in O-glycans by comparing indicative glycan signals such as neutral O-glycan signals at m/z 771 and 917 as described in the Examples. The inventors found that compared to other cell types analyzed in the present invention, hESC had low relative abundance of neutral O-glycan signal at m/z 917 compared to 771, indicating low fucosylation degree of the O-glycan sequences corresponding to the signal at m/z 771 and containing terminal β1,4-linked Gal. Another difference was the occurrence of abundant signal at m/z 552 in hESC, corresponding to Hex₁,HexNAc₁dHex₁, including α1,2-fucosylated Core 1 O-glycan sequence. In contrast, in CB MNC the glycan signal at m/z 917 is relatively abundant, indicating high fucosylation degree of the O-glycan sequences corresponding to the signal at m/z 771 and containing terminal β1,4-linked Gal. The other cell types analyzed in the present invention also had characteristic fucosylation degree between these two cell types.

Especially, the present invention is directed to analyzing terminal epitopes associated with poly-LacNAc in stem cells, more preferably when these epitopes are presented in the context of a poly-LacNAc chain, most preferably in O-glycans or glycosphingolipids. The present invention is further directed to analyzing such characteristic poly-LacNAc, terminal epitope, and fucosylation profiles according to the methods of the present invention, in glycan structural characterization and specific glycosylation type identification, and other uses of the present invention; especially when this analysis is done based on endo-β-galactosidase digestion, by studying the non-reducing terminal fragments and their profile, and/or by studying the reducing terminal fragments and their profile, as described in the Examples of the present invention. The inventors found that cell-type specific glycosylation features are efficiently reflected in the endo-β-galactosidase reaction products and their profiles. The present invention is further directed to such reaction product profiles and their analysis according to the present invention.

The inventors further found that all three most thoroughly analyzed cellular glycan classes, N-glycans, O-glycans, and glycosphingolipid glycans, were differently regulated compared to each other, especially with regard to non-reducing terminal glycan epitopes and poly-LacNAc sequences as described in the Examples and Tables of the present invention. Therefore, combining quantitative glycan profile analysis data from more than one glycan class will yield significantly more information. The present invention is especially directed to combining glycan data obtained by the methods of the present invention, from more than one glycan class selected from the group of N-glycans, O-glycans, and glycosphingolipid glycans; more preferably, all three classes are analyzed; and use of this information according to the present invention. In a preferred embodiment, N-glycan data is combined with O-glycan data; and in a further preferred embodiment, N-glycan data is combined with glycosphingolipid glycan data.

General. There seems not to be a single specific glycan epitope analyzed absolutely specific only for one total population of HSCs exactly like the traditional CD34+ population but there is closely similar labelling e.g. by anti-SLex antibodies. Instead there seems to be enrichment of certain glycan epitopes in stem cells and in differentiated cells.

In some cases the antibodies recognize epitopes, which are highly or several fold enriched in a specific cell type or present above the current FACS detection limit in a part of a cell population but not in the other corresponding cell populations. It is realized that such antibodies are especially useful for specific recognition of the specific cell population. Furthermore, combination of several antibodies recognizing independent populations of specific cell types is useful for recognition of a larger cell population in a positive or negative manner.

The present invention provides reagents common to hematopoietic cell populations in general or for specific differentiation stage of hematopoietic cells. Furthermore the invention reveals specific marker structures for hematopoietic stem cells derived from specific tissue types such as cord blood or bone marrow.

The invention is further directed to the use of the target structures and specific glycan target structures for screening of additional binders preferably specific antibodies or lectins recognizing the terminal glycan structures and the use of the binders produced by the screening according to the invention. A preferred tool for the screening is glycan array comprising one or several hematopoietic stem cells glycan epitopes according to the invention and additional control glycans. The invention is directed to screening of known antibodies or searching information of their published specificities in order to find high specificity antibodies. Furthermore the invention is directed to the search of the structures from phage display libraries.

It is further realized that the individual marker recognizable on major part of the cells can be used for the recognition and/or isolation of the cells when the associated cells in the context does not express the specific glycan epitope. These markers may be used for example isolation of the cell populations from biological materials such as tissues or cell cultures, when the expression of the marker is low or non-existent in the associated cells.

It is realized that tissues comprising stem cells usually contain these in primitive stem cell stage and highly expressed markers according can be optimised or selected for the cell isolation. In a preferred embodiment the invention is directed to selection of hematopoietic stem cells from cord blood from CD34− type cells by the binders according to the invention such as by poly-lactosamine recognizing binders including preferably STA or sialyl-Lewis x recognizing proteins including preferably monoclonal antibodies recognizing the glycan epitopes according the invention (Table 23). In a separate embodiments the invention is directed to the use of selectins or selectin homologous proteins optimized for the reconition.

It is possible to select cell cultivation conditions to preserve specific differentiation status and present antibodies recognizing major or practically total cell population are useful for the analysis or isolation of cells in these contexts.

The methods such as FACS analysis allows quantitative determination of the structures on cells and thus the antibodies recognizing part of the cell population are also characteristic for the cell population.

### Combinations

Combination of several antibodies for specific analysis of a hematoppietic or associated population for cell population would characterize the cell population. In a preferred embodiment at least one “effectively binding antibody”, recognizing major part (over 35%) or most (50%) of the cell population (preferably more than 30%, an in order of increasing preference more than 40%, 50%, 60%, 70%, 80% and most preferably more than 90%), are selected for the analytic method in combination with at least one “non-binding antibody”, recognizing preferably minor part (preferably from detection limit of the method to low level of recognition, in order of preference less than 10%, 7%, 5%, 2% or 1% of cells, e.g. 0.2-10% of cells, more preferably 0.2-5% of the cells, and even more preferably 0.5-2% or most preferably 0.5%-1.0%) or no part of the cell population (under or at the detection limit e.g. in order of preference less than 5%, 2%, 1%, 0.5%, and 0.2%) and more preferably practically no part of the cell population according to the invention. In yet another embodiment the combination method includes use of “moderately binding antibody”, which recognize substantial part of the cells, being preferably from 5 to 50%, more preferably from 7% to 40% and most preferably from 10 to 35%.

The invention is directed to the use of several reagents recognizing terminal epitopes together, preferably at least two reagents, more preferably at least three epitopes, even more preferably at least four, even more preferably at least five, even more preferably at least six, even more preferably at least seven, and most preferably at least 8 to recognize enough positive and negative targets together. It is realized that with high specificity binders selectively and specifically recognizing elongated epitopes, less binders may be needed e.g. these would be preferably used as combinations of at least two reagents, more preferably at least three epitopes, even more preferably at least four, even more preferably at least five, most preferably at least six antibodies. The high specificity binders selectively and specifically recognizing elongated epitopes binds one of the elongated epitopes at least in order of increasing preference, 5, 10, 20, 50, or 100 fold affinity, methods for measuring the antibody binding affinities are well known in the art. The invention is also directed to the use of lower specificity antibodies capable of effective recognition of one elongated epitope but also at least one, preferably only one additional elongated epitope with same terminal structure

The reagents are preferably used in arrays comprising in order of increasing preference 5, 10, 20, 40 or 70 or all reagents shown in cell labelling experiments.

The invention is further directed to combinations of fucosylated and/or sialylated structures with structures devoid of these modifications. Combinations of type 1 N-acetyllactosamine with type 2 structures with type 1 (Galβ3GlcNAc) structures and/or with mucin type and/or glyccolipids structures. In a preferred combination at least one binding antibody is combined with non-binding antibody recognizing different structure type

The antibodies recognize certain glycan epitopes revealed as target structures according to the invention. It is realized that specificities and affinities of the antibodies vary between the clones. It was realized that certain clones known to recognize certain glycan structure does not necessarily recognize the same cell population.

### Specific Targets

Preferred binder structures for the selection of binder for the cell culture associated use

The invention revealed several blood derived stem cell associated structures such N-acetyllactosamine structures bound to protein linked N-glycans and O-glycans and glycolipids.

Preferred terminal epitopes has been represented in Formulas according to the invention ormiulas and TABLES specifically in Table 23, derived from the extensive structural data of the examples. The invention revealed novel elongated binder target epitopes which are preferably recognized by a binder, preferably by a high specificity binder not recognizing effectively the same terminal structure on other carrier structures. The invention is especially directed to the use of specific binder for enrichment and/or cultivation of hematopoietic stem cells such as blood derived CD34+, or CD133+ (or LIN−) cells, preferred structures for this are indicated on left column after structure in Table 23 and structures more enriched and the enrichmens with non-hematopietic associated cells such as blood derived mononuclear CD34−, CD133− (or LIN+ cells), indicated on the right hand column Table 23 for negative selection to enrich and/or cultivate hematopoietic stem cells. The invention is further directed to the recognition of terminal epitomes wherein the terminal N-glycan epitopes are β2-linked to mannose, O-glycan N-acetyllactosamine based epitopes are β6-linked to GalNAc and glycolipid N-acetyllactosamine based epitopes are β3-linked to Gal.

The preferred structures for binding and positive selection of cells in context of cultivation of hematopoietic stem cells especially cord blood hematopoietic cells such as CD34+ includes specific

### Fucosylated Structures

### i) α3-Fucosylated Structures,

Preferred α3-fucosylated structures includes especially Lewis x and sialyl-Lewis x. The invention is in a preferred embodiment directed to blood derived stem cell populations enriched by binding to α3-fucosylated structures on the cell surfaces by specific binder reagents.

The invention is further directed to complex of α3-fucose specific binder reagent and blood derived stem cells, especially for the use of cell cultivation.

Specific sialyl-Lewis x structures were revealed to be effectively cord blood CD34+ cell specific and useful for binding and manipulation of the cells.

The preferred binding reagent for sLex includes GF 526, and GF307, especially recognizing major part or practically all CD34+ cells from cord blood and GF 516 recognizing substantial subpopulation of about 40% of the cells.

In a preferred embodiment the sialyl Lewis x specific reagent bind especially core II sLex [SAα3Galβ4(Fucα3)GlcNAcβ6(R1Galβ3)GalNAcαSer/Thr, wherein R1 i.e. sialic acid (SAα3) or nothing.] as the antibody GF526. The invention is especially directed to the selection of sLex and core II sLEx positive cells by specific binder regions from material comprising blood derived stem cells such as cord blood or bone marrow, most preferably cord blood and especially for the culture of stem cells. In a preferred embodiment the cell sorting system is FACS or solid phase comprising the binders.

It is realized that in cord blood hematopoietic cells (especially CD34+ cells) there is individual specific variation especially in Lewis x expression and part of the Lewis x antibody binders also recognize non-hematopoietic CD34− cells (e.g. antibodies GF 515 and GF 525 (a CD15 antibody)), but especially GF305 and GF517 and GF518 recognizes effectively Lewis x on certain individuals in CD34+ cell preparations.

The invention is especially directed to the selection of specific Lewis x, and preferred subtype thereof, positive cells by specific binder reagens from material comprising blood derived stem cells such as cord blood or bone marrow, most preferably cord blood and especially for the culture of stem cells. In a preferred embodiment the cell sorting system is FACS or solid phase comprising the binders.

Lotus tetragonolobus agglutinin LTA is an example of a lower specificity reagent which binds strongly to divalent or oligovalent Lewis x and is therefore useful for selection of cell with higher complex α3-fucosylation.

Treatment of human cord blood mononuclear cells with the LTA lectin coated magnetic beads produced a novel cell population with high enrichment of stem cell marker CD34.

### ii) α2-Fucosylated Structures,

Preferred α2-fucosylated structures includes especially H-type structures recognizable by antibodies recognizing substantial cord blood CD34+ cell populations, GF 288 and GF 394 (globo H). The invention is in a preferred embodiment directed to blood derived stem cell populations enriched by binding to α2-fucosylated structures on the cell surfaces by specific binder reagents. The invention is further directed to complex of α2-fucose specific binder reagent and blood derived stem cells, especially for the use of cell cultivation.

The invention is further directed to specific lower specificity reagents effectively recognizing H-epitopes of blood derived stem cells, a preferred region is the lectin UEA, in a preferred embodiment the lectin is aimed for the use of the lectin in context of cell culture and selection or manipulation of blood derive d stem cells.

iii) Non-Fucosylated Sialyl-Lactosamines

The invention revealed sialylated N-acetyllactosamine structures (SAα3Galβ4GlcNAcβ) recognizing lectin MAA (Maackia amuriensis agglutinin) as a useful reagent for isolation of stem cell, especially negative isolation from human cord blood. The lectin binds most of the cord blood cells but less effectively CD34+ cells.

### Gal/GalNAc/GalNAcα-Comprising Structures

### iv) Galβ3GalNAc Structures

The invention revealed that blood derived stem cells, especially CD34+ express high levels of TF (Thomssen-Friedenreich) Galβ3GalNAcα more preferably Galβ3GalNAcαSer/Thr expressed especially as O-glycan on mucin type structure. The invention further revealed that an asialo GM1 antibody recognizing asialo-GM1 comprising Galβ3GalNAcα was not effectively recognizing blood derived stem cells.

The invention is in a preferred embodiment directed to blood derived stem cell populations enriched by binding to Galβ3GalNAcα structures on the cell surfaces by specific binder reagents, especially for the use of cell cultivation.

The invention is further directed to complex of Galβ3GalNAcα-specific binder reagent and blood derived stem cells, especially for the use of cell cultivation.

The preferred binding reagents for the structures includes GF280, GF281 and GF365, which are monoclonal antibodies, especially GF280 is preferred for the recognition of about 40% of cord blood CD34+ cells. In another preferred embodiment a lower specificity Galβ3GalNAcα-specific binder reagent is PNA (peanut agglutinin).

The Galβ3GalNAcα-specific binder reagents are especially preferred for separation of subpopulations from cord blood.

### v) GalNAcα Structures

The invention revealed that blood derived stem cells, especially CD34+ express high levels of TN GalNAcα, more preferably GalNAcαSer/Thr expressed especially as O-glycan on mucin type structure.

The invention is in a preferred embodiment directed to blood derived stem cell populations enriched by binding to GalNAcα structures on the cell surfaces by specific binder reagents, especially for the use of cell cultivation.

The invention is further directed to complex of GalNAcα-specific binder reagent and blood derived stem cells, especially for the use of cell cultivation.

The preferred binding reagents for the structures includes GF278, and VPU006, which are monoclonal antibodies, which are preferred for the recognition of about 40% of cord blood CD34+ cells. In another preferred embodiment a lower specificity GalNAcα-specific binder reagent is GalNAc specific lectin e.g. DBA (Dolichos biflorus agglutinin), especially ones known to recognize Tn structures are preferred.

The GalNAcα-specific binder reagents are especially preferred for separation and enrichment of stem cell subpopulations from cord blood.

### vi) Poly-N-Acetyllactosamine Structures

The invention revealed poly-N-acetyllactosamine structures (Galβ4GlcNAcβ3), recognizing lectin STA (Solanum tuberosum agglutinin, potato lectin) as a useful reagent for isolation and enrichment of stem cell, especially from human cord blood.

vii) Specific Mannose Structures

The invention revealed mannose structures (Manα) recognizing lectin NPA as a useful reagent for isolation and enrichment of stem cell, especially from human cord blood.

Release of Binders or Binder Conjugates from the Cells by Carbohydrate Inhibition

The invention is in a preferred embodiment directed to the release of glycans from binders. This is preferred for several methods including:
a) release of cells from soluble binders after enrichment or isolation of cells by a method invlogin a binder
b) release from solid phase bound binders after enrichment or isolation of cells or during cell cultivation e.g. for passaging of the cells

The inhibition carbohydrate is selected to correspond to the binding epitope of the lectin or part(s) thereof. The preferred carbohydrates includes oligosaccharides, monosaccharides and conjugates thereof. The preferred concentrations of carbohydrates includes concentrations tolerable by the cells from 1 mM to 500 mM, more preferably 10 mM to 250 mM and even more preferably 10-100 mM, higher concentrations are preferred for monosaccharides and method involving solid phase bound binders. Preferred oligosaccharide sequences including oligosaccharides and reducing end conjugates includes Galβ4Glc, Galβ4GlcNAc, Galβ3GlcNAc, Galβ3GalNAc, and sialylated and fucosylated variants of these as described in TABLEs and formulas according to the invention,

The preferred reducing enstructure in conjugates is

AR, wherein A is anomeric structure preferably beta for Galβ4Glc, Galβ4GlcNAc, Galβ3GlcNAc, and alfa for Galβ3GalNAc and R is organic residue linked glycosidically to the saccharide, and preferably alkyl such as method, ethyl or propyl or ring structure such as a cyclohexyl or aromatic ring structure optionally modified with further functional group.

Preferred monosaccharides includes terminal or two or three terminal monosaccharides of the binding epitope such as Fuc, Gal, GalNAc, GlcNAc, Man, preferably as anomeric conjugates: as FucαR, GalβR, GalNAcβR, GalNAcαR GlcNAcβR, ManαR. For example PNA lectin is preferably inhibited by Galβ3GalNAc or lactose or Gal, STA is inhibited by Galβ4Glc, Galβ4GlcNAc or oligomers or poly-LacNAc epitopes derived thereof and LTA is inhibited by fucosylalactose Galβ4(Fucα3)Glc, Galβ4(Fucα3)GlcNAc or Fuc or FucαR. Examples of monovalent inhibition condition are shown in Venable A. et al. (2005) BMC Developmental biology, for inhibition when the cells are bound to polyvalently to solid phase larger epitopes and/or concentrations or multi/polyvalent conjugates are preferred.

The invention is further directed to methods of release of binders by protease digestion similarly as known for release of cells from CD34+ magnetic beads.

### Immobilized Binders Preferably Binder Proteins Protein

The present invention is directed to the use of the specific binder for or in context of cultivation of the stem cells wherein the binder is immobilized.

The immobilization includes non-covalent immobilization and covalent bond including immobilization method and further site specific immobilization and unspecific immobilization.

A preferred non-covalent immobilization methods includes passive adsorption methods. In a preferred method a surface such as plastic surface of a cell culture dish or well is passively absorbed with the binder. The preferred method includes absorbtion of the binder protein in a solvent or humid condition to the surface, preferably evenly on the surface. The preferred even distribution is produced using slight shaking during the absorption period preferably form 10 min to 3 days, more preferably from 1 hour to 1 day, and most preferably over night for about 8 to 20 hours. The washing steps of the immobilization are preferably performed gently with slow liquid flow to avoid detachment of the lectin.

### Specific Immobilization

The specific immobilization aims for immobilization from protein regions which does not disturb the binding of the binding site of the binder to its ligand glycand such as the specific cell surface glycans of stem cells according to the invention.

Preferred specific immobilization methods includes chemical conjugation from specific aminoacid residues from the surface of the binder protein/peptide. In a preferred method specific amino acid residue such as cysteine is cloned to the site of immobilization and the conjugation is performed from the cystein, in another preferred method N-terminal cytsteine is oxidized by periodic acid and conjugated to aldehyde reactive reagents such as amino-oxy-methyl hydroxylamine or hydrazine structures, further preferred chemistries includes “click” chemistry marketed by Invitrogen and aminoacid specific coupling reagents marketed by Pierce and Molecular probes.

A preferred specific immobilization occurs from protein linked carbohydrate such as O- or N-glycan of the binder, preferably when the glycan is not close to the binding site or longer specar is used.

### Glycan Immobilized Binder Protein

Preferred glycan immobilization occurs through a reactive chemoselective ligation group R1 of the glycans, wherein the chemical group can be specifically conjugated to second chemoselective ligation group R2 without major or binding destructive changes to the protein part of the binder. Chemoselective groups reacting with aldehydes and ketones includes as amino-oxy-methyl hydroxylamine or hydrazine structures. A preferred R1-group is a carbonyl such as an aldehyde or a ketone chemically synthesized on the surface of the protein. Other preferred chemoselective groups includes maleimide and thiol; and “Click”-reagents including azide and reactive group to it. Preferred synthesis steps includes
a) chemical oxidation by carbohydrate selectively oxidizing chemical, preferably by periodic acid or
b) enzymatic oxidation by non-reducing end terminal monosaccharide oxidizing enzyme such as galactose oxidase or by transferring a modified monosaccharide residue to the terminal monosaccharide of the glycan.

Use of oxidative enzymes or periodic acid are known in the art has been described in patent application directed conjugating HES-polysaccharide to recombinant protein by Kabi-Frensenius (WO2005EP02637, WO2004EP08821, WO2004EP08820, WO2003EP08829, WO2003EP08858, WO2005092391, WO2005014024 included fully as reference) and a German research institute. Preferred methods for the transferring the terminal monosaccharide reside includes use of mutant galactosyltransferase as described in patent application by part of the inventors US2005014718 (included fully as reference) or by Qasba and Ramakrishman and colleagues US2007258986 (included fully as reference) or by using method described in glycopegylation patenting of Neose (US2004132640, included fully as reference).

### Conjugates Including High Specificity Chemical Tag

In a preferred embodiment the binder is, specifically or non-specifically conjugated to a tag, referred as T, specifically recognizable by a ligand L, examples of tag includes such as biotin biding ligand (strept)avidin or a fluorocarbonyl binding to another fluorocarbonyl or peptide/antigen and specific antibody for the peptide/antigen

### Preferred Conjugate Structures

The preferred conjugate structures are according to the

B-(G-)ₘR1-R2-(S1-)ₙT-, Formula CONJ

wherein B is the binder, G is glycan (when the binder is glycan conjugated),
R1 and R2 are chemoselective ligation groups, T is tag, preferably biotin, L is specifically binding ligand for the tag; S1 is an optional spacer group, preferably C₁-C₁₀ alkyls, m and n are integers being either 0 or 1, independently.

### Complex of Binder

The invention id further directed to complexes in of the binders involving conjugation to surface including solid phase or a matrix including polymers and like. It is realized that it is especially useful to conjugate the binder from the glycan because preventing cross binding of binders or effects of the binders to cells.

A complex comprising structure according to the

B-(G-)ₘR1-R2-(S1-)ₙ(T-)ₚ(L-)ᵣ-(S2)ₛ-SOL, Formula COMP
wherein B is the binder, SOL is solid phase or matrix or surface or Label (may be also Ligand conjugated label), G is glycan (when the binder is glycan conjugated), R1 and R2 are chemoselective ligation groups, T is tag, preferably biotin, L is specifically binding ligand for the tag; S1 and S2 are optional spacer groups, preferably C₁-C₁₀ alkyls, m, n, p, r and s are integers being either 0 or 1, independently.

### Preferred Elongated Epitopes

It is realized that elongated glycan epitopes are useful for recognition of the embryonic type stem cells according to the invention. The invention is directed to use part of the structures for characterizing all the cell types, while certain structural motives are more common on specific differentiation stage.

It is further realized that part of the terminal structures are especially highly expressed and thus especially useful for the recognition of one or several types of the cells.

The terminal epitopes and the longesglycan types are listed in Table 23, based on the structural analysis of the glycan types following preferred elongated structural epitopes are preferred as novel markers for embryonal type stem cells and for the uses according to the invention.

### Preferred Terminal Galβ3/4 Structures

### Type II N-Acetyllactosamine Based Structures

Terminal Type II N-Acetyllactosamine structures

The invention revealed preferred type II N-acetyllactosamines including specific O-glycan, N-aglycan and glycolipid epitopes. The invention is in a preferred embodiment especially directed to abundant O-glycan and N-glycan epitopes. The invention is further directed to recognition of characteristic glycolipid type II LacNAc terminal. The invention is especially directed to the use of the Type II LacNAc for recognition of non-differentiated embryonal type stem cells (stage I) and similar cells or for analysis of the differentiation stage. It is however realized that substantial amount of the structures are present in the more differentiated cells.

Elongated type II LacNAc structures are especially expressed on N-glycans. Preferred type II LacNAc structures are β2-linked to biantennary N-glycan core structure, Galβ4GlcNAcβ2Manα3/6Manβ4

The invention further revealed novel O-glycan epitopes with terminal type II N-acetyllactosamine structures expressed effectively the embryonal type cells. The analysis of O-glycan structures revealed especially core II N-acetyllactosamines with the terminal structure. The preferred elongated type II N-acetyllactosamines thus includes Galβ4GlcNAcβ6GalNAc, Galβ4GlcNAcβ6GalNAcα, Galβ4GlcNAcβ6(Galβ3)GalNAc, and Galβ4GlcNAcβ6(Galβ3)GalNAcα.

The invention further revealed presence of type II LacNAc on glycolipids. The present invention reveals for the first time terminal type N-acetyllactosamine on glycolipids. The neolacto glycolipid family is an important glycolipid family characteristically expressed on certain tissue but not on others.

The preferred glycolipid structures includes epitopes, preferably non-reducing end terminal epitopes of linear neolactoteraosyl ceramide and elongated variants thereof. Galβ4GlcNAcβ3 Gal, Galβ4GlcNAcβ3Galβ4, Galβ4GlcNAcβ3Galβ4Glc(NAc), Galβ4GlcNAcβ3Galβ4Glc, and Galβ4GlcNAcβ3Galβ4GlcNAc. It is further realized that specific reagents recognizing the linear polylactosamines can be sued for the recognition of the structures, when these are linked to protein linked glycans. In a preferred embodiment the invention is directed to the poly-N-acetyllactosamines linked to N-glycans, preferably β2-linked structures such as Galβ4GlcNAcβ3Galβ4GlcNAcβ2Man on N-glycans. The invention is further directed to the characterization of the poly-N-acetyllactosamine structures of the preferred cells and their modification by SAα3, SAα6, Fucα2 to non-reducing end Gal and by Fucα3 to GlcNAc residues.

The invention is preferably directed to recognition of tetrasaccharides, hexasaccharides, and octasaccharides. The invention further revealed branched glycolipid polylactosamines including terminal type II lacNAc epitopes, preferably these includes Galβ4GlcNAcβ6Gal, Galβ4GlcNAcβ6Galβ, Galβ4GlcNAcβ6(Galβ4GlcNAcβ3)Gal, and Galβ4GlcNAcβ6(Galβ4GlcNAcβ3)Galβ3, Galβ4GlcNAcβ6(Galβ4GlcNAcβ3)Galβ4Glc(NAc), Galβ4GlcNAcβ6(Galβ4GlcNAcβ3)Galβ4Glc, and Galβ4GlcNAcβ6(Galβ4GlcNAcβ3)Galβ4GlcNAc.

It is realized that antibodies specifically binding to the linear branched poly-N-acetyllactosamines are well known in the art. The invention is further directed to reagents recognizing both branched polyLacNAcs and core II O-glycans with similar β6Gal(NAc) epitopes.

### Lewis x Structures

Elongated Lewis x structures are especially expressed on N-glycans. Preferred Lewis x structures are β2-linked to biantennary N-glycan core structure, Gal(Fucα3)β4GlcNAcβ2Manα3/6Manβ4

The invention further revealed presence of Lewis x on glycolipids. The preferred glycolipid structures includes Gal(Fucα3)β4GlcNAcβ3Gal, Galβ4(Fucα3)GlcNAcβ3Gal, Galβ4(Fucα3)GlcNAcβ3 Galβ4, Galβ4(Fucα3)GlcNAcβ3 Galβ4Glc(NAc), Galβ4(Fucα3)GlcNAcβ3 Galβ4Glc, and Galβ4(Fucα3)GlcNAcβ3 Galβ4GlcNAc.

The invention further revealed presence of Lewis x on O-glycans. The preferred glycolipid structures includes preferably core II structures Galβ4(Fucα3)GlcNAcβ6GAlNAc, Galβ4(Fucα3)GlcNAcβ6GalNAcα, Galβ4(Fucα3)GlcNAcβ6(Galβ3)GalNAc, and Galβ4(Fucα3)GlcNAcβ6(Galβ3)GalNAcα.

### H Type II Structures

Specific elongated H type II structure epitopes are especially expressed on N-glycans. Preferred H type II structures are β2-linked to biantennary N-glycan core structure, Fucα2Galβ4GlcNAcβ2Manα3/6Manβ4

The invention further revealed presence of H type II on glycolipids. The preferred glycolipid structures includes Fucα2Galβ4GlcNAcβ3Gal, Fucα2Galβ4GlcNAcβ3Gal, Fucα2Galβ4GlcNAcβ3 Galβ4, Fucα2Galβ4GlcNAcβ3Galβ4Glc(NAc), Fucα2Galβ4GlcNAcβ3Galβ4Glc, and Fucα2Galβ4GlcNAcβ3Galβ4GlcNAc.

The invention further revealed presence of H type II on O-glycans. The preferred glycolipid structures includes preferably core II structures Fucα2Galβ4GlcNAcβ6GAlNAc, Fucα2Galβ4GlcNAcβ6GalNAcα, Fucα2Galβ4GlcNAcβ6(Galβ3)GalNAc, and Fucα2Galβ4GlcNAcβ6(Galβ3)GalNAcα.

### Sialylated Type II N-Acetyllactosamine Structures

The invention revealed preferred sialylated type II N-acetyllactosamines including specific O-glycan, and N-aglycan and glycolipid epitopes. The invention is in a preferred embodiment especially directed to abundant O-glycan and N-glycan epitopes. SA refers here to sialic acid preferably Neu5Ac or Neu5Gc, more preferably Neu5Ac. The sialic acid residues are SAα3Gal or SAα6Gal, it is realized that these structures when presented as specific elongated epitopes form characteristic terminal structures on glycans.

Sialylated type II LacNAc structure epitopes are especially expressed on N-glycans. Preferred type II LacNAc structures are β2-linked to biantennary N-glycan core structure, including the preferred terminal epitopes SAα3/6Galβ4GlcNAcβ2Man, SAα3/6Galβ4GlcNAcβ2Manα, and SAα3/6Galβ4GlcNAcβ2Manα3/6Manβ4. The invention is directed to both SAα3-structures (SAα3 Galβ4GlcNAcβ2Man, SAα3Galβ4GlcNAcβ2Manα, and SAα3 Galβ4GlcNAcβ2Manα3/6Manβ4) and SAα6-epitopes (SAα6Galβ4GlcNAcβ2Man, SAα6Galβ4GlcNAcβ2Manα, and SAα6Galβ4GlcNAcβ2Manα3/6Manβ4) on N-glycans.

The SAα3-N-glycan epitopes are preferred for analysis of the non-differentiated stage I embryonic type cells. The SAα6-N-glycan epitopes are preferred for analysis of the differentiated/or differentiating embryonic type cells, such as stage II and stage III, embryonic type cells. It is realized that the combined analysis of the both types of the N-glycans is useful for the characterization of the embryonic type stem cells.

The invention further revealed novel O-glycan epitopes with terminal sialylated type II N-acetyllactosamine structures expressed effectively the embryonal type cells. The analysis of O-glycan structures revealed especially core II N-acetyllactosamines with the terminal structure. The preferred elongated type II sialylated N-acetyllactosamines thus includes SAα3/6Galβ4GlcNAcβ6GalNAc, SAα3/6Galβ4GlcNAcβ6GalNAcα, SAα3/6Galβ4GlcNAcβ6(Galβ3)GalNAc, and SAα3/6Galβ4GlcNAcβ6(Galβ3)GalNAcα. The SAα3-structures were revealed as preferred structures in context of the O-glycans including SAα3 Galβ4GlcNAcβ6GalNAc, SAα3Galβ4GlcNAcβ6GalNAcα, SAα3Galβ4GlcNAcβ6(Galβ3)GalNAc, and SAα3Galβ4GlcNAcβ6(Galβ3)GalNAc(X.

### Specific Preferred Tetrasaccharide Type II Lactosamine Epitopes

It is realized that highly effective reagents can in a preferred embodiment recognize epitopes which are larger that trisaccharide. Therefore the invention is further directed to branched terminal type II lactosamine derivatives Lewis y Fucα2Galβ4(Fucα3)GlcNAc and sialyl-Lewis x SAα3Galβ4(Fucα3)GlcNAc as preferred elongated or large glycan structure epitopes. It realized that the structures are combinations of preferred termina trisaccharide sialyl-lactosamine, H-type II and Lewis x epitopes. The analysis of the epitopes is preferred as additionally useful method in context of analysis of other terminal type II epitopes. The invention is especially directed to the further defining the core structures carrying the type Lewis y and sialyl-Lewis x epitopes on various types of glycans and optimizing the recognition of the structures by including recognition of preferred glycan core structures.

### Structures Analogous to the Type II Lactosamines

The invention is further directed to the recognition of elongated epitopes analogous to the type II N-acetyllactosamines including LacdiNAc especially on N-glycans and lactosylceramide (Galβ4Glc, Cer) glycolipid structure. These share similarity with LacNAc with only difference in number of NAc residues on position of the monosaccharide residues.

### LacdiNAc Structures

It is realized that LacdiNac is relatively rare and characteristic glycan structure and it is this especially preferred for the characterization of the embryonic type cells. The invention revealed presence of LacdiNAc on N-glycans with at least β2-linkage. The structures were characterized by specific glycosidase cleavage. The LacdiNAc structures have same mass as structures with two terminal present GlcNAc containing structures in structural Table 13, indicating only single isomeric structure for a specific mass number. The preferred elongated LacdiNAc epitopes thus includes GalNAcβ4GlcNAcβ2Man, GalNAcβ4GlcNAcβ2Manα, and GalNAcβ4GlcNAcβ2Manα3/6Manβ4. The invention further revealed fucosylation LacdiNAc containing glycan structures and the preferred epitopes thus further includes GalNAcβ4(Fucα3)GlcNAcβ2Man, GalNAcβ4(Fucα3)GlcNAcβ2Manα, GalNAcβ4(Fucα3)GlcNAcβ2Manα3/6Manβ4Gal(Fucα3)β4GlcNAcβ2Manα3/6Manβ4. It is realized that presence of α6-linked sialic acid of LacNac of structure with mass number 2263, table 13 indicates that at least part of the fucose is present on the LacdiNAc arm of the molecule based on the competing nature of α6-sialylation and α3-fucosylation.

### Type I N-Acetyllactosamine Based Structures

### Terminal Type I N-Acetyllactosamine Structures

The invention revealed preferred type I N-acetyllactosamines including specific O-glycan, N-glycan and glycolipid epitopes. The invention is in a preferred embodiment especially directed to abundant glycolipid epitopes. The invention is further directed to recognition of characteristic O-glycan type I LacNAc terminal.

The invention is especially directed to the use of the Type I LacNAc for recognition of non-differentiated embryonal type stem cells (stage I) and similar cells or for analysis of the differentiation stage. It is however realized that substantial amount of the structures are present in the more differentiated cells.

The invention further revealed novel O-glycan epitopes with terminal type I N-acetyllactosamine structures expressed effectively the embryonal type cells. The analysis of O-glycan structures revealed especially core II N-acetyllactosamines with the terminal structure on type II lactosamine. The preferred elongated type I N-acetyllactosamines thus includes Galβ3GlcNAcβ3Galβ4GlcNAcβ6GalNAc, Galβ3GlcNAcβ3Galβ4GlcNAcβ6GalNAcα, Galβ3GlcNAcβ3GalGlcNAcβ6(Galβ3)GalNAc, and Galβ3GlcNAcβ3Gal B4GlcNAcβ6(Galβ3)GalNAcα.

The invention further revealed presence of type I LacNAc on glycolipids. The present invention reveals for the first time terminal type I N-acetyllactosamine on glycolipids. The Lacto glycolipid family is an important glycolipid family characteristically expressed on certain tissue but not on others.

The preferred glycolipid structures includes epitopes, preferably non-reducing end terminal epitopes of linear neolactoteraosyl ceramide and elongated variants thereof. Galβ3GlcNAcβ3Gal, Galβ3GlcNAcβ3 Galβ4, Galβ3GlcNAcβ3 Galβ4Glc(NAc), Galβ3GlcNAcβ3Galβ4Glc, and Galβ3GlcNAcβ3Galβ4GlcNAc. It is further realized that specific reagents recognizing the linear polylactosamines can be used for the recognition of the structures, when these are linked to protein linked glycans. It is especially realized that the terminal tri- and tetrasaccharide epitopes on the preferred O-glycans and glycolipids are essentially the same. The invention is in a preferred embodiment directed to the recognition of the both structures by the same binding reagent such as monoclonal antibody

The invention is further directed to the characterization of the terminal type I poly-N-acetyllactosmine structures of the preferred cells and their modification by SAα3, Fucα2 to non-reducing end Gal and by SAα6 or Fucα3 to GlcNAc residues and other core glycan structures of the derivatized type I N-acetyllactosamines.

A preferred elongated type I LacNAc structure is expressed on N-glycans. Preferred type I LacNAc structures are β2-linked to biantennary N-glycan core structure, with preferred epitopes Galβ3GlcNAcβ2Man, Galβ3GlcNAcβ2Manα and Galβ3GlcNAcβ2Manα3/6Manβ4.

HSC Binder Target Table for Selecting Effective Positive and/or Negative Binders and Combinations Thereof

Table 23 describes combined results of the inventors' structural assignments of HSC and differentiated cell specific glycosylation (Examples of the present invention describing mass spectrometric profiling, NMR, glycosidase, and glycan fragmentation experiments), biosynthetic information including knowledge of biosynthetic pathways and glycosylation gene expression, as well as binder specificities as described in the present invention (Examples of the present invention describing lectin, antibody, and other binder molecule binding to specific cell types and molecule classes).

Table 23 describes suitable binder targets in specific cell types by q, +/−, +, and ++ codes, especially preferably by + and ++ codes; as well as useful absence or low expression by −, q, and +/− codes, especially preferably by − and +/− codes. The inventors realized that such data can be used to recognize specifically selected cell types. The invention is directed to such use with various different principles as specific embodiments of the present invention: positive selection using binders recognizing specific cell type associated targets, negative selection by utilizing targets with low abundance on specific cells, as well as combined positive and negative selection, or further combined use of more than one positive and/or negative targets to increase specificity and/or efficiency according to the present invention.

Below are described especially preferred targets for binders according to the present invention.

1) HSC (including CD34+ and/or CD133+ Cells) Binder Structures:

The invention is directed to recognizing HSC based on terminal glycan epitopes as indicated in Table 23, preferably selected from:

Lex, more preferentially in O-glycan structure Lexβ6(R-Galβ3)GalNAc,
sLex, more preferentially in O-glycan structure sLexβ6(R-Galβ3)GalNAc,
SAα3Galβ4GlcNAc, more preferentially in N-glycan structure s3LNβ2Manα3/6, more preferably in N-glycan structure s3LNβ2Manα3(s3LNβ2Manα6)Man,

### Galβ3 GalNAcα,

Fucα2Galβ3GalNAcβ, more preferably in glycolipid backbone according to the present invention, GalNAcα, more preferably in Tn antigen,
large high-mannose type N-glycans, more specifically containing Manα2Man terminal epitopes, glucosylated N-glycans, more specifically containing Glcα, preferably terminal Glcα3Manα, core-fucosylated N-glycans, and/or
non-reducing terminal GlcNAcβ, preferably as GNβ2Manα3/6 and/or GNβ4Manα3 in N-glycan structure, more preferably in GNβ2Manα3(GNβ2Manα6)Man N-glycan structure;
an especially preferred binder structure is sLex, more specifically O-glycan structure sLexβ6(R-Galβ3)GalNAc, optionally together with one or more other epitopes from the list above.
2) Binder Structures Directed to Cells Differentiated from HSC (Including CD34− and/or CD133− cells)

The invention is directed to specific recognition of cells differentiated from HSC, based on terminal glycan epitopes as indicated in Table 23, preferably selected from:

LNβ4Manα3/6, more preferably in branched N-glycan structure

### LNβ2(LNβ4)Manα3(LNβ2Manα6)Man,

s3LNβ4Manα3,
Galβ3GalNAcβ, more preferably in asialo-GM1 and/or Gb5 (SSEA-3),

### SAα3 Galβ3GalNAcβ3Galα4Galβ4Glc (SSEA-5),

GalNAcβ, more preferably asialo-GM2 and/or Gb4,

### Galβ4Glc,

### Gb3,

### GalNAcα3GalNAcβ

SAα6GalNAcα, more preferably in sialyl-Tn epitope, and/or
low-mannose, small high-mannose, or hybrid-type N-glycans, preferably containing terminal Manα3Man, and/or Manα6Man,
wherein especially preferred binder structures are one or more of asialo-GM1, asialo-GM2, and/or sialyl-Tn;
optionally together with one or more other epitopes from the full list above.

### Preferred Lex/sLex Antibody Binders

The inventors found that specific cell types carry Lex/sLex epitopes on different glycan backbones according to the invention. Useful such reagents are described in the present invention, and further useful reagents are listed below. The invention is specifically directed to use of one or more of listed antibodies for structure-specific recognition of Lex/sLex epitopes in different cell types and on different glycan backbones. The list is ordered according to preferred glycan backbone specificities. Suitable binders against Lex and/or sLex on each backbone can be selected according to the present invention for different cell types.

| Code | Producer code | Manufacturer/reference | Clone |
|---|---|---|---|
| | Anti-Lex antibodies: | | |
| GF 305 | CBL144 (anti CD15) Le^{x} | Chemicon | 28 |
| GF 517 | ab34200 (CD15) | Abcam | TG-1 |
| GF 515 | 557895 anti-human CD15 | BD Pharmingen | W6D3 |
| GF 525 | ab17080-1 (CD15) | | MMA |
| | ab20138 | Abcam | 29 |
| | ab1252 | Abcam | BRA4F1 |
| | ab49758 | Abcam | BY87 |
| | ab51369 | Abcam | CLB- |
| | | | gran/2, |
| | | | B4 |
| | ab13453 | Abcam | DU- |
| | | | HL60-3 |
| | ab53997 | Abcam | LeuM1 |
| | ab6414 | Abcam | MC-1 |
| | ab665 | Abcam | MEM- |
| | | | 158 |
| | ab754 | Abcam | MY-1 |
| | ab15614 | Abcam | VIM-C6 |
| | Lewis x | Abcam | |
| | ab3358 | Abcam | P12 |
| | anti CD15 | Beckman Coulter | 80H5 |
| | anti CD15 | BioLegend | HI98 |
| | anti CD15 | Chemicon | ZC-18C |
| | anti CD15 | Chemicon | MCS-1 |
| | anti CD15 | Chemicon | DT07 & |
| | | | BC97 |
| | anti CD15 | Labvision | 15C02 |
| | anti CD15 | Labvision | SPM490 |
| | anti CD15 | Ancell | AHN1.1 |
| | anti CD15 | Quartett Immunodiagnostika, Berlin | Tu9 |
| | anti CD15 | Patricell | B-H8 |
| | anti CD15 | Patricell | HIM . . . |
| | anti CD15 | Santa Cruz | C3D-1 |
| | anti CD15 | Santa Cruz | 3G75 |
| | anti Lewis x | Santa Cruz | 4C9 |
| | anti CD15 | ScyTek Laboratories | FR4A5 |
| | antiCD15 | USBio | SF17 |
| | antiCD15 | USBio | 8.S.288 |
| | anti CD15 | USBio | 0.N.80 |
| | Anti-Lex antibodies with poly- | | |
| | LacNAc and/or glycolipid- | | |
| | specificity: | | |
| GF 518 | ab16285 (SSEA1) | Abcam | MC480 |
| | Anti-Lex antibodies for N- | | |
| | glycans: | | |
| | Anti-Lex in neutral N-glycan | Lucka et al. Glycobiology 15: 87-100, | L5 |
| | | 2005 | |
| | Anti-Lex in neutral N-glycan | Lanctot et al. Current Opinion in | 3A8 |
| | | Chemical Biology 11, Issue 4, 2007, | |
| | | 373-380; Lanctot et al. 2006, Poster | |
| | | presentation in Glycobiology Society | |
| | | Meeting, Universal City, CA, poster | |
| | | 238 | |
| | Anti-Lex antibodies for Core 2 O- | | |
| | glycans: | | |
| | Anti-Lex in Core 2 O-glycan | Sekine et al. Eur. J. Biochem. | SA024 |
| | | 268: 1129-1135, 2001 | |
| | Anti-sulfo-Lex antibodies: | | |
| | antiCD15u = sulfoCD15 | USBio | 5F18 |
| | Anti-sLex antibodies: | | |
| GF 516 | 551344 anti-human CD15s | BD Pharmingen | CSLEX1 |
| GF 307 | MAB2096 (anti-sLewis X) | Chemicon | KM93 |
| | anti sLex | Seikagaku | 73-30 |
| | anti sLex | Meridianlifesciences | 258-12767 |
| | anti sLex | USBio | 2Q539 |
| | Anti-sLex antibodies for Core2 O- | | |
| | glycans: | | |
| GF 526 | MAB996 (anti-hP-selectin- | R&D systems | CHO131 |
| | glycoprotein ligand 1 ab) | | |

### EXAMPLES

### Example 1

### N-Glycosylation of Human Cord Blood-Derived Stem Cells

Cell surface glycans contribute to the adhesion capacity of cells and are essential in cellular signal transduction. Yet, the glycosylation of hematopoietic stem cells, such as CD133+ cells, is poorly explored. In this study, we analyzed N-glycan structures of CD133+ and CD133− cells with mass spectrometric profiling and exoglycosidases digestion; cell surface glycan epitopes with lectin binding assay; and expression of N-glycan biosynthesis-related genes with microarray. Over 10% difference was demonstrated in the N-glycan profiles of CD133+ and CD133− cells. Biantennary complex-type N-glycans were enriched in CD133+ cells. Of the genes regulating the synthesis of these structures, CD133+ cells overexpressed MGAT2 and underexpressed MGAT4. Moreover, the amount of high-mannose type N-glycans and terminal α2,3-sialylation was increased in CD133+ cells. Elevated α2,3-sialylation was supported by the overexpression of ST3GAL6. The new knowledge of hematopoietic stem cell-specific N-glycosylation advances their identification and provides tools promote stem cell homing and mobilization or targeting to specific tissues.

### Introduction

More than half of human proteins are estimated to be glycosylated. In other words, glycosylation is more common post-translational modification than phosphorylation (1). Glycans cover the entire cell surface as the glycocalyx and they function as structural components and signal transducers. Glycans are essential for many biological processes including cellular response to oxidative stress, resistance to innate immunity and cell-cell or cell-matrix communication (2,3). In hematopoietic stem cells, such as CD133+ cells, cell type-specific glycosylation may contribute to maintenance, differentiation, homing and mobilization.

Cord blood is a convenient source of stem cells; they are easy to obtain and they have better tolerance for histocompatibility mismatches than stem cell grafts from other sources. Cord blood transplantations are often used when perfect HLA-matched donor is not available. The number of cells available in one cord blood unit is often considered adequate only for pediatric patients and numerous methods have been attempted to expand stem cells in vitro. The hematopoietic stem cells essential for therapy are often characterized based on the expression of cell surface glycoproteins CD34 and CD133. Nearly all (99.8%) of CD133+ cells are also CD34 positive (4). During differentiation, the CD133 molecule is lost from the cell surface earlier than CD34.

Understanding hematopoietic stem cell glycobiology offers new techniques for better stem cell engraftment, ex vivo or in vivo expansion and targeting to specific tissue (5-7). Characterization of CD133+ cell N-glycome would also better the identification of hematopoietic stem cells. However, N-glycosylation is a complex event, and so far the analysis of human stem cell glycome has been lacking suitable technology to analyze samples with limited cell number. N-glycan biosynthesis is controlled by expression of glycosyltransferase and glycosidase enzymes and isozymes which compete for the same glycan substrates. In addition, formation of glycan molecules, their precursor biosynthesis, transport, and localization mechanisms, are entwined with other biosynthetic pathways (8,9). A change in the activity of one single glycan biosynthetic enzyme can have a drastic effect on the appearance and the function of the cell. However, the identification of specific genes involved in the certain glycosylation process requires that the expression level of glycosylation-related genes are compared to glycan structures. Recently, dramatic N-glycome changes with differential expression of only few genes have been described in activated murine T cells (10-12). Differential expression of genes encoding sialyltransferases have been shown to differentially contribute to the B lymphocyte response to immune signaling (13).

In the present study, we characterized N-glycosylation events typical for CD133+ cells by combining data from N-glycan structure analysis and expression profiling of genes encoding glycosyltransferases and glycosidases. The results of CD133+ cells were compared to mature leucocytes (CD 133−) to identify N-glycosylation specific for CD133+ cells. Our work presents new information on the characters of stem cells. The results may help to develop their use in therapeutic applications. Engineering cell glycosylation could be used to enhance stem cell homing and mobilization or to design cell products targeted to specific tissues.

### Materials and Methods

### Cells

Cord blood was obtained from the Helsinki University Central Hospital, Department of Obstetrics and Gynecology, and Helsinki Maternity Hospital. All donors gave informed consent and the study was approved by ethical review board of the Helsinki University Central Hospital and the Finnish Red Cross Blood Service. Collection and processing of the fresh cord blood was performed as described earlier (14). Ficoll-Hypaque density gradient (Amersham Biosciences, New Jersey, USA, www1.amerschambiosciences.com) was used to isolate leucocytes that are mononuclear cells. Leucocytes can be obtained in quantities adequate for NMR analysis. In addition, leucocytes were used in lectin labeling assay. Stem cell fraction was sorted from the leucocyte fraction with anti-CD133 microbeads in magnetic affinity cell sorting (Miltenyi Biotec, Bergisch Gladbach, Germany, www.miltenyibiotec.com) (15). Mature leucocytes (CD133− cells) were collected for control purposes. Altogether 11 cord blood units were used. In the preparation of samples to mass spectrometric analysis, to avoid olicosaccharide contamination, ultra pure bovine serum albumin (at least 99% pure, Sigma-Aldrich Chemie GmbH, Steinheim, Germany, www.sigmaaldrich.com) was used.

### N-Glycan Isolation

N-glycans were detached from cellular glycoproteins by F. meningosepticum N-glycosidase F digestion (Calbiochem, USA) essentially as described (Nyman et al., 1998). Cellular contaminations were removed by precipitating the glycans with 80-90% (v/v) aqueous acetone at −20° C. and extracting them with 60% (v/v) ice-cold methanol (Verostek et al., 2000). The glycans were then passed in water through C18 silica resin (BondElut, Varian, USA) and adsorbed to porous graphitized carbon (Carbograph, Alltech, USA). The carbon column was washed with water, and then the neutral glycans were eluted with 25% acetonitrile in water (v/v) and the sialylated glycans with 0.05% (v/v) trifluoroacetic acid in 25% acetonitrile in water (v/v). Both glycan fractions were additionally passed in water through strong cation-exchange resin (Bio-Rad, USA) and C18 silica resin (ZipTip, Millipore, USA). The sialylated glycans were further purified by adsorbing them to microcrystalline cellulose in n-butanol:ethanol:water (10:1:2, v/v), washing with the same solvent, and eluting by 50% ethanol:water (v/v). All the above steps were performed on miniaturized chromatography columns and small elution and handling volumes were used.

### Mass Spectrometry

MALDI-TOF mass spectrometry was performed with a Bruker Ultraflex TOF/TOF instrument (Bruker, Germany) and the samples were prepared for the analysis essentially as described (22). Neutral N-glycans were detected in positive ion reflector mode as [M+Na]+ ions and sialylated N-glycans were detected in positive ion reflector or linear mode as [M-H]− ions. Relative molar abundances of neutral and sialylated glycan components were assigned based on their relative signal intensities in the mass spectra when analyzed separately as the neutral and sialylated N-glycan fractions (Saarinen, 1999. Harvey, 1993, Naven, 1996, Papac, 1996). The mass spectrometric raw data was transformed into the present glycan profiles by removing the effect of isotopic pattern overlapping, multiple alkali metal adduct signals, products of elimination of water from the reducing oligosaccharides, and other interfering mass spectrometric signals not arising from the glycan components in the sample. The resulting glycan signals in the presented glycan profiles were normalized to 100% to allow comparison between samples.

Quantitative difference between two glycan profiles (%) was calculated according to Equation 1:

$$difference = \frac{1}{2} {\sum }_{i = 1}^{n} \vert {p}_{i , a} - {p}_{i , b} \vert , \qquad ( 1 )$$

wherein p is the abundance (%) of glycan signal i in profile a or b, and n is the total number of glycan signals.

Relative difference in a glycan feature between two glycan profiles was calculated according to Equation 2:

relative

$$difference = {x ( \frac{{P}_{a}}{{P}_{b}} )}^{x} , \qquad ( 2 )$$

wherein P is the sum of the abundances (%) of the glycan signals with the glycan feature in profile a orb, x is 1 when a≧b, and x is −1 when a<b.

### NMR Spectroscopy

The isolated glycans were further purified for NMR spectroscopy by gel filtration high-pressure liquid chromatography in water or 50 mM ammonium bicarbonate to separate neutral and sialylated glycan fractions, respectively. The NMR analysis was performed as previously descripted (Weikkolainen et al. Glycoconj. J. 2007 in press) with Variant Unity NMR spectrometer at 800 MHz using a cryo-probe for enhanced sensitivity. Prior to proton NMR analysis, the purified glycans were dissolved in 99.996% deuterium oxide and dried to omit water and to exchange sample protons.

### Exoglycosidase Analysis

Analysis of non-reducing glycan epitopes present in N-glycan fractions was performed by digestion with specific exoglycosidase enzymes. Enzyme specificity towards isomeric structures was controlled in parallel reactions with defined oligosaccharides as detailed below. The employed exoglycosidase enzymes were: β1,4-galactosidase from S. pneumoniae (recombinant in E. coli, Calbiochem) digested the β1,4-linked galactose of lacto-N-hexaose, β1,3-galactosidase from X. manihotis (recombinant in E. coli, Calbiochem) digested the P1,3-linked galactose of lacto-N-hexaose, α2,3-sialidase from S. pneumoniae (recombinant in E. coli, Calbiochem) digested α2,3-but not α2,6-sialyl N-acetyllactosamine, broad-range sialidase from A. ureafaciens (recombinant in E. coli, Calbiochem) digested both α-2,3- and α-2,6-sialyl N-acetyllactosamine, and α-mannosidase from Jack beans (C. ensiformis; Sigma-Aldrich) digested the Man5-Man9 high-mannose type N-glycans present in oligosaccharide mixture isolated from human cells. The reactions were carried out by overnight digestion at +37° C. in 50 mM sodium acetate buffer, pH 5.5. The digested glycan fractions were purified for analysis by solid-phase extraction with graphitized carbon and analyzed by MALDI-TOF mass spectrometry as described above.

### Microarray

RNA purified from CD133+ and CD133− cells was hybridized on Affymetrix Human Genome U133 Plus 2.0 arrays, and the data was analyzed using Affymetrix GeneChip Operating Software as previously described (14). When applicable, the same probes were selected for analysis that are represented on the Affymetrix glycogene chip provided by the Gene Microarray Core of Consortium for Functional Glycomics. A transcript was considered differentially expressed when at least 1.5-fold increase or decrease in the expression was demonstrated.

### Lectin Binding Analysis by Flow Cytometry

To prevent hemolysis or hemagglutination of erythrocyte precursors by lectins which would disturb the flow cytometric analysis, MNCs were GlyA depleted using Glycophorin A MicroBeads (Miltenyi Biotec). The cells were labeled with phycoerythrin (PE)-conjugated CD34 monoclonal antibody (Miltenyi Biotec) to show the stem cell population and with one of the fluorescein isothiocyanate (FITC)-conjugated lectins PSA from Pisum sativum for α-mannose and glucose; HHA from Hippeastrum hybrid for internal and terminal α1,3- or α1,6-linked mannose, and GNA from Galanthus nivalis for α1,3-mannose residues; PHA-L from Phaseolus vulgaris L for large complex-type N-glycans; RCA-I from Ricinus communis I for β-galactose; SNA from Sambucus nigra and MAA from Maackia amurensis for α2,6- and α2,3-linked sialic acid, LTA from Lotus tetragonolobus and UEA-J from Ulex europaeus I for (1-fucose; EY Laboratories, Inc. San Mateo, Calif., USA, www.eylabs.com; Vector Laboratories, Burlingame, Calif., USA, www.vectorlabs.com). Flow cytometry was performed on Becton Dickinson FACSCalibur™ and fluorescence was measured using 530/30 nm and 575/25 nm bandpass filters. The labeling results of MNCs show the overall frequency of specific glycosylation events. The double-labeled cell fraction specifies the glycans on the cell surface of stem cells.

### Results

### Structural Analysis

For the structural analysis, neutral and sialylated N-glycan fractions from total leucocytes were subjected to NMR. The NMR analyses yielded detailed data about the most abundant N-glycan structures present in leucocytes (unseparated mononuclear cells) (Supplementary Fig. NMR and Supplementary Tables NMR1 and NMR2). High-mannose type N-glycans were detected in neutral N-glycan fraction, whereas the N-glycan backbone with α2,6- and α2,3-sialylated biantennary complex-type N-glycans were the major structures in the sialylated N-glycan fraction. Moreover, quantitative analysis of the spectrum showed that α2,6-sialylation was more abundant than α2,3-sialylation, and type 2 N-acetyllactosamine (Galβ4GlcNAc, 100%) dominated over type 1 N-acetyllactosamine (Galβ3GlcNAc, not detected) in the N-glycan antennae. β1,4-branched triantennary N-glycans and α1,6-fucosylated N-glycan core were also detected.

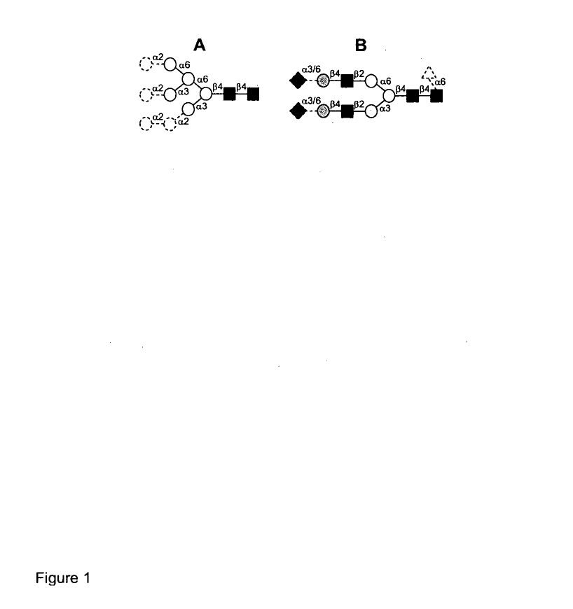

To compare the quality and quantity of N-glycans on stem cells and mature leucocytes, CD133+ and CD133− cells were separately analyzed by MALDI-TOF mass spectrometry. The data from NMR was used to qualify structures presented in the mass spectrometric analysis. Over 80 signals containing some multiple isomeric structures were detected in both cell types (FIGS. 2A and 3A). The profile of sialylated N-glycans was more divergent between CD133+ and CD133− cells (FIG. 1B, 17% difference) than the neutral N-glycan profiles (FIG. 1A, 9% difference). Major N-glycans in CD133+ and CD133− cells were high-mannose and biantennary complex-type structures (Figure). CD133+ and CD133− cells also had monoantennary, hybrid, low-mannose and large complex-type N-glycans (FIGS. 2 and 3). To analyze the differences between CD133+ and CD133− cells, the proposed monosaccharide compositions assigned to each detected glycan signal (FIGS. 2 and 3; A and B) were quantitatively analyzed by grouping them into the major N-glycan classes (FIGS. 2C and 3C) and by comparing the proportion of different major N-glycan classes between CD133+ and CD133− cells. The CD133+ cell N-glycome showed polarization towards high-mannose type N-glycans (FIG. 2C), biantennary complex-type N-glycans with core composition 5-hexose 4-N-acetyhexosamine and sialylated monoantennary N-glycans (FIG. 3C). In contrast, CD133− cells had increased amounts of large complex-type N-glycans with core composition 6-hexose 5-N-acetylhexosamine or larger, sialylated hybrid-type N-glycans and low-mannose type N-glycans.

The CD133− cell population presents an average of the phenotypes of multiple cell types. To compare the results with an independently isolated differentiated cell population, the CD8+ and CD8− cells were analyzed. CD8+ cells showed an N-glycosylation phenotype similar to CD133− cells. Especially the proportion of large complex-type N-glycans was elevated in these cells (data not shown). This indicates that demonstrated N-glycome in CD133+ cells is typical for the cell type.

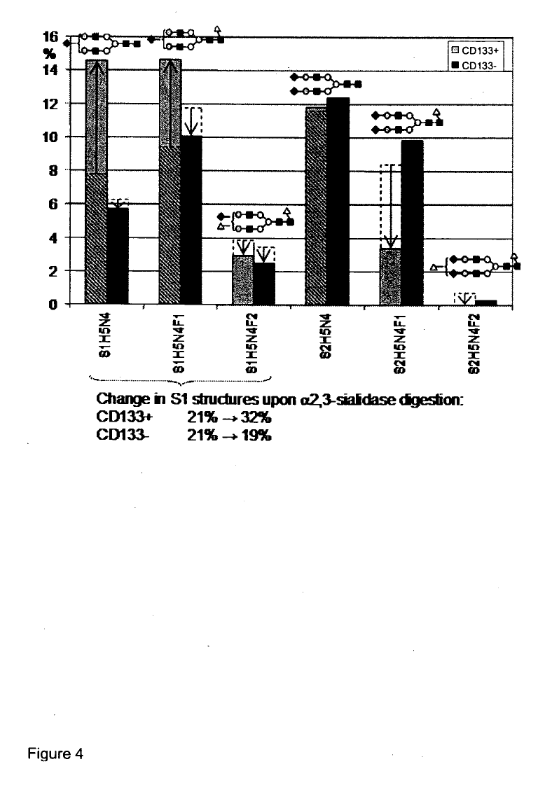

To characterize terminal epitope profile on CD133+ and CD133− cells, specific exoglycosidase digestions was combined with mass spectrometric analysis. α-mannose, β1,4-galactose, and β-N-acetylglucosamine residues were found abundant in both cell types, whereas β1,3-linked galactose residues were not detected in significant amounts. The majority of both CD133+ and CD133+ cells carried α2,6-linked sialic acids, as demonstrated in α2,3-sialidase treatment. Neutral that is completely desialylated glycan components were produced from all sialylated N-glycan species from CD133+ cells, whereas CD133− cells contained minor components completely resistant to the α2,3-sialidase treatment. Further, the acidic glycan profile change during the specific sialidase treatment was quantitatively larger in CD133+ cells compared CD133− cells (FIG. 4). Taken together, the proportions of the N-glycan signals affected to α2,3-sialidase in CD133+ and CD133− cells were different showing enrichment in CD133+ cell α2,3-sialylated N-glycans (FIG. 4).

### Biosynthetic Pathways of N-Glycosylation

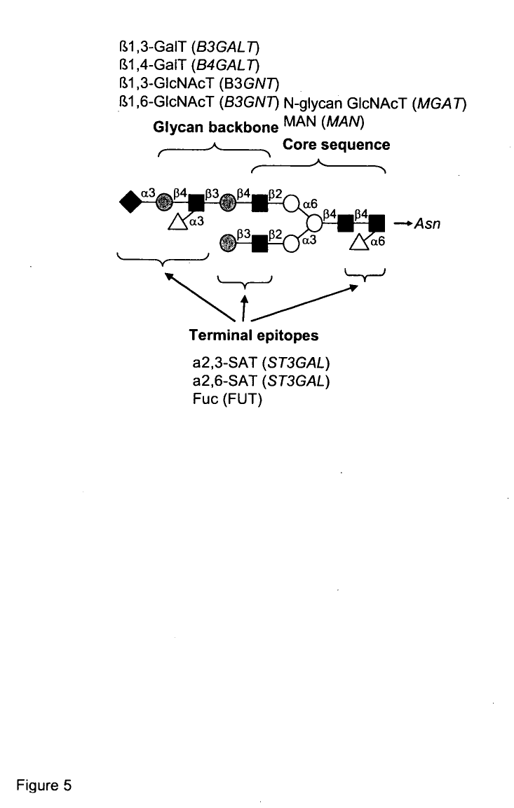

After glycan profiling, expression of genes encoding enzymes that modify N-glycan structures were studied. N-glycan biosynthesis is controlled with several glycosyltransferase and glycosidase enzyme families that act on different regions of the N-glycan chain; N-glycan core, backbone and terminal regions (FIG. 5). Biosynthesis of other important glycan classes such as O-glycans and glycolipids partly overlap with N-glycan biosynthesis, but different members of enzyme families are often specialized to synthesize certain glycan types. The target glycan classes for the gene products and the expression results of N-glycan structure-associated genes are shown in table 1.

### N-Glycan Core Sequence

N-glycan core structures are formed by specialized mannosidase (MAN) and N-acetylglucosaminyltransfrerase (GlcNAcT) enzymes (16) (FIG. 4). MANs shape high-mannose and low-mannose type N-glycan structures and form the starting points for the other N-glycan types (8). MAN1 enzymes control the conversion from high-mannose to hybrid-type and monoantennary N-glycans, and MAN 2 enzymes control the further conversion to complex-type structures. GlcNAcTs determine the branching modes of hybrid, monoantennary, and complex-type N-glycans (17).

High-mannose type N-glycans were the prevalent neutral N-glycan group. The relative amounts of neutral α-mannosylated N-glycans were similar in CD133+ and CD133− cells (FIG. 4). However, terminal α-mannose was enriched in high-mannose type glycans in CD133+ cells, whereas terminal α-mannose was broadly found in low-mannose, hybrid, and monoantennary-type N-glycans in CD133− cells. The presence of α-mannose on the cell surface was further demonstrated by lectin labeling (Table 2). α-mannose and N-glycan core sequence-binding lectins PSA and HHA labeled 96-99% of mature leucocytes and the stem cell population. GNA labeled 73% of the mature leucocytes but only few stem cells. GNA has highest affinity towards low-mannose type N-glycans with terminal α1,3-mannose residues. Lectin labeling result suggests differential α-mannosylation for stem cells like the observations from structural analysis.

High-mannose type N-glycans are processed into other N-glycan types by glycosidase families MAN1 and MAN2 (8,16) (Table 1). Three of the four known MAN1 family genes MAN1A1, MAN1A2 and MAN1B1 and all five known MAN2 family genes MAN2A1, MAN2A2, MAN2B1, MAN2B2 and MAN2C1 were similarly expressed in CD133+ and CD133− cells. The fourth member of MAN1 gene family, MAN1C1, was expressed in CD133− cells only. Its specific role within the MAN1 family is not known. However, In vitro the MAN1C1 encoded enzyme prefers removal of the GlcNAcT blocking mannose residues in the α1,3 branch (21).

The amount of N-glycan structures larger than biantennary complex-type N-glycans was decreased in CD133+ cells according to structural analysis. PHA-L that binds to branched complex-type N-glycans labeled 98% leucocytes and most of the stem cells (Table 2). The labeling result shows that dispute the quantitative difference in the large complex-type N-glycans between mature leucocytes and stem cells, these structures are typical for both cell types.

The biosynthesis of hybrid-type and complex-type N-glycans is controlled by a family of N-glycan core GlcNAcTs encoded by MGAT genes (Table 1). MGAT1, MGAT2 and MGAT4A/MGAT4B encode GlcNAcT1, GlcNAcT2 and GlcNAcT4, respectively. These genes were expressed in CD133+ and CD133− cells, but differences in their expression levels were demonstrated. In CD133+ cells MGAT2 was overexpressed by 1.9-fold and MGAT4A was underexpressed by 2.8-fold.

Together, both MAN1C1 and MGAT2 expression patterns in CD133+ cells indicates increased biosynthesis of high-mannose type and complex-type N-glycans, and decreased biosynthesis of hybrid-type and monoantennary N-glycans. In addition, underexpression of MGAT4A may result in the reduction of triantennary and larger N-glycans in stem cells.

### N-Glycan Backbone

Glycan backbone structures include short antennae and extended poly-N-acetyllactosamine (poly-LacNAc) chains formed by the concerted action of galactosyltransferases (GalT; antennae and poly-LacNAc) and GlcNAcTs (poly-LacNAc) (FIG. 5). The present study focused on GalTs, because the short antennae-type structures dominated over poly-LacNAc in leucocytes. The terminal galactose residues were shown to be β1,4-linked, whereas β1,3-linked galactose was not detected. Lectin RCA-I that is specific for type 2 LacNAc labelled 91% of the leucocytes as well as the stem cells.

Genes encoding the β1,4-GalTs synthesizing type 2 LacNAc epitopes, such as B4GALT1, B4GALT3 and B4GATL4 were expressed in both CD133+ and CD133− cells (Table 1). However, the expression of B4GALT3 was decreased in CD133+ cells by 2.3-fold. Further, the expression of B4GALT2 was only seen in CD 133+ cells. Type 1 LacNAc synthesizing β1,3-GalTs, encoded by B3GALT2 and B3GALT5 were absent in CD133+ and CD133− cells, as were the potential glycan products.

### N-Glycan Terminal Epitopes

The terminal epitopes are added on the N-glycan structures during the final phase of the synthesis (FIG. 5). Common glycan moieties in terminal modifications of N-glycans include sialic acid and fucose residues. Sialyltransferase families α2,3SATs and α2,6SATs transfer sialic acids to terminal galactose residues. Such epitopes were found in CD133+ and CD133− cells. In addition, all known human fucosyltransferase synthetic pathways were analysed.

The α2,3-sialidase profiling revealed that α2,3-sialylated N-glycans were more common in CD133+ cells than in CD133− cells (FIG. 4), whereas α2,6-sialyl-LacNAc was common for both cell types. Lectin SNA was used to detect α2,6-sialylation, the product of ST6GAL1 on cell surface. SNA ligands were detected on 98% of the leucocytes including the stem cells. Labeling with MAA showed that α2,3-sialyl-LacNAc structures were present on only 62% of the leucocytes, and similarly in stem cells. This suggests that enriched α2,3-sialylation of CD133+ cells may be related to N-glycans only. ST6GAL1 encoding α2,6-SAT and ST3GAL6 encoding α2,3-SAT were expressed in CD133+ and CD133− cells (Table 1). 3.9-fold overexpression of ST3GAL6 was detected in CD133+ cells.

N-glycan core structures of CD133+ and CD133− cells were often α1,6-fucosylated as shown by mass spectrometric analysis. In addition, presence of two or more fucose residues on each N-glycan chain was observed in CD133+ and CD133− cells (FIGS. 2 and 3). Since type 1 LacNAc was prevalent neither in CD133+ or CD133− cells, the fucosylated epitopes were expected to carry α1,3- or α1,2-linked fucose residues. Lectin LTA has specificity towards α1,3-linked fucose, that is part of the Lex antigen. It labeled only 6% of the leucocytes. No labeling of stem cell population was shown. Lectin UEA-I with α1,2-linked fucose specificity recognized 53% of the leucocytes and the stem cells.

The expression of FUT4 that encodes the myeloid type α1,3-FucT4 (18,19) was found in both CD133+ and CD133− cells. FucT4 synthesizes the Lex (CD15) or sLex antigens by fucosylation of type 2 LacNAc or α3-sialyl LacNAc, respectively. FUT1 encoding α1,2-FucT was not expressed in CD133+ or CD133− cells. Moreover, only CD133+ cells expressed detectable levels of FUT8 encoding the N-glycan core α1,6-FucT a glycosylation abundantly detected in the structural analysis of CD133+ and CD133− cells. FUT8 is the only known gene encoding a glycosyltransferase promoting α1,6-fucosylation, yet previous reports show that an increase in α1,6-fucosylation can not be explained by the up-regulation of α1,6-FucT alone (20).

### Discussion

The present work uses a new approach to characterize CD133+ cells. CD133+ cell-specific N-glycosylation and the transcriptional regulation of the glycosylation events were linked together to gather the expressed genes producing key N-glycan entities different between stem cells and mature leucocytes. In addition, lectin binding assay revealed divergences on the cell surface glycosylation between stem cells and mature leucocytes.

Although rare N-glycan structures may not be detected by MALDI-TOF and NMR analysis, the method allows quantitative analysis of glycan compositions between different cell types. Enrichment of high-mannose type glycans were representative of stem cells, also on the cell surface as shown with lectin labeling. Mature leucocytes contained more large complex-type N-glycans, whereas complex N-glycans were often biantennary in CD133+ cells. The gene expression seems to support the core glycosylation typical for the cell type. Putative role for the absence of MAN1C1 is suggested as slowing the conversion from high-mannose type to hybrid-type and monoantennary glycans.

The structures present in CD133+ cells, such as high-mannose and complex type N-glycans, are found on CD164 epitope (24). The function of the CD164 molecule is indeed N-glycan-dependent and modulates the CXCL12-mediated migration of cord blood-derived CD133+ cells (24,25). It also negatively regulates stem cell proliferation (26,27). Complex N-glycan determinants are also part of other adhesion molecules common to hematopoietic stem cells, such as the CD34+ cell-specific glycoform of CD44 molecule.

Different β1,4-galactosylation-related genes were involved in the β1,4-galactosylation of CD133+ and CD133− cells. No change in their glycan profiles was detected. However, these genes might galactosylate N-glycan backbones of single glycoproteins.

B4GALT2 expressed only in CD133+ cells has restricted expression pattern to fetal brain, adult heart, muscle and pancreas (28), whereas B4GALT3 is widely expressed in most tissues (28). B4GALT2 and B4GALT3 encoded enzymes have almost identical substrate specificity and they may substitute each other (29). Both B4GALT2 and B4GALT3 galactosylate biantennary and larger complex-type N-glycans. The expression of B4GALT2 in CD133+ cells may be compensated with the underexpression of B4GALT3. However, changes in glycoproteins present on lower abundances might not be detected by present methods therefore it is possible that differential glycosylation exist on single glycoproteins. B4GALTs synthesize the glycan backbones of selectin ligands, although selectin adhesion is regulated trough terminal glycosylation. Galactosylation has an important role in the proliferation and differentiation of epithelial cells in mice (30). If the differential biosynthetic pathways of CD133+ and CD133− cells have an influence on β1,4-galactosylation of certain glycoproteins, the significance of β1,4-galactosylated structures could participate in controlling the proliferation and differentiation of CD133+ cells. This interesting hypothesis requires closer examination.

α2,6-sialylation dominates the cell surface glycans of human bone marrow and peripheral blood-derived CD34+ and CD34− cells (31) similarly as in cord blood-derived CD133+ and CD133− cells. Moreover, granulocyte colony-stimulating factor mobilized CD34+ cells in peripheral blood and bone marrow-derived CD34+ cells have higher expression of ST6GAL1 with elevated α2,6-sialylation on the cell surface than noninduced peripheral blood-derived CD34+ cells indicating that α2,6-sialylation of CD34+ cells is dependent of granulocyte colony-stimulating factor in their environment (12). α2,6-sialylation of CD34+ cells might participate regulating their cellular adherence. α2,6-linked sialic acid, product of ST6GAL1 is crucial for homing process of CD22+B-cells (32). Expression of ST6GAL1 reduces galectin-1 binding to cells (33). Galectin-1 stimulates stem cell expansion (34). Galectin-1 is abundantly secreted by mesenchymal stem cells (35), but its expression is not detected in CD133+ cells (gene expression profile in (14)). Hematopoietic stem cells expand and remain their long-term reconstruction capacity longer when they are co-cultured with mesenchymal stem cells (36). Mesenchymal and hematopoietic stem cell interaction in co-cultures could be assisted by galectin-1 binding.

In sialylated glycan biosynthesis, α2,3- and α2,6-SATs can compete for the same N-glycan substrates. In the present study we show enriched α2,3-sialylation in CD133+ cells, accompanied with overexpression of ST3GAL6. Previously lower proportion of α2,6-SAT1 together with lower α2,6-sialylation of N-glycans was demonstrated in murine T cell activation (11). The authors suggested that this may be due to α1,3-GalT expression competing from the same substrate with α2,6-SAT1. However, α1,3-GalT is not present in human and therefore, the similar substrate competition is not relevant. The present results show that in human CD133+ cells lower relative abundance of α2,6-sialylation is instead caused by increased α2,3-sialylation. Gene expression data strongly suggests that ST3GAL6 overexpression is responsible for the increased α2,3-sialylation in these cells. ST3GAL6 has got restricted substrate specificity which lead to suggest it is involvement to synthesis of sialyl-paragloboside, a precursor structure of sialyl-Lewis X determinant (37). However, the expression of ST3GAL6 was not shown to correlate with expression of sialyl-Lewis X.

CD34+ cells (also CD133+ cells), but not mature leucocytes, display a hematopoietic cell L and E-selectin ligand, a glycoform of the CD44 antigen, critically dependent on N-glycan sialylation(38-40). Selectin-ligand interactions promote homing of stem cells and may also control their proliferation. L-selectins present on CD34+ cells have been associated with faster hematopoietic recovery after stem cell transplantation (38). The α2,3-sialylation of N-glycans negatively regulates the ability of CD44 molecule to bind extracellular matrix (41). The main role of CD44 is binding to hyaluronic acid (42), yet only small amount of CD34+ cells carrying CD44 epitope are bound to hyaluronic acid in bone marrow (43). Therefore, α2,3-sialylation is probably at least needed to assist both the homing and proliferation of stem cells.

In addition to N-glycan core α1,6-fucosylation, small amounts of α1,2- or α1,3-linked fucose residues were present. The expression of FUT genes indicate the synthesis of myeloid type α1,3-linked fucose. However, the presence of α1,3-fucosylation was detected very low on cord blood-derived leucocytes, including stem cells. On the other hand, α1,2-linked fucose was detected on cell surface even expression of FUT1 processing α1,2-fucosylation was absent. FUT7 product is a key enzyme responsible for the synthesis of sLex that binds to selectins (44). In addition, FUT1 expression has been shown to inhibit sLex expression (45). cord blood-derived stem cells have been shown to have impaired α1,3-fucosylation trough reduced α1,3-fucosyltransferase expression which contribute to lower selectin binding and may delay engraftment of cord blood-derived cells in transplantation (5,7). During embryogenesis, only FUT4 and FUT9 are expressed. FUT4 expression has been shown to compensate low or absent FUT7 expression and production of such as sLe x required in selecting binding in adults with deficient FUT7 expression (46). At least two attempts to enforce fucosylation of stem cells have been performed (5,7), in both cases fucosylation was successful, and in one of them could show improved homing to bone marrow of noneobese diabetic/severe combined immune deficient mice (7). If defect in FUT7 expression in cord blood-derived cells cause delay in stem cell engraftment to human bone marrow, cell engineering techniques could be used to enhance stem cell fucosylation.

Taken together, the critical genes associated to characteristic N-glycosylation of CD133+ cells were, overexpression of MGAT2 and ST3GAL6, underexpression of MGAT4A and the absence of MAN1C1. In addition, β1,4-galactosylation was on molecular level regulated differently between CD133+ and CD133− cells with unknown function that is a matter of further investigation. CD34+ and CD133+ cells have highly similar genome-wide gene expression profile (47). It was expected that if the genes-related to N-glycosylation in CD133+ cells are pivotal to stem cell N-glycome, the genes should be similarly expressed in CD34+ cells as well. Expression of N-glycosylation-related genes in CD34+ cells was proved to be similar with CD133+ cells (gene expression results collected from published CD34+expression profile (47)). In addition, the same change in the expression pattern was noticed between CD34+ and CD34− cells than between CD133+ and CD133− cells suggesting that N-glycome of cord blood-derived CD34+ cells is very similar to CD133+ cell N-glycome and differing from mature leucocytes.

The characterized N-glycan features in CD133+ cells have crucial role in known glycoproteins such as CD164, hematopoietic stem cell and progenitor specific CD44 glycoform, and binding of E-selectin, P-selectin and galectin ligands that are required for cell migration, proliferation, cell recognition and homing to BM. The N-glycome of CD 133+ cells may also be involved in many yet unknown functions. Combined information from changes in gene expression and glycan structures between CD133+ and CD133− cells allowed identification of novel genes regulating CD133+ cell-specific N-glycan biosynthesis. The new knowledge of hematopoietic stem cell-specific N-glycosylation helps to engineer novel therapeutic applications or to improve current protocols. Changing the glycosylation in vitro or in vivo can be used to enhance the natural properties of stem cells or to modify N-glycome that would target stem cells to specific tissues. References of Example 1 and Table 1.

### REFERENCES

1. Apweiler R, Hermjakob H and Sharon N. (1999). On the frequency of protein glycosylation, as deduced from analysis of the SWISS-PROT database. Biochim Biophys Acta 1473:4-8.
2. Varki A. (1993). Biological roles of oligosaccharides: all of the theories are correct. Glycobiology 3:97-130.
3. Varki A. (2006). Nothing in glycobiology makes sense, except in the light of evolution. Cell 126:841-845.
4. Gallacher L, Murdoch B, Wu D M, Karanu F N, Keeney M and Bhatia M. (2000). Isolation and characterization of human CD34(−)Lin(−) and CD34(+)Lin(−) hematopoietic stem cells using cell surface markers AC133 and CD7. Blood 95:2813-2820.
5. Hidalgo A, Frenette P S. (2005). Enforced fucosylation of neonatal CD34+ cells generates selectin ligands that enhance the initial interactions with microvessels but not homing to bone marrow. Blood 105:567-575.
6. Sampathkumar S G, Li A V, Jones M B, Sun Z and Yarema K J. (2006). Metabolic installation of thiols into sialic acid modulates adhesion and stem cell biology. Nat Chem Biol 2:149-152.
7. Xia L, McDaniel J M, Yago T, Doeden A and McEver R P. (2004). Surface fucosylation of human cord blood cells augments binding to P-selectin and E-selectin and enhances engraftment in bone marrow. Blood 104:3091-3096.
8. Herscovics A. (1999). Importance of glycosidases in mammalian glycoprotein biosynthesis. Biochim Biophys Acta 1473:96-107.
9. Lowe J B. (2002). Glycosylation in the control of selectin counter-receptor structure and function. Immunol Rev 186:19-36:19-36.
10. Le Marer N, Skacel P O. (1999). Up-regulation of alpha2,6 sialylation during myeloid maturation: a potential role in myeloid cell release from the bone marrow. J Cell Physiol 179:315-324.
11. Comelli E M, Sutton-Smith M, Yan Q, Amado M, Panico M, Gilmartin T, Whisenant T, Lanigan C M, Head S R, Goldberg D, Morris H R, Dell A and Paulson J C. (2006). Activation of murine CD4+ and CD8+ T lymphocytes leads to dramatic remodeling of N-linked glycans. J Immunol 177:2431-2440.
12. Hui N, Le Marer N. (2001). Alpha-2,6-sialylation regulation in CD34+ progenitor cells in the human bone marrow and granulocyte colony-stimulating factor mobilization. J Hematother Stem Cell Res 10:661-668.
13. Marino J H, Hoffman M, Meyer M and Miller K S. (2004). Sialyltransferase mRNA abundances in B cells are strictly controlled, correlated with cognate lectin binding, and differentially responsive to immune signaling in vitro. Glycobiology 14: 1265-1274.
14. Jaatinen T, Hemmoranta H, Hautaniemi S, Niemi J, Nicorici D, Laine J, Yli-Harja O and Partanen J. (2006). Global gene expression profile of human cord blood-derived CD133+ cells. Stem Cells 24:631-641.
15. Kekarainen T, Mannelin S, Laine J and Jaatinen T. (2006). Optimization of immunomagnetic separation for cord blood-derived hematopoietic stem cells. BMC Cell Biol 7:30:30.
16. Kornfeld R, Komfeld S. (1985). Assembly of asparagine-linked oligosaccharides. Annu Rev Biochem 54:631-664.
17. Schachter H. (1991). The ‘yellow brick road’ to branched complex N-glycans. Glycobiology 1:453-461.
18. Mollicone R, Gibaud A, Francois A, Ratcliffe M and Oriol R. (1990). Acceptor specificity and tissue distribution of three human alpha-3-fucosyltransferases. Eur J Biochem 191:169-176.
19. Mollicone R, Candelier J J, Mennesson B, Couillin P, Venot A P and Oriol R. (1992). Five specificity patterns of (1-3)-alpha-L-fucosyltransferase activity defined by use of synthetic oligosaccharide acceptors. Differential expression of the enzymes during human embryonic development and in adult tissues. Carbohydr Res 228:265-276.
20. Miyoshi E, Noda K, Yamaguchi Y, Inoue S, Ikeda Y, Wang W, K o J H, Uozumi N, Li W and Taniguchi N. (1999). The alpha1-6-fucosyltransferase gene and its biological significance. Biochim Biophys Acta 1473:9-20.
21. Herscovics A. (2001). Structure and function of Class I alpha 1,2-mannosidases involved in glycoprotein synthesis and endoplasmic reticulum quality control. Biochimie 83:757-762.
22. Lal A, Pang P, Kalelkar S, Romero P A, Herscovics A and Moremen K W. (1998). Substrate specificities of recombinant murine Golgi alpha1, 2-mannosidases IA and IB and comparison with endoplasmic reticulum and Golgi processing alpha1,2-mannosidases. Glycobiology 8:981-995.
23. Yoshida A, Minowa M T, Takamatsu S, Hara T, Oguri S, Ikenaga H and Takeuchi M. (1999). Tissue specific expression and chromosomal mapping of a human UDP-N-acetylglucosamine: alpha-1,3-d-mannoside beta1,4-N-acetylglucosaminyltransferase. Glycobiology 9:303-310.
24. Forde S P, Jorgensen T B, Newey S E, Roubelakis M, Smythe J, McGuckin C P, Pettengell R and Watt S M. (2006). Endolyn (CD164) Modulates the CXCL12-Mediated Migration of Umbilical Cord Blood CD133+ Cells. Blood.:
25. Doyonnas R, Yi-Hsin C J, Butler L H, Rappold I, Lee-Prudhoe J E, Zannettino A C, Simmons P J, Buhring H J, Levesque J P and Watt S M. (2000). CD164 monoclonal antibodies that block hemopoietic progenitor cell adhesion and proliferation interact with the first mucin domain of the CD164 receptor. J Immunol 165:840-851.
26. Watt S M, Buhring H J, Rappold I, Chan J Y, Lee-Prudhoe J, Jones T, Zannettino A C, Simmons P J, Doyonnas R, Sheer D and Butler L H. (1998). CD164, a novel sialomucin on CD34(+) and erythroid subsets, is located on human chromosome 6q21. Blood 92:849-866.
27. Zannettino A C, Buhring H J, Niutta S, Watt S M, Benton M A and Simmons P J. (1998). The sialomucin CD164 (MGC-24v) is an adhesive glycoprotein expressed by human hematopoietic progenitors and bone marrow stromal cells that serves as a potent negative regulator of hematopoiesis. Blood 92:2613-2628.
28. Lo N W, Shaper J H, Pevsner J and Shaper N L. (1998). The expanding beta 4-galactosyltransferase gene family: messages from the databanks. Glycobiology 8:517-526.
29. Almeida R, Amado M, David L, Levery S B, Holmes E H, Merkx G, van Kessel A G, Rygaard E, Hassan H, Bennett E and Clausen H. (1997). A family of human beta4-galactosyltransferases. Cloning and expression of two novel UDP-galactose:beta-n-acetylglucosamine beta1, 4-galactosyltransferases, beta4Gal-T2 and beta4Gal-T3. J Biol Chem 272:31979-31991.
30. Asano M, Furukawa K, Kido M, Matsumoto S, Umesaki Y, Kochibe N and Jwakura Y. (1997). Growth retardation and early death of beta-1,4-galactosyltransferase knockout mice with augmented proliferation and abnormal differentiation of epithelial cells. EMBO J. 16:1850-1857.
31. Schwartz-Albiez R, Merling A, Martin S, Haas R and Gross H J. (2004). Cell surface sialylation and ecto-sialyltransferase activity of human CD34 progenitors from peripheral blood and bone marrow. Glycoconj J21:451-459.
32. Ghosh S, Bandulet C and Nitschke L. (2006). Regulation of B cell development and B cell signalling by CD22 and its ligands alpha-2,6-linked sialic acids. Int Immunol 18:603-611.
33. Amano M, Galvan M, He J and Baum L G. (2003). The ST6Gal I sialyltransferase selectively modifies N-glycans on CD45 to negatively regulate galectin-1-induced CD45 clustering, phosphatase modulation, and T cell death. J Biol Chem 278:7469-7475.
34. Kiss J, Kunstar A, Fajka-Boja R, Dudics V, Tovari J, Legradi A, Monostori E and Uher F. (2007). A novel anti-inflammatory function of human galectin-1: inhibition of hematopoietic progenitor cell mobilization. Exp Hematol 35:305-313.
35. Kadri T, Lataillade J J, Doucet C, Marie A, Ernou I, Bourin P, Joubert-Caron R, Caron M and Lutomski D. (2005). Proteomic study of Galectin-1 expression in human mesenchymal stem cells. Stem Cells Dev 14:204-212.
36. Robinson S N, Ng J, Niu T, Yang H, McMannis J D, Karandish S, Kaur I, Fu P, Del Angel M, Messinger R, Flagge F, de Lima M, Decker W, Xing D, Champlin R and Shpall E J. (2006). Superior ex vivo cord blood expansion following co-culture with bone marrow-derived mesenchymal stem cells. Bone Marrow Transplant 37:359-366.
37. Okajima T, Fukumoto S, Miyazaki H, Ishida H, Kiso M, Furukawa K, Urano T and Furukawa K. (1999). Molecular cloning of a novel alpha-2,3-sialyltransferase (ST3Gal VI) that sialylates type II lactosamine structures on glycoproteins and glycolipids. J Biol Chem 274:11479-11486.
38. Dercksen M W, Gerritsen W R, Rodenhuis S, Dirkson M K, Slaper-Cortenbach I C, Schaasberg W P, Pinedo H M, dem Borne A E and van der Schoot C E. (1995). Expression of adhesion molecules on CD34+ cells: CD34+L-selectin+ cells predict a rapid platelet recovery after peripheral blood stem cell transplantation. Blood 85:3313-3319.
39. Dimitroff C J, Lee J Y, Fuhlbrigge R C and Sackstein R. (2000). A distinct glycoform of CD44 is an L-selectin ligand on human hematopoietic cells. Proc Natl Acad Sci USA 97:13841-13846.
40. Dimitroff C J, Lee J Y, Rafii S, Fuhlbrigge R C and Sackstein R. (2001). CD44 is a major E-selectin ligand on human hematopoietic progenitor cells. J Cell Biol 153:1277-1286.
41. Skelton T P, Zeng C, Nocks A and Stamenkovic I. (1998). Glycosylation provides both stimulatory and inhibitory effects on cell surface and soluble CD44 binding to hyaluronan. J Cell Biol 140:431-446.
42. Aruffo A, Stamenkovic I, Melnick M, Underhill C B and Seed B. (1990). CD44 is the principal cell surface receptor for hyaluronate. Cell 61:1303-1313.
43. Peled A, Grabovsky V, Habler L, Sandbank J, Arenzana-Seisdedos F, Petit I, Ben Hur H, Lapidot T and Alon R. (1999). The chemokine SDF-1 stimulates integrin-mediated arrest of CD34(+) cells on vascular endothelium under shear flow. J Clin Invest 104:1199-1211.
44. Sasaki K, Kurata K, Funayama K, Nagata M, Watanabe E, Ohta S, Hanai N and Nishi T. (1994). Expression cloning of a novel alpha 1,3-fucosyltransferase that is involved in biosynthesis of the sialyl Lewis x carbohydrate determinants in leukocytes. J Biol Chem %20; 269:14730-14737.
45. Zerfaoui M, Fukuda M, Sbarra V, Lombardo D and El Battari A. (2000). alpha(1,2)-fucosylation prevents sialyl Lewis x expression and E-selectin-mediated adhesion of fucosyltransferase VII-transfected cells. Eur J Biochem 267:53-61.
46. Bengtson P, Lundblad A, Larson G and Pahlsson P. (2002). Polymorphonuclear leukocytes from individuals carrying the G329A mutation in the alpha 1,3-fucosyltransferase VII gene (FUT7) roll on E- and P-selectins. J Immunol 169:3940-3946.
47. Hemmoranta H, Hautaniemi S, Niemi J, Nicorici D, Laine J, Yli-Harja O, Partanen J and Jaatinen T. (2006). Transcriptional profiling reflects shared and unique characters for CD34+ and CD133+ cells. Stem Cells Dev 15:839-851.
48. Bause E, Bieberich E, Rolfs A, Volker C and Schmidt B. (1993). Molecular cloning and primary structure of Man9-mannosidase from human kidney. Eur J Biochem 217:535-540.
49. Misago M, Liao Y F, Kudo S, Eto S, Mattei M G, Moremen K W and Fukuda M N. (1995). Molecular cloning and expression of cDNAs encoding human alpha-mannosidase II and a previously unrecognized alpha-mannosidase IIx isozyme. Proc Natl Acad Sci USA 92:11766-11770.
50. Moremen K W, Robbins P W. (1991). Isolation, characterization, and expression of cDNAs encoding murine alpha-mannosidase II, a Golgi enzyme that controls conversion of high mannose to complex N-glycans. J Cell Biol 115:1521-1534.
51. Nebes V L, Schmidt M C. (1994). Human lysosomal alpha-mannosidase: isolation and nucleotide sequence of the full-length cDNA. Biochem Biophys Res Commun 200:239-245.
52. Saito S I Aoki H, Ito A, Ueno S, Wada T, Mitsuzuka K, Satoh M, Arai Y and Miyagi T. (2003). Human alpha2,3-sialyltransferase (ST3Gal II) is a stage-specific embryonic antigen-4 synthase. J Biol Chem 278:26474-26479.
53. Tremblay L O, Campbell D N and Herscovics A. (1998). Molecular cloning, chromosomal mapping and tissue-specific expression of a novel human alpha-1,2-mannosidase gene involved in N-glycan maturation. Glycobiology 8:585-595.
54. Tremblay L O, Herscovics A. (1999). Cloning and expression of a specific human alpha 1,2-mannosidase that trims Man9GlcNAc2 to Man8GlcNAc2 isomer B during N-glycan biosynthesis. Glycobiology 9:1073-1078.
55. Amado M, Almeida R, Carneiro F, Levery S B, Holmes E H, Nomoto M, Hollingsworth M A, Hassan H, Schwientek T, Nielsen P A, Bennett E P and Clausen H. (1998). A family of human beta3-galactosyltransferases. Characterization of four members of a UDP-galactose:beta-N-acetyl-glucosamine/beta-nacetyl-galactosamine beta-1,3-galactosyltransferase family. J Biol Chem 273: 12770-12778.
56. Bai X, Zhou D, Brown J R, Crawford B E, Hennet T and Esko J D. (2001). Biosynthesis of the linkage region of glycosaminoglycans: cloning and activity of galactosyltransferase II, the sixth member of the beta 1,3-galactosyltransferase family (beta 3GalT6). J Biol Chem 276:48189-48195.
57. Isshiki S, Togayachi A, Kudo T, Nishihara S, Watanabe M, Kubota T, Kitajima M, Shiraishi N, Sasaki K, Andoh T and Narimatsu H. (1999). Cloning, expression, and characterization of a novel UDP-galactose:beta-N-acetylglucosamine beta-1,3-galactosyltransferase (beta3Gal-T5) responsible for synthesis of type 1 chain in colorectal and pancreatic epithelia and tumor cells derived therefrom. J Biol Chem 274:12499-12507.
58. Kolbinger F, Streiff M B and Katopodis A G. (1998). Cloning of a human UDP-galactose:2-acetamido-2-deoxy-D-glucose 3beta-galactosyltransferase catalyzing the formation of type 1 chains. J Biol Chem 273:433-440.
59. Okajima T, Nakamura Y, Uchikawa M, Haslam D B, Numata S I, Furukawa K, Urano T and Furukawa K. (2000). Expression cloning of human globoside synthase cDNAs. Identification of beta 3Gal-T3 as UDP-N-acetylgalactosamine:globotriaosylceramide beta 1,3-N-acetylgalactosaminyltransferase. Biol Chem 275:40498-40503.
60. Zhou D, Henion T R, Jungalwala F B, Berger E G and Hennet T. (2000). The beta 1,3-galactosyltransferase beta 3GalT-V is a stage-specific embryonic antigen-3 (SSEA-3) synthase. J Biol Chem 275:22631-22634.
61. Almeida R, Levery S B, Mandel U, Kresse H, Schwientek T, Bennett E P and Clausen H. (1999). Cloning and expression of a proteoglycan UDP-galactose:beta-xylose beta-1,4-galactosyltransferase I. A seventh member of the human beta4-galactosyltransferase gene family. J Biol Chem 274:26165-26171.
62. Amado M, Almeida R, Schwientek T and Clausen H. (1999). Identification and characterization of large galactosyltransferase gene families: galactosyltransferases for all functions. Biochim Biophys Acta 1473:35-53.
63. Okajima T, Yoshida K, Kondo T and Furukawa K. (1999). Human homolog of Caenorhabditis elegans sqv-3 gene is galactosyltransferase I involved in the biosynthesis of the glycosaminoglycan-protein linkage region of proteoglycans. J Biol Chem 274:22915-22918.
64. Schwientek T, Almeida R, Levery S B, Holmes E H, Bennett E and Clausen H. (1998). Cloning of a novel member of the UDP-galactose:beta-N-acetylglucosamine beta-1,4-galactosyltransferase family, beta4Gal-T4, involved in glycosphingolipid biosynthesis. J Biol Chem 273:29331-29340.
65. van D, I, van Tetering A, Schiphorst W E, Sato T, Furukawa K and van den Eijnden D H. (1999). The acceptor substrate specificity of human beta4-galactosyltransferase V indicates its potential function in O-glycosylation. FEBS Lett 450:52-56.
66. Grundmann U, Nerlich C, Rein T and Zettlmeissl G. (1990). Complete cDNA sequence encoding human beta-galactoside alpha-2,6-sialyltransferase. Nucleic Acids Res 18:667.
67. Takashima S, Tsuji S and Tsujimoto M. (2002). Characterization of the second type of human beta-galactoside alpha 2,6-sialyltransferase (ST6Gal II), which sialylates Galbeta 1,4GlcNAc structures on oligosaccharides preferentially. Genomic analysis of human sialyltransferase genes. J Biol Chem 277:45719-45728.
68. Gillespie W, Kelm S and Paulson J C. (1992). Cloning and expression of the Gal beta 1,3GalNAc alpha 2,3-sialyltransferase. J Biol Chem 267:21004-21010.
69. Kitagawa H, Paulson J C. (1994). Cloning of a novel alpha 2,3-sialyltransferase that sialylates glycoprotein and glycolipid carbohydrate groups. J Biol Chem 269:1394-1401.
70. Kitagawa H, Mattei M G and Paulson J C. (1996). Genomic organization and chromosomal mapping of the Gal beta 1,3GalNAc/Gal beta 1,4GlcNAc alpha 2,3-sialyltransferase. J Biol Chem 271:931-938.
71. Kono M, Takashima S, Liu H, Inoue M, Kojima N, Lee Y C, Hamamoto T and Tsuji S. (1998). Molecular cloning and functional expression of a fifth-type alpha 2,3-sialyltransferase (mST3Gal V: GM3 synthase). Biochem Biophys Res Commun 253: 170-175.
72. Larsen R D, Ernst L K, Nair R P and Lowe J B. (1990). Molecular cloning, sequence, and expression of a human GDP-L-fucose:beta-D-galactoside 2-alpha-L-fucosyltransferase cDNA that can form the H blood group antigen. Proc Natl Acad Sci USA 87:6674-6678.
73. Koda Y, Kimura H and Mekada E. (1993). Analysis of Lewis fucosyltransferase genes from the human gastric mucosa of Lewis-positive and -negative individuals. Blood 82:2915-2919.
74. Kelly R J, Rouquier S, Giorgi D, Lennon G G and Lowe J B. (1995). Sequence and expression of a candidate for the human Secretor blood group alpha(1,2)fucosyltransferase gene (FUT2). Homozygosity for an enzyme-inactivating nonsense mutation commonly correlates with the non-secretor phenotype. J Biol Chem 270:4640-4649.
75. Couillin P, Mollicone R, Grisard M C, Gibaud A, Ravise N, Feingold J and Oriol R. (1991). Chromosome 11q localization of one of the three expected genes for the human alpha-3-fucosyltransferases, by somatic hybridization. Cytogenet Cell Genet. 56:108-111.
76. Weston B W, Nair R P, Larsen R D and Lowe J B. (1992). Isolation of a novel human alpha (1,3)fucosyltransferase gene and molecular comparison to the human Lewis blood group alpha (1,3/1,4)fucosyltransferase gene. Syntenic, homologous, nonallelic genes encoding enzymes with distinct acceptor substrate specificities. J Biol Chem 267:4152-4160.
77. Koszdin K L, Bowen B R. (1992). The cloning and expression of a human alpha-1,3 fucosyltransferase capable of forming the E-selectin ligand. Biochem Biophys Res Commun 187:152-157.
78. Natsuka S, Gersten K M, Zenita K, Kannagi R and Lowe J B. (1994). Molecular cloning of a cDNA encoding a novel human leukocyte alpha-1,3-fucosyltransferase capable of synthesizing the sialyl Lewis x determinant. J Biol Chem 269:16789-16794.
79. Yamaguchi Y, Fujii J, Inoue S, Uozumi N, Yanagidani S, Ikeda Y, Egashira M, Miyoshi O, Niikawa N and Taniguchi N. (1999). Mapping of the alpha-1,6-fucosyltransferase gene, FUT8, to human chromosome 14q24.3. Cytogenet Cell Genet. 84:58-60.
80. Kaneko M, Kudo T, Iwasaki H, Ikehara Y, Nishihara S, Nakagawa S, Sasaki K, Shiina T, Inoko H, Saitou N and Narimatsu H. (1999). Alpha-1,3-fucosyltransferase IX (Fuc-TIX) is very highly conserved between human and mouse; molecular cloning, characterization and tissue distribution of human Fuc-TIX. FEBS Lett 452:237-242.

### Example 2

### Evaluation of Cord Blood CD133+ and CD133− Cell Associated N-Glycans

N-glycan profile data was characterized from human cord blood hematopoietic CD133+ and CD133− cells as described in Example 1. The data was evaluated according to the relative association of each glycan signal to either cell type as described in the legends of Tables 3 and 4, and sorted accordingly into CD133+ and CD133− associated glycan signals in Tables 3 and 4 for neutral and sialylated N-glycan signals, respectively. In this calculation, three groups of glycan signals were obtained for each cell type: over 2-fold difference (significant association), between 2 and 1.5-fold difference (substantial association), and below 1.5-fold difference (small but detected association). The data demonstrated that in addition to glycan signal groups identified in Example 1, also the other glycan signals were associated with either CD133+ or CD133− cells.

### Example 3

### Evaluation of Individual Variation in Cord Blood CD133+ and CD133− Cell N-Glycans

N-glycan profile data was characterized from human cord blood hematopoietic CD133+ and CD133− cells as described in Example 1, and data shown in Tables 5 and 6 was collected from several cord blood units to evaluate individual variation for each glycan signal as described in the legends of Tables 5 and 6, and sorted accordingly into glycan signal groups. In this calculation, three groups of glycan signals were obtained: over 100% average deviation (large individual variation), between 50-100% average deviation (substantial individual variation), and between 0-50% average deviation (little individual variation). The data demonstrated that there was both glycan signal-associated and glycan signal group associated differences in individual variation of glycan signals.

### Example 4

### Enzymatic Modification of Cell Surface Glycan Structures

### Experimental Procedures

Enzymatic modifications. Sialyltransferase reaction: Human cord blood mononuclear cells (3×10⁶ cells) were modified with 60 mU α2,3-(N)-sialyltransferase (rat, recombinant in S. frugiperda, Calbiochem), 1.6 μmol CMP-Neu5Ac in 50 mM sodium 3-morpholinopropanesulfonic acid (MOPS) buffer pH 7.4, 150 mM NaCl at total volume of 100 μl for up to 12 hours. Fucosyltransferase reaction: Human cord blood mononuclear cells (3×10⁶ cells) were modified with 4 mU α-1,3-fucosyltransferase VI (human, recombinant in S. frugiperda, Calbiochem), 1 μmol GDP-Fuc in 50 mM MOPS buffer pH 7.2, 150 mM NaCl at total volume of 100 μl for up to 3 hours. Broad-range sialidase reaction: Human cord blood mononuclear cells (3×10⁶ cells) were modified with 5 mU sialidase (A. ureafaciens, Glyko, UK) in 50 mM sodium acetate buffer pH 5.5, 150 mM NaCl at total volume of 1001l for up to 12 hours. α2,3-specific sialidase reaction: Cells were modified with α2,3-sialidase (S. pneumoniae, recombinant in E. coli) in 50 mM sodium acetate buffer pH 5.5, 150 mM NaCl at total volume of 100 μl. Sequential enzymatic modifications: Between sequential reactions cells were pelleted with centrifugation and supernatant was discarded, after which the next modification enzyme in appropriate buffer and substrate solution was applied to the cells as described above. Washing procedure: After modification, cells were washed with phosphate buffered saline.

Glycan analysis. After washing the cells, total cellular glycoproteins were subjected to N-glycosidase digestion, and sialylated and neutral N-glycans isolated and analyzed with mass spectrometry as described above. For O-glycan analysis, the glycoproteins were subjected to reducing alkaline β-elimination essentially as described previously (Nyman et al., 1998), after which sialylated and neutral glycan alditol fractions were isolated and analyzed with mass spectrometry as described above.

### Glycans Remodeled by Glycosyltransferases/Glycosyltransfer

The present invention is further directed to special glycan controlled reagent produced by process including steps
1) Optionally partially depleting glycan structure as described by the invention, the partially depleted glycan structure may be also a non-animal structure as described for group 2 of glycan depleted reagents or a glycosylated protein from a prokaryote.
2) Transferring an acceptable or non-harmful glycan to glycan of reagent. Such process is known as glycoprotein remodelling for certain therapeutic proteins. The inventors revealed that there is a need for a remodelling process for specific reagents present in cell culture processes.
   Furthermore the inventors were able to show glycan depletion and/or remodelling of large protein mixtures even for total serum involving numerous factors potentially inhibiting transfer reactions.

### Results

Sialidase digestion. Upon broad-range sialidase catalyzed desialylation of living cord blood mononuclear cells, sialylated N-glycan structures as well as O-glycan structures (data not shown) were desialylated, as indicated by increase in relative amounts of corresponding neutral N-glycan structures, for example Hex₆HexNAc₃, Hex₅HexNAc₄dHex₀₋₂, and Hex₆HexNAc₅dHex₀₋₁ monosaccharide compositions (Table 9). In general, a shift in glycosylation profiles towards glycan structures with less sialic acid residues was observed in sialylated N-glycan analyses upon broad-range sialidase treatment. The shift in glycan profiles of the cells upon the reaction served as an effective means to characterize the reaction results. It is concluded that the resulting modified cells contained less sialic acid residues and more terminal galactose residues at their surface after the reaction.

α2,3-specific sialidase digestion. Similarly, upon α2,3-specific sialidase catalyzed desilylation of living mononuclear cells, sialylated N-glycan structures were desilylated, as indicated by increase in relative amounts of corresponding neutral N-glycan structures (data not shown). In general, a shift in glycosylation profiles towards glycan structures with less sialic acid residues was observed in sialylated N-glycan analyses upon α2,3-specific sialidase treatment. The shift in glycan profiles of the cells upon the reaction served as an effective means to characterize the reaction results. It is concluded that the resulting modified cells contained less α2,3-linked sialic acid residues and more terminal galactose residues at their surface after the reaction.

Sialyltransferase reaction. Upon α2,3-sialyltransferase catalyzed sialylation of living cord blood mononuclear cells, numerous neutral (Table 9) and sialylated N-glycan (Table 8) structures as well as O-glycan structures (data not shown) were sialylated, as indicated by decrease in relative amounts of neutral N-glycan structures (Hex₅HexNAc₄dHex₀₋₃ and Hex₆HexNAc₅dHex₀₋₂ monosaccharide compositions in Table 9) and increase in the corresponding sialylated structures (for example the NeuAc₂Hex₅HexNAc₄dHex, glycan in Table 8). In general, a shift in glycosylation profiles towards glycan structures with more sialic acid residues was observed both in N-glycan and O-glycan analyses. It is concluded that the resulting modified cells contained more α2,3-linked sialic acid residues and less terminal galactose residues at their surface after the reaction.

Fucosyltransferase reaction. Upon α1,3-fucosyltransferase catalyzed fucosylation of living cord blood mononuclear cells, numerous neutral (Table 9) and sialylated N-glycan structures as well as O-glycan structures (see below) were fucosylated, as indicated by decrease in relative amounts of nonfucosylated glycan structures (without dHex in the proposed monosaccharide compositions) and increase in the corresponding fucosylated structures (with n_{dHex}>0 in the proposed monosaccharide compositions). For example, before fucosylation O-glycan alditol signals at m/z 773, corresponding to the [M+Na]⁺ ion of Hex₂HexNAc₂ alditol, and at m/z 919, corresponding to the [M+Na]⁺ ion of Hex₂HexNAc₂dHex, alditol, were observed in approximate relative proportions 9:1, respectively (data not shown). After fucosylation, the approximate relative proportions of the signals were 3:1, indicating that significant fucosylation of neutral O-glycans had occurred. Some fucosylated N-glycan structures were even observed after the reaction that had not been observed in the original cells, for example neutral N-glycans with proposed structures Hex₆HexNAc₅dHex, and Hex₆HexNAc₅dHex₂ (Table 9), indicating that in α1,3-fucosyltransferase reaction the cell surface of living cells can be modified with increased amounts or extraordinary structure types of fucosylated glycans, especially terminal Lewis x epitopes in protein-linked N-glycans as well as in O-glycans.

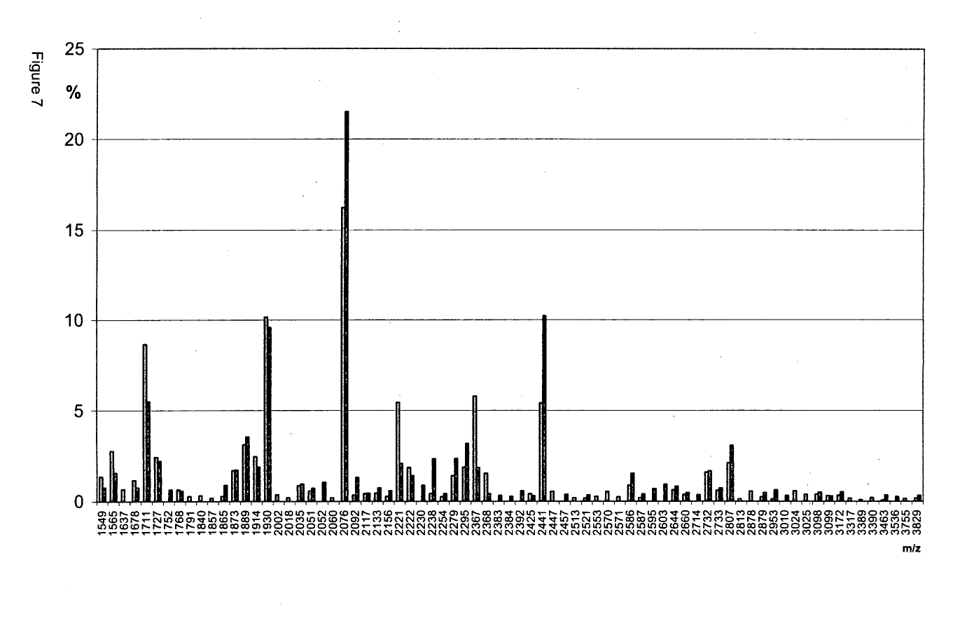

Sialidase digestion followed by sialyltransferase reaction. Cord blood mononuclear cells were subjected to broad-range sialidase reaction, after which α2,3-sialyltransferase and CMP-Neu5Ac were added to the same reaction, as described under Experimental procedures. The effects of this reaction sequence on the N-glycan profiles of the cells are described in FIG. 7. The sialylated N-glycan profile was also analyzed between the reaction steps, and the result clearly indicated that sialic acids were first removed from the sialylated N-glycans (indicated for example by appearance of increased amounts of neutral N-glycans), and then replaced by α2,3-linked sialic acid residues (indicated for example by disappearance of the newly formed neutral N-glycans; data not shown). It is concluded that the resulting modified cells contained more α2,3-linked sialic acid residues after the reaction.

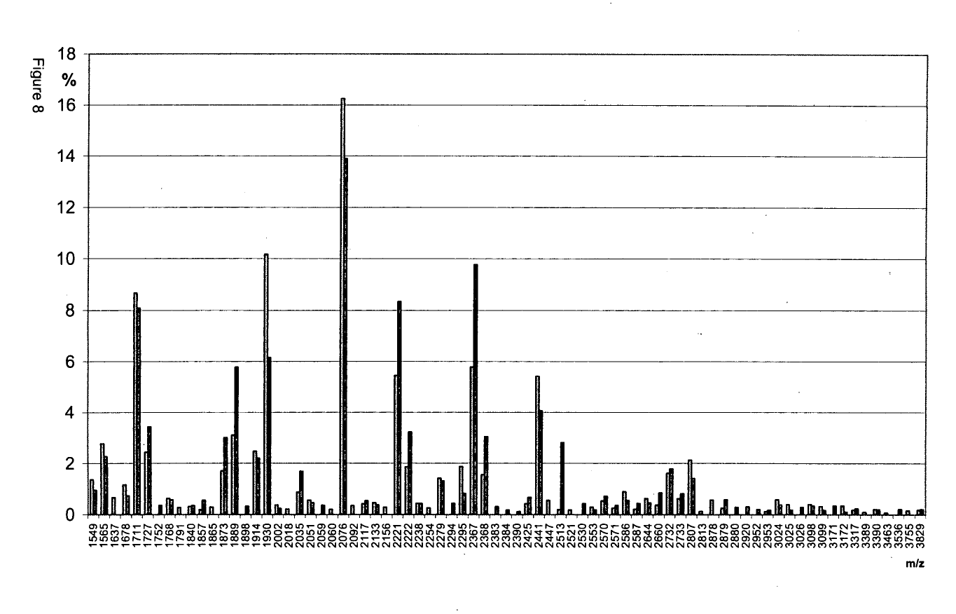

Sialyltransferase reaction followed by fucosyltransferase reaction. Cord blood mononuclear cells were subjected to α2,3-sialyltransferase reaction, after which α1,3-fucosyltransferase and GDP-fucose were added to the same reaction, as described under Experimental procedures. The effects of this reaction sequence on the sialylated N-glycan profiles of the cells are described in FIG. 8. The results show that a major part of the glycan signals (detailed in Table 7) have undergone changes in their relative intensities, indicating that a major part of the sialylated N-glycans present in the cells were substrates of the enzymes. It was also clear that the combination of the enzymatic reaction steps resulted in different result than either one of the reaction steps alone.

Different from the α1,3-fucosyltransferase reaction described above, sialylation before fucosylation apparently sialylated the neutral fucosyltransferase acceptor glycan structures present on cord blood mononuclear cell surfaces, resulting in no detectable formation of the neutral fucosylated N-glycan structures that had emerged after α1,3-fucosyltransferase reaction alone (discussed above; Table 9).

α-mannosidase reaction. α-mannosidase reaction of whole cells showed a minor reduction of glycan signals including those indicated to contain α-mannose residues in other examples. The invention further revealed that the cells are viable under the enzymatic modification conditions according to the invention, Table 18.

The invention is especially directed to the methods according to the invention for analysis of hematopoietic cells when the cells are modified by enzymatic reaction, preferably sialyltransferase, fucosyltransferase, galactosyltransferase (e.g., β4-GalT) or glycosidases according to the invention capable of modifying glycans, preferably cell surface glycans of hematopoietic cells, preferably sialidase or mannosidase modifying terminal GlcNAc residues, and preferably the cells are cell surface modified under condition in which they are viable cells to avoid intracellular reaction with broken cells. The preferred binder reagents, such as antibodies or lectins, are selected to recognize the cell surface eptioes synthesized by the enzymes such as Galβ4GlcNAc, sialylα3/6Galβ3/4GlcNAc, more preferably sialylα3/6Galβ4GlcNAc or sialyl-Lewis x, alternatively the glycans are analyzed by mass spectrometric profiling.

Glycosyltransferase-derived glycan structures. We detected that glycosylated glycosyltransferase enzymes can contaminate cells in modification reactions. For example, when cells were incubated with recombinant fucosyltransferase or sialyltransferase enzymes produced in S. frugiperda cells, N-glycosidase and mass spectrometric analysis of cellular and/or cell-associated glycoproteins resulted in detection of an abundant neutral N-glycan signal at m/z 1079, corresponding to [M+Na]⁺ ion of Hex₃HexNAc₂dHex, glycan component (calc. m/z 1079.38). Typically, in recombinant glycosyltransferase treated cells, this glycan signal was more abundant than or at least comparable to the cells' own glycan signals, indicating that insect-derived glycoconjugates are a very potent contaminant associated with recombinant glycan-modified enzymes produced in insect cells. Moreover, this glycan contamination persisted even after washing of the cells, indicating that the insect-type glycoconjugate corresponding to or associated with the glycosyltransferase enzymes has affinity towards cells or has tendency to resist washing from cells. To confirm the origin of the glycan signal, we analyzed glycan contents of commercial recombinant fucosyltransferase and sialyltransferase enzyme preparations and found that the m/z 1079 glycan signal was a major N-glycan signal associated with these enzymes. Corresponding N-glycan structures, e.g. Manα3(Manα6)Manβ4GlcNAc(Fucα3/6)GlcNAc(β-N-Asn), have been described previously from glycoproteins produced in S. frugiperda cells (Staudacher et al., 1992; Kretzchmar et al., 1994; Kubelka et al., 1994; Altmann et al., 1999). As described in the literature, these glycan structures, as well as other glycan structures potentially contaminating cells treated with recombinant or purified enzymes, especially insect-derived products, are potentially immunogenic in humans and/or otherwise harmful to the use of the modified cells. It is concluded that glycan-modifying enzymes must be carefully selected for modification of human cells, especially for clinical use, not to contain immunogenic glycan epitopes, non-human glycan structures, and/or other glycan structures potentially having unwanted biological effects.

### Example 5

Analysis of stability and cultivation properties of glycosidase or glycosyltransferase modified cells Stability and cultivation properties of neuraminidase and glycosyltransferase (sialyltransferase and fucosyltransferase) modified cells from previous example were analyzed in CFU cell culture assay and viability assay as described in (Kekarainen et al BMC Cell Biol (2006) 7, 30).

The invention revealed that the modified cord blood mononuclear cells with quantitatively reduced sialic acid levels gave in CFU cell culture assay higher colony counts. The invention is especially directed to the use of the desialylated hematopoietic cells for cultivation of blood cell populations, especially for cultivation of hematopoietic cells (Table 18).

### Example 6

### Analysis of N-Glycan Composition Groups with Terminal HexNAc in Stem Cells and Differentiated Cells

Methods. To analyze the presence of terminal HexNAc containing N-glycans characterized by the formulae: n_{HexNAc}=n_{Hex}≧5 and n_{dHex}≧1 (group I), and to compare their occurrence to terminal HexNAc containing N-glycans characterized by the formulae: n_{HexNAc}=n_{Hex}≧5 and n_{dHex}=0 (group II), N-glycans were isolated, purified and analyzed by MALDI-TOF mass spectrometry as described in the preceding Examples. They were assigned monosaccharide compositions and their relative proportions within the obtained glycan profiles were determined by quantitative profile analysis as described above. The following glycan signals were used as indicators of the specific glycan groups (monoisotopic masses):

Ia, Hex₅HexNAc₅dHex₁: m/z for [M+Na]+ ion 2012.7
Ib, NeuAc₁Hex₅HexNAc₅dHex₁: m/z for [M-H]− ion 2279.8
Ic, NeuAc₂Hex₅HexNAc₅dHex₁: m/z for [M-H]− ion 2570.9
Id, NeuAc₁Hex₅HexNAc₅dHex₂: m/z for [M-H]− ion 2425.9

### IIa, NeuAc₁Hex₅HexNAc₅: m/z for [M-H]− ion 2133.8

Further, relative expression of glycan signals Hex₃HexNAc₅: m/z for [M+Na]+ ion 1542.6 and Hex₃HexNAc₅dHex₁: m/z for [M+Na]⁺ ion 1688.6 was also analyzed.

Results. As an indicator of group I glycans, Ib was detected in various N-glycan samples isolated from stem cell samples, including CB MSC, BM MSC, and CD34+ CB HSC, as well as in differentiated cell samples, including EB and st.3 differentiated cells, adipocyte differentiated cells (from CB MSC), osteoblast differentiated cells (from BM MSC), and CD34− CB MNC.

CB HSC: Ib and Ic were overexpressed in CB CD34− cells when compared to CD34+ cells, whereas Id was overexpressed in CD34+ cells. Ia was expressed in both CD34+ and CD34− cells. Ia and Ic were not expressed. Hex₃HexNAc₅dHex, was observed in both CB CD34+ and CB CD34− cells, but not in adult peripheral blood CD34+ cells. Hex₃HexNAc₅dHex, was overexpressed in CD133+ and lin− cells when compared to CD133− and lin+ cells, respectively.

CB and BM MSC: Of Ia-d and IIa, only Ib was expressed in CB MSC, whereas Ia, Ib, and Id were overexpressed in osteoblast differentiated cells. Of Ia-d and Ia, only Ia and Ib were expressed in BM MSC, whereas Ia, Ib, and Id were overexpressed in adipocyte differentiated cells. Hex₃HexNAc₅dHex, was expressed in MSC.

### Example 7

### Examples of Cell Sample Production

### Cord Blood Derived Mesenchymal Stem Cell Lines

Collection of umbilical cord blood. Human term umbilical cord blood (UCB) units were collected after delivery with informed consent of the mothers and the UCB was processed within 24 hours of the collection. The mononuclear cells (MNCs) were isolated from each UCB unit diluting the UCB 1:1 with phosphate-buffered saline (PBS) followed by Ficoll-Paque Plus (Amersham Biosciences, Uppsala, Sweden) density gradient centrifugation (400 g/40 min). The mononuclear cell fragment was collected from the gradient and washed twice with PBS.

Umbilical cord blood cell isolation and culture. CD45/Glycophorin A (GlyA) negative cell selection was performed using immunolabeled magnetic beads (Miltenyi Biotec). MNCs were incubated simultaneously with both CD45 and GlyA magnetic microbeads for 30 minutes and negatively selected using LD columns following the manufacturer's instructions (Miltenyi Biotec). Both CD45/GlyA negative elution fraction and positive fraction were collected, suspended in culture media and counted. CD45/GlyA positive cells were plated on fibronectin (FN) coated six-well plates at the density of 1×10⁶/cm². CD45/GlyA negative cells were plated on FN coated 96-well plates (Nunc) about 1×10⁴ cells/well. Most of the non-adherent cells were removed as the medium was replaced next day. The rest of the non-adherent cells were removed during subsequent twice weekly medium replacements.

The cells were initially cultured in media consisting of 56% DMEM low glucose (DMEM-LG, Gibco, http://www.invitrogen.com) 40% MCDB-201 (Sigma-Aldrich) 2% fetal calf serum (FCS), 1× penicillin-streptomycin (both form Gibco), 1×ITS liquid media supplement (insulin-transferrin-selenium), 1× linoleic acid-BSA, 5×10⁻⁸ M dexamethasone, 0.1 mM L-ascorbic acid-2-phosphate (all three from Sigma-Aldrich), 10 nM PDGF (R&D systems, http://www.RnDSystems.com) and 10 nM EGF (Sigma-Aldrich). In later passages (after passage 7) the cells were also cultured in the same proliferation medium except the FCS concentration was increased to 10%.

Plates were screened for colonies and when the cells in the colonies were 80-90% confluent the cells were subcultured. At the first passages when the cell number was still low the cells were detached with minimal amount of trypsin/EDTA (0.25%/1 mM, Gibco) at room temperature and trypsin was inhibited with FCS. Cells were flushed with serum free culture medium and suspended in normal culture medium adjusting the serum concentration to 2%. The cells were plated about 2000-3000/cm². In later passages the cells were detached with trypsin/EDTA from defined area at defined time points, counted with hematocytometer and replated at density of 2000-3000 cells/cm².

### Bone Marrow Derived Mesenchymal Stem Cell Lines

Isolation and culture of bone marrow derived stem cells. Bone marrow (BM)—derived MSCs were obtained as described by Leskela et al. (2003). Briefly, bone marrow obtained during orthopedic surgery was cultured in Minimum Essential Alpha-Medium (α-MEM), supplemented with 20 mM HEPES, 10% FCS, 1× penicillin-streptomycin and 2 mM L-glutamine (all from Gibco). After a cell attachment period of 2 days the cells were washed with Ca²⁺ and Mg²⁺ free PBS (Gibco), subcultured further by plating the cells at a density of 2000-3000 cells/cm2 in the same media and removing half of the media and replacing it with fresh media twice a week until near confluence.

### Experimental Procedures

Flow cytometric analysis of mesenchymal stem cell phenotype. Both UBC and BM derived mesenchymal stem cells were phenotyped by flow cytometry (FACSCalibur, Becton Dickinson). Fluorescein isothicyanate (FITC) or phycoerythrin (PE) conjugated antibodies against CD13, CD14, CD29, CD34, CD44, CD45, CD49e, CD73 and HLA-ABC (all from BD Biosciences, San Jose, Calif., http://www.bdbiosciences.com), CD105 (Abcam Ltd., Cambridge, UK, http://www.abcam.com) and CD133 (Miltenyi Biotec) were used for direct labeling. Appropriate FITC- and PE-conjugated isotypic controls (BD Biosciences) were used. Unconjugated antibodies against CD90 and HLA-DR (both from BD Biosciences) were used for indirect labeling. For indirect labeling FITC-conjugated goat anti-mouse IgG antibody (Sigma-aldrich) was used as a secondary antibody.

The UBC derived cells were negative for the hematopoietic markers CD34, CD45, CD14 and CD133. The cells stained positively for the CD13 (aminopeptidase N), CD29 (β1-integrin), CD44 (hyaluronate receptor), CD73 (SH3), CD90 (Thy1), CD105 (SH2/endoglin) and CD 49e. The cells stained also positively for HLA-ABC but were negative for HLA-DR. BM-derived cells showed to have similar phenotype. They were negative for CD 14, CD34, CD45 and HLA-DR and positive for CD13, CD29, CD44, CD90, CD105 and HLA-ABC.

Adipogenic differentiation. To assess the adipogenic potential of the UCB-derived MSCs the cells were seeded at the density of 3×10³/cm² in 24-well plates (Nunc) in three replicate wells. UCB-derived MSCs were cultured for five weeks in adipogenic inducing medium which consisted of DMEM low glucose, 2% FCS (both from Gibco), 10 μg/ml insulin, 0.1 mM indomethacin, 0.1 μM dexamethasone (Sigma-Aldrich) and penicillin-streptomycin (Gibco) before samples were prepared for glycome analysis. The medium was changed twice a week during differentiation culture.

Osteogenic differentiation. To induce the osteogenic differentiation of the BM-derived MSCs the cells were seeded in their normal proliferation medium at a density of 3×10³/cm² on 24-well plates (Nunc). The next day the medium was changed to osteogenic induction medium which consisted of α-MEM (Gibco) supplemented with 10% FBS (Gibco), 0.1 μM dexamethasone, 10 mM β-glycerophosphate, 0.05 mM L-ascorbic acid-2-phosphate (Sigma-Aldrich) and penicillin-streptomycin (Gibco). BM-derived MSCs were cultured for three weeks changing the medium twice a week before preparing samples for glycome analysis.

Cell harvesting for glycome analysis. 1 ml of cell culture medium was saved for glycome analysis and the rest of the medium removed by aspiration. Cell culture plates were washed with PBS buffer pH 7.2. PBS was aspirated and cells scraped and collected with 5 ml of PBS (repeated two times). At this point small cell fraction (10 μl) was taken for cell-counting and the rest of the sample centrifuged for 5 minutes at 400 g. The supernatant was aspirated and the pellet washed in PBS for an additional 2 times.

The cells were collected with 1.5 ml of PBS, transferred from 50 ml tube into 1.5 ml collection tube and centrifuged for 7 minutes at 5400 rpm. The supernatant was aspirated and washing repeated one more time. Cell pellet was stored at −70° C. and used for glycome analysis.

### Example 8

### Lectin and Antibody Profiling of Human Cord Blood Cell Populations

Collection of umbilical cord blood. Human term umbilical cord blood (UCB) units were collected after delivery with informed consent of the mothers and the UCB was processed within 24 hours of the collection. The mononuclear cells (MNCs) were isolated from each UCB unit diluting the UCB 1:1 with phosphate-buffered saline (PBS) followed by Ficoll-Paque Plus (Amersham Biosciences, Uppsala, Sweden) density gradient centrifugation (400 g/40 min). The mononuclear cell fragment was collected from the gradient and washed twice with PBS.

Umbilical cord blood cell isolation. CD45/Glycophorin A (GlyA) negative cell selection was performed using immunolabeled magnetic beads (Miltenyi Biotec). MNCs were incubated simultaneously with both CD45 and GlyA magnetic microbeads for 30 minutes and negatively selected using LD columns following the manufacturer's instructions (Miltenyi Biotec). Both CD45/GlyA negative elution fraction and positive fraction were collected, suspended in culture media and counted. CD45/GlyA positive cells were plated on fibronectin (FN) coated six-well plates at the density of 1×10⁶/cm². CD45/GlyA negative cells were plated on FN coated 96-well plates (Nunc) about 1×10⁴ cells/well. Most of the non-adherent cells were removed as the medium was replaced next day. The rest of the non-adherent cells were removed during subsequent twice weekly medium replacements. CD34+ and CD133+ were enriched essentially as described in Jaatinen T and Laine J. in Current Protocols in Stem cell Biology 2A.2.1-2A.2.9

### Results and Discussion

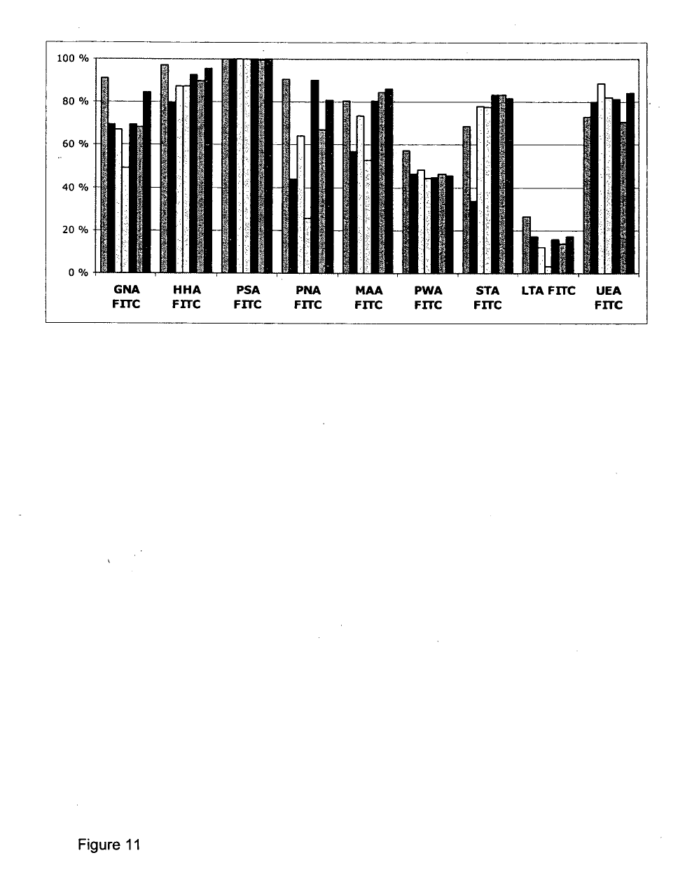

FIG. 11 shows the results of FACS analysis of FITC-labelled lectin binding to seven individual cord blood mononuclear cell (CB MNC) preparations (experiments performed as described above). Strong binding was observed in all samples by GNA, HHA, PSA, MAA, STA, and UEA FITC-labelled lectins, indicating the presence of their specific ligand structures on the CB MNC cell surfaces. Also mediocre binding (PWA), variable binding between CB samples (PNA), and low binding (LTA) was observed, indicating that the ligands for these lectins are either variable or more rare on the CB MNC cell surfaces as the lectins above.

### Example 9

### Analysis of Total N-Glycomes of Human Stem Cells and Cell Populations

### Experimental Procedures

Cell and glycan samples were prepared as described in the preceding Examples.

MALDI-TOF mass spectrometric glycan profiling was performed as described e.g. in

Relative proportions of neutral and acidic N-glycan fractions were studied by desialylating isolated acidic glycan fraction with A. ureafaciens sialidase as described in the preceding Examples and then combining the desialylated glycans with neutral glycans isolated from the same sample. Then the combined glycan fractions were analyzed by positive ion mode MALDI-TOF mass spectrometry as described in the preceding Examples. The proportion of sialylated N-glycans of the combined N-glycans was calculated by calculating the percentual decrease in the relative intensity of neutral N-glycans in the combined N-glycan fraction compared to the original neutral N-glycan fraction, according to the equation:

$$proportion = \frac{{I}^{neutral} - {I}^{combined}}{{I}^{neutral}} \times 100 \% ,$$

wherein I^{neutral} and I^{combined} correspond to the sum of relative intensities of the five high-mannose type N-glycan [M+Na]⁺ ion signals at m/z 1257, 1419, 1581, 1743, and 1905 in the neutral and combined N-glycan fractions, respectively.

### Results and Discussion

The relative proportions of acidic N-glycan fractions in studied stem cell types were as follows: in human embryonic stem cells (hESC) approximately 35% (proportion of sialylated and neutral N-glycans is approximately 1:2), in human bone marrow derived mesenchymal stem cells (BM MSC) approximately 19% (proportion of sialylated and neutral N-glycans is approximately 1:4), in osteoblast-differentiated BM MSC approximately 28% (proportion of sialylated and neutral N-glycans is approximately 1:3), and in human cord blood (CB) CD133+ cells approximately 38% (proportion of sialylated and neutral N-glycans is approximately 2:3).

In conclusion, BM MSC differ from hESC and CB CD133+ cells in that they contain significantly lower amounts of sialylated N-glycans compared to neutral N-glycans. However, after osteoblast differentiation of the BM MSC the proportion of sialylated N-glycans increases.

### Example 10

### Glycosphingolipid Glycans of Human Stem Cells

### Experimental Procedures

### Results and Discussion

### Human Cord Blood Mononuclear Cells (CB MNC)

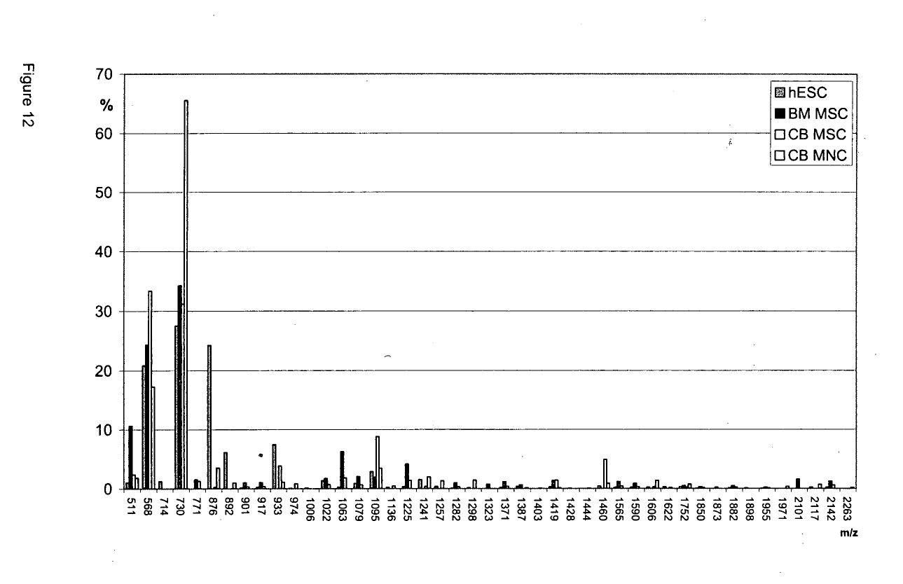

CB MNC neutral lipid glycans. The analyzed mass spectrometric profile of the CB MNC glycosphingolipid neutral glycan fraction is shown in FIG. 12. The five major glycan signals, together comprising more than 91% of the total glycan signal intensity, corresponded to monosaccharide compositions Hex₃HexNAc, (730), Hex₂HexNAc₁ (568), Hex₃HexNAc₁dHex₁ (876), Hex₄HexNAc₂ (1095), and Hex₄HexNAc₂dHex₁ (1241).

In β1,4-galactosidase digestion, the relative signal intensities of 730 and 1095 were reduced by about 50% and 90%, respectively. This suggests that the signals contained major components with non-reducing terminal β1,4-Gal epitopes, preferably including the structures Galβ4GlcNAcβLac and Galβ4GlcNAcβ[Hex₁HexNAc₁]Lac. Further, the glycan signal Hex₅HexNAc₃ (1460) was digested to Hex₄HexNAc₃ (1298) and Hex₃HexNAc₃ (1136), indicating that the original signal contained glycan structures containing either one or two β1,4-Gal.

The experimental structures of the major CB MNC glycosphingolipid neutral glycan signals were thus determined (‘>’ indicates the order of preference among the lipid glycan structures of hESC; ‘[ ]’ indicates that the oligosaccharide sequence in brackets may be either branched or unbranched; ‘( )’ indicates a branch in the structure):
730 Hex₃HexNAc₁>Hex₁HexNAc₁Lac>Galβ4GlcNAcLac
568 Hex₂HexNAc₁>HecNAcLac
876 Hex₃HexNAc₁dHex₁>[Hex₁HecNAc₁dHex₁]Lac>Fuc[Hex₁HecNAc₁]Lac
1095 Hex₄HexNAc₂>[Hex₂HecNAc₂]Lac>Galβ4GlcNAc[Hex₁HecNAc₁]Lac
1241 Hex₄HexNAc₂dHex₁>[Hex₂HecNAc₂dHex₁]Lac>Fuc[Hex₂HecNAc₂]Lac
1460 Hex₅HexNAc₃>[Hex₃HecNAc₃]Lac>Galβ4GlcNAc[Hex₂HecNAc₂]Lac>Galβ4GlcNAc(Galβ4GlcNAc)[Hex₁HecNAc₁]Lac

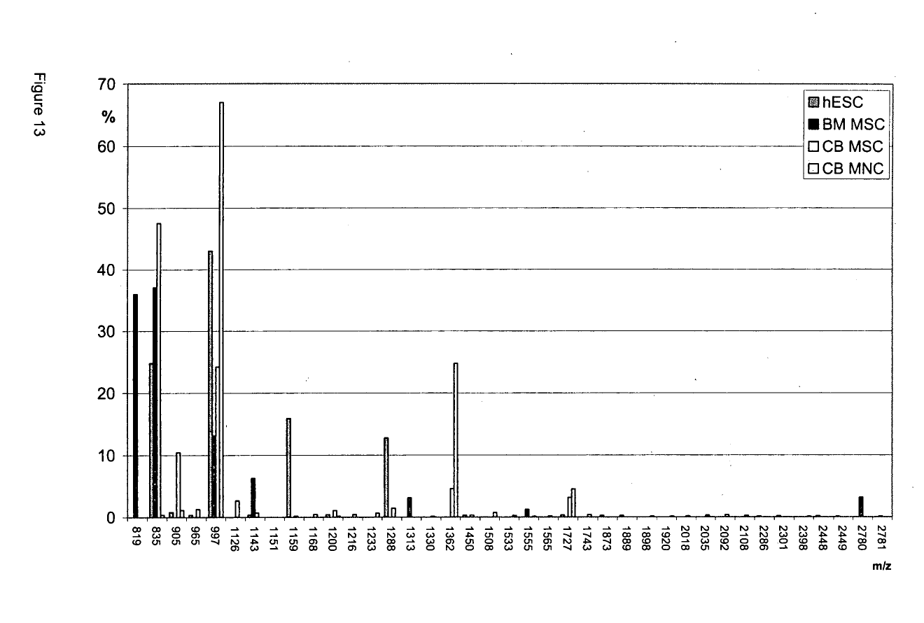

Sialylated lipid glycans. The analyzed mass spectrometric profile of the CB MNC glycosphingolipid sialylated glycan fraction is shown in FIG. 13. The three major glycan signals of CB MNC, together comprising more than 96% of the total glycan signal intensity, corresponded to monosaccharide compositions NeuAc₁Hex₃HexNAc, (997), NeuAc₁Hex₄HexNAc₂ (1362), and NeuAc₁Hex₅HexNAc₃ (1727).

### Overview of Human Stem Cell Glycosphingolipid Glycan Profiles

The neutral glycan fractions of all the present sample types altogether comprised 45 glycan signals. The proposed monosaccharide compositions of the signals were composed of 2-7 Hex, 0-5 HexNAc, and 0-4 dHex. Glycan signals were detected at monoisotopic m/z values between 511 and 2263 (for [M+Na]⁺ ion).

Major neutral glycan signals common to all the sample types were 730, 568, 1095, and 933, corresponding to the glycan structure groups Hex₀₋₁HexNAc₁Lac (568 or 730) and Hex₁₋₂HexNAc₂Lac (933 or 1095), of which the former glycans were more abundant and the latter less abundant. A general formula of these common glycans is HexₘHexNAcₙLac, wherein m is either n or n−1, and n is either 1 or 2.

### Neutral Glycolipid Profiles of Human Stem Cell Types:

Glycan signals typical to CB MNC preferentially include compositions dHexy₀₋₁[HexHexNAc]₁₋₂Lac, more preferentially high relative amounts of 730 compared to other signals; and fucosylated structures; and glycan profiles with less variability and/or complexity than other stem cell types.

The acidic glycan fractions of all the present sample types altogether comprised 38 glycan signals. The proposed monosaccharide compositions of the signals were composed of 0-2 NeuAc, 2-9 Hex, 0-6 HexNAc, 0-3 dHex, and/or 0-1 sulphate or phosphate esters. Glycan signals were detected at monoisotopic m/z values between 786 and 2781 (for [M-H]⁻ ion).

The acidic glycosphingolipid glycans of CB MNC were mainly composed of NeuAc₁Hexₙ₊₂HexNAcₙ, wherein 1≦n≦3, indicating that their structures were NeuAc₁[HexHexNAc]₁₋₃Lac.

Terminal glycan epitopes that were demonstrated in the present experiments in stem cell glycosphingolipid glycans include:

### Gal

### Galβ4Glc (Lac)

Galβ4GlcNAc (LacNAc type 2)

### Galβ3

Non-reducing terminal HexNAc

### Fuc

### α1,2-Fuc

α1,3-Fuc

### Fucα2Gal

Fucα2Galβ4GlcNAc (H type 2)
Fucα2Galβ4Glc (2′-fucosyllactose)

### Fucα3GlcNAc

### Galβ4(Fucα3)GlcNAc (Lex)

### Fucα3Glc

Galβ4(Fucα3)Glc (3-fucosyllactose)

### Neu5Ac

### Neu5Acα2,3

### Neu5Acα2,6

### Example 11

### Lectin Based Selection of CB MNC Cell Populations

The FACS experiments with fluorescein-labeled lectins and CB MNC were performed essentially similarly to as described in Examples. Double stainings were performed with CD34 specific monoclonal antibody (Jaatinen et al., 2006) with complementary fluorescent dye. Erythroblast depletion from CD MNC fraction was performed by anti-glycophorin A (GlyA) monoclonal antibody negative selection.

### Results and Discussion

Compared to the CB MNC fraction, GlyA depleted CB MNC showed decreased staining in FACS with the following lectins (the decrease in % in parenthesis): PWA (48%), LTA (59%), UEA (34%), STA, MAA, and PNA (all latter three less than 23%); indicating that GlyA depletion increased the resolving power of the lectins in cell sorting.

In FACS double staining with both fluorescein-labeled lectins and anti-CD34 antibody, the following lectins colocalized with CD34+ cells: STA (3/3 samples), HHA (3/3 samples), PSA (3/3 samples), RCA (3/3 samples), and partly also NPA (2/3 samples). In contrast, the following lectins did not colocalize with CD34+ cells: GNA (3/3 samples) and PWA (3/3 samples), and partly also LTA (2/3 samples), WFA (2/3 samples), and GS-II (2/3 samples).

Taken together with the results of Example 8, the present results indicate that lectins can enrich CD34+ cells from CB MNC by both negative and positive selection, for example:
1) GNA binds to about 70% of CB MNC but not to CD34+ cells, leading to about 3× enrichment in negative selection of CB MNC in CD34+ cell isolation.
2) STA binds to about 50% of CB MNC and also to CD34+ cells, leading to about 2× enrichment in positive selection of CB MNC in CD34+ cell isolation.
3) UEA binds to about 50% of CB MNC and also to CD34+ cells, leading to about 2× enrichment in positive selection of CB MNC in CD34+ cell isolation.

### Example 12

### Galectin Gene Expression Profiles of Stem Cells

### Experimental Procedures

Gene expression analysis of CB CD133+ cells has been described (Jaatinen et al., 2006) and the present analysis was performed essentially similarly. The galectins whose gene expression profile was analyzed included (corresponding Affymetrix codes in parenthesis): Galectin-1 (201105_at), galectin-2 (208450_at), galectin-3 (208949_s_at), galectin-4 (204272_at), galectin-6 (200923_at), galectin-7 (206400_at), galectin-8 (208933_s_at), galectin-9 (203236_s_at), galectin-10 (206207_at), galectin-13 (220158_at).

### Results and Discussion

In CB CD133+ versus CD133−, as well as CD34+ versus CD34− CB MNC cells, the galectin gene expression profile was as follows: Overall, galectins 1, 2, 3, 6, 8, 9, and 10 showed gene expression in both CD34+/CD133+ cells. Galectins 1, 2, and 3 were downregulated in both CD34+/CD133+ cells with respect to CD34−/CD133− cells, and in addition galectin 10 was downregulated in CD133+ cells with respect to CD133− cells. In contrast, in both CD34+/CD133+ cells galectin 8 was upregulated with respect to CD34-/CD133− cells.

In hESC versus EB samples, the galectin gene expression profile was as follows: Overall, galectins 1, 3, 6, 8, and 13 showed gene expression in hESC. Galectin 3 was clearly downregulated with respect to EB, and in addition galectin 13 was downregulated in 2 out of 4 hESC lines. In contrast, galectin 1 was clearly upregulated in all hESC lines.

The results indicate that both CB CD34+/CD133+ stem cell populations and hESC have an interesting and distinct galectin expression profiles, leading to different galectin ligand affinity profiles (Hirabayashi et al., 2002). The results further correlate with the glycan analysis results showing abundant galectin ligand expression in these stem cells, especially non-reducing terminal β-Gal and type II LacNAc, poly-LacNAc, β1,6-branched poly-LacNAc, and complex-type N-glycan expression.

### Example 13

### Immunohistochemical Staining of Stem Cells

After rinsing with PBS the stem cell cultures/sections are incubated in 3% highly purified BSA in PBS for 30 minutes at RT to block nonspecific binding sites. Primary antibodies (GF279, 288, 287, 284, 285, 283, 286, 290 and 289) were diluted (1:10) in PBS containing 1% BSA-PBS and incubated 1 hour at RT. After rinsing three times with PBS, the sections are incubated with biotinylated rabbit anti-mouse, secondary antibody (Zymed Laboratories, San Francisco, Calif., USA) in PBS for 30 minutes at RT, rinsed in PBS and incubated with peroxidase conjugated streptavidin (Zymed Laboratories) diluted in PBS. The sections are finally developed with AEC substrate (3-amino-9-ethyl carbazole; Lab Vision Corporation, Fremont, Calif., USA). After rinsing with water counterstaining is performed with Mayer's hemalum solution.

Antibodies, their antigens/epitopes and codes for immunostainings.

| Producer code | Manufact | Clone | Specificity | Code | Target stucture(s) | Host/isotype |
|---|---|---|---|---|---|---|
| MAB-S206 (Globo-H) | Glycotope | A69-A/E8 | Globo-H | GF288 | Fucα2Galβ3GalNAcβ3GalαLacCer | mouse/IgM |
| MAB-S201 CD174 | Glycotope | A70-C/C8 | CD174 | GF289 | Fucα2Galβ4(Fucα3)GlcNAc | mouse/IgM |
| (Lewis y) | | | (Lewis y) | | | |
| MAB-S204 H type 2 | Glycotope | A51-B/A6 | H type 2 | GF290 | Fucα2Galβ4GlcNAc | mouse/IgA |
| DM3122: 0.1 mg | Acris | 2-25LE | Lewis b | GF283 | Fucα2Galβ3(Fucα4)GlcNAc | mouse/IgG |
| (Lewis b) | | | | | | |
| DM3015: 0.15 mg | Acris | B393 | H Type 2 | GF284 | Fucα2Galβ4GlcNAc | mouse/IgM |
| DM3014: 0.15 mg | Acris | B389 | H Type 2, | GF285 | Fucα2Galβ4GlcNAc, | mouse/IgG1 |
| | | | Le b, Ley | | Fucα2Galβ3(Fucα4)GlcNAc, | |
| | | | | | Fucα2Galβ4(Fucα3)GLcNAc | |
| BM258P: 0.2 mg | Acris | BRIC 231 | H Type 2 | GF286 | Fucα2Galβ4GlcNAc | mouse/IgG1 |
| ab3355 (blood group | Abcam | 17-206 | H type 1 | GF287 | Fucα2Galβ3GlcNAc | mouse/IgG3 |
| antigen H1) | | | | | | |
| ab3352 (pLN) | Abcam | K21 | Lewis c | GF279 | Galβ3GlcNAcβ(3Lac) | mouse/IgM |
| | | | Gb3GN | | | |

### Detection of Carbohydrate Structures on Cell Surfaces in Stem Cell Samples by Specific Antibodies

### Materials and Methods

### Antibodies.

Immunostainings. General hematopoietic cells are rinsed 5 times with PBS (10 mM sodium phosphate, pH 7.2, 140 mM NaCl) and fixed with 4% PBS-buffered paraformaldehyde pH 7.2 at room temperature (RT) for 10-15 minutes, followed by washings 3 times 5 minutes with PBS. Non-specific binding sites are blocked with 3% HSA-PBS (FRC Blood Service, Finland) for 30 minutes at RT. Primary antibodies are diluted in 1% HSA-PBS (1:10-1:200) and incubated for 60 minutes at RT, followed by washings 3 times 10 minutes with PBS. Secondary antibodies, Alexa Fluor 488 goat anti-mouse IgG (H+L; 1:1000) (Invitrogen), Alexa Fluor 488 goat anti-rabbit IgG (H+L; 1:1000) (Invitrogen) or FITC-conjugated rabbit anti-rat IgG (1:320) (Sigma) in 1% HSA-PBS and incubated for 60 minutes at RT in the dark. Furthermore, cells are washed 3 times 10 minutes with PBS and mounted in Vectashield mounting medium containing DAPI-stain (Vector Laboratories, UK). Immunostainings were observed with Zeiss Axioskop 2 plus-fluorescence microscope (Carl Zeiss Vision GmbH, Germany) with FITC and DAPI filters. Images were taken with Zeiss AxioCam MRc-camera and with AxioVision Software 3.1/4.0 (Carl Zeiss) with the 400× magnification.

Fluorescence activated cell sorting (FACS) analysis. Proliferating SCs on passage 12 are detached from culture plates by 0.02% Versene solution (pH 7.4) for 45 minutes at 37° C. Cells are washed twice with 0.3% HSA-PBS solution before antibody labelling. Primary antibodies are incubated (4 μl/100 μl cell suspension/50 000 cells) for 30 minutes at RT and washed once with 0.3% HSA-PBS before secondary antibody detection with Alexa Fluor 488 goat anti-mouse (1:500) for 30 minutes at RT in the dark. As a negative control cells are incubated without primary antibody and otherwise treated similar to labelled cells. Cells are analysed with BD FACSAria (Becton Dickinson) using FITC detector at wavelength 488. Results are analysed with BD FACSDiva software version 5.0.1 (Becton Dickinson).

Examples of antibodies, their antigens/epitopes and codes used in the immunostainings.

| | | | Dilution | | | |
|---|---|---|---|---|---|---|
| Code | Antigen | Host | I HC | Class | Manufact | Cat No |
| GF274 | PNAd (peripheral lymph node addressin; | Rat anti- | 5-20 μg/ml | IgM, κ | BD | 553863 |
| | CD62L ligand) closely associated with L- | mouse | | | Pharmingen | |
| | selectin (CD34, GlyCAM-1, MAdCAM-1), | | | | | |
| | sulfo-mucin | | | | | |
| GF275 | CA15-3 (Cancer antigen 15-3; sialylated | Mouse anti- | | IgG1 | Acris | BM3359 |
| | carbohydrate epitope of the MUC-1 | human | | | Antibodies | |
| | glycoprotein) | | | | | |
| GF276 | oncofetal antigen, tumor associated | Mouse anti- | 1:20-1:50 | IgG1 | Acris | DM288 |
| | glycoprotein (TAG-72) or CA 72-4 | human | | | Antibodies | |
| GF277 | human sialosyl-Tn antigen (STn, | Mouse anti- | 1:50-1:100 | IgG1 | Acris | DM3197 |
| | sCD175) | human | (4-8 μg/ml) | | Antibodies | |
| GF278 | human Tn antigen (Tn, CD175 B1.1) | Mouse anti- | 1:50 (4 μg/ml) | IgM | Acris | DM3218 |
| | | human | | | Antibodies | |

| | | | Dilution | | | |
|---|---|---|---|---|---|---|
| Koodi | Antigen | Host | IHC | Class | Manufact | Cat No |
| GF295 | Blood group antigen precursor (BG1), | Mouse anti- | 01:40 | IgM | Abcam | ab3352 |
| | Lewis c Gb3GN (pLN) | human | | | | |
| GF280 | TF-antigen isoform (Nemod TF2) | Mouse anti-? | | IgM | | MAB- |
| | | | | | | S301 |
| GF281 | TF-antigen isoform (A68-E/E3) | Mouse anti-? | | IgG1 | | MAB- |
| | | | | | | S305 |
| GF296 | asialoganglioside GM1 | Rabbit anti- | 1:100-1:400 | polycl. | Acris | BP282 |
| | | bovine | ELISA | | Antibodies | |
| GF297 | Globoside GL4 | Rabbit anti- | 1:50-1:100 | polycl. | Abcam | ab23949 |
| | | several | ELISA | IgG | | |
| | | species | | | | |
| GF298 | Human CD77 (=blood group substance | Rat anti- | | IgM | Acris | SM1160P |
| | pk), GB3 | human | | | Antibodies | |
| GF299 | Forssman antigen, glycosphingolipid (FOGSL) | Rat anti- | 1:100-1:1000 | IgG | Acris | BM4091 |
| | differentiation ag | mouse | | | Antibodies | |
| | | (human ??) | | | | |
| GF300 | Asialo GM2 | Rabbit anti- | 1:100-1:400 | polycl. | Acris | BP283 |
| | | bovine | ELISA | | Antibodies | |

| | | | Dilution | | | |
|---|---|---|---|---|---|---|
| Code | Antigen | Host | IHC | Class | Producer | Cat no |
| GF301 | Lewis b blood group antigen | Mouse anti- | | IgG1 | Acris | SM3092P |
| | | human | | | Antibodies | |
| GF302 | H type 2 blood group antigen | Mouse anti- | | IgM | Acris | DM3015 |
| | | human | | | Antibodies | |
| GF303 | Blood group H1(O) antigen (BG4) | Mouse anti- | | IgG3 | Abcam | ab3355 |
| | | human | | | | |
| GF288 | Globo-H | Mouse anti-? | | IgM | | MAB- |
| | | | | | | S206 |

| | | | Dilution | | | |
|---|---|---|---|---|---|---|
| Code | Antigen | Host | IHC | Class | Producer | Cat no |
| GF304 | Lewis a | Mouse anti- | | IgG1 | Chemicon int. | CBL205 |
| | | human | | | | |
| GF305 | Lewis x, CD15, 3-FAL, SSEA-1,3- | Mouse anti- | | IgM | Chemicon int. | CBL144 |
| | fucosyl-N-acetyllactosamine | human | | | | |
| GF306 | Sialyl Lewis a | Mouse anti- | 01:40 | IgG1 | Chemicon int. | MAB2095 |
| | | human | | | | |
| GF307 | Sialyl Lewis x | Mouse anti- | 01:40 | IgM | Chemicon int. | MAB2096 |
| | | human | | | | |

| | | | Dilution | | | |
|---|---|---|---|---|---|---|
| Code | Antigen | Host | IHC | Class | Producer | Cat no |
| GF353 | SSEA-3 (stage-specific embryonic | Rat anti- | 10-20 μg/ml | IgM | Chemicon int. | MAB4303 |
| | antigen-3) | mouse/human | | | | |
| GF354 | SSEA-4 (stage-specific embryonic | Mouse anti- | 10-20 μg/ml | IgG3 | Chemicon int. | MAB4304 |
| | antigen-4) | human | | | | |
| GF355 | Galactose-a(1,3)galactose | Baboon | 1:500 | serum | Chemicon int. | AB2052 |
| | | anti- | | | | |
| | | porcine/rat | | | | |
| GF365 | Nemod TF1, DC176, GalB1-3GalNAc | Mouse anti- | | IgM, k | Glycotope | Lot 31-2006 |
| | | human | | | | |

### Example 14

### Glycosidase Profiling of Cord Blood Mononuclear Cell N-Glycans

### Experimental Procedures

Exoglycosidase digestions. Neutral N-glycan fractions were isolated from cord blood mononuclear cell populations as described above. Exoglycosidase reactions were performed essentially after manufacturers' instructions and as described in (Saarinen et al., 1999). The different reactions were; α-Man: α-mannosidase from Jack beans (C. ensiformis; Sigma, USA); β1,4-Gal: β1,4-galactosidase from S. pneumoniae (recombinant in E. coli; Calbiochem, USA); β1,3-Gal: recombinant β1,3-galactosidase (Calbiochem, USA); β-GlcNAc: β-glucosaminidase from S. pneumoniae (Calbiochem, USA); α2,3-SA: α2,3-sialidase from S. pneumoniae (Calbiochem, USA). The analytical reactions were carefully controlled for specificity with synthetic oligosaccharides in parallel control reactions that were analyzed by MALDI-TOF mass spectrometry. The sialic acid linkage specificity of α2,3-SA was controlled with synthetic oligosaccharides in parallel control reactions, and it was confirmed that in the reaction conditions the enzyme hydrolyzed α2,3-linked but not α2,6-linked sialic acids. The analysis was performed by MALDI-TOF mass spectrometry as described in the preceding examples. Digestion results were analyzed by comparing glycan profiles before and after the reaction.

RESULTS Glycosidase profiling of neutral N-glycans. Neutral N-glycan fractions from affinity-purified CD34+, CD34−, CD133+, CD133−, Lin+, and Lin− cell samples from cord blood mononuclear cells were isolated as described above. The glycan samples were subjected to parallel glycosidase digestions as described under Experimental procedures. Profiling results are summarized in Table 11 (CD34+ and CD34− cells), Table 12 (CD133+ and CD133− cells), and Table 13 (Lin− and Lin+ cells). The present results show that several neutral N-glycan signals are individually sensitive towards all the exoglycosidases, indicating that in all the cell types several neutral N-glycans contain specific substrate glycan structures in their non-reducing termini. The results also show clear differences between the cell types in both the sensitivity of individual glycan signals towards each enzyme and also profile-wide differences between cell types, as detailed in the Tables cited above.

Glycosidase profiling of sialylated N-glycans. Sialylated N-glycan fractions from affinity-purified CD133+ and CD133− cell samples from cord blood mononuclear cells were isolated as described above. The glycan samples were subjected to parallel glycosidase digestions as described under Experimental procedures. Profiling results by α2,3-sialidase are shown in Table 14. The results show significant differences between the glycan profiles of the analyzed cell types in the sialylated and neutral glycan fractions resulting in the reaction. The present results show that differences are seen in multiple signals in a profile-wide fashion. Also individual signals differ between cell types, as discussed below.

Cord blood CD133⁺ and CD133⁻ cell N-glycans are differentially α2,3-sialylated. Sialylated N-glycans from cord blood CD133+ and CD133⁻ cells were treated with α2,3-sialidase, after which the resulting glycans were divided into sialylated and non-sialylated fractions, as described under Experimental procedures. Both α2,3-sialidase resistant and sensitive sialylated N-glycans were observed, i.e. after the sialidase treatment sialylated glycans were observed in the sialylated N-glycan fraction and desialylated glycans were observed in the neutral N-glycan fraction. The results indicate that cord blood CD133⁺ and CD133⁻ cells are differentially α2,3-sialylated. For example, after α2,3-sialidase treatment the relative proportions of monosialylated (SA₁) glycan signal at m/z 2076, corresponding to the [M-H]⁻ ion of NeuAc₁Hex₅HexNAc₄dHex₁, and the disialylated (SA₂) glycan signal at m/z 2367, corresponding to the [M-H]⁻ ion of NeuAc₂Hex₅HexNAc₄dHex₁, indicate that α2,3-sialidase resistant disialylated N-glycans are relatively more abundant in CD133⁻ than in CD133+ cells, when compared to α2,3-sialidase resistant monosialylated N-glycans. It is concluded that N-glycan α2,3-sialylation in relation to other sialic acid linkages including especially α2,6-sialylation, is more abundant in cord blood CD133+ cells than in CD133⁻ cells.

In cord blood CD133⁻ cells, several sialylated N-glycans were observed that were resistant to α2,3-sialidase treatment, i.e. neutral glycans were not observed that would correspond to the desialylated forms of the original sialylated glycans. The results revealing differential α2,3-sialylation of individual N-glycan structures between cord blood CD133+ and CD133 cells are presented in Table 14. The present results indicate that N-glycan α2,3-sialylation in relation to other sialic acid linkages is more abundant in cord blood CD133+ cells than in CD133⁻ cells.

Sialidase analysis. The sialylated N-glycan fraction isolated from a cord blood mononuclear cell population (CB MNC) was digested with broad-range sialidase as described in the preceding Examples. After the reaction, it was observed by MALDI-TOF mass spectrometry that the vast majority of the sialylated N-glycans were desialylated and transformed into corresponding neutral N-glycans, indicating that they had contained sialic acid residues (NeuAc and/or NeuGc) as suggested by the proposed monosaccharide compositions. Combined glycan profiles of neutral and desialylated (originally sialylated) N-glycan fractions of a CB MNC population was produced. The profiles correspond to total N-glycan profiles isolated from the cell samples (in desialylated form). It is calculated that approximately 25% of the N-glycan signals correspond to high-mannose type N-glycan monosaccharide compositions, and 28% to low-mannose type N-glycans, 34% to complex-type N-glycans, and 13% to hybrid-type or monoantennary N-glycans monosaccharide compositions.

CONCLUSIONS The present results suggest that 1) the glycosidase profiling method can be used to analyze structural features of individual glycan signals, as well as differences in individual glycans between cell types, 2) different cell types differ from each other with respect to both individual glycan signals' and glycan profiles' susceptibility to glycosidases, and 3) glycosidase profiling can be used as a further means to distinguish different cell types, and in such case the parameters for comparison are both individual signals and profile-wide differences.

### Example 15

### Enrichment of Glycan Structure of Formula (I) Expressing Stem Cells

The FACS analysis is performed essentially as described in Venable et al. (2005) but living cells are used instead and FACSAria™ cell sorter (BD).

Human HSCs are harvested into single cell suspensions using collagenase and cell dissociation solution (Sigma) or mechanical release of cells or Versene. Then, cells are placed in sterile tube in aliquots 10⁶ cells each and stained with one of the GF antibody in 1:100 solution. Cells are washed 3 times with PBS and then stained with secondary antibodies (antigoat mouse IgG or IgM FITC conjugated). Unstained HSC used as control. The FITC positive cells are collected into cell culture media (in +4° C.) (according to BD instructions).

Then, cells are placed on CFU assay or other cell culture and monitored for clonal or cell lineage. To check the undifferentiation stage, the gene expression of sorted cells are analyzed with real-time PCR.

Alternatively, FACS enriched cells are let to spontaneously differentiate on gelatin. Immunohistochemistry is performed with various tissue specific antibodies as described in Mikkola et al. (2006) or analysed with PCR.

### Example 16

### Isolation and Characterization of Protease Released Glycopeptides Comprising Specific Binder Target Structures

Glycopeptides are released by treatment of stem cells by protease such as trypsin. The glycopeptides are isolated chromatographically, a preferred method uses gel filtration chromatography in Superdex (Amersham Pharmacia(GE)) column (Superdex peptide or superdex 75), the peptides can be observed in chromatogram by tagging the peptides with specific labels or by UV absorbance of the peptide (or glycans). Preferred samples for the method includes hematopoietic stem cells in relatively large amounts (millions of cells) and preferred antibodies, which are used in this example includes antibodies or other binders such as lectins according to the invention and binding to the cells.

The isolated glycopeptides are then run through a column of immobilized antibody (e.g. antibody immobilized to cyanogens promide activated column of Amersham Pharmacia(GE healthcare division or antibody immobilized as described by Pierce catalog)). The bound and/or weakly bound and chromatographically retarded fraction(s) is(are) collected as target peptide fraction. In case of high affinity binding the glycan is eluted with 100-1000 mM monosaccharide or monosaccharides corresponding to the target epitope of the antibody or by mixture of monosaccharides or oligosaccharides and/or with high salt concentration such as 500-1000 mM NaCl. The glycopeptides are analysed by glycoproteomic methods using mass spectrometry to obtain molecular mass and preferably also fragmentation mass spectrometry in order to sequence the peptide and/or the glycan of the glycopeptide.

In alternative method the glycopeptides are isolated by single affinity chromatography step by the binder affinity chromatography and analysed by mass spectrometry essentially similarity as described e.g. in Wang Y et al (2006) Glycobiology 16 (6) 514-23, but lectin affinity chromatography is replaced by affinity chromatography by immobilized antibodies, such as preferred antibodies or binder described above in this example.

### Example 17

### Glycolipid and O-Glycan Analysis of Cellular Glycan Types

The glycosphingolipid glycan and reducing O-glycan samples were isolated from studied cell types, analyzed by mass spectrometry, and further analyzed by expoglycosidase digestions combined with mass spectrometry as described in the present invention and the preceding Examples. Non-reducing terminal epitopes were analyzed by digestion of the glycan samples with S. pneumoniae β1,4-galactosidase (Calbiochem), bovine testes β-galactosidase (Sigma), A. ureafaciens sialidase (Calbiochem), S. pneumoniae α2,3-sialidase (Calbiochem), S. pneumoniae β-N-acetylglucosaminidase (Calbiochem), X. manihotis α1,3/4-fucosidase (Calbiochem), and α1,2-fucosidase (Calbiochem). The results were analyzed by quantitative mass spectrometric profiling data analysis as described in the present invention. The results with glycosphingolipid glycans are summarized in Table 17 including also core structure classification determined based on proposed monosaccharide compositions as described in the footnotes of the Table. Analysis of neutral O-glycan fractions revealed quantitative differences in terminal epitope glycosylation as follows: non-reducing terminal type 1 LacNAc (β1,3-linked Gal) had above 5% proportion only in hESC and non-reducing terminal type 2 LacNAc (β1,4-linked Gal) had above 95% proportion in CB MNC, CB MSC, and BM MSC. Fucosylation degree of type 2 LacNAc containing O-glycan signals at m/z 771 (Hex₂HexNAc₂) and 917 (Hex₂HexNAc₂dHex₁) was 64% in CB MNC, 47% in CB MSC, and 28% in hESC.

In conclusion, these results from O-glycans and glycosphingolipid glycans demonstrated significant cell type specific differences and also were significantly different from N-glycan terminal epitopes within each cell type analyzed in the present invention.

### Example 18

### Endo-β-Galactosidase Analysis of Cellular Glycan Types

### Endo-β-Galactosidase Reaction Conditions

The substrate glycans were dried in 0.5 ml reaction tubes. The endo-β-galactosidase (E. freundii, Seikagaku Corporation, cat no 100455, 2.5 mU/reaction) reactions were carried out in 50 mM Na-acetate buffer, pH 5.5 at 37° C. for 20 hours. After the incubation the reactions mixtures were boiled for 3 minutes to stop the reactions. The substrate glycans were purified using chromatographic methods according to the present invention, and analyzed with MALDI-TOF mass spectrometry as described in the preceding Examples.

In similar reaction conditions with 2 nmol of each defined oligosaccharide control, the reaction produced signal at m/z 568 (Hex₂HexNAc₁) as the major reaction product from lacto-N-neotetraose and para-lacto-N-neohexaose, but not from lacto-N-neohexaose or para-lacto-N-neohexaose monofucosylated at the 3-position of the inner GlcNAc residue; and sialylated signal corresponding to NeuAc₁Hex₂HexNAc, from α3′-sialyl-lacto-N-neotetraose. These results confirmed the reported specificities for the enzyme in the employed reaction conditions.

Results with Cellular Glycan Types

CB MNC glycosphingolipid glycans. The major digestion product in CB MNC neutral glycosphingolipid glycans was the signal at m/z 568 (Hex₂HexNAc₁), indicating the presence of non-fucosylated poly-LacNAc sequences. Further, signals at 714 (Hex₂HexNAcidHex₁) and 1225 (Hex₃HexNAc₂dHex₂) indicated the presence of fucosylated poly-LacNAc sequences.

Major sensitive signals included 1095 (Hex₄HexNAc₂), 1241 (Hex₄HexNAc₂dHex₁), 876 (Hex₃HexNAc₃dHex₁), 1606 (Hex₅HexNAc₃dHex₁), 1460 (Hex₅HexNAc₃), and 933 (Hex₃HexNAc₂), indicating presence of both linear non-fucosylated and multifucosylated poly-LacNAc. Residual signals left in the sensitive signals after digestion indicated presence of lesser amounts of also branched poly-LacNAc sequences.

CB MSC glycosphingolipid glycans. The major digestion product in CB MSC neutral glycosphingolipid glycans was the signal at m/z 568 (Hex₂HexNAc₁), indicating the presence of non-fucosylated poly-LacNAc sequences. Major sensitive signals were signals at m/z 1095 (H4N2), 933 (Hex₃HexNAc₂), and 1460 (Hex₅HexNAc₃). Compared to CB MNC results, CB MSC had less sensitive structures although the glycan profiles contained same original signals than CB MNC, indicating that in CB MSC the poly-N-acetyllactosamine sequences of glycosphingolipid glycans were more branched than in CB MNC.

hESC glycosphingolipid glycans. The major digestion product in hESC neutral glycosphingolipid glycans were the signals at m/z 568 (Hex₂HexNAc₁) and 714 (Hex₂HexNAc₁dHex₁) indicating the presence of non-fucosylated and fucosylated poly-LacNAc sequences. Further, the signals at m/z 1428 (Hex₃HexNAc₃dHex₂) and 1282 (Hex₃HexNAc₃dHex₁) were products, indicating the presence of different glycan terminal sequences with non-reducing terminal HexNAc than in the abovementioned cell types. Major sensitive signals were signals at m/z 730, 876, 933, 1095, and 1241 with similar interpretation as with CB MNC above.

In conclusion, the profiles of endo-β-galactosidase reaction products efficiently reflected cell type specific glycosylation features as described in the preceding Examples and they represent an alternative and complementary method for analysis of cellular glycan types. Further, the present results demonstrated the presence of linear, branched, and fucosylated poly-LacNAc in all studied cell types and in different glycan types including N- and O-glycans and glycosphingolipid glycans; and further quantitative and cell-type specific proportions of these in each cell type, which are characteristic to each cell type.

### Example 19

### Selection of Cord Blood Mononuclear Cells by Immobilized Binders and Culture of the Cells Together with Binders

### Materials and Methods

### Preparation of Lectin Coated Dynabeads

To study the capacity of lectin coated microparticles to bind hematopoietic stem cells (HSC) we used Dynabeads® M-280 Streptavidin Dynabeads (Invitrogen, Dynal) and coated them with biotinylated lectin molecules. Beads were washed according to manufacturers instructions using PBS-0.1% BSA. 10 μg of biotinylated lectins were incubated with 1 mg of Dynabead particles for 30 minutes in room temperature with gentle rotation. Coated beades were then washed 3 times with 0.1% BSA-PBS and used in cell binding assay.

Dynal MPC-E Magnetic Particle Concentrator for Microtubes of Eppendorf Type (Dynal AS, Norway) was used for harvesting.

### Separation of Lin− Population of MNC

Lin negative cell population was separated from CB Mononuclear cell using StemSep Human Progenitor Enrichment coctail (StemCell Technologies). 75000000 cells/ml were suspended with 0.5% BSA-PBS. Lin Human Progenitor Enrichment Coctail was added to the suspension and incubated 15 minutes at RT. After incubation Magnetic beads were mixed with cell suspension and incubated for another 15 minutes at RT.

Lin− cells were separated using Miltenyi LD Magnetic Column (Miltenyi Biotec) according to manufacturer's instructions.

Lin− cells were suspended with lectin coated particles in dilution of 10 000 cells/10 μg Dynabeads for culture.

### Binding of Cord Blood Derived Mononuclear Cells to Lectin Coated Dynabeads

A frozen Cord Blood (CB) mononuclear cell (MNC) fraction previously isolated by density gradient centrifugation using Ficoll-Hypaque solution was used to study the binding capacity of lectin coated microparticles. Thawed CB MNC cells were diluted in 0.1% BSA-PBS-2 mM EDTA and suspended with lectin coated beads (Dynabeads® M-280 Streptavidin Dynabeads (Invitrogen), coated with biotinylated lectins, EY laboratories, Inc. San Mateo, Calif., USA, www.eylabs.com) in dilution of 6.3×10⁶ mononuclear cells/100 μg of lectin coated beads. Uncoated beads were used as controls. Cells were incubated with magnetic beads for 1 hour with gentle rotation in +6° C. After incubation, unbound cells were collected as supernatant and Dynabeads were washed twice with 0.1% BSA-PBS. Dynabeads with bound cells were harvested using Dynal MPC-E Magnetic Particle Concentrator. The number of both unbound and Dynabead-bounded cells were calculated with Bürker Chamber.

TABLE
Lectins immobilized on beads used in binding assay

GF 707 PNA, peanut agglutinin
GF 708 DBA, Dolichos biflorus agglutinin
GF 709 LTA, Lotus tetragonolobus agglutinin
GF 710 MAA, Maackia amuriensis agglutinin
GF 711 NPA,
GF 712 STA, Solanum
tuberosum agglutinin
GF 713 UEA, Ulex
europaeus agglutinin
Control, no lectin on
beads

### Flow Cytometric Analysis

MNC Cells bound to lectin coated or control beads were washed with PBS centrifuged at 600×g for five minutes at room temperature. Cell pellet was washed twice with 0.3% BSA-PBS, centrifuged at 600×g and resuspended in 0.3% BSA-PBS. Cells were placed in conical tubes in aliquots of 100 000 cells each. Cell aliquots were incubated with antibodies (Table below) in dilution of 2 μl/10⁵ cells for 30 minutes at +4° C. in the dark. After incubation cells were washed with 0.3% BSA-PBS, centrifuged and resuspended in 0.3% BSA-PBS.

Unlabeled cells, cells which were not bound to lectin coated beads, and cells without beads were also analyzed. Antibody binding was detected by flow cytometry (FACSAria, Becton Dickinson). Data analysis was made with FACSDiva™ Flow Cytometry Software Version 5.02.

TABLE
Antibodies used to characterize MNC fraction

| | |
|---|---|
| CD 34 | FITC |
| CD 133 | PE |
| CD 90 | PE-Cy5 |
| CD 3 | FITC |
| CD 14 | FITC |

TABLE
Lectins immobilized on beads used in binding assay

GF 707 PNA, peanut agglutinin
GF 708 DBA, Dolichos biflorus agglutinin
GF 709 LTA, Lotus tetragonolobus agglutinin
GF 710 MAA, Maackia amuriensis agglutinin
GF 711 NPA,
GF 712 STA, Solanum
tuberosum agglutinin
GF 713 UEA, Ulex
europaeus agglutinin
Control, no lectin on
beads

### Results

A variety of amount of MN cells bound to lectin coated beads GF710 bound 90%, GF 711 about 11% of the cells and other molecules bound substantial amounts but less than 5% of the cells, TABLE 19. Dynabeads without lectin coating did not bind mononuclear cells.

MNC bound to lectin coated Dynabeads were stained with antibodies against CD 34, CD 90, CD133, CD 3 and CD 14 and analyzed with FACSAria. Based on these results we can not say that lectin coated particles enrich certain homogenous cell populations, but they cell populations that were attached to lecctin coated particles seemed to be more positive for CD34 and CD 133 than control populations (native cells and cells that were not bound to beads).

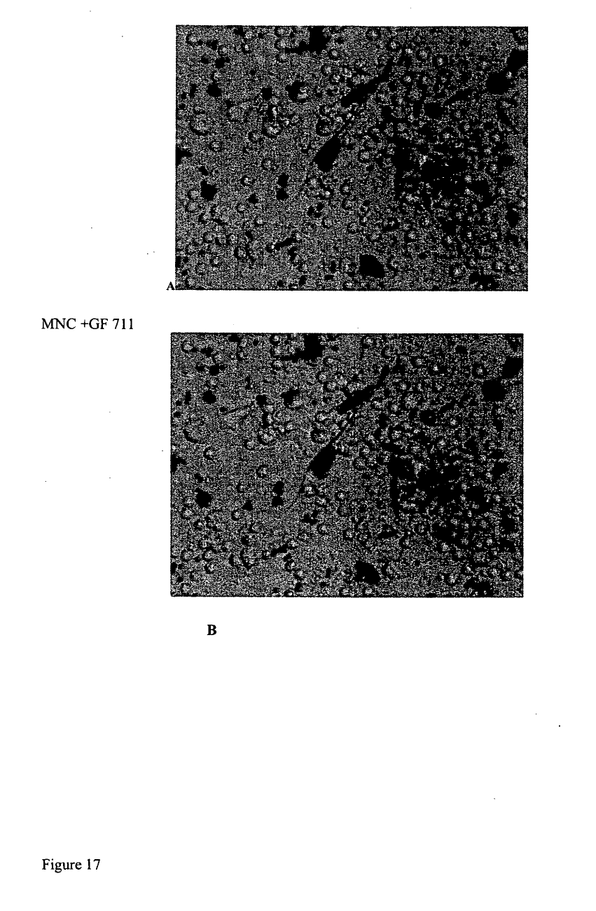

MNCs together with beads coated with GF711 are shown in FIG. 17 in panel A. Lineage negative cells selected from CB MNCs by standard method as in other examples bound to the lectin coated beads, e.g GF 710, FIG. 17 B. Lin-negative cell produced from CB MNC cells by standard methods as described in Examples.

### Example 20

### Experimental Procedures

Extraction of mononuclear cells (MNCs) from umbilical cord blood. Human term umbilical cord blood (CB) units were collected after delivery with informed consent of the mothers and the CB was processed within 24 hours of the collection. The mononuclear cells (MNCs) were isolated from each CB unit diluting the CB 1:1 with phosphate-buffered saline (PBS) followed by Ficoll-Paque Plus (Amersham Biosciences, Uppsala, Sweden) density gradient centrifugation (400×g/40 min). The mononuclear cell fragment was collected from the gradient and washed twice with PBS.

Depletion of red blood cell precursors by magnetic microbeads conjugated with anti-Glycophorin A (anti-CD235a). MNCs (107) were suspended in 80 μl of 0.5% ultra pure BSA, 2 mM EDTA-PBS buffer. Red blood cell precursors were depleted with magnetic microbeads conjugated with anti-CD235a (Glycophorin a, Miltenyi Biotec) by adding 20 μl of magnetic microbead suspension/10⁷ cells and by incubating for 15 min at 4° C. Cell suspension was washed with 1-2 ml of buffer/10⁷ cells followed by centrifugation at 300×g for 10 min. Cells were resuspended 1.25×10⁸ cells/500 μl of buffer. MACS LD column (Miltenyi Biotec) was placed in a magnetic field and rinsed with 2 ml of buffer. Cell suspension was applied to the column and cells passing through were collected. Column was washed two times with 1 ml of buffer and total effluent was collected. Cells were centrifuged for 10 min at 300×g and resuspended in 10 ml of buffer. All together eight CB units were used for following antibody staining.

Staining with anti-glycan antibodies. MNCs were aliquoted to FACS tubes in a small volume, i.e. 0.5×10⁶ cells/500 μl of 0.3% ultra pure BSA (Sigma), 2 mM EDTA-PBS buffer. Ten microliters of primary antibody (list of primary antibodies is presented in Table 22) was added to cell suspension, vortexed and cells were incubated for 30 min at room temperature. Cells were washed with 2 ml of buffer and centrifuged at 500×g for 5 min. AlexaFluor 488-conjugated anti-mouse (1:500, Invitrogen) and anti-rabbit (1:500, Molecular Probes) and FITC-conjugated anti-rat (1:320, Sigma) secondary antibodies were used for appropriated primary antibodies. Secondary antibodies were diluted in 0.3% ultra pure BSA, 2 mM EDTA-PBS buffer and 200 μl of dilution was added to the cell suspension. Samples were incubated for 30 min at room temperature in the dark. Cells were washed with 2 ml of buffer and centrifuged at 500×g for 5 min. As a negative control cells were incubated without primary antibody and otherwise treated similarly to labelled cells.

Double staining with PE-conjugated anti-CD34-antibody. After staining with anti-glycan antibodies, a double staining with PE-conjugated anti-CD34 antibody (BD Biosciences) was performed. Cells were suspended in 500 μl of buffer and 10 μl of anti-CD34 antibody was added and incubated for 30 min at +4° C. in dark. After incubation cells were washed with 2 ml of buffer and centrifugation at 500×g for 5 min. Supernatant was removed and cells were resuspended in 300 μl of buffer and stored at 4° C. overnight in the dark.

Flow cytometric analysis. The next day cells were analysed with flow cytometer BD FACSAria (BD Biosciences) using FITC and PE detectors. Approximately 250 000-300 000 cells were counted for each anti-glycan antibody. Data was analysed with BD FACSDiva Software version 5.0.2 (BD Biosciences).

### Results and Discussion

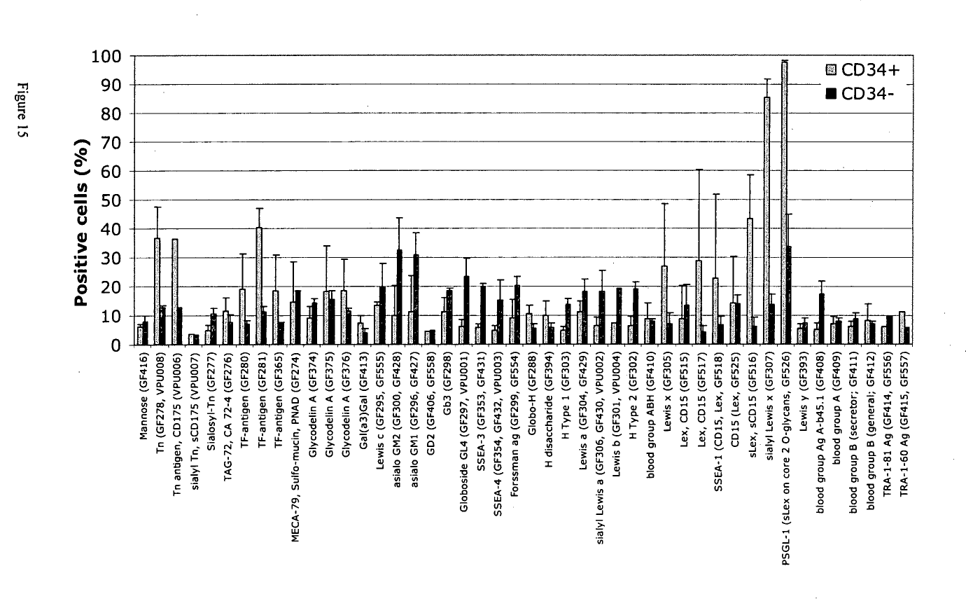

Results from CB-HSC FACS analysis are shown in FIG. 15 and Table 21 and antibodies are indicated in Table 22. Some glycan structures, e.g. Tn, TF, Lewis x and sialyl Lewis x, are enriched in HSCs (CD34+) when compared to mature blood cells (CD34−). This was shown with several anti-glycan antibodies against same epitope and even between different CB units. The highest variations were observed with anti-Lex antibodies between distinct CB units. The glycan structures enriched with mature blood cells (CD34−) were asialo GM1, asialo GM2, Globoside GL4 and Lewis a.

### Example 21

### Experimental Procedures

Extraction of mononuclear cells (MNCs) from umbilical cord blood. Human term umbilical cord blood (CB) units were collected after delivery with informed consent of the mothers and the CB was processed within 24 hours of the collection. The mononuclear cells (MNCs) were isolated from each CB unit diluting the CB 1:1 with phosphate-buffered saline (PBS) followed by Ficoll-Paque Plus (Amersham Biosciences, Uppsala, Sweden) density gradient centrifugation (400×g/40 min). The mononuclear cell fragment was collected from the gradient and washed twice with PBS.

Staining with Fluorescein (FITC)-conjugated lectins. MNCs were aliquoted to FACS tubes in a small volume, i.e. 0.5×10⁶ cells/500 μl of 0.3% ultra pure BSA (Sigma), 2 mM EDTA-PBS buffer. Ten microliters of FITC-conjugated lectin (Table 20) was added to cell suspension, vortexed and cells were incubated for 30 min at room temperature. Cells were washed with 2 ml of buffer and centrifuged at 500×g for 5 min. As a negative control cells were incubated without lectin and otherwise treated similarly to labelled cells.

Double staining with PE-conjugated anti-CD34-antibody. After staining with FITC-conjugated lectins, a double staining with PE-conjugated anti-CD34 antibody (BD Biosciences) was performed. Cells were suspended in 500 μl of buffer and 10 μl of anti-CD34 antibody was added and incubated for 30 min at +4° C. in dark. After incubation cells were washed with 2 ml of buffer and centrifugation at 500×g for 5 min. Supernatant was removed and cells were resuspended in 300 μl of buffer and stored at 4° C. overnight in the dark.

Flow cytometric analysis. The next day cells were analysed with flow cytometer BD FACSAria (BD Biosciences) using FITC and PE detectors. Approximately 250 000-300 000 cells were counted for each anti-glycan antibody. Data was analysed with BD FACSDiva Software version 5.0.2 (BD Biosciences).

### Results and Discussion

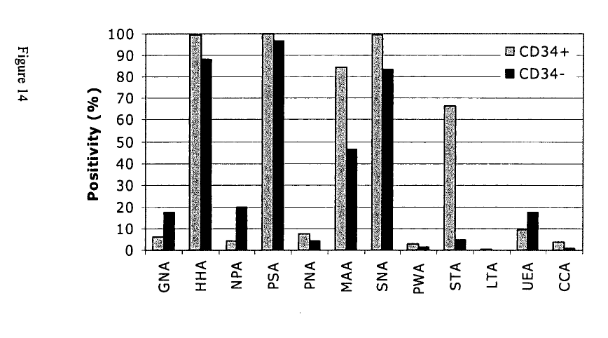

Results from CB-HSC (CD34+/−) lectin staining are shown in Table 20 and in FIG. 14. The data revealed that part of binders are especially useful for enrichment or isolation of hematopoietic CD34+ stem cells.

### Example 22

### Fragmentation Analysis of Permethylated Glycan Structures

Cord blood CD133+ and CD133− cells were gathered, their cellular N-glycans isolated, permethylated, essentially as described in the preceding Examples, and analyzed by MS/MS analysis (fragmentation mass spectrometry). In the following result listings, the fragments are mainly Na+ adduct ions unless otherwise specified and [ ] indicates undefined monosaccharide sequence.

When cord blood CD133+ cell acidic N-glycans were analyzed, the following glycans produced structure-indicating signals (nomenclature is as described by Domon and Costello, 1988, Glycoconjugate J.).

m/z 1532.78 (NeuAcHex3HexNAc2) yielded fragments: B, (m/z 375.69 with H⁺ adduct ion), B₃/Y₅ or B₄/Y₄ (m/z 471.79 with Na⁺ adduct ion), Y₂ (m/z 503.88), Y₃ (m/z 707.99), B₃(m/z 847.00) and Y₅ (m/z 1157.51), corresponding to linear structure Neuac-[Hex-HexNAc]-Hex-[Hex-HexNAc], possibly corresponding to linear structure Neuac-Hex-HexNAc-Hex-Hex-HexNAc, more preferentially N-glycan structure NeuAcα2-3/6Galβ1-3/4GlcNAcβ1-2Manα1-3/6Manβ1-4GlcNAc, wherein the underlined linkage is preferentially α1-3.

m/z 2156.03 (NeuAcHex4HexNAc3dHex) yielded fragments: B_{1α} (m/z 375.86 with H⁺ adduct ion), B_{3α}/Y_{6α} (m/z 471.90 with Na⁺ adduct ion), B₃ (m/z 846.90), Y_{4α} (m/z 1331.71) and Y_{6α} (m/z 1781.62), corresponding to a structure with identical monosaccharide sequence as the structure NeuAcα2-3/6Galβ1-3/4GlcNAcβ1-2Manα1-3/6(Manα1-6/3)Manβ1-4GlcNAcβ 1-4(Fucα1-6)GlcNAc, wherein the underlined linkage is preferentially α1-3.

m/z 2431.14 (NeuAcHex5HexNAc4) yielded fragments: B_{3α}/Y_{6α} (m/z 471.87 with Na⁺ adduction), B_{3α} (m/z 846.65), Y_{4α}/Y_{3β} (m/z 939.09), Y_{6α}/Y_{4β} (m/z 1591.61) and Y_{4α}/Y_{6β} (m/z 1606), possibly corresponding the structure NeuAcα2-3/6Galβ1-3/4GlcNAcβ 1-2Manα1-3/6(Galβ1-3/4GlcNAcβ1-2Manα1-3/6)Manβ1-4GlcNAcβ 1-4GlcNAc.

m/z 2605.22 (NeuAcHex5HexNAc4dHex) yielded fragments: B_{3α} (m/z 847.42 with Na⁺ adduct ion) and Y_{4α}/Y_{6β} (m/z 1782.06), corresponding to a structure with identical monosaccharide sequence as the structure NeuAcα2-3/6Galβ1-3/4GlcNAcβ 1-2Manα1-3/6(Galβ1-3/4GlcNAcβ1-2Manα1-3/6)Manβ1-4GlcNAcβ 1-4(Fucα1-6)GlcNAc.

m/z 2779.3 (NeuAcHex5HexNAc4dHex2) yielded fragments: B_{3α} (m/z 847.79 with Na⁺ adduct ion) and B_{6α}/Y_{6α} (m/z 1970.21), corresponding to a structure with identical monosaccharide sequence as structure NeuAcα2-3/6Galβ1-3/4GlcNAcβ1-2Manα1-3/6([Fucα1-2′/3/4][Galβ1-3/4GlcNAcβ1-2]Manα1-3/6)Manβ1-4GlcNAcβ1-4(Fucα1-6)GlcNAc.

Taken together, the present results yielded especially direct evidence for the following specific structures in CD133+ cell N-glycans: N-glycan monoantennary core structure, N-glycan biantennary core structure, hybrid-type N-glycan core structure, and non-reducing terminal Lex on sialylated biantennary N-glycan non-sialylated antenna, further verifying structural assignments according to the invention.

When cord blood CD133+ cell acidic N-glycans were analyzed, the following glycans produced structure-indicating signals:

m/z 1532.77 (NeuAcHex3HexNAc2) yielded fragments: B₁ (m/z 375.95 with H⁺ adduct ion), B₃/Y₅ or B₄/Y₄ (m/z 471.91 with Na⁺ adduct ion), Y₂ (m/z 503.89), Y₃ (m/z 708.13), B₃(m/z 847.15) and Y₅ (m/z 1157.52), corresponding to a structure with identical monosaccharide sequence as structure NeuAcα2-3/6Galβ1-3/4GlcNAcβ 1-2Manα1-3/6Manβ1-4GlcNAc.

m/z 2156.01 (NeuAcHex4HexNAc3dHex) yielded fragments: B_{3α} (m/z 846.97 with Na⁺ adduct ion), Y_{4α} (m/z 1331.29) and Y_{6α} (m/z 1781.92), corresponding to a structure with identical monosaccharide sequence as structure NeuAcα2-3/6Galβ1-3/4GlcNAcβ 1-2Manα1-3/6(Manα1-3/6)Manβ1-4GlcNAcβ 1-4(Fucα1-6)GlcNAc.

m/z 2605.30 (NeuAcHex5HexNAc4dHex) yielded fragments: B_{3α}/Y_{6α} (m/z 472.23 with Na⁺ adduct ion) and Y_{4α}/Y_{6β} (m/z 1780.60), corresponding to a structure with identical monosaccharide sequence as structure NeuAcα2-3/6Galβ1-3/4GlcNAcβ1-2Manα1-3/6(Galβ1-3/4GlcNAcβ 1-2Manα1-3/6)Manβ1-4GlcNAcβ 1-4(Fucα1-6)GlcNAc.

m/z 3054.52 (NeuAcHex6HexNAc5dHex) yielded fragments: B_{1α} (m/z 375.82 with H⁺ adduct ion), B_{3α}/Y_{6α} (m/z 471.99 with Na⁺ adduct ion), B₃, (m/z 846.58), corresponding to a structure with identical monosaccharide sequence as structure NeuAcα2-3/6 {Galβ1-3/4GlcNAcβ1-2Manα1-3/6[Galβ1-3/4GlcNAcβ1-2(Galβ1-3/4GlcNAcβ1-4)Manα1-3/6]Manβ1-4GlcNAcβ1-4(Fucα1-6)GlcNAc}.

Taken together, the present results yielded especially direct evidence for the following specific structures in cord blood cell N-glycans: N-glycan monoantennary core structure, N-glycan biantennary core structure, hybrid-type N-glycan core structure, and non-reducing terminal LacNAc on sialylated triantennary N-glycan non-sialylated antenna, further verifying structural assignments according to the invention.

TABLE 1
Expression of the genes encoding glycosyltransferases and glycosidases
involved in the biosynthesis of N-glycans in CD133+ and CD133− cells.
In addition, gene name encoding glycosyltransferases and glycosidases of the
same family along with their glycan class and structure specifity is represented.

| Gene | | | | |
|---|---|---|---|---|
| expression | | Gene | Glycan | |
| CD133+ | CD133− | name | class | Structure specificity |

α-mannosidase (MAN) families I and II (21, 48-54)

| | | | | |
|---|---|---|---|---|
| P | P | MAN1A1 | N | α2MAN belonging to the MAN I family |
| P | P | MAN1A2 | N | |
| P | P | MAN1B1 | N | |
| A | P | MAN1C1 | N | |
| P | P | MAN2A1 | N | α3/6MAN belongning to the MAN II |
| P | P | MAN2A2 | N | family |
| P | P | MAN2B1 | N | |
| P | P | MAN2B2 | N | |
| P | P | MAN2C1 | N | |

N-glycan branching β-N-acetylglucosaminyltransferases (MGAT) (17)

| | | | | |
|---|---|---|---|---|
| P | P | MGAT1 | N | N-glycan branching enzymes; |
| P, I | P | MGAT2 | N | see also FIG. 4. |
| A | A | MGAT3 | N | |
| P, D | P | MGAT4A | N | |
| P | P | MGAT4B | N | |
| A | A | MGAT5 | N | |
| *NP | *NP | MGAT6 | N | |

β1,3-galactosyltransferases (β3GalT) (55-60)

| | | | | |
|---|---|---|---|---|
| A | P | B3GALT1 | N, O, L | |
| A | A | B3GALT2 | N, O, L | |
| | | B3GALT3 | L | globoside synthase |
| | | B3GALT4 | L | GM1 synthase |
| A | A | B3GALT5 | N, O, L | O-glycan Core 3 elongation |
| | | B3GALT6 | G | GAG GalT2 |
| | | B3GALT7 | (1 | |

β1,4-galactosyltransferases (β4GalT) (29, 61-65)

| | | | | |
|---|---|---|---|---|
| P, I | P | B4GALT1 | N, O, L | lactose/N-acetyllactosamine synthase |
| P | A | B4GALT2 | N, O, L | Lactose/N-acetyllactosamine synthase |
| P, D | P | B4GALT3 | N, O, L | |
| P | P | B4GALT4 | N, O, L | 6-sulfo-GlcNAc GalT |
| A | A | B4GALT5 | O > N, L | O-glycan Core 2 elongation |
| | | B4GALT6 | L | lactosylceramide synthase |
| | | B4GALT7 | G | GAG GalT1 |

α2,3-sialyltransferases (α3SAT) (33, 66, 67)

| | | | | |
|---|---|---|---|---|
| | | ST3GAL1 | O | O-glycan Core 1 sialylation |
| A | A | ST3GAL2 | N, O, L | |
| A | A | ST3GAL3 | N, O, L | type 1 LacNAc sialylation |
| A | A | ST3GAL4 | N, O, L | type 2 LacNAc sialylation |
| | | ST3GAL5 | L | GM3 synthase |
| P, I | P | ST3GAL6 | N, O, L | type 2 LacNAc sialylation |

α2,6-sialyltransferases (α6SAT) 37, 52, 68-71

| | | | | |
|---|---|---|---|---|
| P | P | ST6GAL1 | N, O, L | type 2 LacNAc sialylation |
| A | A | ST6GAL2 | N, O, L | type 2 LacNAc sialylation |

α1,2-/α1,3-/α1,4-/α1,6-fucosyltransferases (FucT) (18, 19, 44, 72, 73, 73-80)

| | | | | |
|---|---|---|---|---|
| A | A | FUT1 | N, O, L | α1,2-FucT (H-2 synthesis) |
| *NP | *NP | FUT2 | N, O, L | α1,2-FucT (Secretor, H-1 synthesis) |
| A | A | FUT3 | N, O, L | α1,3/4-FucT |
| P | P | FUT4 | N, O, L | α1,3-FucT (Lex/sLex synthesis) |
| A | A | FUT5 | N, O, L | α1,3-FucT (Lex/sLex synthesis) |
| A | A | FUT6 | N, O, L | α1,3-FucT (Lex/sLex synthesis) |
| A | A | FUT7 | N, O, L | α1,3-FucT (Lex/sLex synthesis) |
| P | A | FUT8 | N | α1,6-FucT (N-glycan core fucosylation) |
| A | A | FUT9 | N, O, L | α1,3-FucT (Lex/sLex synthesis) |

1) May be a false annotation, should be B3GNT1
Abreviations:
A; gene not expressed,
P; gene expression;
I; increased expression in CD133+ cells,
D; decreased gene expression in CD133+ cells,
*NP; no probe available,
N; N-glycan,
O; O-glycan,
L; glycosphingolipids;
G, glycosaminoglycans.

TABLE 2
Cell surface glycan epitope assay with lectins.

| | | | stem |
|---|---|---|---|
| Lectin | Specificity | leucocytes | cells |
| PSA | α-mannose, N-glycan core structure | 96% | +++ |
| HHA | α-mannose | 99% | +++ |
| GNA | α-mannose, less to α1,3-linked mannose | 73% | + |
| | residues | | |
| PHA-L | large complex-type N-glycans with β1,6- | 98% | ++ |
| | branch | | |
| RCA-I | β1,4-linked galactose, type 2 LacNAc | 91% | +++ |
| SNA | α2,6-linked sialic acid | 98% | +++ |
| MAA | α2,3-linked sialic acid in type 2 LacNAc | 62% | ++ |
| LTA | α1,3-linked fucose (Lex) | 6% | − |
| UEA-I | α1,2-linked fucose in type 2 LacNAc (H-2) | 53% | +++ |

TABLE 3
Neutral N-glycan difference analysis.

| | | | |
|---|---|---|---|
| composition¹⁾ | m/z²⁾ | class³⁾ | fold⁴⁾ |

+++ CD133+⁵⁾

| | | | |
|---|---|---|---|
| H2N3 | 974 | H | +∞ |
| H4N5 | 1704 | CT | +∞ |

++ CD133+

| | | | |
|---|---|---|---|
| H3N5 | 1542 | CT | 1.91 |
| H5N4F3 | 2101 | CE | 1.91 |
| H4N4F1 | 1647 | CF | 1.76 |
| H3N5F1 | 1688 | CFT | 1.55 |
| H1N2F1 | 755 | LF | 1.50 |

+ CD133+

| H3N3F2 | 1428 | HE | 1.49 |
|---|---|---|---|
| H2N3F1 | 1120 | HF | 1.46 |
| H5N4F2 | 1955 | CE | 1.36 |
| H4N4F2 | 1793 | CE | 1.34 |
| H5N3F2 | 1752 | HE | 1.33 |
| H5N2 | 1257 | M | 1.30 |
| H4N3F2 | 1590 | HE | 1.27 |
| H5N4F1 | 1809 | CF | 1.22 |
| H5N3F1 | 1606 | HF | 1.21 |
| H4N3F1 | 1444 | HF | 1.21 |
| H6N2F1 | 1565 | MF | 1.19 |
| H9N2 | 1905 | M | 1.18 |
| H8N2 | 1743 | M | 1.12 |
| H3N3F1 | 1282 | HF | 1.08 |
| H6N3F1 | 1768 | HF | 1.05 |
| H5N2F1 | 1403 | MF | 1.03 |
| H4N5F3 | 2142 | CET | 1.03 |
| H6N5 | 2028 | CR | 1.02 |
| H6N5F1 | 2174 | CFR | 1.01 |
| composition | m/z | class | fold |

+ CD133−

| | | | |
|---|---|---|---|
| H3N4F1 | 1485 | CFT | −1.02 |
| H4N2 | 1095 | L | −1.03 |
| H10N2 | 2067 | MG | −1.03 |
| H7N2 | 1581 | M | −1.05 |
| H6N2 | 1419 | M | −1.07 |
| H2N2F1 | 917 | LF | −1.10 |
| H6N3 | 1622 | H | −1.18 |
| H4N2F1 | 1241 | LF | −1.19 |
| H5N4 | 1663 | C | −1.40 |
| H5N3 | 1460 | H | −1.41 |

++ CD133−

| | | | |
|---|---|---|---|
| H3N2F1 | 1079 | LF | −1.53 |
| H2N2 | 771 | L | −1.54 |
| H3N2 | 933 | L | −1.56 |
| H3N3 | 1136 | H | −1.63 |
| H4N3 | 1298 | H | −1.67 |
| H1N2 | 609 | L | −1.77 |

+++ CD133−

| | | | |
|---|---|---|---|
| HSN5 | 1866 | CT | −∞ |

¹⁾Proposed composition wherein the monosaccharide symbols are: H, Hex;
N, HexNAc; F, dHex.
²⁾Calculated m/z for [M + Na]+ ion rounded down to next integer.
³⁾N-glycan class symbols are: M, high-mannose type; L, low-mannose
type; H, hybrid-type or monoantennary; C, complex-type; O, other type; F,
fucosylated; E, complex-fucosylated, wherein at least one fucose residue
is α1.2-, α1.3- or α1.4-linked; R, large complex-type; G, glucosylated;
T, non-reducing terminal HexNAc.
⁴⁾‘fold’ is calculated according to the equation: $fold = {x ( \frac{{P}_{a}}{{P}_{b}} )}^{x} ,$ wherein P is the relative abundancy (%) of the glycan signal in profile a or b, x is 1 when Pₐ ≧ P_{b}, and x is −1 when a < b; +∞, detected only in CD133+ cells; −∞, not detected in CD133+ cells.
⁵⁾Association with human cord blood mononuclear cell type based on fold
calculation: + low association, ++ substantial association,
+++ high association.

TABLE 4
Sialylated N-glycan difference analysis.

| | | | |
|---|---|---|---|
| composition¹⁾ | m/z²⁾ | class³⁾ | fold⁴⁾ |

+++ CD133+⁵⁾

| | | | |
|---|---|---|---|
| S1H3N3 | 1403 | H | +∞ |
| S1H4N3F1P | 1791 | HFP | +∞ |
| S4H3N3 | 1856 | H | +∞ |
| S3H4N3F1 | 2293 | HF | +∞ |
| S1H7N6F2 | 2953 | CER | +∞ |

++ CD133+

| | | | |
|---|---|---|---|
| S2H5N4 | 2221 | C | 1.55 |
| S2H5N4F1 | 2367 | CF | 1.53 |
| S1H3N3F1 | 1549 | HF | 1.51 |

+ CD133+

| S1H3N2 | 1200 | | 1.39 |
|---|---|---|---|
| S1H5N4F3 | 2368 | CE | 1.35 |
| S1H5N4F2 | 2222 | CE | 1.26 |
| S1H5N4 | 1930 | C | 1.20 |
| S1H4N4F1 | 1914 | CF | 1.13 |
| S1H4N4 | 1768 | C | 1.08 |
| composition | m/z | class | fold |

+CD133−

| | | | |
|---|---|---|---|
| S1H5N4F1 | 2076 | CF | −1.02 |
| S1H4N3F1 | 1711 | HF | −1.02 |
| S1H5N3F1 | 1873 | HF | −1.11 |
| S1H4N3 | 1565 | H | −1.20 |
| S2H6N5F1 | 2732 | CFR | −1.22 |
| S2H5N4F4 | 2806 | CE | −1.32 |
| S1H7N6F3 | 3099 | CER | −1.36 |
| S1H5N3 | 1727 | H | −1.43 |

++CD133−

| | | | |
|---|---|---|---|
| S1H5N5F1 | 2279 | CFT | −1.60 |
| S1H6N3 | 1889 | H | −1.61 |
| S1H6N5F1 | 2441 | CFR | −1.82 |

+++CD133−

| | | | |
|---|---|---|---|
| S1H7N6F1 | 2807 | CFR | −7.60 |
| S1H5N5 | 2133 | CT | −∞ |
| S1H6N5 | 2295 | CR | −∞ |
| S1H6N5F2 | 2587 | CER | −∞ |
| S1H6N5F3 | 2733 | CER | −∞ |
| S3H6N5F1 | 3024 | CFR | −∞ |
| S2H7N6F1 | 3098 | CFR | −∞ |
| S2H7N6F3 | 3390 | CER | −∞ |

¹⁾Proposed composition wherein the monosaccharide symbols are: S,
NeuAc; H, Hex; N, HexNAc; F, dHex; P, SP = sulphate or phosphate
ester.
²⁾Calculated m/z for [M − H]− ion rounded down to next integer.
³⁾N-glycan class symbols are: H, hybrid-type or monoantennary; C,
complex-type; O, other type; F, fucosylated; E, complex-fucosylated,
wherein at least one fucose residue is α1.2-, α1.3- or α1.4-linked; R,
large complex-type; T, non-reducing terminal HexNAc.
⁴⁾‘fold’ is calculated according to the equation: $fold = {x ( \frac{{P}_{a}}{{P}_{b}} )}^{x} ,$ wherein P is the relative abundancy (%) of the glycan signal in profile a or b, x is 1 when Pₐ ≧ P_{b}, and x is −1 when a < b; +∞, detected only in CD133+ cells; −∞, not detected in CD133+ cells.
⁵⁾Association with human cord blood mononuclear cell type based on fold
calculation: + low association, ++ substantial association,
+++ high association.

TABLE 5
Individual variation in human cord blood CD133+ cell neutral
N-glycan profiles.

Large* individual variation:
H3N3, H5N3F1, H4N5, H1N2, H1N2F1, H5N5, H5N4F3
Substantial individual variation:
H4N4F1, H4N2F1
Little individual variation:
H3N5F1, H5N4F1, H3N4F1, H10N2, H3N3F1, H2N2, H5N3, H2N2F1,
H5N2, H3N2, H3N2F1, H5N2F1, H6N3, H6N2, H4N2, H7N2, H9N2,
H8N2, H2N3F1, H3N3F2, H4N3F1, H3N5, H6N2F1, H4N3F2,
H5N4, H4N4F2, H5N4F2, H6N5
*The variation was evaluated by calculating the proportion of standard deviation from average value for each glycan signal in a panel of individual CD133+ N-glycan analyses from several cord blood units, and classifying the proportion as follows: large, >100%; substantial, 50-100%; little, 0-50%.

TABLE 6
Individual variation in human cord blood CD133+ cell sialylated
N-glycan profiles.

Large* individual variation:
S2H8N7F3, S1H3N3, S3H7N6F3, S1H3N2 (m/z 1200), S1H3N3F1,
S2H6N5F3, S1H8N7F3
Substantial individual variation:
S1H5N3, S2H6N5F2, S2H7N6F1, S1H6N5, S1H5N4F3, S3H7N6F1,
S3H6N5F1
Little individual variation:
S1H6N5F2, S1H6N5F3, S1H6N3, S1H5N5F1, S1H4N3F1, S1H4N4F1,
S1H4N3, S2H6N5F1, S1H7N6F1, S2H5N4F1, S1H5N4F2, S1H5N3F1,
S1H6N5F1, S2H5N4, S1H5N4, S1H4N4, S1H5N4F1, S3H6N5F1P1,
S2H7N6F3
*The variation was evaluated by calculating the proportion of standard deviation from average value for each glycan signal in a panel of individual CD133+ N-glycan analyses from several cord blood units, and classifying the proportion as follows: large, >100%; substantial, 50-100%; little, 0-50%.

TABLE 7
Cord blood mononuclear cell sialylated N-glycan signals. The m/z values
refer to monoisotopic masses of [M − H]⁻ ions.

| Proposed monosaccharide composition | m/z (calculated) | |
|---|---|---|
| NeuAcHex3HexNAc3dHex | 1549.55 | 1549 |
| NeuAcHex4HexNAc3 | 1565.55 | 1565 |
| NeuAc2Hex3HexNAc2dHex | 1637.57 | 1637 |
| NeuAc2Hex2HexNAc3dHex | 1678.60 | 1678 |
| NeuAcHex4HexNAc3dHex | 1711.61 | 1711 |
| NeuAcHex5HexNAc3 | 1727.60 | 1727 |
| NeuAcHex3HexNAc4dHex | 1752.63 | 1752 |
| NeuAcHex4HexNAc4 | 1768.57 | 1768 |
| NeuAcHex4HexNAc3dHexSO3 | 1791.56 | 1791 |
| NeuAc2Hex3HexNAc3dHex | 1840.65 | 1840 |
| NeuAcHex4HexNAc3dHex2 | 1857.66 | 1857 |
| Hex5HexNAc4dHexSO3 | 1865.60 | 1865 |
| NeuAcHex5HexNAc3dHex | 1873.66 | 1873 |
| NeuAcHex6HexNAc3 | 1889.65 | 1889 |
| NeuAcHex3HexNAc4dHex2 | 1898.69 | 1898 |
| NeuAcHex4HexNAc4dHex | 1914.68 | 1914 |
| NeuAcHex5HexNAc4 | 1930.68 | 1930 |
| NeuAc2Hex4HexNAc3dHex/ | 2002.70 | 2002 |
| Hex8HexNAc3SO3 | | |
| NeuAc2Hex5HexNAc3 | 2018.70 | 2018 |
| NeuAcHex6HexNAc3dHex | 2035.71 | 2035 |
| NeuAcHex7HexNAc3 | 2051.71 | 2051 |
| Hex4HexNAc5dHex2SO3 | 2052.68 | 2052 |
| NeuAc2Hex4HexNAc4 | 2059.72 | 2059 |
| NeuAcHex4HexNAc4dHex2 | 2060.74 | 2060 |
| NeuAcHex5HexNAc4dHex | 2076.74 | 2076 |
| NeuAcHex6HexNAc4 | 2092.73 | 2092 |
| NeuAcHex4HexNAc5dHex | 2117.76 | 2117 |
| NeuAcHex5HexNAc5 | 2133.76 | 2133 |
| NeuAcHex8HexNAc2dHex/ | 2156.74/2156.69 | 2156 |
| NeuAcHex5HexNAc4dHexSO3 | | |
| NeuAc2Hex5HexNAc4 | 2221.78 | 2221 |
| NeuAcHex5HexNAc4dHex2 | 2222.80 | 2222 |
| Hex6HexNAc5dHexSO3 | 2230.73 | 2230 |
| NeuAcHex6HexNAc4dHex/ | 2238.79 | 2238 |
| NeuGcHex5HexNAc4dHex2 | | |
| NeuAcHex7HexNAc4/ | 2254.79 | 2254 |
| NeuGcHex6HexNAc4dHex | | |
| NeuAcHex5HexNAc5dHex | 2279.82 | 2279 |
| NeuAc2Hex4HexNAc3dHex3 | 2294.82 | 2294 |
| NeuAcHex6HexNAc5 | 2295.81 | 2295 |
| NeuAc2Hex5HexNAc4dHex | 2367.83 | 2367 |
| NeuAcHex5HexNAc4dHex3 | 2368.86 | 2368 |
| NeuAc2Hex6HexNAc4 | 2383.83 | 2383 |
| NeuAcHex6HexNAc4dHex2 | 2384.85 | 2384 |
| NeuAc2Hex5HexNAc3dHexSO3 | 2390.77 | 2390 |
| NeuAc2Hex3HexNAc5dHex2 | 2392.86 | 2392 |
| NeuAcHex5HexNAc5dHex2 | 2425.87 | 2425 |
| NeuAcHex6HexNAc5dHex | 2441.87 | 2441 |
| NeuAc2Hex8HexNAc2dHex/ | 2447.83/2447.79 | 2447 |
| NeuAc2Hex5HexNAc4dHexSO3 | | |
| NeuAcHex7HexNAc5 | 2457.86 | 2457 |
| NeuAc2Hex5HexNAc4dHex2 | 2513.89 | 2513 |
| NeuAcHex6HexNAc5dHexSO3 | 2521.83 | 2521 |
| NeuAcHex6HexNAc4dHex3 | 2530.91 | 2530 |
| NeuAc3Hex4HexNAc5 | 2553.90 | 2553 |
| NeuAc2Hex5HexNAc5dHex | 2570.91 | 2570 |
| NeuAcHex5HexNAc5dHex3 | 2571.93 | 2571 |
| NeuAc2Hex6HexNAc5 | 2586.91 | 2586 |
| NeuAcHex6HexNAc5dHex2 | 2587.93 | 2587 |
| Hex7HexNAc6dHexSO3 | 2595.86 | 2595 |
| NeuAcHex7HexNAc5dHex | 2603.92 | 2603 |
| NeuAcHex6HexNAc6dHex | 2644.95 | 2644 |
| NeuAcHex7HexNAc6 | 2660.94 | 2660 |
| NeuAc2Hex4HexNAc5dHex2(SO3)2 | 2714.83 | 2714 |
| NeuAc2Hex6HexNAc5dHex | 2732.97 | 2732 |
| NeuAcHex6HexNAc5dHex3 | 2733.99 | 2733 |
| NeuAcHex7HexNAc6dHex | 2807.00 | 2807 |
| NeuAcHex6HexNAc5dHex3SO3 | 2813.94 | 2813 |
| NeuAc3Hex6HexNAc5 | 2878.00 | 2878 |
| NeuAc2Hex6HexNAc5dHex2 | 2879.02 | 2879 |
| NeuAcHex6HexNAc5dHex4 | 2880.04 | 2880 |
| NeuAc2Hex5HexNAc6dHex2 | 2920.05 | 2920 |
| NeuAc2Hex7HexNAc6 | 2952.04 | 2952 |
| NeuAcHex7HexNAc6dHex2 | 2953.06 | 2953 |
| NeuAcHex7HexNAc7dHex | 3010.08 | 3010 |
| NeuAc3Hex6HexNAc5dHex | 3024.06 | 3024 |
| NeuAc2Hex6HexNAc5dHex3 | 3025.09 | 3025 |
| NeuAcHex8HexNAc7 | 3026.08 | 3026 |
| NeuAc2Hex7HexNAc6dHex | 3098.10 | 3098 |
| NeuAcHex7HexNAc6dHex3 | 3099.12 | 3099 |
| NeuAc2Hex6HexNAc5dHex4 | 3171.14 | 3171 |
| NeuAcHex8HexNAc7dHex | 3172.13 | 3172 |

TABLE 8
Mass spectrometric analysis results of sialylated N-glycans with
monosaccharide compositions NeuAc₁₋₂Hex₅HexNAc₄dHex₀₋₃ in
sequential enzymatic modification steps of human cord blood
mononuclear cells. The columns show relative glycan signal
intensities (% of the tabled signals) before the modification reactions
(MNC), after α2,3-sialyltransferase reaction (α2,3SAT), and
after sequential α2,3-sialyltransferase and α1,3-fuscosyltransferase
reactions (α2,3SAT + α1,3FucT). The sum of the glycan signal
intensities in each column has been normalized to 100% for clarity.

| | calc m/z | | | α2,3SAT + |
|---|---|---|---|---|
| Proposed monosaccharide composition | [M − H]⁻ | MNC | α2,3SAT | α1,3FucT |
| NeuAcHex5HexNAc4 | 1930.68 | 24.64 | 12.80 | 13.04 |
| NeuAcHex5HexNAc4dHex | 2076.74 | 39.37 | 30.11 | 29.40 |
| NeuAcHex5HexNAc4dHex2 | 2222.8 | 4.51 | 8.60 | 6.83 |
| NeuAcHex5HexNAc4dHex3 | 2368.85 | 3.77 | 6.34 | 6.45 |
| NeuAc2Hex5HexNAc4 | 2221.78 | 13.20 | 12.86 | 17.63 |
| NeuAc2Hex5HexNAc4dHex | 2367.83 | 14.04 | 29.28 | 20.71 |
| NeuAc2Hex5HexNAc4dHex2 | 2513.89 | 0.47 | n.d. | 5.94 |

TABLE 9
Mass spectrometric analysis results of selected neutral N-glycans in enzymatic
modification steps of human cord blood mononuclear cells. The columns show relative glycan
signal intensities (% of the total glycan signals) before the modification reactions (MNC), after
broad-range sialidase reaction (SA'se), after α2,3-sialyltransferase reaction (α2,3SAT), after
α1,3-fucosyltransferase reaction (α1,3FucT), and after sequential α2,3-sialyltransferase and
α1,3-fucosyltransferase reactions (α2,3SAT + α1,3FucT).

| | calc m/z | | | | | α2,3SAT + |
|---|---|---|---|---|---|---|
| Proposed monosaccharide composition | [M + H]⁺ | MNC | SA'ase | α2,3SAT | α1,3FucT | α1,3FucT |
| Hex5HexNAc2 | 1257.42 | 11.94 | 14.11 | 14.16 | 13.54 | 9.75 |
| Hex3HexNAc4dHex | 1485.53 | 0.76 | 0.63 | 0.78 | 0.90 | 0.78 |
| Hex6HexNAc3 | 1622.56 | 0.61 | 1.99 | 0.62 | 0.51 | 0.40 |
| Hex5HexNAc4 | 1663.58 | 0.44 | 4.81 | 0.00 | 0.06 | 0.03 |
| Hex5HexNac4dHex | 1809.64 | 0.19 | 1.43 | 0.00 | 0.25 | 0.00 |
| Hex5HexNac4dHex2 | 1955.7 | 0.13 | 0.22 | 0.00 | 0.22 | 0.00 |
| Hex6HexNAc5 | 2028.71 | 0.07 | 1.14 | 0.00 | 0.00 | 0.00 |
| Hex5HexNAc4dHex3 | 2101.76 | 0.12 | 0.09 | 0.00 | 0.22 | 0.00 |
| Hex6HexNAc5dHex | 2174.77 | 0.00 | 0.51 | 0.00 | 0.14 | 0.00 |
| Hex6HexNAc5dHex2 | 2320.83 | 0.00 | 0.00 | 0.00 | 0.08 | 0.00 |

TABLE 10
Neutral N-glycan grouping of cord blood cell populations, cord blood mononuclear
cells (CB MNC), and peripheral blood mononuclear cells (PB MNC).

| Neutral N-glycan Grouping: | | CD | | CD | CD | | | CB | PB |
|---|---|---|---|---|---|---|---|---|---|
| Composition | Glycan Grouping | 34+ | CD 34− | 133+ | 133− | LIN− | LIN+ | MNC | MNC |

General N-glycan grouping:

| | | | | | | | | | |
|---|---|---|---|---|---|---|---|---|---|
| Hex₅₋₁₂HexNAc₂ | high-mannose | 56.3 | 52.9 | 67.0 | 55.1 | 58.9 | 61.2 | 65.4 | 62.7 |
| Hex₁₋₄HexNAc₂dHex₀₋₁ | low-mannose | 33.1 | 35.5 | 25.6 | 32.8 | 21.1 | 24.5 | 26.5 | 29.6 |
| n_{HexNAc} = 3 and n_{Hex} ≧ 2 | hybrid/monoant. | 5.5 | 6.4 | 2.4 | 5.6 | 8.6 | 5.5 | 4.3 | 3.7 |
| n_{HexNAc} ≧ 4 and n_{Hex} ≧ 2 | complex | 4.3 | 4.8 | 4.5 | 5.9 | 11.0 | 8.0 | 3.1 | 3.3 |
| Other types | — | 0.8 | 0.4 | 0.6 | 0.7 | 0.5 | 0.7 | 0.7 | 0.7 |

Complex/hybrid/monoantennary N-glycan grouping:

| | | | | | | | | | |
|---|---|---|---|---|---|---|---|---|---|
| n_{dHex} ≧ 1 | fucosylated | 67.8 | 70.6 | 81.2 | 66.4 | 49.0 | 66.8 | 58.8 | 56.4 |
| n_{dHex} ≧ 2 | α2/3/4-linked Fuc | 18.8 | 21.3 | 0.5 | 11.5 | 0 | 5.4 | 12.2 | 4.9 |
| n_{HexNAc} > n_{Hex} ≧ 2 | terminal HexNAc | 21.3 | 18.3 | 50.8 | 32.1 | 38.7 | 34.2 | 22.7 | 26.9 |
| n_{HexNAc} = n_{Hex} ≧ 5 | bisecting GlcNAc | 0 | 0 | 0.8 | 0.8 | 0.4 | 2.0 | 0.4 | 0 |

Complex N-glycan grouping:

| | | | | | | | | | |
|---|---|---|---|---|---|---|---|---|---|
| n_{HexNAc} ≧ 5 and n_{Hex} ≧ 6 | large N-glycans | 1.8 | 6.0 | 0 | 2.5 | 0 | 4.0 | 3.8 | 2.4 |

TABLE 11
Exoglycosidase profiling of cord blood CD34+ and CD34− cell neutral N-glycan
fraction.

| | | α-Man | | β1,4-Gal | | β1,3-Gal | | β-GlcNAc | |
|---|---|---|---|---|---|---|---|---|---|
| Proposed composition | m/z | CD 34+ | CD 34− | CD 34+ | CD 34− | CD 34+ | CD 34− | CD 34+ | CD 34− |
| Hex2HexNAc | 568 | | −− | +++ | +++ | +++ | +++ | | |
| HexHexNAc2 | 609 | +++ | +++ | | +++ | | +++ | | |
| Hex3HexNAc | 730 | | −−− | −− | | − | | | |
| HexHexNAc2dHex | 755 | +++ | ++ | − | − | − | −− | | |
| Hex2HexNAc2 | 771 | ++ | −− | −− | −− | −− | −− | −− | |
| Hex4HexNAc | 892 | −−− | −−− | − | | − | | | |
| Hex2HexNAc2dHex | 917 | −− | −− | −− | | −− | −− | −− | |
| Hex3HexNAc2 | 933 | −−− | −− | − | −− | −− | −− | | |
| HexHexNAc3dHex | 958 | | +++ | | | | | | |
| Hex2HexNAc3 | 974 | | | | +++ | | +++ | | |
| Hex5HexNAc | 1054 | −−− | −− | + | | | + | − | |
| Hex3HexNAc2dHex | 1079 | −− | −− | | −− | − | −− | + | |
| Hex4HexNAc2 | 1095 | −−− | −−− | | | | | | |
| Hex2HexNAc3dHex | 1120 | | + | | + | | | | |
| Hex3HexNAc3 | 1136 | −−− | | | | | − | −−− | |
| Hex6HexNAc | 1216 | −−− | −− | | − | | − | − | |
| Hex4HexNAc2dHex | 1241 | | −−− | | − | − | − | − | |
| Hex5HexNAc2 | 1257 | −−− | −− | + | + | + | + | | |
| Hex3HexNAc3dHex | 1282 | −−− | + | | | − | − | −− | |
| Hex4HexNAc3 | 1298 | −−− | | −−− | | | − | | |
| Hex2HexNAc4dHex | 1323 | | | +++ | | | | | |
| Hex3HexNAc4 | 1339 | | +++ | | +++ | | | | |
| Hex7HexNAc | 1378 | | −−− | + | | + | | | |
| Hex5HexNAc2dHex | 1403 | | −−− | | | | | +++ | |
| Hex6HexNAc2 | 1419 | −−− | −− | ++ | ++ | ++ | ++ | ++ | |
| Hex3HexNAc3dHex2 | 1428 | −−− | ++ | | +++ | | +++ | | |
| Hex4HexNAc3dHex | 1444 | −−− | | − | −− | | −− | + | |
| Hex5HexNAc3 | 1460 | −−− | − | +++ | | +++ | | −−− | |
| Hex3HexNAc4dHex | 1485 | − | | | + | | | −−− | |
| Hex4HexNAc4 | 1501 | −−− | | | −−− | | −−− | −−− | |
| Hex8HexNAc | 1540 | −−− | −−− | −−− | +++ | −−− | +++ | −−− | |
| Hex3HexNAc5 | 1542 | | +++ | | +++ | | +++ | | |
| Hex6HexNAc2dHex | 1565 | | | | | | +++ | | |
| Hex7HexNAc2 | 1581 | −−− | −− | | ++ | ++ | ++ | ++ | |
| Hex4HexNAc3dHex2 | 1590 | −−− | | −−− | − | | − | + | |
| Hex5HexNAc3dHex | 1606 | | −−− | −−− | +++ | | +++ | +++ | |
| Hex6HexNAc3 | 1622 | −−− | −−− | −−− | −−− | | | −−− | |
| Hex4HexNAc4dHex | 1647 | −−− | | | | | − | −−− | |
| Hex5HexNAc4 | 1663 | −−− | | −−− | −−− | −−− | −− | −−− | |
| Hex3HexNAc5dHex | 1688 | | | | +++ | | +++ | | |
| Hex9HexNAc | 1702 | −−− | −−− | | +++ | +++ | +++ | | |
| Hex4HexNAc5 | 1704 | | +++ | | | | | | |
| Hex8HexNAc2 | 1743 | −−− | −−− | +++ | + | +++ | ++ | ++ | |
| Hex5HexNAc3dHex2 | 1752 | | −−− | | +++ | | +++ | +++ | |
| Hex6HexNAc3dHex | 1768 | | | | | +++ | | +++ | |
| Hex7HexNAc3 | 1784 | −−− | | | | | | −−− | |
| Hex4HexNAc4dHex2 | 1793 | | | −− | +++ | −− | +++ | | |
| Hex5HexNAc4dHex | 1809 | −−− | | | −−− | +++ | − | | |
| Hex6HexNAc4 | 1825 | | +++ | | | | | | |
| Hex3HexNAc6dHex | 1891 | | +++ | | | | | | |
| Hex9HexNAc2 | 1905 | −−− | −−− | − | | + | ++ | ++ | |
| Hex5HexNAc4dHex2 | 1955 | −−− | | −−− | −− | | −− | | |
| Hex10HexNAc2 | 2067 | | −−− | − | | | | +++ | |
| Hex5HexNAc4dHex3 | 2101 | | | − | − | | − | +++ | |
| Hex5HexNAc5dHex2 | 2158 | +++ | | | +++ | | | | |
| Hex6HexNAc5dHex | 2174 | | | | | | +++ | | |
| Hex6HexNAc5dHex3 | 2466 | | | | +++ | | | | |

α-Man, β1,4-Gal, β1,3-Gal, and β-GlcNAc refer to specific exoglycosidase enzymes as described in the text.
Code for profiling results, when compared to the profile before the reaction;
+++: new signal appears;
++: signal is significantly increased;
+: signal is increased;
−: signal is decreased;
−−: signal is significantly decreased;
−−−: signal disappears;
blank: no change.

TABLE 12
Exoglycosidase profiling of cord blood CD133+ and CD133− cell neutral N-glycan
fraction.

| | | α-Man | | β1,4-Gal | | β1,3-Gal | | β-GlcNAc | |
|---|---|---|---|---|---|---|---|---|---|
| Proposed composition | m/z | CD 133+ | CD 133− | CD 133+ | CD 133− | CD 133+ | CD 133− | CD 133+ | CD 133− |
| Hex2HexNAc | 568 | | | | + | | + | | +++ |
| HexHexNAc2 | 609 | +++ | ++ | | | | | | −−− |
| Hex3HexNAc | 730 | −−− | −−− | +++ | ++ | +++ | ++ | | ++ |
| HexHexNAc2dHex | 755 | +++ | ++ | −−− | | −−− | | | |
| Hex2HexNAc2 | 771 | + | −− | ++ | ++ | + | + | | + |
| Hex4HexNAc | 892 | −−− | −−− | + | ++ | | ++ | | + |
| Hex2HexNAc2dHex | 917 | −−− | −− | ++ | ++ | ++ | + | | |
| Hex3HexNAc2 | 933 | | −− | + | + | − | | | + |
| Hex2HexNAc3 | 974 | | | | +++ | | | | |
| Hex5HexNAc | 1054 | −−− | −− | + | ++ | + | ++ | | + |
| Hex3HexNAc2dHex | 1079 | −−− | −− | ++ | + | + | | | ++ |
| Hex2HexNAc3dHex | 1120 | +++ | ++ | ++ | + | ++ | + | | −−− |
| Hex3HexNAc3 | 1136 | +++ | + | | + | | | | −−− |
| Hex6HexNAc | 1216 | −−− | − | + | | + | + | | |
| Hex4HexNAc2dHex | 1241 | −−− | −−− | + | | | | | |
| Hex5HexNAc2 | 1257 | −− | −− | − | | | | | |
| Hex3HexNAc3dHex | 1282 | | | | | | | | −− |
| Hex4HexNAc3 | 1298 | ++ | + | + | | + | | | |
| Hex3HexNAc4 | 1339 | | | | +++ | | | | −−− |
| Hex7HexNAc | 1378 | −−− | −−− | | − | +++ | | | + |
| Hex5HexNAc2dHex | 1403 | −−− | −−− | −−− | | − | | | |
| Hex6HexNAc2 | 1419 | −− | −− | −− | − | − | −− | | |
| Hex3HexNAc3dHex2 | 1428 | | +++ | | − | | − | | |
| Hex4HexNAc3dHex | 1444 | | − | − | − | | | | |
| Hex5HexNAc3 | 1460 | −−− | − | + | + | | | | |
| Hex3HexNAc4dHex | 1485 | −− | | + | + | | − | | −−− |
| Hex4HexNAc4 | 1501 | | −−− | | | | +++ | | −−− |
| Hex8HexNAc | 1540 | −−− | −−− | −−− | | | | | ++ |
| Hex3HexNAc5 | 1542 | | −−− | | + | | − | | −−− |
| Hex6HexNAc2dHex | 1565 | | | −−− | | −−− | +++ | | |
| Hex7HexNAc2 | 1581 | −−− | −− | −− | −− | − | −− | | |
| Hex4HexNAc3dHex2 | 1590 | | −−− | − | − | − | − | | + |
| Hex5HexNAc3dHex | 1606 | −−− | −−− | + | | | | | −−− |
| Hex6HexNAc3 | 1622 | −−− | −−− | −−− | −− | | | | − |
| Hex4HexNAc4dHex | 1647 | −−− | | −−− | − | | | | −−− |
| Hex5HexNAc4 | 1663 | | | −−− | − | −− | − | | − |
| Hex3HexNAc5dHex | 1688 | −−− | + | | | | −−− | | −−− |
| Hex9HexNAc | 1702 | | | | | | | | + |
| Hex4HexNAc5 | 1704 | −−− | | | −−− | | | | |
| Hex8HexNAc2 | 1743 | −−− | −−− | −− | −− | − | −− | | |
| Hex5HexNAc3dHex2 | 1752 | | | | − | | | | +++ |
| Hex6HexNAc3dHex | 1768 | | | | | | | | |
| Hex4HexNAc4dHex2 | 1793 | | | | | | | | |
| Hex5HexNAc4dHex | 1809 | −−− | | −−− | −−− | | − | | − |
| Hex6HexNAc4 | 1825 | | | | − | | | | −−− |
| Hex5HexNAc5 | 1866 | −−− | −−− | | −−− | | | | −−− |
| Hex3HexNAc6dHex | 1891 | | | | | | | | −−− |
| Hex9HexNAc2 | 1905 | −−− | −−− | −− | −− | − | −− | | |
| Hex6HexNAc3dHex2 | 1914 | | | | −−− | | −−− | | |
| Hex5HexNAc4dHex2 | 1955 | | | | −− | | − | | −−− |
| Hex6HexNAc4dHex | 1971 | | −−− | | −−− | | | | −−− |
| Hex7HexNAc4 | 1987 | | | | −−− | | | | −−− |
| Hex5HexNAc5dHex | 2012 | | | | | | +++ | | |
| Hex6HexNAc5 | 2028 | | −−− | | −−− | | | | −−− |
| Hex10HexNAc2 | 2067 | −−− | −−− | − | − | | | | |
| Hex5HexNAc4dHex3 | 2101 | | | − | − | | − | | |
| Hex6HexNAc4dHex2 | 2117 | | −−− | | −−− | −−− | | | −−− |
| Hex7HexNAc4dHex | 2133 | | | | −−− | | | | |
| Hex6HexNAc5dHex | 2174 | | −−− | | −−− | | | | −−− |
| Hex5HexNAc6dHex | 2215 | | | | −−− | | | | |
| Hex6HexNAc4dHex3 | 2263 | | | | −−− | | −−− | | |
| Hex6HexNAc5dHex2 | 2320 | | | | −−− | | | | |
| Hex6HexNAc5dHex3 | 2466 | | | | −−− | | | | |

α-Man, β1,4-Gal, β1,3-Gal, and β-GlcNAc refer to specific exoglycosidase enzymes as described in the text.
Code for profiling results, when compared to the profile before the reaction;
+++: new signal appears;
++: signal is significantly increased;
+: signal is increased;
−: signal is decreased;
−−: signal is significantly decreased;
−−−: signal disappears;
blank: no change.

TABLE 13
Exoglycosidase profiling of cord blood Lin+ and Lin− cell neutral N-glycan fraction.

| | | α-Man | | β1,4-Gal | | β1,3-Gal | | β-GlcNAc | |
|---|---|---|---|---|---|---|---|---|---|
| Proposed composition | m/z | LIN+ | LIN− | LIN+ | LIN− | LIN+ | LIN− | LIN+ | LIN− |
| Hex2HexNAc | 568 | −−− | +++ | + | | + | | − | |
| HexHexNAc2 | 609 | +++ | +++ | | | | +++ | | |
| Hex2HexNAcdHex | 714 | | | +++ | | | | | |
| Hex3HexNAc | 730 | −−− | +++ | ++ | +++ | + | +++ | + | |
| HexHexNAc2dHex | 755 | +++ | +++ | + | | + | | +++ | |
| Hex2HexNAc2 | 771 | + | + | + | + | + | | + | |
| Hex4HexNAc | 892 | −−− | −−− | ++ | + | ++ | | + | + |
| Hex2HexNAc2dHex | 917 | −− | −−− | + | ++ | | − | | − |
| Hex3HexNAc2 | 933 | − | | + | + | + | − | | + |
| Hex2HexNAc3 | 974 | +++ | | | | | | | |
| Hex5HexNAc | 1054 | −− | −−− | ++ | | − | | − | |
| Hex3HexNAc2dHex | 1079 | −− | −−− | | ++ | | − | ++ | ++ |
| Hex4HexNAc2 | 1095 | −− | −−− | | | | − | | |
| Hex2HexNAc3dHex | 1120 | +++ | | | | | | | |
| Hex3HexNAc3 | 1136 | +++ | + | + | + | | − | +++ | −−− |
| Hex6HexNAc | 1216 | − | −−− | + | | + | + | | + |
| Hex4HexNAc2dHex | 1241 | −−− | −−− | | + | | + | | −−− |
| Hex5HexNAc2 | 1257 | −− | −−− | | ++ | − | − | − | + |
| Hex3HexNAc3dHex | 1282 | + | | | | | | −− | −−− |
| Hex4HexNAc3 | 1298 | + | | | | | | | |
| Hex2HexNAc4dHex | 1323 | | | | +++ | | +++ | | |
| Hex3HexNAc4 | 1339 | | −−− | ++ | + | | −− | | −−− |
| Hex7HexNAc | 1378 | −−− | −−− | | | + | ++ | | |
| Hex5HexNAc2dHex | 1403 | −−− | −−− | | | | + | | |
| Hex6HexNAc2 | 1419 | −− | −− | | −− | − | − | − | |
| Hex3HexNAc3dHex2 | 1428 | +++ | | −−− | | −−− | | +++ | |
| Hex4HexNAc3dHex | 1444 | | −−− | − | + | | + | | |
| Hex5HexNAc3 | 1460 | | −−− | | | | | | |
| Hex3HexNAc4dHex | 1485 | | | | | | −− | −−− | −−− |
| Hex4HexNAc4 | 1501 | + | −−− | + | − | −−− | −− | −−− | −−− |
| Hex8HexNAc | 1540 | −−− | −−− | | −−− | + | ++ | | |
| Hex3HexNAc5 | 1542 | +++ | | ++ | + | ++ | − | | |
| Hex6HexNAc2dHex | 1565 | | −−− | −−− | | | | | −−− |
| Hex7HexNAc2 | 1581 | −− | −−− | −− | −− | | − | | |
| Hex4HexNAc3dHex2 | 1590 | | | − | | | | +++ | |
| Hex5HexNAc3dHex | 1606 | −−− | −−− | − | −−− | | −−− | | −−− |
| Hex2HexNAc4dHex3 | 1615 | | | | | | +++ | | |
| Hex6HexNAc3 | 1622 | −−− | −−− | −−− | −−− | | | | |
| Hex4HexNAc4dHex | 1647 | | −−− | −− | −−− | | | −−− | −−− |
| Hex5HexNAc4 | 1663 | | −−− | −− | −− | | − | − | −− |
| Hex3HexNAc5dHex | 1688 | | | − | | | | −−− | −−− |
| Hex9HexNAc | 1702 | −−− | −−− | | | | | | |
| Hex4HexNAc5 | 1704 | +++ | | | −−− | | | | |
| Hex8HexNAc2 | 1743 | −− | −−− | −− | −− | | − | | |
| Hex5HexNAc3dHex2 | 1752 | | | −−− | | | | +++ | |
| Hex6HexNAc3dHex | 1768 | | | −−− | | | | | |
| Hex3HexNAc4dHex3 | 1777 | | | | | | +++ | | |
| Hex7HexNAc3 | 1784 | | | −−− | | | | | |
| Hex4HexNAc4dHex2 | 1793 | +++ | | | | | | | |
| Hex5HexNAc4dHex | 1809 | + | −−− | −− | −−− | | | | −− |
| Hex6HexNAc4 | 1825 | +++ | | − | −−− | | −− | +++ | |
| Hex4HexNAc5dHex | 1850 | | | +++ | | +++ | | | |
| Hex5HexNAc5 | 1866 | +++ | | −−− | | | | | |
| Hex3HexNAc6dHex | 1891 | | −−− | − | | | | | |
| Hex9HexNAc2 | 1905 | −−− | −−− | −− | −− | | − | | |
| Hex4HexNAc4dHex3 | 1939 | | | | | | +++ | | |
| Hex5HexNAc4dHex2 | 1955 | | | −−− | | | | +++ | |
| Hex6HexNAc4dHex | 1971 | | | −−− | | | | | |
| Hex7HexNAc4 | 1987 | | | −−− | | | | +++ | |
| Hex5HexNAc5dHex | 2012 | +++ | | −−− | | | | | |
| Hex6HexNAc5 | 2028 | | | −−− | | | | | |
| Hex10HexNAc2 | 2067 | −−− | −−− | − | | | ++ | + | |
| Hex5HexNAc4dHex3 | 2101 | | | | | | | +++ | |
| Hex8HexNAc4 | 2149 | | −−− | | | | | | |
| Hex6HexNAc5dHex | 2174 | | | −−− | | | | − | |
| Hex5HexNAc6dHex | 2215 | | | −−− | | −−− | | | |
| Hex11HexNAc2 | 2229 | | | | | | +++ | | |
| Hex6HexNAc6 | 2231 | | | −−− | | −−− | | | |
| Hex6HexNAc5dHex2 | 2320 | | | −−− | | −−− | | | |
| Hex12HexNAc2 | 2391 | | | +++ | | +++ | +++ | | |
| Hex7HexNAc6 | 2393 | | | −−− | | −−− | | | |
| Hex6HexNAc5dHex3 | 2466 | | | −−− | | −−− | | | |
| Hex7HexNAc6dHex | 2539 | +++ | | | | | | | |

TABLE 14
Differential effect of α2,3-sialidase treatment on isolated sialylated N-glycans from
cord blood CD133⁺ and CD133⁻ cells. The neutral N-glycan columns
show that neutral N-glycans corresponding to the listed sialylated N-glycans
appear in analysis of CD133⁺ cell N-glycans but not CD133⁻ cell
N-glycans. Proposed glycan compositions outside parenthesis are visible in the
neutral N-glycan fraction after α2,3-sialidase digestion of CD133⁺ cell
sialylated N-glycans.

| | Proposed monosaccharide | Sialylated N-glycan | | Neutral N-glycan | |
|---|---|---|---|---|---|
| m/z | composition | CD133⁺ | CD133⁻ | CD133⁺ | CD133⁻ |
| 1768 | (NeuAc₁)Hex₄HexNAc₄ | + | + | + | − |
| 2156 | (NeuAc₁)Hex₈HexNAc₂dHex₁/ | + | + | + | − |
| | (NeuAc₁Hex₅HexNAc₄dHex₁SO₃) | | | | |
| 2222 | (NeuAc₁)Hex₅HexNAc₄dHex₂ | + | + | + | − |
| 2238 | (NeuAc₁Hex₆HexNAc₄dHex₁/ | + | + | + | − |
| | (NeuGc₁)Hex₅HexNAc₄dHex₂ | | | | |
| 2254 | (NeuAc₁)Hex₇HexNAc₄/ | + | + | + | − |
| | (NeuGc₁)Hex₆HexNAc₄dHex₁ | | | | |
| 2368 | (NeuAc₁)Hex₅HexNAc₄dHex₃ | + | + | + | − |
| 2447 | (NeuAc₂)Hex₈HexNAc₂dHex₁/ | + | + | + | − |
| | (NeuAc₂Hex₅HexNAc₄dHex₁SO₃) | | | | |
| 2448 | (NeuAc₁)Hex₈HexNAc₂dHex₃/ | + | + | + | − |
| | (NeuAc₁Hex₅HexNAc₄dHex₃SO₃) | | | | |
| 2513 | (NeuAc₂)Hex₅HexNAc₄dHex₂ | + | + | + | − |
| 2733 | (NeuAc₁)Hex₆HexNAc₅dHex₃ | + | + | + | − |
| 2953 | (NeuAc₁)Hex7HexNAc₆dHex₂ | + | + | + | − |

TABLE 15

| | | | | | CB CD34+ | | BM & CB | |
|---|---|---|---|---|---|---|---|---|
| Trivial name | Terminal epitope | hESC 1) | EB | st.3 | & CD133+ | CB MNC | MSC | adipo/osteo |
| LN type 1, Le^{c} | Galβ3GlcNAc | N+ | 2) | +/− | q | N+/− | q | |
| | | O+ | +/− | | | O+/− | | |
| | | L++ | | | | L+ | | |
| Lea | Galβ3(Fucα4)GlcNAc | L+ | +/− | +/− | +/− | +/− | +/− | +/− |
| H type 1 | Fucα2Galβ3GlcNAc | L++ | +/− | +/− | +/− | +/− | +/− | +/− |
| Leb | Fucα2Galβ3(Fucα4)GlcNAc | + | +/− | +/− | +/− | +/− | +/− | +/− |
| sialyl Le^{a} | SAα3Galβ3(Fucα4)GlcNAc | | +/− | +/− | | | | |
| α3′-sialyl Le^{c} | SAα3Galβ3GlcNAc | | | | +/− | +/− | +/− | +/− |
| LN type 2 | Galβ4GlcNAc | N++ | + | + | N+ | N+ | N++ | N++ |
| | | O++ | | | O+ | O+ | O+ | |
| | | L+/− | | | | L+ | L++ | |
| Le^{x} | Galβ4(Fucα3)GlcNAc | N++ | +/− | +/− | N+ | N+/− | +/− | +/− |
| | | O+/− | | | O+ | O+ | | |
| | | L+/− | | | | L+/− | | |
| H type 2 | Fucα2Galβ4GlcNAc | N+ | +/− | +/− | N+ | +/− | +/− | +/− |
| | | O+/− | | | | | | |
| | | L+/− | | | | | | |
| Le^{y} | Fucα2Galβ4(Fucα3)GlcNAc | + | +/− | +/− | | | | |
| sialyl Le^{x} | SAα3Galβ4(Fucα3)GlcNAc | + | +/− | +/− | +/− | +/− | +/− | +/− |
| α3′-sialyl LN | SAα3Galβ4GlcNAc | N++ | N+ | N+ | N++ | N+ | N++ | N++ |
| | | O+ | | | O+ | O+ | O+ | |
| α6′-sialyl LN | SAα6Galβ4GlcNAc | N+ | N++ | N++ | N+ | N++ | +/− | |
| Core 1 | Galβ3GalNAcα | O+ | +/− | +/− | O+ | O+ | O+ | |
| H type 3 | Fucα2Galβ3GalNAcα | O+ | +/− | +/− | +/− | +/− | +/− | |
| sialyl Core 1 | SAα3Galβ3GalNAcα | O+ | | | O+ | O+ | O+ | |
| disialyl Core 1 | SAα3Galβ3Saα6GalNAcα | O+ | | | O+ | O+ | O+ | |
| type 4 chain | Galβ3GalNAcβ | L+ | +/− | +/− | +/− | L+ | L+ | |
| H type 4 | Fucα2Galβ3GalNAcβ | L+ | +/− | +/− | +/− | +/− | +/− | |
| α3′-sialyl type 4 | SAα3Galβ3GalNAcβ | L++ | +/− | +/− | +/− | +/− | +/− | |
| LecdiNAc | GalNAcβ4GlcNAc | N+ | +/− | +/− | +/− | +/− | +/− | +/− |
| Lac | Galβ4Glc | L+ | q | q | q | L+ | L+ | |
| GlcNAcβ | GlcNAcβ | N+/− | q | q | N+ | +/− | +/− | q |
| | | L+ | | | | | | |
| Tn | GalNAcα | q | | | q | q | O+ | |
| sialyl Tn | SAα6GalNAcα | | | | | | O+ | |
| GalNAcβ | GalNAcβ | L+ | q | q | +/− | +/− | N+/− | |
| | | N+ | | | | | L+ | |
| poly-LN, i | repeats of Galβ4GlcNAcβ3 | + | q | q | + | + | ++ | q |
| poly-LN, I | Galβ4GlcNAcβ3(Galβ4GlcNAcβ6)Gal | L+ | +/− | +/− | +/− | L+ | L+ | q |

1) Stem cell and differentiated cell types are abbreviated as in other parts of the present document; st.3 indicates stage 3 differentiated, preferentially neuronal-type differentiated cells; adipo/osteo indicates cells differentiated into adipocyte or osteoblast direction from MSC.
2) Occurrence of terminal epitopes in glycoconjugates and/or specifically in N-glycans (N), O-glycans (O), and/or glycosphingolipids (L). Code: q, qualitative data; +/−, low expression; +, common; ++, abundant.

TABLE 16

| | | Neutral | | | Sialylated | | |
|---|---|---|---|---|---|---|---|
| | | glycans | | | glycans | | |
| Class | Definition | hESC | MSC | CB MNC | hESC | MSC | CB MNC |
| Examples of | | | | | | | |
| glycosphingolipid glycan classification | | | | | | | |
| Lac | n_{Hex} = 2 | 1 | 1 | 2 | 1 | a) | |
| Ltri | n_{Hex} = 2 and n_{HexNAc} = 1 | 18 | 33 | 12 | 25 | | |
| L1 | n_{Hex} = 3 and n_{HexNAc} = 1 | 46 | 32 | 46 | 56 | | |
| L2 | 3 ≦ n_{Hex} ≦ 4 and n_{HexNAc} = 2 | 11 | 15 | 4 | <1 | | |
| L3+ | i + 1 ≦ n_{Hex} ≦ i + 2 and n_{HexNAc} = i ≧ 3 | 1 | 7 | 3 | 1 | | |
| Gb | n_{Hex} = 4 and n_{HexNAc} = 1 | 20 | 1 | 1 | 16 | | |
| O | other types | 23 | 11 | 34 | 1 | | |
| F | fucosylated, n_{dHex} ≧ 1 | 43 | 12 | 7 | 1 | | |
| T | non-reducing terminal HexNAc, | 27 | 47 | 12 | 26 | | |
| | n_{Hex} ≦ n_{HexNAc} + 1 | | | | | | |
| SA1 | monosialylated, n_{Neu5Ac} = 1 | | | | 86 | | |
| SA2 | disialylated, n_{Neu5Ac} = 2 | | | | 14 | | |
| SP | sulphated or phosphorylated, +80 Da | | | | <1 | | |
| Examples of O-linked glycan classification | | | | | | | |
| O1 | n_{Hex} = 1 and n_{HexNAc} = 1 | a) | a) | | 43 | a) | |
| O2 | n_{Hex} = 2 and n_{HexNAc} = 2 | 53 | | | 35 | | |
| O3+ | n_{Hex} = i and n_{HexNAc} = i ≧ 3 | 13 | | | 13 | | |
| O | other types | 34 | | | 9 | | |
| F | fucosylated, n_{dHex} ≧ 1 | 1 | 47 | 64 | 5 | 15 | 15 |

| T | non-reducing terminal HexNAc, | 12 | a) | <1 | a) |
|---|---|---|---|---|---|
| | n_{Hex} ≦ n_{HexNAc} + 1 | | | | |
| SA1 | monosialylated, n_{Neu5Ac} = 1 | | | 39 | |
| SA2 | disialylated, n_{Neu5Ac} = 2 | | | 52 | |
| SP | sulphated or phosphorylated, +80 Da | | | 8 | 21 |

a) not included in present quantitative analysis.

TABLE 17

| | CB | CB | |
|---|---|---|---|
| | MNC | MSC | hESC |
| Neutral | | | |
| glycosphingolipid | | | |
| glycans^{#} | | | |
| L1 | 1^{§} | 2 | 1 |
| L2 | 49 | 74 | 64 |
| L3 | 7 | 10 | 12 |
| L4 | 4 | 6 | 1 |
| L5+ | 2 | 0.5 | 0.5 |
| Gb | 0.5 | 0.5 | 20 |
| O | 37 | 8 | 2 |
| fucosylated | 11 | 8 | 43 |
| α1,2-Fuc | 11 | 6 | 39 |
| α1,3/4-Fuc | 6 | 2 | 3 |
| β1,4-Gal | 89 | 72 | 4 |
| β1,3-Gal | 48 | 68 | 50 |
| term. HexNAc | 10 | 27 | 27 |
| Acidic | | | |
| glycosphingolipid | | | |
| glycans^{#} | | | |
| L1 | 1^{§} | 10 | n.d. |
| L2 | 62 | 77 | 81 |
| L3 | 26 | 6 | 0.5 |
| L4 | 11 | 4 | 0.5 |
| L5+ | <0.5 | 0.5 | 0.5 |
| Gb | — | 0.5 | 16 |
| O | — | 2 | <0.5 |
| α-NeuAc | 100 | 100 | 100 |
| α2,3-NeuAc | 97 | 86 | 81 |
| fucosylated | 4 | 2 | 1 |
| β1,4-Gal | 97 | 32 | n.d. |

^{#}Abbreviations: L1-6, glycosphingolipid glycan type Li, wherein n_{HexNAc} + 1 ≦ n_{Hex} ≦ n_{HexNAc} + 2, and i = n_{HexNAc} + 1; Gb, (iso)globopentaose, wherein n_{Hex} = 4 and n_{HecNAc} = 1; term. HexNAc, terminal HexNAc in L1-6, wherein n_{HexNAc} + 1 = n_{Hex}; O, other types; n.d., not determined.
^{§}Figures indicate percentage of total detected glycan signals.

TABLE 18
Effect of sialylation, desialyation and fucosylation to viability and differentiation of blood stem cells.

| | | | | EXP 1 | EXP 2 |
|---|---|---|---|---|---|
| Condition | Buffer | Cell number | Incubation time | Viability (%) | Viability (%) |
| Original cell suspension | — | — | — | 92.7 | 86.5 |
| Control (Buffer) | HBSS - 1% HSA - 10 mM | 10 × 10⁶ | 60 min | 95.7 | 95.2 |
| Fucosyltransferase treatment | HBSS - 1% HSA - 10 mM | 10 × 10⁶ | 60 min | 97.1 | 92.5 |
| Sialylation treatment | HBSS - 1% HSA - 10 mM | 10 × 10⁶ | 60 min | 96.3 | 92.3 |
| Neuraminidase treatment | HBSS - 1% HSA - 10 mM | 10 × 10⁶ | 60 min | 96.9 | 95.7 |

| | EXP 1 | EXP 1 |
|---|---|---|
| Condition | CFU tot mean | CFU tot mean |
| Control (Buffer) | 82 | 137.5 |
| Fucosyltransferase | 58.5 | 138 |
| Sialylation | 75.5 | 110 |
| Neuraminidase | 109.5 | 196.5 |

TABLE 19
Enrichment of cells by binder coated magnetic particles

| | Bound | | | | |
|---|---|---|---|---|---|
| Lectin | cells | CD 34% | CD 133 | CD90 | CD3 + CD14 |
| GF707 PNA | 280,000 | 54.4 | 56.1 | 50.2 | |
| GF708 DBA | 255,000 | 60.2 | 71.6 | 59.1 | 59.6 |
| GF709 LTA | 85,000 | 60.4 | 75.0 | 58.8 | |
| GF710 MAA | 5,860,000 | | | | |
| GF711 NPA | 685,000 | 53.7 | | | |
| GF712 STA | 125,000 | 58.9 | | | |
| GF713 UEA | 227,500 | 46.2 | | | |
| Control, no | 425 | | | | |
| binder | | | | | |
| Control, no | | 13.5 | 3.4 | 9.1 | 63.7 |
| beads | | | | | |

TABLE 20
Lectin staining of cord blood hematopoietic stem cells (CB-HSCs, CD34+) and
mature blood cells (CD34−).

| | | | CB-HSC | |
|---|---|---|---|---|
| | | | CD34+ | CD34− |
| Lectin | Epitope | Structure | (% positive) | (% positive) |
| GNA | α-mannose | 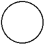 | 6.0 | 17.5 |
| HHA | α-mannose (internal + terminal) | 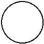 | 99.4 | 88.0 |
| NPA | α mannose | 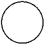 | 4.4 | 19.7 |
| PSA | terminal α-D-mannosyl | 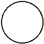 | 100.0 | 96.6 |
| PNA | Gal(β3)GalNAc | 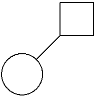 | 7.7 | 4.3 |
| MAA | SAα(2,3)Gal | 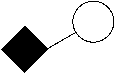 | 84.4 | 46.4 |
| SNA | SAα(2,6)Gal/GalNAc | 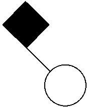 | 99.4 | 83.4 |
| PWA | branched Gal(β4)GlcNAc oligomers, polyLN (I) | 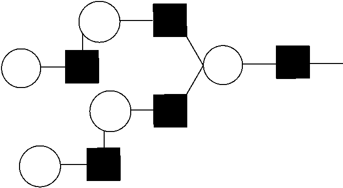 | 2.9 | 1.5 |
| STA | linear Gal(β4)GlcNAc oligomers, polyLN (i) | 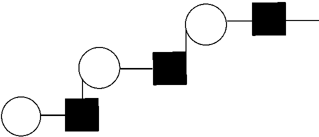 | 66.3 | 4.8 |
| LTA | Lewis x | 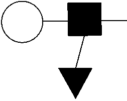 | 0.3 | 0.1 |
| UEA | H type 2 | 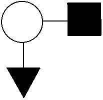 | 9.2 | 17.7 |
| CCA | O-acetyl sialic acid | | 3.8 | 0.7 |

TABLE 21
Flow cytometric (FACS) analysis of cord blood hematopoietic stem cells (CB-HSCs,
CD34+) and mature blood cells (CD34−).

| | | | | CB-HSC | | CB-HSC | |
|---|---|---|---|---|---|---|---|
| Code | Trivial name | Structure | Terminal epitope | CD34+ | SD | CD34− | SD |
| GF 416 | Mannose | 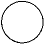 | Man | 6.0 | 1.1 | 7.7 | 2.3 |
| GF 278 | Tn | 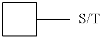 | GalNAcαS/T | 36.6 | 11.0 | 12.8 | 0.5 |
| VPU 006 | Tn antigen, CD175 | 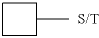 | GalNAcαS/T | 36.5 | | 12.6 | |
| VPU 007 | sialyl Tn, sCD175 | 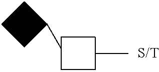 | SA(α6)GalNAcαS/T | 3.8 | | 3.2 | |
| GF 277 | Sialosyl-Tn | 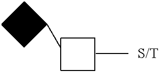 | SA(α6)GalNAcαS/T | 4.7 | 1.9 | 10.7 | 1.8 |
| GF 276 | TAG-72, CA 72-4 | 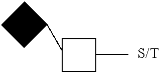 | | 11.7 | 4.4 | 7.6 | 2.8 |
| GF 280 | TF-antigen | 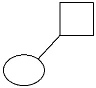 | Gal(β3)GalNAc(α/β) | 19.1 | 12.1 | 7.0 | 1.0 |
| GF 281 | TF-antigen | 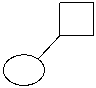 | Gal(β3)GalNAc(α/β) | 40.2 | 6.8 | 11.1 | 1.9 |
| GF 365 | TF-antigen | 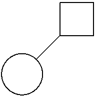 | Gal(β3)GalNAc(α/β) | 18.6 | 12.4 | 7.2 | 0.5 |
| GF 274 | MECA-79, Sulfo- mucin, PNAD | 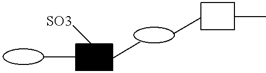 | Sulfo-mucin | 14.6 | 14.0 | 18.4 | 0.1 |
| GF 374 | Glycodelin A | 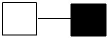 | LacdiNAc | 9.0 | 3.9 | 14.2 | 1.6 |
| GF 375 | Glycodelin A | 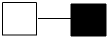 | LacdiNAc | 18.3 | 15.5 | 15.4 | 3.2 |
| GF 376 | Glycodelin A | 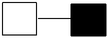 | LacdiNAc | 18.5 | 11.0 | 11.5 | 0.8 |
| GF 413 | Gal(α3)Gal | 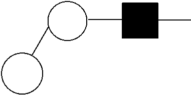 | Gal(α3)Gal | 7.3 | 2.8 | 4.1 | 1.5 |
| GF 295 | Lewis c | 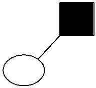 | Gal(β3)GlcNAcβ(3Lac) | 13.2 | 1.4 | 19.6 | 8.2 |
| GF 300 GF 428 | asialo GM2 | 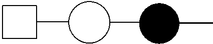 | GalNAc(β4)Gal(β4)GlcβCer | 10.0 | 10.1 | 32.4 | 11.2 |
| GF 296 GF 427 | asialo GM1 | 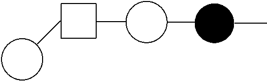 | Gal(β3)GalNAc(β4)Gal(β4) GlcβCer | 11.1 | 12.5 | 30.8 | 7.7 |
| GF 406 | GD2 | 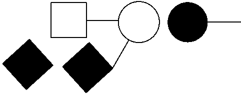 | | 4.5 | | 5.0 | |
| GF 298 | Gb3 | 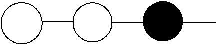 | Gal(α4)Gal(β4)GlcβCer | 11.3 | 4.7 | 18.5 | 0.7 |
| GF 297 VPU 001 | Globoside GL4 | 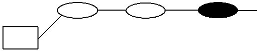 | GalNAc(β3)Gal(α4)Gal(β4) GlcβCer | 6.0 | 2.4 | 23.2 | 6.5 |
| GF 353 | SSEA-3 | 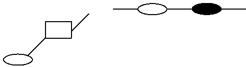 | Gal(β3)GalNAc(β3)Gal | 5.9 | 1.2 | 20.1 | 0.7 |
| GF 354 | SSEA-4 | 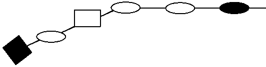 | SA(α3)Gal(β3)GalNAc(β3)Gal | 4.9 | 1.6 | 15.1 | 7.0 |
| GF 299 | Forssman ag | 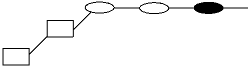 | GalNAc(α3)GalNAc(β4)Gal (α4)Gal(β4)GlcβCer, GalNAc(α3)GalNAcβ-R | 9.1 | 6.4 | 20.2 | 3.2 |
| GF 288 | Globo-H | 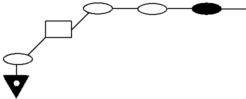 | Fuc(α2)Gal(β3)GalNAc(β3) Gal(α4)Gal(β4)GlcβCer | 10.6 | 2.8 | 5.4 | 1.5 |
| GF 394 | H disaccharide | 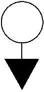 | Fuc(α2)Galβ | 10.1 | 4.8 | 5.7 | 1.6 |
| GF 303 | H Type 1 | 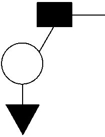 | Fuc(α2)Gal(β3)GlcNAc | 4.7 | 1.3 | 13.7 | 2.0 |
| GF 304 | Lewis a | 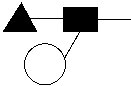 | Gal(β3)[Fuc(β4)]GlcNAc | 11.2 | 3.8 | 18.1 | 4.3 |
| GF 306 | sialyl Lewis a | 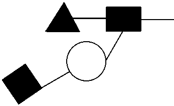 | SA(α3)Gal(β3)[Fuc(α4)] GlcNAc | 6.4 | 2.9 | 18.1 | 7.3 |
| GF 301 | Lewis b | 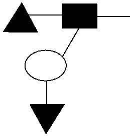 | Fuc(α2)Gal(β3)[Fuc(α4)] GlcNAc | 7.3 | | 19.3 | |
| GF 302 | H Type 2 | 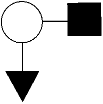 | Fuc(α2)Gal(β4)GlcNAc | 6.2 | 3.5 | 19.1 | 2.6 |
| GF 410 | blood group ABH | 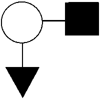 | | 8.9 | 5.4 | 7.8 | 1.1 |
| GF 305 | Lewis x | 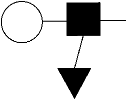 | Gal(β4)[Fuc(α3)]GlcNAc | 26.9 | 21.7 | 6.9 | 3.9 |
| GF 515 | Lex, CD15 | 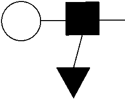 | Gal(β4)[Fuc(α3)]GlcNAc | 8.8 | 11.4 | 13.2 | 7.6 |
| GF 517 | Lex, CD15 | 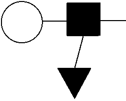 | Gal(β4)[Fuc(α3)]GlcNAc | 28.7 | 31.5 | 4.2 | 2.1 |
| GF 518 | SSEA-1 (CD15, Lex) | 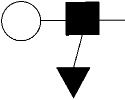 | Gal(β4)[Fuc(α3)]GlcNAc | 22.7 | 29.2 | 6.7 | 3.0 |
| GF 525 | CD15 (Lex) | 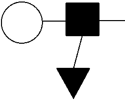 | Gal(β4)[Fuc(α3)]GlcNAc | 14.3 | 16.1 | 13.8 | 3.1 |
| GF 516 | sLex, sCD15 | 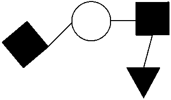 | SA(α3)Gal(β4)[Fuc(α3)] GlcNAc | 43.4 | 15.1 | 5.9 | 3.6 |
| GF 307 | sialyl Lewis x | 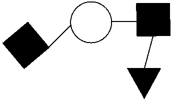 | SA(α3)Gal(β4)[Fuc(α3)] GlcNAc | 85.5 | 6.3 | 13.7 | 3.4 |
| GF 526 | PSGL-1 (sLex on core 2 O-glycans) | 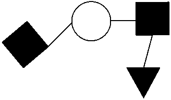 | SA(α3)Gal(β4)[Fuc(α3)] GlcNAc | 97.6 | 0.4 | 33.7 | 11.1 |
| GF 393 | Lewis y | 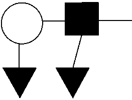 | Fuc(α2)Gal(β4)[Fuc(α3)] GlcNAcβ | 5.5 | 1.3 | 7.1 | 1.9 |
| GF 408 | blood group Ag A-b45.1 | 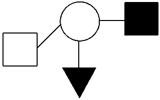 | GalNAc(α3)Fuc(α2)Galβ | 5.1 | 2.3 | 17.1 | 4.6 |
| GF 409 | blood group A | 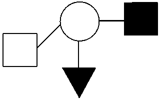 | | 6.8 | 3.0 | 8.0 | 1.0 |
| GF 411 | blood group B (secretor) | 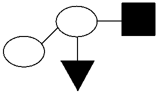 | | 5.9 | 1.9 | 8.7 | 2.3 |
| GF 412 | blood group B (general) | 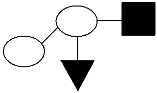 | | 8.0 | 5.8 | 6.9 | 1.0 |
| GF 414 | TRA-1-81 Ag | | | 6.1 | | 9.7 | |
| GF 415 | TRA-1-60 Ag | | | 11.2 | | 5.6 | |

TABLE 22
Detailed information of the primary anti-glycan antibodies used in these examples.
Alternative antibody clones in italics.

| Code | Epitope | Terminal structure | Company | Cat number | Clone | Host/Class |
|---|---|---|---|---|---|---|
| GF 274 | Sulfo-mucin, PNAD, | Sulfo-mucin | BD | 553863 | MECA-79 | rat/IgM |
| | MECA-79, CD62L, | | Pharmingen | | | |
| | extended core 1 | | | | | |
| GF 275 | Ca15-3 sialyted | SAα-mucin | Acris | BM3359 | 695 | mouse/IgG1 |
| GF 553 | epitope | | | | | |
| GF 276 | TAG-72, CA 72-4, | | Acris | DM288 | B72.3 | mouse/IgG1 |
| | cancer glycoprotein | | | | | |
| GF 277 | Sialosyl-Tn, sCD175 | SA(α6)GalNAcαS/T | Acris | DM3197 | B35.1 | mouse/IgG1 |
| GF 372 | | | | | | |
| GF 278 | Tn, CD175 | GalNAcαS/T | Acris | DM3218 | B1.1 | mouse/IgM |
| VPU008 | | | | | | |
| GF 280 | TF-antigen isoform, | Gal(β3)GalNAc(α/β) (α40x > β) | Glycotope | MAB-S301 | Nemod | mouse/IgM |
| | CD176 | | | | TF2 | |
| GF 281 | TF-antigen isoform, | Gal(β3)GalNAcβ | Glycotope | MAB-S305 | A68-E/E3 | mouse/IgG1 |
| | CD176 | | | | | |
| GF 285 | H Type 2, Lewis b, | Fuc(α2)Gal, Fuc(a2)Gal(β4)GlcNAc, | Acris | DM3014 | B389 | mouse/IgG1 |
| | Lewis y | Fuc(α2)Gal(β4)[Fuc(α3)]GlcNAc | | | | |
| GF 286 | H Type 2, CD173 | Fuc(α2)Gal(β4)GlcNAc | Acris | BM258P | BRIC 231 | mouse/IgG1 |
| GF 288 | Globo-H | Fuc(α2)Gal(β3)GalNAc(β3)Gal(α4)Gal(β4)GlcβCer | Glycotope | MAB-S206 | A69-A/E8 | mouse/IgM |
| GF 403 | | | | | | |
| GF 295, | Lewis c, pLN, | Gal(β3)GlcNAcβ(3Lac) | Abcam | ab3352 | K21 | mouse/IgM |
| GF 279 | Gal(β3)GlcNAc | | | | | |
| GF 555 | | | | | | |
| GF 296, | asialo GM1 | Gal(β3)GalNAc(β4)Gal(β4)GlcβCer | Acris | BP282 | polyclonal | rabbit |
| GF 282 | | | | | | |
| GF 427 | | | | | | |
| GF 297, | Globoside Gb4, GL4, | GalNAc(β3)Gal(α4)Gal(β4)GlcβCer | Abcam | ab23949 | polyclonal | rabbit/IgG |
| GF 366 | globotetraose | | | | | |
| VPU001 | | | | | | |
| GF 298 | Globoside Gb3, | Gal(α4)Gal(β4)GlcβCer | Acris | SM1160P | 38-13 | rat/IgM |
| GF 367 | globotriose, CD77, | | | | | |
| | blood group pk | | | | | |
| GF 299, | Forssman ag, | GalNAc(α3)GalNAc(β4)Gal(α4)Gal(β4)GlcβCer, | Acris | BM4091 | FOM-1 | rat/IgM |
| GF 401 | glycosphingolipid | GalNAc(α3)GalNAcβ-R | | | | |
| GF 554 | | | | | | |
| GF 300 | asialo GM2 | GalNAc(β4)Gal(β4)GlcβCer | Acris | BP283 | polyclonal | rabbit |
| GF 428 | | | | | | |
| GF 301, | Lewis b | Fuc(α2)Gal(β3)[Fuc(α4)]GlcNAc | Acris | SM3092P | 2-25LE | mouse/IgG1 |
| GF 283 | | | | DM3122 | | |
| VPU004 | | | | | | |
| GF 302 | H Type 2 | Fuc(α2)Gal(β4)GlcNAc | Acris | DM3015 | B393 | mouse/IgM |
| GF 284 | | | | | | |
| GF 303 | H Type 1, blood group | Fuc(α2)Gal(β3)GlcNAc | Abcam | ab3355 | 17-206 | mouse/IgG3 |
| GF 287 | antigen H1 | | | | | |
| GF 304 | Lewis a | Gal(β3)[Fuc(α4)]GlcNAc | Chemicon | CBL205 | PR5C5 | mouse/IgG1 |
| GF 429 | | | Abcam | Ab3967 | 7LE | |
| | | | | Ab3356 | T174 | |
| | | | Genetex | GTX28602 | B369 | |
| GF 305 | Lewis x, CD15, | Gal(β4)[Fuc(α3)]GlcNAc | Chemicon | CBL144 | 28 | mouse/IgM |
| | SSEA-1 | | | | | |
| GF 306, | sialyl Lewis a | SA(α3)Gal(β3)[Fuc(α4)]GlcNAc | Chemicon | MAB2095 | KM231 | mouse/IgG1 |
| GF 430 | | | Invitrogen | 18-7240 | 116-NS- | |
| VPU002 | | | | | 19-9 | |
| | | | BioGenex | MU424-UC | C241:5:1:4 | |
| | sialyl Lewis a, c | | Seikagaku | 270443 | 2D3 | mouse/IgM |
| GF 307 | sialyl Lewis x | SA(α3)Gal(β4)[Fuc(α3)]GlcNAc | Chemicon | MAB2096 | KM93 | mouse/IgM |
| GF 353 | SSEA-3, | Gal(β3)GalNAc(β3)Gal | Chemicon | MAB4303 | MC-631 | rat/IgM |
| GF 431 | galactosylgloboside | | | | | |
| GF 354, | SSEA-4, | SA(α3)Gal(β3)GalNAc(β3)Gal | Chemicon | MAB4304 | MC-813- | mouse/IgG3 |
| GF 432 | sialyl- | | | | 70 | |
| VPU003 | galactosylgloboside | | | | | |
| GF 355 | Gal(α3)Gal | Gal(α3)Gal | Chemicon | AB2052 | | baboon |
| GF 365 | TF-antigen isoform, | Gal(β3)GalNAc(α/β) (α10x > β) | Glycotope | MAB-S302 | Nemod | mouse/IgM |
| | CD176 | | | | TF1 | |
| GF 368 | LacdiNAc | GalNAc(β4)GlcNAc | LUMC | anti-LDN | 259-2A1 | IgG3 |
| | | | (Leiden Univ | mAb | | |
| | | | Medical | | | |
| | | | Center) | | | |
| GF 369 | LacdiNAc | GalNAc(β4)GlcNAc | LUMC | anti-LDN | 273-3F2 | IgM |
| | | | (Leiden Univ | mAb | | |
| | | | Medical | | | |
| | | | Center) | | | |
| GF 370 | α3-Fuc-LacdiNAc | GalNAc(β4)[Fuc(α3)]GlcNAc | LUMC | anti LDN-F | 290-2E6 | IgM |
| | | | (Leiden Univ | mAb | | |
| | | | Medical | | | |
| | | | Center) | | | |
| GF 371 | α3-Fuc-LacdiNAc | GalNAc(β4)[Fuc(α3)]GlcNAc | LUMC | anti LDN-F | 291-3E9 | IgM |
| | | | (Leiden Univ | mAb | | |
| | | | Medical | | | |
| | | | Center) | | | |
| GF 374 | Glycodelin A, isoform | LacdiNAc | Glycotope | MAB-S901 | A87-D/C5 | mouse/IgG1, |
| | | | | | | IgG2b, IgM |
| GF 375 | Glycodelin A, isoform | LacdiNAc | Glycotope | MAB-S902 | A87-D/F4 | mouse/IgG1 |
| GF 376 | Glycodelin A, isoform | LacdiNAc | Glycotope | MAB-S903 | A87-B/D2 | mouse/IgG1 |
| GF 377 | PN-15 renal gp200, | | Acris | DM3184P | PN-15 | mouse/IgG1 |
| GF 373 | cancer glycoprotein | | | | | |
| GF 393 | Lewis y, CD174 | Fuc(α2)Gal(β4)[Fuc(α3)]GlcNAcβ | Glycotope | MAB-S201 | A70-C/C8 | mouse/IgM |
| GF 289 | | | | | | |
| GF 394 | H disaccharide | Fuc(α2)Galβ | Glycotope | MAB-S204 | A51-B/A6 | mouse/IgA |
| GF 290 | | | | | | |
| GF 406 | GD2 | GalNAc(β4)(SA(α8)SA)(α3)Gal(β4)Glc | Chemicon | MAB4309 | VIN-2PB- | mouse/IgM |
| GF 558 | | | | | 22 | |
| GF 407 | GD3 | SA(α8)SA(α3)Gal(β4)Glc | Chemicon | MAB4308 | VIN-IS-56 | mouse/IgM |
| GF 559 | | | | | | |
| GF 408 | blood group Ag | GalNAc(α3)Fuc(α2)Galβ | Acris | DM3108 | B480 | mouse/IgG1 |
| | A-b45.1 (A1, A2) | | | | | |
| GF 409 | blood group A | | Acris | BM255 | HE-195 | mouse/IgM |
| | (A3, Ax, A3B, AxB) | | | | | |
| GF 410 | blood group ABH | | Acris | SM3004 | HE-10 | mouse/IgM |
| GF 411 | blood group B | | Acris | BM256 | HEB-29 | mouse/IgM |
| | (secretor) | | | | | |
| GF 412 | blood group Ag B | | Acris | DM3012 | B460 | mouse/IgM |
| | (general) | | | | | |
| GF 413 | Gal(α3)Gal | Gal(α3)Gal(β4)GlcNAc-R | Alexis | ALX-801- | M86 | mouse/IgM |
| | | | Biochemicals | 090 | | |
| GF 414 | TRA-1-81 Ag | | Chemicon | MAB4381 | TRA-1-81 | mouse/IgM |
| GF 556 | | | | | | |
| GF 415 | TRA-1-60 Ag | | Chemicon | MAB4360 | TRA-1-60 | mouse/IgM |
| GF 557 | | | | | | |
| GF 416 | Mannose | Man | | | | mouse/IgM |
| GF 418 | Globo-H | Fuc(α2)Gal(β3)GalNAc(β3)Gal(α4)Gal(β4)GlcβCer | Alexis | ALX-804- | MBr1 | mouse/IgM |
| | | | biochemicals | 550-C050 | | |
| GF 515 | CD15, Lewis x | Gal(β4)[Fuc(α3)]GlcNAc | BD | 557895 | W6D3 | mouse/IgG1, |
| | | | Pharmingen | | | k |
| GF 516 | sCD15, sialyl Lewis x | SA(α3)Gal(β4)[Fuc(α3)]GlcNAc | BD | 551344 | CSLEX1 | mouse/IgM, |
| | | | Pharmingen | | | k |
| GF 517 | CD15, Lewis x | Gal(β4)[Fuc(α3)]GlcNAc | Abcam | ab34200 | TG-1 | mouse/IgM |
| GF 518 | SSEA-1 | Gal(β4)[Fuc(α3)]GlcNAc | Abcam | ab16285 | MC480 | mouse/IgM |
| GF 525 | CD15, reacts with 220 | Gal(β4)[Fuc(α3)]GlcNAc | Abcam | ab17080 | MMA | mouse/IgM |
| | kD protein | | | | | |
| GF 526 | PSGL-1, sLex on core | SA(α3)Gal(β4)[Fuc(α3)]GlcNAc | R&D | MAB996 | CHO131 | mouse/IgM |
| | 2 O-glycans | | Systems | | | |
| GF 621 | GD3 | SA(α8)SA(α3)Gal(β4)Glc | BD | 554274 | MB3.6 | mouse/IgG3 |
| | | | Pharmingen | | | |
| GF 622 | GD2 | GalNAc(β4)(SA(α8)SA)(α3)Gal(β4)Glc | BD | 554272 | 14.G2a | mouse/IgG2 |
| | | | Pharmingen | | | |
| GF 623 | GT1b | | US Biological | G2006-90A | 3C96 | mouse/IgM |
| GF 624 | GD1b | | US Biological | G2004-90B | 2S1 | mouse/IgG3 |
| GF 625 | GD2 | GalNAc(β4)(SA(α8)SA)(α3)Gal(β4)Glc | US Biological | G2205-02 | 2Q549 | mouse/IgG2 |
| GF 626 | GD3 | SA(α8)SA(α3)Gal(β4)Glc | Covalab | mab0014 | 4F6 | mouse/IgG3 |
| GF 627 | OAcGD3 | | US Biological | G2005-67 | 4i283 | mouse/IgG3 |
| GF 628 | A2B5 | | Chemicon | MAB312R | A2B5-105 | mouse/IgM |
| VPU005 | GD3 | SA(α8)SA(α3)Gal | Seikagaku | 270554 | S2-566 | mouse/IgM |
| VPU006 | Tn antigen, CD175 | GalNAcαS/T | Abcam | ab31775 | 0.BG.12 | mouse/IgG |
| VPU007 | sialyl Tn, sCD175 | SA(α6)GalNAcαS/T | Abcam | ab24005 | BRIC111 | mouse/IgG |
| VPU009 | SSEA-3, | Gal(β3)GalNAc(β3)Gal | R&D | MAB1434 | MC-631 | rat/IgM |
| | galactosylgloboside | | Systems | | | |
| | GlcNAcβ1-6R | | Jeffersson | FE-J1 | | mouse/IgM |
| | | | medical | | | |
| | | | college | | | |
| | Galβ1-4GlcNAcβ1-3R | | Jeffersson | FE-A5 | | mouse/IgM |
| | | | medical | | | |
| | | | college | | | |
| | Galβ1-4GlcNAcβ1-6R | | Jeffersson | FE-A6 | | mouse/IgM |
| | | | medical | | | |
| | | | college | | | |

TABLE 23
HSC binder target table based on structural analyses and binder specificities.
See explanation of terms in footnotes 1) and 2).

| | | CD34+, | CD34−, |
|---|---|---|---|
| Trivial name | Terminal epitope | CD133+ | CD133− |
| LN type 1, Lec | Galβ3GlcNAcβ | +/− | +/− |
| | | L+ | L+ |
| | Lecβ3Galβ4Glc[NAc]β | +/− | +/− |
| Lea | Galβ3(Fucα4)GlcNAcβ | +/− | +/− |
| | | L+ | L+ |
| | Leaβ3Galβ4Glc[NAc]β | +/− | +/− |
| H type 1, H1 | Fucα2Galβ3GlcNAc | | |
| | H1β3Galβ4Glc[NAc]β | | |
| Leb | Fucα2Galβ3(Fucα4)GlcNAcβ | q | q |
| sialyl Lea, sLea | SAα3Galβ3(Fucα4)GlcNAcβ | +/− | + |
| | | Lq | Lq |
| α3′-sialyl Lec | SAα3Galβ3GlcNAcβ | q | q |
| LN type 2, LN | Galβ4GlcNAc | + | + |
| | | N+ | N+ |
| | | O+ | O+ |
| | | Lq | Lq |
| | LNβ2Manα3/6 | + | + |
| | LNβ4Manα3 | +/− | + |
| | LNβ2Manα3(LNβ2Manα6)Man | + | + |
| | LNβ2(LNβ4)Manα3(LNβ2Manα6)Man | q | +/− |
| | LNβ6(R-Galβ3)GalNAc | + | + |
| | LNβ3Galβ4Glc[NAc]β | q | q |
| | LNβ6(R-GlcNAcβ3)Galβ4Glc[NAc]β | q | q |
| | LNβ3(R-GlcNAcβ6)Galβ4Glc[NAc]β | q | q |
| | LNβ3(LNβ6)Galβ4Glc[NAc]β | q | q |
| Lex | Galβ4(Fucα3)GlcNAc | + | + |
| | | Nq | Nq |
| | | Oq | Oq |
| | | Lq | Lq |
| | Lexβ2Manα3/6 | +/− | +/− |
| | Lexβ6(R-Galβ3)GalNAc | + | q |
| | Lexβ3Galβ4Glc[NAc]β | q | q |
| | Lexβ2Manα3(Lexβ2Manα6)Man | q | q |
| H type 2, H2 | Fucα2Galβ4GlcNAc | +/− | + |
| | | Nq | Nq |
| | H2β2Manα3/6 | q | q |
| | H2β3Galβ4Glc[NAc]β | q | q |
| Ley | Fucα2Galβ4(Fucα3)GlcNAc | +/− | +/− |
| | | Lq | Lq |
| | Leyβ3Galβ4Glc[NAc]β | q | q |
| sialyl Lex, sLex | SAα3Galβ4(Fucα3)GlcNAc | ++ | + |
| | | Nq | Nq |
| | | O++ | O+ |
| | | Lq | Lq |
| | sLexβ2Manα3/6 | q | q |
| | sLexβ6(R-Galβ3)GalNAc | ++ | + |
| | sLexβ3Galβ4Glc[NAc]β | q | q |
| α3′-sialyl LN, | SAα3Galβ4GlcNAc | ++ | + |
| s3LN | | N++ | N+ |
| | | O+ | O+ |
| | | Lq | Lq |
| | s3LNβ2Manα3/6 | ++ | + |
| | s3LNβ4Manα3 | +/− | + |
| | s3LNβ2Manα3(s3LNβ2Manα6)Man | ++ | + |
| | s3LNβ6(R-Galβ3)GalNAc | + | + |
| | s3LNβ3Galβ4Glc[NAc]β | q | q |
| | s3LNβ6(R-GlcNAcβ3)Galβ4Glc[NAc]β | q | q |
| | s3LNβ3(R-GlcNAcβ6)Galβ4Glc[NAc]β | q | q |
| α6′-sialyl LN, | SAα3Galβ4GlcNAc | q | q |
| s6LN | | Nq | Nq |
| | s6LNβ2Manα3/6 | q | q |
| | s6LNβ4Manα3 | q | q |
| | s6LNβ2Manα3(s6LNβ2Manα6)Man | q | q |
| | s6LNβ3Galβ4Glc[NAc]β | q | q |
| Core 1 | Galβ3GalNAcα | + | +/− |
| H type 3 | Fucα2Galβ3GalNAcα | − | − |
| sialyl Core 1 | SAα3Galβ3GalNAcα | + | + |
| disialyl Core 1 | SAα3Galβ3Saα6GalNAcα | + | + |
| type 4 chain | Galβ3GalNAcβ | +/− | + |
| | | L+ | L+ |
| asialo-GM1 | Galβ3GalNAcβ4Galβ4Glc | +/− | + |
| Gb5, “SSEA-3” | Galβ3GalNAcβ3Galα4Galβ4Glc | +/− | + |
| H type4, “Globo H” | Fucα2Galβ3GalNAcβ | +/− | q |
| α3′-sialyl type 4 | SAα3Galβ3GalNAcβ | q | q |
| | | L+ | L+ |
| “SSEA-4” | SAα3Galβ3GalNAcβ3Galα4Galβ4Glc | +/− | + |
| GalNAcβ | GalNAcβ | +/− | + |
| asialo-GM2 | GalNAcβ4Galβ4Glc | +/− | + |
| Gb4 | GalNAcβ3Galα4Galβ4Glc | +/− | + |
| LacdiNAc | GalNAcβ4GlcNAc | | |
| Galα | Galβ4Glc | +/− | + |
| Gb3 | Galα4Galβ4Glc | +/− | + |
| Lac | Galβ4Glc | q | q |
| GalNAcα, “Tn” | GalNAcα | +/− | q |
| Forssman | GalNAcα3GalNAcβ | +/− | + |
| sialyl Tn | SAα6GalNAcα | q | +/− |
| oligosialic acid | NeuAcα8NeuAcα | q | q |
| | | Lq | Lq |
| GD3 | NeuAcα8NeuAcα2Galβ4Glc | | |
| GD2 | NeuAcα8NeuAcα2(GalNAcβ4)Galβ4Glc | q | q |
| GD1b | NeuAcα8NeuAcα2(Galβ3GalNAcβ4)Galβ4Glc | | |
| GT1b | SAα8SAα2(Saα3Galβ3GalNAcβ4)Galβ4Glc | | |
| Manα | Manα | ++ | ++ |
| | Manα2Manα | ++ | + |
| | Manα3Manα6/β4 | + | ++ |
| | Manα6Manα6/β4 | + | ++ |
| | Manα3(Manα6)Manα6/β6 | + | ++ |
| | Manα3(Manα6)Manβ4GlcNAc[β4GlcNAc] | +/− | +/− |
| Manβ | Manβ | +/− | +/− |
| | Manβ4GlcNAc | +/− | +/− |
| Glcα | Glcα | + | +/− |
| | Glcα3Manα | + | +/− |
| | Glcα2Glcα3[Glcα3Manα] | +/− | +/− |
| core-Fuc | Fucα6GlcNAc | N+ | N+/− |
| | Fucα6(R-GlcNAcβ4)GlcNAc | + | +/− |
| GlcNAcβ, Gn | GlcNAcβ | + | +/− |
| | | N+ | Nq |
| | Gnβ2Manα3/6 | + | q |
| | Gnβ4Manα3 | + | q |
| | Gnβ2Manα3(Gnβ2Manα6)Man | + | q |
| | Gnβ4Gn | q | q |
| | Gnβ4(Fucα6)Gn | q | q |
| | Gnβ3Galβ4Glc[NAc]β | q | q |
| | Gnβ6(R-GlcNAcβ3)Galβ4Glc[NAc]β | q | q |
| | Gnβ3(R-GlcNAcβ6)Galβ4Glc[NAc]β | q | q |

1) Stem cell and differentiated cell types are abbreviated as in other parts of the present document; CD34+/CD133+ indicates HSC derived from cord blood, peripheral blood, or bone marrow; CD34−/CD133− indicates differentiated cells from the same source MNC fraction.
2) Occurrence of terminal epitopes in glycoconjugates and/or specifically in N-glycans (N), O-glycans (O), and/or glycosphingolipids (L). Code: q, qualitative data; +/−, low expression; +, common; ++, abundant.

### REFERENCES

Altmann, F., et al. (1999) Glycoconj. J. 16:109-23
Harvey, D. J., et al. (1993) Rapid Commun. Mass Spectrum. 7(7):614-9
Hirabayashi, J., et al. (2002) Biochim. Biophys. Acta. 1572:232-54.
Jaatinen, T., et al. (2006) Stem cells. 24:631-41.
Karlsson, H., et al. (2000) Glycobiology 10(12):1291-309
Kretzchmar, E., et al. (1994) Biol. Chem. Hoppe Seyler 375(5):23-7
Kubelka, V., et al. (1994) Arch. Biochem. Biophys. 308(1):148-57
Leskela, H., et al. (2003) Biochem. Biophys. Res. Commun. 311:1008-13
Miller-Podraza, H., et al. (2000) Glycobiologyy. 10:975-982
Moore (1999) Trends Cell Biol. 9:441-6
Naven, T. J. & Harvey, D. J. (1996) Rapid Commun. Mass Spectrom. 10(11): 1361-6
Nyman, T. A., et al. (1998) Eur. J. Biochem. 253(2):485-93
Papac, D., et al. (1996) Anal. Chem. 68(18):3215-23
Saarinen, J., et al. (1999) Eur. J. Biochem. 259(3):829-40
Skottman, H. et al. (2005) Stem cells
Staudacher, E., et al. (1992) Eur. J. Biochem. 207(3):987-93
Thomson, J. A., et al. (1998) Science 282:1145-7
Venable et al. (2005) BMC Developmental biology.

1-82. (canceled)

83. A method of evaluating the status of a hematopoietic stem cell preparation comprising the step of detecting the presence of an elongated glycan structure or a group, at least two, of glycan structures in said preparation, wherein said glycan structure or a group of glycan structures is according to Formula T1 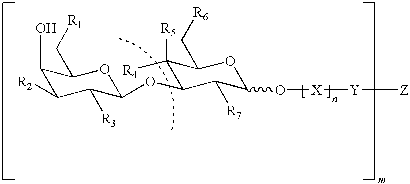
wherein
R₁, R₂, and R₆ are OH or glycosidically linked monosaccharide residue sialic acid, preferably Neu5Acα2 or Neu5Gc α2, most preferably Neu5Acα2 or
R₃, is OH or glycosidically linked monosaccharide residue Fuccl (L-fucose) or N-acetyl (N-acetamido, NCOCH₃);
R₄, is H, OH or glycosidically linked monosaccharide residue Fucα1 (L-fucose),
R₅ is OH, when R₄ is H, and R₅ is H, when R₄ is not H;
R7 is N-acetyl or OH;
X is natural oligosaccharide backbone structure from the cells, preferably N-glycan, O-glycan or glycolipid structure; or X is nothing, when n is 0,
Y is linker group preferably oxygen for O-glycans and O-linked terminal oligosaccharides and glycolipids and N for N-glycans or nothing when n is 0;
Z is the carrier structure, preferably natural carrier produced by the cells, such as protein or lipid, which is preferably a ceramide or branched glycan core structure on the carrier or H;
the arch indicates that the linkage from the galactopyranosyl is either to position 3 or to position 4 of the residue on the left and that the R4 structure is in the other position 4 or 3;
n is an integer 0 or 1, and m is an integer from 1 to 1000, preferably 1 to 100, and most preferably 1 to 10 (the number of the glycans on the carrier),
with the provisions that one of R2 and R3 is OH or R3 is N-acetyl,
R6 is OH, when the first residue on left is linked to position 4 of the residue on right: and
the glycan structure is an elongated structure, wherein the binder binds to the structure and additionally to at least one reducing end elongation epitope, which is a monosaccharide epitope replacing X or being a part of X, said monosaccharide epitope being according to Formula E1: AxHex(NAc)ₙ, wherein A is anomeric structure alfa or beta, x is linkage position 2, 3, or 6; and Hex is hexopyranosyl residue Gal, or Man, and n is integer being 0 or 1,
with the provisions that
when n is 1 then AxHexNAc is β4GalNAc or β6GalNAc,
when Hex is Man, then AxHex is β2Man, and
when Hex is Gal, then AxHex is β3Gal or β6Gal or α3Gal or α4Gal; or
the binder epitope binds additionally to reducing end elongation epitope
Ser/Thr linked to reducing end GalNAcα-comprising structures or
βCer linked to Galβ4Glc comprising structures, and the glycan structure is the stem cell population determined structure or from associated or contaminating cell population,
and optionally wherein the structure is used together with at least one terminal ManαMan-structure.
with the provisions that
i) the hematopoietic stem cells are not cells of a cancer cell line and
ii) if cells are hematopoietic CD34⁺ cells and the structure is comprises N-acetyllactosamine it is specific elongated structure being fucosylated or not SAα3Galβ4GlcNAcβ3Gal structure.

84. The method according to claim 83 wherein terminal epitope selected from the group Galβ4GlcNAc, Galβ4(Fucα3)GlcNAc, Fucα2Galβ4GlcNAc, SAα3/6Galβ4GlcNAc, and SAα3Galβ4GlcNAc, SAα3Galβ4(Fucα3)GlcNAc linked to an elongation structure according to
Formula E1:
AxHex(NAc)ₙ, wherein A is anomeric structure alfa or beta, X is linkage position 2, 3, or 6; and Hex is hexopyranosyl residue Gal, or Man, and n is integer being 0 or 1,
with the provisions that
when n is 1 then AxHexNAc is β6GalNAc,
when Hex is Man, then AxHex is β2Man, and
when Hex is Gal, then AxHex is β3Gal or β6Gal, with proviso that SAα3Galβ4GlcNAc is not linked to β3Gal.

85. The method according to claim 83, wherein said binding agent recognizes structure according to Formula T8Ebeta
[Mα]ₘGalβ1-3/4[Nα]nGlcNAcβxHex(NAc)ₚ
wherein x is linkage position 2, 3, or 6;
m, n and p are integers 0, or 1, independently; and
M and N are monosaccharide residues being
i) independently nothing (free hydroxyl groups at the positions) and/or
ii) SA which is Sialic acid linked to 3-position of Gal or/and 6-position of GlcNAc and/or
iii) Fuc (L-fucose) residue linked to 2-position of Gal and/or 3 or 4 position of GlcNAc,
when Gal is linked to the other position (4 or 3) of GlcNAc,
with the provision that m, n and p are 0 or 1, independently.
Hex is hexopyranosyl residue Gal, or Man,
with the provisions that when p is 1 then βxHexNAc is β6GalNAc,
when p is 0
then Hex is Man and βxHex is β2Man, or Hex is Gal and βxHex is β3Gal or β6Gal.

86. The method according to claim 83, wherein said binding agent recognizes type II Lactosmine based structures according to
[Mα]ₘGalβ1-4[Nα]ₙGlcNAcβxHex(NAc)ₚ Formula T10E
with the provisions that when p is 1 then βxHexNAc is β6GalNAc,
when p is 0, then Hex is Man and βxHex is β2Man, or Hex is Gal and βxHex is β6Gal.

87. The method according to claim 86, wherein said binding agent recognizes type II Lactosmine based structures according to
[Mα]ₘGalβ1-4[Nα]ₙGlcNAcβ2Man, Formula T10EMan:
wherein m and n are integers 0 or 1, independently; and
M and N are monosaccharide residues being
i) independently nothing (free hydroxyl groups at the positions) and/or
ii) SA which is Sialic acid linked to 3-position of Gal or/and 6-position of GlcNAc and/or
iii) Fuc (L-fucose) residue linked to 2-position of Gal and/or 3 or 4 position of GlcNAc,
when Gal is linked to the other position (4 or 3) of GlcNAc.

88. The method according to claim 86, wherein said binding agent recognizes type II Lactosmines according to
[Mα]ₘGalβ1-4[Nα]ₙGlcNAcβ6Gal(NAc)ₚ Formula T10EGal(NAc):
wherein m, n and p are integers 0 or 1, independently; and
M and N are monosaccharide residues being
i) independently nothing (free hydroxyl groups at the positions) and/or
ii) SA which is Sialic acid linked to 3-position of Gal or/and 6-position of GlcNAc and/or
iii) Fuc (L-fucose) residue linked to 2-position of Gal and/or 3 or 4 position of GlcNAc,
when Gal is linked to the other position (4 or 3) of GlcNAc.

89. The method according to claim 88, wherein the structure is O-glycan core II sialyl-Lewis x structure SAα3Galβ4(Fucα3)GlcNAcβ6(RGalβ3)GalNAc and it is recognized by antibody CHO131, and optionally wherein the antibody recognized over 50% of the hematopoietic cells.

90. The method according to claim 83, wherein said binding agent recognizes type I Lactosmine based structures according to
[Mα]ₘGalβ1-3[Nα]ₙGlcNAcβ3Gal Formula T9E

91. The method according to claim 83, wherein said binding agent recognizes type II Lactosamine based structures according to
Formula
[Mα]ₘGalβ1-4[Nα]ₙGlcNAcβ3Gal, with the proviso that structure is not SAα3Galβ4GlcNAcβ3Gal.

92. The method of claim 83, wherein the structure is SAα3Galβ4(Fucα3)GlcNAcβ3Gal to analyze the status of hematopoietic cells using antibody KM93 or CSLEX.

93. The method according to claim 83, wherein the detection is performed by a binder being a recombinant protein selected from the group consisting of monoclonal antibody, glycosidase, glycosyl transferring enzyme, plant lectin, animal lectin and a peptide mimetic thereof.

94. The method according to claim 83, wherein the binder is used for sorting or selecting human stem cells from biological materials or samples including cell materials comprising other cell types.

95. A cell population obtained by the method according to claim 94.

96. The method according to claim 83, wherein the glycan structure is present in a O-glycan subglycome comprising O-Glycans with O-glycan core structure, or the glycan structure is present in a glycolipid subglycome comprising glycolipids with glycolipid core structure and the glycans are releasable by glycosylceramidase or in a N-glycan subglycome comprising N-Glycans with N-glycan core structure and said N-Glycans being releasable from cells by N-glycosidase.

97. The method according to claim 83, wherein the presence or absence of cell surface glycomes of said cell preparation is detected.

98. The method according to claim 83, wherein said cell preparation is evaluated/detected with regard to a contaminating structure in a cell population of said cell preparation, time dependent changes or a change in the status of the cell population by glycosylation analysis using mass spectrometric analysis of glycans in said cell preparation

99. The method evaluate hematopoietic stem cells with regard to two terminal epitopes as defined by Formula I in claim 83, wherein the one of the following combinations of binder reagents are used, said reagents recognizing type I and type II acetyllactosamines and fucosylated variants or non-sialylated fucosylated variants thereof; or fucosylated type I and type II N-acetyllactosamine structures preferably comprising Fucα2-terminal and/or Fucα3/4-branch structure; or fucosylated type I and type II N-acetyllactosamine structures preferably comprising Fucα2-terminal.

100. A composition comprising glycan structure as defined in claim 83 derived from a stem cell and a binder that binds to said glycan structure.

101. The composition according to claim 100, wherein the composition is used in method for identifying a selective stem cell binder to said glycan structure, which comprises:
selecting a glycan structure exhibiting specific expression in/on stem cells and absence of expression in/on feeder cells and/or differentiated somatic cells; and
confirming the binding of the binder to the glycan structure in/on stem cells.

102. The composition according to the claim 100, wherein the composition is used in a kit for enrichment and detection of stem cells within a specimen; the kit comprising: at least one reagent comprising a binder to detect said glycan structure; and instructions for performing stem cell enrichment using the reagent, optionally including means for performing stem cell enrichment or
wherein the composition is for isolation of cellular components from stem cells comprising the novel target/marker structures.
